SEQUENCE LISTING (294 sequences)

SEQ ID NO: 1 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 2 - protein, 83 aa, synthetic construct
MHSKFSHAGRICGAKFKVGEPIYRCKECSFDDTCVLCVNCFNPKDHLGHHVYTTICTEFNNGECDCGDKTAWNHTLFCKAEEG

SEQ ID NO: 3 - protein, 200 aa, synthetic construct
MNGLSAQHERILPARHECVYTPCYSEENVYKLCEHIKTSKRCPLGDVYAVFISNERKMVPIWKQKSGRGERVVIWDYQVILLHDCHKEQTFIYDLDTTLPFPCPFDTYVKEAFKSDNYINPRFWRKLRVVPADVFLQNFASDRSHMKDASGGWRMPPPPYPCIETAESRMNLDDFISMNPSVGWGHVYTLEEFVQHFGKT

SEQ ID NO: 4 - protein, 200 aa, synthetic construct
MNGLSAQHERILPARHECVYTPCYSEENVYKLCEHIKTSKRCPLGDVYAVFISNERKMVPIWKQRSGRGERVVIWDYQVILLHDCHKEQTFIYDLDTTLPFPCPFDTYVKEAFKSDNYINPRFWRKLRVVRADVFLQNFASDRSHMKDASGGWRMPPPPYPCIETAESRMNLDDFISMNPSVGWGHVYTLEEFVQHFGKT

SEQ ID NO: 5 - protein, 200 aa, synthetic construct
MNGLSAQHERILPARHECVYTSCYSEENVWKLCEHIKTSKRCPLGDVYAVFISNERKMVPIWKQKSGRGERLVIWDYQVILLHDCHKEQTFIYDLDTTLPFPCPFDTYVKEAFKSDNYINPRFWRKLRVVPADVFLQNFASDRSHMKDASGGWRMPPPPYPCIETAESRMNLDDFISMNPSVGWGHVYTLEEFVQHFGKT

SEQ ID NO: 6 - protein, 200 aa, synthetic construct
MNGLSAQHERILPARHECVYTEFYSEENVWKLCEHIKTSKRCPLGDVYAVFISNERKMVPIWKQKSGRGERPVIWDYQVILLHDCHKEQTFIYDLDTTLPFPCPFDTYVKEAFKSDNYINPRFWRKLRVVPADVFLQNFASDRSHMKDASGGWRMPPPPYPCIETAESRMNLDDFISMNPSVGWGHVYTLEEFVQHFGKT

SEQ ID NO: 7 - protein, 200 aa, synthetic construct
MNGLSAQHERIAPARHECVYTPCYSEENVWKLCEHIKTSKRCPLGDVYAVFISNERKMVPIWKQRSGRGERPVIWDYQVILLHDCHKEQTFIYDLDTTLPFPCPFDTYVKEAFKSDNYINPRFWRKLRVVPADVFLQNFASDRSHMKDASGGWRMPPPPYPCIETAESRMNLDDFISMNPSVGWGHVYTLEEFVQHFGKT

SEQ ID NO: 8 - protein, 200 aa, synthetic construct
MNGLSAQHERILPARHECVYTECYSEENVWKLCEHIKTSKRCPLGDVYAVFISNERKMVPIWKQKSGRGERVVIWDYQVIMLHDCHKEQTFIYDLDTTLPFPCPFDTYVKEAFKSDNYINPRFWRKLRVVPADVFLQNFASDRSHMKDASGGWRMPPPPYPCIETAESRMNLDDFISMNPSVGWGHVYTLEEFVQHFGKT

SEQ ID NO: 9 - protein, 200 aa, synthetic construct
MNGLSAQHERILPARHECVYTSCYSEENVWKLCEHIKTSKRCPLGDVYAVFISNERKMVPIWKQKSGRGERPVIWDYQVILLHDCHKEQTFIYDLDTTLPFPCPFDTYVKEAFKSDNYINPRFWRKLRVVPADVFLQNFASDRSHMKDASGGWRMPPPPYPCIETAESRMNLDDFISMNPSVGWGHVYTLEEFVQHFGKT

SEQ ID NO: 10 - protein, 200 aa, synthetic construct
MNGLSAQHERILPARHECVYTSCYSEENVWKLCEHIKTSKRCPLGDVYAVFISNERKMVPIWKQRSGRGERVVIWDYQVILLHDCHKEQTFIYDLDTTLPFPCPFDTYVKEAFKSDNYINPRFWRKLRVVPADVFLQNFASDRSHMKDASGGWRMPPPPYPCIETAESRMNLDDFISMNPSVGWGHVYTLEEFVQHFGKT

SEQ ID NO: 11 - protein, 200 aa, synthetic construct
MNGLSAQHERILPARHECVYTECYSEENVWKLCEHIKTSKRCPLGDVYAVFISNERKMVPIWKQKSGRGERPVIWDYQVILLHDCHKEQTFIYDLDTTLPFPCPFDTYVKEAFKSDNYINPRFWRKLRVVPADVFLQNFASDRSHMKDASGGWRMPPPPYPCIETAESRMNLDDFISMNPSVGWGHVYTLEEFVQHFGKT

SEQ ID NO: 12 - protein, 200 aa, synthetic construct
MNGLSAQHERILPARHECVYTEFYSEENVWKLCEHIKTSKRCPLGDVYAVFISNERKMVPIWKQKSGRGERPVIWDYQVILLHDCHKEQTFIYDLDTTLPFPCPFDTYVKEAFKSDNYIRPRFWRKLRVVPADVFLQNFASDRSHMKDASGGWRMPPPPYPCIETAESRMNLDDFISMNPSVGWGHVYTLEEFVQHFGKT

SEQ ID NO: 13 - protein, 200 aa, synthetic construct
MNGLSAQHERILPARHECVYTPCYSEENVWKLCEHIKTQKRCPLGDVYAVFISNERKMVPIWKQKVGRGEEPVIWDYQVILLHDCHKEQTFIYDLDTTLPFPCPFDTYVKEAFKSDNYINPRFWRKLRVVPADVFLQNFASDRSHMKDASGGWRMPPPPYPCIETAESRMNLDDFISMNPSVGWGHVYTLEEFVQHFGKT

SEQ ID NO: 14 - protein, 200 aa, synthetic construct
MNGLSAQHERILPARHECVYTSCYSEENVYKLCEHIKTSKRCPLGDVYAVFISNERKMVPIWKQRSGRGERVVIWDYQVILLHDCHKEQSFIYDLDTTLPFPCPFDTYVKEAFKSDNYINPRFWRKLRVVPADVFLQNFASDRSHMKDASGGWRMPPPPYPCIETAESRMNLDDFISMNPSVGWGHVYTLEEFVQHFGKT

SEQ ID NO: 15 - protein, 200 aa, synthetic construct
MNGLSAQHERILPARHECVYTPCYSEENVYKLCEHIKTSKRCPLGDVYAVFISNERKMVPIWKQKSGRGERVVIWDYQVIMLHDCHKEQTFIYDLDTTLPFPCPFDTYVKEAFKSDNYINPRFWRKLRVVRADVFLQNFASDRSHMKDASGGWRMPPPPYPCIETAESRMNLDDFISMNPSVGWGHVYTLEEFVQHFGKT

SEQ ID NO: 16 - protein, 200 aa, synthetic construct
MNGLSAQHERILPARHECVYTPCYSEENVWKLCEHIKTSKRCPLGDVYAVFISNERKMVPIWKQRSGRGERVVIWDYQVILLHDCHKEQTFIYDLDTTLPFPCPFDTYVKEAFKSDNYINPRFWRKLRVVPADVFLQNFASDRSHMKDASGGWRMPPPPYPCIETAESRMNLDDFISMNPSVGWGHVYTLEEFVQHFGKT

SEQ ID NO: 17 - protein, 200 aa, synthetic construct
MNGLSAQHERILPARHECVYTPCYSEENVWKLCEHIKTSKRCPLGDVYAVFISNERKMVPIWKQKSGRGEEFVIWDYQVILLHDCHKEQTFIYDLDTTLPFPCPFDTYVKEAFKSDNYINPRFWRKLRVVPADVFLQNFASDRSHMKDASGGWRMPPPPYPCIETAESRMNLDDFISMNPSVGWGHVYTLEEFVQHFGKT

SEQ ID NO: 18 - protein, 200 aa, synthetic construct
MNGLSAQHERILPARHECVYTSCYSEENVWKLCEHIKTQKRCPLGDVYAVFISNERKMVPIWKQKVGRGEEPVIWDYQVILLHDCHKEQTFIYDLDTTLPFPCPFDTYVKEAFKSDNYIRPRFWRKLRVVPADVFLQNFASDRSHMKDASGGWRMPPPPYPCIETAESRMNLDDFISMNPSVGWGHVYTLEEFVQHFGKT

SEQ ID NO: 19 - protein, 200 aa, synthetic construct
MNGLSAQHERIAPARHECVYTPGYSEENVWKLCQHIKTSKRCPLGDVYAVFISNERKMVPIWKQRSGRGEEPLIWDYKVILLHDCHKEQTFIYDLDTTLPFPCPFDTYVKEAFKSDNYINPRFWRKLRVVPADVFLQNFASDRSHMKDSSGGWRMPPPPYPCIETAESRMNLDDFISMNPSVGWGHVYTLEEFVQHFGKT

SEQ ID NO: 20 - protein, 200 aa, synthetic construct
MNGLSAQHERIAPARHECVYTPGYSEENVWKLCQHIKTSKRCPLGDVYAVFISNERKMVPIWKQRSGRGEEPLIWDYKVILLHDCHKEQTFIYDLDTTLPFPCPFDTYVKEAFKSDNYINPRFWRKLRVVPADVFLQNFASDRSHMKDASGGWRMPPPPYPCIETAESRMNLDDFISMNPSVGWGHVYTLEEFVQHFGKT

SEQ ID NO: 21 - protein, 200 aa, synthetic construct
MNGLSAQHERILPARHECVYTSGYSEENVWKLCEHIKTSKRCPLGDVYAVFISNERKMVPIWKQKSGRGEEPVIWDYKVQLLHDCHKEQTFIYDLDTTLPFPCPFDTYVKEAFKSDNYINPRFWRKLRVVPADVFLQNFASDRSHMKDDLGGWRMPPPPYPCIETAESRMNLDDFISMNPSVGWGHVYTLEEFVQHFGKT

SEQ ID NO: 22 - protein, 200 aa, synthetic construct
MNGLSAQHERIAPARHECVYTPGYGEENVWKLCQHIKTSKRCPLGDVYAVFISNERKMVPIWKQRSGRGEEPLIWDYRVILLHDCHKEQTFIYDLDTTLPFPCPFDTYVKEAFRSDNYINPRFWRKLRVVPADVFLQNFASDRSHMKDESGGWRMPPPPYPCIETAESRMNLDDFISMNPSVGWGHVYTLEEFVQHFGKT

SEQ ID NO: 23 - protein, 200 aa, synthetic construct
MNGLSAQHERILPARHECVYTSGYSEENVWKLCEHIKTSKRCPLGDVYAVFISNERKMVPIWKQKSGRGEEPLIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 24 - protein, 200 aa, synthetic construct
MNGLSAQHERILPARHECVYTPCYSEENVWKLCEHIKTSKRCPLGDVYAVFISNERKMVPIWKQKVGRGEEPVIWDYQVILLHDCHKEQTFIYDLDTTLPFPCPFDTYVKEAFKSDNYIRPRFWRKLRVVPADVFLQNFASDRSHMKDASGGWRMPPPPYPCIETAESRMNLDDFISMNPSVGWGHVYTLEEFVQHFGKT

SEQ ID NO: 25 - protein, 200 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 26 - protein, 200 aa, synthetic construct
MNGLSAQHERILPARHECVYTPGYSEENVWKLCEHIKTSKRCPLGVVYAVFISNERKMVPIWKQRSGRGEEPLIWDYQVILLHDCHKEQTFIYDLDTTLPFPCPFDTYVKEAFRSDNYINPRFWRKLRVVPADVFLQNFASDRSHMKDERGGWRMPPPPYPCIETAESRMNLDDFISMNPSVGWGHVYTLEEFVQHFGKT

SEQ ID NO: 27 - protein, 200 aa, synthetic construct
MNGLSAQHERILPARHECVYTPCYSEENVWKLCQHIKTSKRCPLGDVYAVFISNERKMVPIWKQRSGRGEEPLIWDYQVILLHDCHKEQTFIYDLDTTLPFPCPFDTYVKEAFKSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 28 - protein, 200 aa, synthetic construct
MNGLSAQHERILPARHECVYTPQYSEENVWKLCEHIKTSKRFPLGDVYAVFISNERKMVPIWKQRSGRGEEPVIWDYKVILLHDCHKEQTFIYDLDTTLPFPCPFDTYVKEAFKSDNYINPRFWRKLRVVPADVFLQNFASDRSHMKDSSGGWRMPPPPYPCIETAESRMNLDDFISMNPSVGWGHVYTLEEFVQHFGKT

SEQ ID NO: 29 - protein, 200 aa, synthetic construct
MNGLSAQHERILPARHECVYTPQYSEENVWKLCEHIKTSKRCPLGDVYAVFISNERKMVPIWKQRSGRGEEPIIWDYKVFLLHDCHKEQTFIYDLDTTLPFPCPFDTYVKEAFKSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 30 - protein, 200 aa, synthetic construct
MNGLSAQHERILPARHECVYTPGYGEENVWKLCEHIKTSKRCPLGGVYAVFISNERKMVPIWKQRSGRGEEPLIWDYRVILLHDCHKEQTFIYDLDTTLPFPCPFDTYVKKAFRSDNYINPRFWRKLRVVPADVFLQNFASDRSHMKDARGGWRMPPPPYPCIETAESRMNLDDFISMNPSVGWGHVYTLEEFVQHFGKT

SEQ ID NO: 31 - protein, 200 aa, synthetic construct
MNGLSAQHERILPARHECVYTPGYGEENVWKLCEHIKTSKRCPLGDVYAVFISNERRMVPIWKQRSGRGEEPLIWDYRVFLLHDCHKEQTFIYDLRTTLPFPCPFDTYVKEAFKSDNYINPRFWRKLRVVPADVFLQNFASDRSHMKDARGGWRMPPPPYPCIETAESRMNLDDFISMNPSVGWGHVYTLEEFVQHFGKT

SEQ ID NO: 32 - protein, 200 aa, synthetic construct
MNGLSAQHERILPARHECVYTPGYSEENVWKLCQHIKTSKRCLLGDVYAVFISNERKMVPIWKQKSGRGEEPLIWDYRVILLHDCHKEQTFIYDLRTTLPFPCPFDTYVKEAFRSDNYINPAFWRKLRVVPADVFLQNFASDRSHMKDARGGWRMPPPPYPCIETAESRMNLDDFISMNPSVGWGHVYTLEEFVQHFGKT

SEQ ID NO: 33 - protein, 200 aa, synthetic construct
MNGLSAQHERILPARHECVYTPGYGEENVWKLCEHIKTSKRCPLGDVYAVFISNERKMVPIWKQRSGRGEEPLIWDYKVILLHDCHKEQTFIYDLDTTLPFPCPFDTYVKEAFKSDNYINPRFWRKLRVVPADVFLQNFASDRSHMKDSRGGWRMPPPPYPCIETAESRMNLDDFISMNPSVGWGHVYTLEELVQHFGKT

SEQ ID NO: 34 - protein, 200 aa, synthetic construct
MNGLSAQHERILPARHECVYTSGYGEENVWKLCQHIKTSKRCPLGDVYAVFISNERKMVPIWKQRSGRGEEPLIWDYKVILLHDCHKEQTFIYDLDTTLPFPCPFDTYVKEAFRSDNYINPRFWRKLRVVPADVFLQNFASDRSHMKDSSGGWRMPPPPYPCIETAESRMNLDDFISMNPSVGWGHVYTLEEFVQHFGKT

SEQ ID NO: 35 - protein, 200 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLDTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 36 - protein, 200 aa, synthetic construct
MNGLSAQHERIAPARHECVYTVGYSEENVWKLCEHIKTVKRCPLGDVYAVFISNERKMVPIWKQKSGRGEEWVWWDYKVILLHDAHKEQTFIYDLGTTLPFPCPFDTYVKEAFKSDNYIHPAFWRKLRVVPADVFLQNFASDRSHMKDSSGGWRMPPPPYPCIETAESRMNLDDFISMNPSVGWGHVYTLEEFVQHFGKT

SEQ ID NO: 37 - protein, 200 aa, synthetic construct
MNGLSAQHERIAPARHECVYTQGYSEENVWKLCEHIKTYKRCPLGDVYAVFISNERKMVPIWKQKSGRGEEYVVWDYKVILLHDCHKEQTFIYDLGTTLPFPCPFDTYVKEAFKSDNYINPAFWRKLRVVPADVFLQNFASDRSHMKDSSGGWRMPPPPYPCIETAESRMNLDDFISMNPSVGWGHVYTLEEFVQHFGKT

SEQ ID NO: 38 - protein, 200 aa, synthetic construct
MNGLSAQHERIAPARHECVYTPGYSEENVWKLCEHIKTSKRCPLGDVYAVFISNERKMVPIWKQKSGRGEEHVLWDYKVILLHDCHKEQTFIYDLGTTLPFPCPFDTYVKEAFKSDNYINPAFWRKLRVVPADVFLQNFASDRSHMKDRSGGWRMPPPPYPCIETAESRMNLDDFISMNPSVGWGHVYTLEEFVQHFGKT

SEQ ID NO: 39 - protein, 200 aa, synthetic construct
MNGLSAQHERIAPARHECVYTQGYSEENVWKLCEHIKTQKRCPLGDVYAVFISNERKMVPIWKQKSGRGEEYVLWDYKVILLHDGHKEQTFIYDLGTTLPFPCPFDTYVKEAFKSDNYINPAFWRKLRVVPADVFLQNFASDRSHMKLASGGWRMPPPPYPCIETAESRMNLDDFISMNPSVGWGHVYTLEEFVQHFGKT

SEQ ID NO: 40 - protein, 200 aa, synthetic construct
MNGLSAQHERIAPARHECVYTLGYSEENVWKLCEHIKTGKRCPLGDVYAVFISNERKMVPIWKQKSGRGEEHVLWDYKVILLHDCHKEQTFIYDLGTTLPFPCPFDTYVKEAFKSDNYINPAFWRKLRVVPADVFLQNFASDRSHMKLASGGWRMPPPPYPCIETAESRMNLDDFISMNPSVGWGHVYTLEEFVQHFGKT

SEQ ID NO: 41 - protein, 200 aa, synthetic construct
MNGLSAQHERIAPARHECVYTQGYSEENVWKLCEHIKTQKRCPLGDVYAVFISNERKMVPIWKQKSGRGEEYVLWDYKVILLHDGHKEQTFIYDLGTTLPFPCPFDTYVKEAFKSDNYINPAFWRKLRVVPADVFLQNFASDRSHMKDKSGGWRMPPPPYPCIETAESRMNLDDFISMNPSVGWGHVYTLEEFVQHFGKT

SEQ ID NO: 42 - protein, 200 aa, synthetic construct
MNGLSAQHERIAPARHECVYTVGYSEENVWKLCEHIKTRKRCPLGDVYAVFISNERKMVPIWKQKSGRGEEHVLWDYKVILLHDCHKEQTFIYDLGTTLPFPCPFDTYVKEAFKSDNYINPAFWRKLRVVPADVFLQNFASDRSHMKTASGGWRMPPPPYPCIETAESRMNLDDFISMNPSVGWGHVYTLEEFVQHFGKT

SEQ ID NO: 43 - protein, 200 aa, synthetic construct
MNGLSAQHERIAPARHECVYTPGYSEENVWKLCEHIKTNKRCPLGDVYAVFISNERKMVPIWKQKSGRGEEWVLWDYKVILLHDRHKEQTFIYDLGTTLPFPCPFDTYVKEAFKSDNYINPAFWRKLRVVPADVFLQNFASDRSHMKDLSGGWRMPPPPYPCIETAESRMNLDDFISMNPSVGWGHVYTLEEFVQHFGKT

SEQ ID NO: 44 - protein, 200 aa, synthetic construct
MNGLSAQHERIAPARHECVYTEGYSEENVWKLCEHIKTRKRCPLGDVYAVFISNERKMVPIWKQKSGRGEEHVVWDYKVILLHDFHKEQTFIYDLGTTLPFPCPFDTYVKEAFKSDNYINPAFWRKLRVVPADVFLQNFASDRSHMKDRSGGWRMPPPPYPCIETAESRMNLDDFISMNPSVGWGHVYTLEEFVQHFGKT

SEQ ID NO: 45 - protein, 200 aa, synthetic construct
MNGLSAQHERIAPARHECVYTSGYSEENVWKLCEHIKTKKRCPLGDVYAVFISNERKMVPIWKQKSGRGEEHVLWDYKVILLHDCHKEQTFIYDLGTTLPFPCPFDTYVKEAFKSDNYINPAFWRKLRVVPADVFLQNFASDRSHMKDSSGGWRMPPPPYPCIETAESRMNLDDFISMNPSVGWGHVYTLEEFVQHFGKT

SEQ ID NO: 46 - protein, 200 aa, synthetic construct
MNGLSAQHERIAPARHECVYTQGYSEENVWKLCEHIKTYKRCPLGDVYAVFISNERKMVPIWKQKSGRGEEYVVWDYKVILLHDCHKEQTFIYDLGTTLPFPCPFDTYVKEAFKSDNYINPAFWRKLRVVPADVFLQNFASDRSHMKLASGGWRMPPPPYPCIETAESRMNLDDFISMNPSVGWGHVYTLEEFVQHFGKT

SEQ ID NO: 47 - protein, 200 aa, synthetic construct
MNGLSAQHERIAPARHECVYTSGYSEENVWKLCEHIKTVKRCPLGDVYAVFISNERKMVPIWKQKSGRGEEWVLWDYKVILLHDCHKEQTFIYDLGTTLPFPCPFDTYVKEAFKSDNYINPAFWRKLRVVPADVFLQNFASDRSHMKLASGGWRMPPPPYPCIETAESRMNLDDFISMNPSVGWGHVYTLEEFVQHFGKT

SEQ ID NO: 48 - protein, 200 aa, synthetic construct
MNGLSAQHERIAPARHECVYTTGYSEENVWKLCEHIKTMKRCPLGDVYAVFISNERKMVPIWKQKSGRGEEHVIWDYKVILLHDCHKEQTFIYDLGTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 49 - protein, 200 aa, synthetic construct
MNGLSAQHERIAPARHECVYTAGYSEENVWKLCEHIKTFKRCPLGDVYAVFISNERKMVPIWKQKSGRGEEHVIWDYKVILLHDIHKEQTFIYDLGTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 50 - protein, 200 aa, synthetic construct
MNGLSAQHERIAPARHECVYTEGYSEENVWKLCEHIKTSKRCPLGDVYAVFISNERKMVPIWKQKSGRGEEHVLWDYKVILLHDCHKEQTFIYDLGTTLPFPCPFDTYVKEAFKSDNYINPAFWRKLRVVPADVFLQNFASDRSHMKDRSGGWRMPPPPYPCIETAESRMNLDDFISMNPSVGWGHVYTLEEFVQHFGKT

SEQ ID NO: 51 - protein, 200 aa, synthetic construct
MNGLSAQHERIAPARHECVYTSGYSEENVWKLCEHIKTKKRCPLGDVYAVFISNERKMVPIWKQKSGRGEEHVLWDYKVILLHDCHKEQTFIYDLGTTLPFPCPFDTYVKEAFKSDNYINPAFWRKLRVVPADVFLQNFASDRSHMKDPSGGWRMPPPPYPCIETAESRMNLDDFISMNPSVGWGHVYTLEEFVQHFGKT

SEQ ID NO: 52 - protein, 200 aa, synthetic construct
MNGLSAQHERIAPARHECVYTPGYSEENVWKLCEHIKTSKRCPLGDVYAVFISNERKMVPIWKQKSGRGEEHVLWDYKVILLHDCHKEQTFIYDLGTTLPFPCPFDTYVKEAFKSDNYINPAFWRKLRVVPADVFLQNFASDRSHMKLASGGWRMPPPPYPCIETAESRMNLDDFISMNPSVGWGHVYTLEEFVQHFGKT

SEQ ID NO: 53 - protein, 200 aa, synthetic construct
MNGLSAQHERIAPARHECVYTSGYSEENVWKLCEHIKTKKRCPLGDVYAVFISNERKMVPIWKQKSGRGEEHVLWDYKVILLHDCHKEQTFIYDLGTTLPFPCPFDTYVKEAFKSDNYINPAFWRKLRVVPADVFLQNFASDRSHMKLASGGWRMPPPPYPCIETAESRMNLDDFISMNPSVGWGHVYTLEEFVQHFGKT

SEQ ID NO: 54 - protein, 200 aa, synthetic construct
MNGLSAQHERIAPARHECVYTEGYSEENVWKLCEHIKTRKRCPLGDVYAVFISNERKMVPIWKQKSGRGEEWVLWDYKVILLHDCHKEQTFIYDLGTTLPFPCPFDTYVKEAFKSDNYINPAFWRKLRVVPADVFLQNFASDRSHMKDTSGGWRMPPPPYPCIETAESRMNLDDFISMNPSVGWGHVYTLEEFVQHFGKT

SEQ ID NO: 55 - protein, 200 aa, synthetic construct
MNGLSAQHERIAPARHECVYTSGYSEENVWKLCEHIKTKKRCPLGDVYAVFISNERKMVPIWKQKSGRGEEHVLWDYKVILLHDCHKEQTFIYDLGTTLPFPCPFDTYVKEAFKSDNYINPAFWRKLRVVPADVFLQNFASDRSHMKDNSGGWRMPPPPYPCIETAESRMNLDDFISMNPSVGWGHVYTLEEFVQHFGKT

SEQ ID NO: 56 - protein, 200 aa, synthetic construct
MNGLSAQHERIAPARHECVYTQGYSEENVWKLCEHIKTVKRCPLGDVYAVFISNERKMVPIWKQKSGRGEEHVLWDYKVILLHDCHKEQTFIYDLGTTLPFPCPFDTYVKEAFKSDNYINPAFWRKLRVVPADVFLQNFASDRSHMKDRSGGWRMPPPPYPCIETAESRMNLDDFISMNPSVGWGHVYTLEEFVQHFGKT

SEQ ID NO: 57 - protein, 200 aa, synthetic construct
MNGLSAQHERIAPARHECVYTQGYSEENVWKLCEHIKTQKRCPLGDVYAVFISNERKMVPIWKQKSGRGEEYVLWDYKVILLHDGHKEQTFIYDLGTTLPFPCPFDTYVKEAFKSDNYINPAFWRKLRVVPADVFLQNFASDRSHMKTASGGWRMPPPPYPCIETAESRMNLDDFISMNPSVGWGHVYTLEEFVQHFGKT

SEQ ID NO: 58 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDKSGGWRMPPPPYPCIETAESRMNLDDFISMNPSVGWGHVYTLEEFVQHFGKT

SEQ ID NO: 59 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDMSGGWRMPPPPYPCIETAESRMNLDDFISMNPSVGWGHVYTLEEFVQHFGKT

SEQ ID NO: 60 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDTSGGWRMPPPPYPCIETAESRMNLDDFISMNPSVGWGHVYTLEEFVQHFGKT

SEQ ID NO: 61 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDPSGGWRMPPPPYPCIETAESRMNLDDFISMNPSVGWGHVYTLEEFVQHFGKT

SEQ ID NO: 62 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKTASGGWRMPPPPYPCIETAESRMNLDDFISMNPSVGWGHVYTLEEFVQHFGKT

SEQ ID NO: 63 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDDSGGWRMPPPPYPCIETAESRMNLDDFISMNPSVGWGHVYTLEEFVQHFGKT

SEQ ID NO: 64 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKSASGGWRMPPPPYPCIETAESRMNLDDFISMNPSVGWGHVYTLEEFVQHFGKT

SEQ ID NO: 65 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDISGGWRMPPPPYPCIETAESRMNLDDFISMNPSVGWGHVYTLEEFVQHFGKT

SEQ ID NO: 66 - protein, 200 aa, synthetic construct
MNGLSAQHERIAPARHECVYTDCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 67 - protein, 200 aa, synthetic construct
MNGLSAQHERIAPARHECVYTSCYSEENVWKLCEHIKTGKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 68 - protein, 200 aa, synthetic construct
MNGLSAQHERIAPARHECVYTDSYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 69 - protein, 200 aa, synthetic construct
MNGLSAQHERIAPARHECVYTSCYSEENVWKLCEHIKTRKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 70 - protein, 200 aa, synthetic construct
MNGLSAQHERIAPARHECVYTSSYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 71 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDSHKEQTFIYDLDTTLPFPCPFDTYVKEAFKSDNYINPAFWRKLRVVPADVFLQNFASDRSHMKDRSGGWRMPPPPYPCIETAESRMNLDDFISMNPSVGWGHVYTLEEFVQHFGKT

SEQ ID NO: 72 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKEASGGWRMPPPPYPCIETAESRMNLDDFISMNPSVGWGHVYTLEEFVQHFGKT

SEQ ID NO: 73 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKYASGGWRMPPPPYPCIETAESRMNLDDFISMNPSVGWGHVYTLEEFVQHFGKT

SEQ ID NO: 74 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDLSGGWRMPPPPYPCIETAESRMNLDDFISMNPSVGWGHVYTLEEFVQHFGKT

SEQ ID NO: 75 - protein, 200 aa, synthetic construct
MNGLSAQHERIAPARHECVYTWCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 76 - protein, 200 aa, synthetic construct
MNGLSAQHERIAPARHECVYTSCYSEENVWKLCEHIKTSKRCPLGDVYAVFISNERKMVPIWKQKSGRGEEMVIWDYQVILLHDCHKEQTFIYDLDTTLPFPCPFDTYVKEAFKSDNYINPAFWRKLRVVPADVFLQNFASDRSHMKDTSGGWRMPPPPYPCIETAESRMNLDDFISMNPSVGWGHVYTLEEFVQHFGKT

SEQ ID NO: 77 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKNASGGWRMPPPPYPCIETAESRMNLDDFISMNPSVGWGHVYTLEEFVQHFGKT

SEQ ID NO: 78 - protein, 200 aa, synthetic construct
MNGLSAQHERIAPARHECVYTSGYSEENVWKLCEHIKTGKRCPLGDVYAVFISNERKMVPIWKQKSGRGEELVIWDYQVILLHDCHKEQTFIYDLDTTLPFPCPFDTYVKEAFKSDNYINPAFWRKLRVVPADVFLQNFASDRSHMKDLSGGWRMPPPPYPCIETAESRMNLDDFISMNPSVGWGHVYTLEEFVQHFGKT

SEQ ID NO: 79 - protein, 200 aa, synthetic construct
MNGLSAQHERIAPARHECVYTSGYSEENVWKLCEHIKTRKRCPLGDVYAVFISNERKMVPIWKQKSGRGEEMVIWDYQVILLHDCHKEQTFIYDLDTTLPFPCPFDTYVKEAFKSDNYINPAFWRKLRVVPADVFLQNFASDRSHMKDRSGGWRMPPPPYPCIETAESRMNLDDFISMNPSVGWGHVYTLEEFVQHFGKT

SEQ ID NO: 80 - protein, 200 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYIRPRFWRKLRVVPADVFLQNFASDRSHMKDSRGGWRMPPPPYPCIETAESRMNLDDFISMNPSVGWGHVYTLEEFVQHFGKT

SEQ ID NO: 81 - protein, 200 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDARGGWRMPPPPYPCIETAESRMNLDDFISMNPSVGWGHVYTLEEFVQHFGKT

SEQ ID NO: 82 - protein, 200 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSSGGWRMPPPPYPCIETAESRMNLDDFISMNPSVGWGHVYTLEEFVQHFGKT

SEQ ID NO: 83 - protein, 200 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDASGGWRMPPPPYPCIETAESRMNLDDFISMNPSVGWGHVYTLEEFVQHFGKT

SEQ ID NO: 84 - protein, 200 aa, synthetic construct
MNGLSAQHERILPARHECVYTPGYSEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 85 - protein, 200 aa, synthetic construct
MNGLSAQHERILPARHECVYTPCYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 86 - protein, 200 aa, synthetic construct
MNGLSAQHERILPARHECVYTSGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 87 - protein, 200 aa, synthetic construct
MNGLSAQHERILPARHECVYTPGYGEENVWKLCEHIKTSKRCPLGDVYAVFISNERKMVPIWKQKSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 88 - protein, 200 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYIRPRFWRKLRVVPADVFLQNFASDRSHMKDARGGWRMPPPPYPCIETAESRMNLDDFISMNPSVGWGHVYTLEEFVQHFGKT

SEQ ID NO: 89 - protein, 200 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYIRPRFWRKLRVVPADVFLQNFASDRSHMKDSRGGWLMPPPPYPCIETAESRMNLDDFISMNPSVGWGHVYTLEEFVQHFGKT

SEQ ID NO: 90 - protein, 200 aa, synthetic construct
MNGLSAQHERILPARHECVYTPGYGEENVWKLCEHIKTSKRCPLGDVYAVFISNERKMVPIWKQRSGRGERPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 91 - protein, 200 aa, synthetic construct
MNGLSAQHERILPARHECVYTEGYGEENVWKLCEHIKTSKRCPLGDVYAVFISNERKMVPIWKQKSGRGEEPVIWDYRVILLHDTHKEQTFIYDLRTTLSFPCPFDTYVKEAFKSDNYIRPRFWRKLRVVPADVFLQNFASDRSHMKDASGGWQMPPPPYPCIETAESRMNLDDFISMNPSVGWGHVYTLEEFVQHFGKT

SEQ ID NO: 92 - protein, 200 aa, synthetic construct
MNGLSAQHERILPARHECVYTEGYGEENVWKLCEHIKTSKRCPLGDVYAVFISNERKMVPIWKQKSGRGERPLIWDYRVILLHDCHKEQTFIYDLRTTLPFPCPFDTYVKEAFKSDNYINPRFWRKLRVVPADVFLQNFASDRSHMKDSRGGWPMPPPPYPCIETAESRMNLDDFISMNPSVGWGHVYTLEEFVQHFGKT

SEQ ID NO: 93 - protein, 200 aa, synthetic construct
MNGLSAQHERILPARHECVYTEGYGEENVWKLCEHIKTSKRCPLGDVYAVFISNERKMVPIWKQKSGRGEEPVIWDYRVILLHDTHKEQTFIYDLRTTLSFPCPFDTYVKEAFKSDNYIRPRFWRKLRVVPADVFLQNFASDRSHMKGASGGWQMPPPPYPCIETAESRMNLDDFISMNPSVGWGHVYTLEEFVQHFGKT

SEQ ID NO: 94 - protein, 200 aa, synthetic construct
MNGLSAQHERILPARHECVYTEGYGEENVWKLCEHIKTSKRCPLGDVYAVFISNERKMVPIWKQKSGRGEEPVIWDYRVILLHDPHKEQTFIYDLRTTLSFPCPFDTYVKEAFKSDNYIRPRFWRKLRVVPADVFLQNFASDRSHMKDASGGWQMPPPPYPCIETAESRMNLDDFISMNPSVGWGHVYTLEEFVQHFGKT

SEQ ID NO: 95 - protein, 200 aa, synthetic construct
MNGLSAQHERILPARHECVYTECYSEENVWKLCEHIKTSKRCPLGDVYAVFISNERKMVPIWKQKSGRGERPVIWDYKVILLHDTHKEQTFIHDLRTTLPFPCPFDTYVKEAFRSDNYINPRFWRKLRVVPADVFLQNFASDRSHMKDSRGGWLMPPPPYPCIETAESRMNLDDFISMNPSVGWGHVYTLEEFVQHFGKT

SEQ ID NO: 96 - protein, 200 aa, synthetic construct
MNGLSAQHERILPARHECVYTPGYSEENVWILCEHIKTSKRCPLGDVYAVFISNERKMVPIWKQKSGRGERPVIWDYRVILLHDTHKEQTFIYDLKTTLPFSCPFDTYVKEAFRSDNYIRPRFWRKLRVVPADVFLQNFASDRSHMKDSSGGWQMPPPPYPCIETAESRMNLDDFISMNPSVGWGHVYTLEEFVQHFGKT

SEQ ID NO: 97 - protein, 200 aa, synthetic construct
MNGLSAQHERILPARHECVYTPCYGEENVWKLCEHIKTSKRCPLGDVYAVFISNERKMVPIWKQKSGRGERPLIWDYRVILLHDTHKEQTFIYDLRTTLPFPCPFDTYVKEAFKSDNYINPRFWRKLRVVPADVFLQNFASDRSHMKDSRGGWLMPPPPYPCIETAESRMNLDDFISMNPSVGWGHVYTLEEFVQHFGKT

SEQ ID NO: 98 - protein, 200 aa, synthetic construct
MNGLSAQHERILPARHECVYTPGYSEENVWKLCEHIKTSKRCPLGDVYAVFISNERKMVPIWKQKSGRGEEPLIWDYRVILLHDCHKEQTFIYDLRTTLPFPCPFDTYVKEAFKSDNYIRPRFWRKLRVVPADVFLQNFASDRSHMKDARGGWRMPPPPYPCIETAESRMNLDDFISMNPSVGWGHVYTLEEFVQHFGKT

SEQ ID NO: 99 - protein, 200 aa, synthetic construct
MNGLSAQHERILPARHECVYTEGYGEENVWKLCEHIETSKRCPLGDVYAVFISNERKMVPIWKQKSGRGEEPVIWDYRVILLHDTHKEQTFIYDLRTTLSFPCPFDTYVKEAFKSDNYIRPRFWRKLRVVPADVFLQNFASDRSHMKDASGGWQMPPPPYPCIETAESRMNLDDFISMNPSVGWGHVYTLEEFVQHFGKT

SEQ ID NO: 100 - protein, 200 aa, synthetic construct
MNGLSAQHERILPARHECVYTPCYSEENVWKLCEHIKTSKRCPLGDVYAVFISNERKMVPIWKQKSGRGERPVIWDYRVILLHDCHKEQTFIHDLDTTLPFPCPFDTYVEEAFRSDNYIRPRFWRKLRVVPADVFLQNFASDRSHMKDARGGWPMPPPPYPCIETAESRMNLDDFISMNPSVGWGHVYTLEEFVQHFGKT

SEQ ID NO: 101 - protein, 200 aa, synthetic construct
MNGLSAQHERILPARHECVYTEGYGEENVWKLCEHIKTSKRCPLGDVYAVFISNERKMVPIWKQKSGRGGRPLIWDYRVILLHDCHKEQTFIYDLRTTLPFPCPFDTYVKEAFKSDNYINPRFWRKLRVVPADVFLQNFASDRSHMKDSRGGWPMPPPPYPCIETAESRMNLDDFISMNPSVGWGHVYTLEEFVQHFGKT

SEQ ID NO: 102 - protein, 200 aa, synthetic construct
MNGLSAQHERILPARHECVYTEGYGEENVWKLCEHIKTSKRCPLGDVYAVFISNERKMVPIWKQKSGRGEEPLIWDYKVILLHDCHKEQTFIYDLDTTLPFPCPFDTYVKEAFRSDNYIRPRFWRKLRVVPADVFLQNFASDRSHMKDASGGWPMPPPPYPCIETAESRMNLDDFISMNPSVGWGHVYTLEEFVQHFGKT

SEQ ID NO: 103 - protein, 200 aa, synthetic construct
MNGLSAQHERILPARHECVYTPGYGEENVWKLCEHIKTSKRCPLGDVYAVFISNERKMVPIWKQKSGRGEEPLIWDYRVILLHDCHKEQTFIYDLRTTLPFPCPFDTYVKEAFKSDNYIRPRFWRKLRVVPADVFLQNFASDRSHMKDARGGWLMPPPPYPCIETAESRMNLDDFISMNPSVGWGHVYTLEEFVQHFGKT

SEQ ID NO: 104 - protein, 200 aa, synthetic construct
MNGLSAQHERILPARHECVYTPGYGEENVWKLCEHIKTSKRCPLGDVYAVFISNERKMVPIWKQKSGRGERPVIWDYRVILLHDTHKEQTFIYDLRTTLPFPCPFDTYVKEAFRSDNYIRPRFWRKLRVVPADVFLQNFASDRSHMKDSRGGWRMPPPPYPCIETAESRMNLDDFISMNPSVGWGHVYTLEEFVQHFGKT

SEQ ID NO: 105 - protein, 200 aa, synthetic construct
MNGLSAQHERILPARHECVYTPGYGEENVWKLCEHIKTSKRCPLGDVYAVFISNERKMVPIWKQKSGRGEEPLIWDYRVILLHDCHKEQTFIYDLRTTLPFPCPFDTYVKEAFRSDNYIRPRFWRKLRVVPADVFLQNFASDRSHMKDSRGGWRMPPPPYPCIETAESRMNLDDFISMNPSVGWGHVYTLEEFVQHFGKT

SEQ ID NO: 106 - protein, 200 aa, synthetic construct
MNGLSAQHERILPARHECVYTPGYGEENVWKLCEHIKTSKRCPLGDVYAVFISNERKMVPIWKQKSGRGERPLIWDYRVILLHDCHKEQTFIYDLRTTLPFPCPFDTYVKEAFRSDNYIRPRFWRKLRVVPADVFLQNFASDRSHMKDSRGGWRMPPPPYPCIETAESRMNLDDFISMNPSVGWGHVYTLEEFVQHFGKT

SEQ ID NO: 107 - protein, 200 aa, synthetic construct
MNGLSAQHERILPARHECVYTPGYGEENVWKLCEHIKTSKRCPLGDVYAVFISNERKMVPIWKQKSGRGERPLIWDYRVILLHDTHKEQTFIYDLRTTLPFPCPFDTYVKEAFRSDNYIRPRFWRKLRVVPADVFLQNFASDRSHMKDSRGGWRMPPPPYPCIETAESRMNLDDFISMNPSVGWGHVYTLEEFVQHFGKT

SEQ ID NO: 108 - protein, 200 aa, synthetic construct
MNGLSAQHERILPARHECVYTPGYGEENVWKLCEHIKTSKRCPLGDVYAVFISNERKMVPIWKQKSGRGEEPLIWDYRVILLHDCHKEQTFIYDLRTTLPFPCPFDTYVKEAFRSDNYINPRFWRKLRVVPADVFLQNFASDRSHMKDARGGWRMPPPPYPCIETAESRMNLDDFISMNPSVGWGHVYTLEEFVQHFGKT

SEQ ID NO: 109 - protein, 200 aa, synthetic construct
MNGLSAQHERILPARHECVYTPGYGEENVWKLCEHIKTSKRCPLGDVYAVFISNERKMVPIWKQKSGRGEEPLIWDYRVILLHDCHKEQTFIYDLRTTLPFPCPFDTYVKEAFRSDNYIRPRFWRKLRVVPADVFLQNFASDRSHMKDARGGWRMPPPPYPCIETAESRMNLDDFISMNPSVGWGHVYTLEEFVQHFGKT

SEQ ID NO: 110 - protein, 200 aa, synthetic construct
MNGLSAQHERILPARHECVYTPGYGEENVWKLCEHIKTSKRCPLGDVYAVFISNERKMVPIWKQKSGRGERPLIWDYRVILLHDCHKEQTFIYDLRTTLPFPCPFDTYVKEAFRSDNYIRPRFWRKLRVVPADVFLQNFASDRSHMKDARGGWRMPPPPYPCIETAESRMNLDDFISMNPSVGWGHVYTLEEFVQHFGKT

SEQ ID NO: 111 - protein, 200 aa, synthetic construct
MNGLSAQHERILPARHECVYTPGYGEENVWKLCEHIKTSKRCPLGDVYAVFISNERKMVPIWKQKSGRGEEPLIWDYRVILLHDCHKEQTFIYDLRTTLPFPCPFDTYVKEAFRSDNYINPRFWRKLRVVPADVFLQNFASDRSHMKDSRGGWLMPPPPYPCIETAESRMNLDDFISMNPSVGWGHVYTLEEFVQHFGKT

SEQ ID NO: 112 - protein, 200 aa, synthetic construct
MNGLSAQHERILPARHECVYTPGYGEENVWKLCEHIKTSKRCPLGDVYAVFISNERKMVPIWKQKSGRGERPLIWDYRVILLHDCHKEQTFIYDLRTTLPFPCPFDTYVKEAFRSDNYIRPRFWRKLRVVPADVFLQNFASDRSHMKDARGGWLMPPPPYPCIETAESRMNLDDFISMNPSVGWGHVYTLEEFVQHFGKT

SEQ ID NO: 113 - protein, 415 aa, synthetic construct
MNGLSAQHERIAPARHECVYTSCYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKTEAAAKEAAAKEAAAKMNGLSAQHERIAPARHECVYTSCYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 114 - protein, 411 aa, synthetic construct
MNGLSAQHERIAPARHECVYTSCYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKTEEEKRKREEEEMNGLSAQHERIAPARHECVYTSCYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 115 - protein, 415 aa, synthetic construct
MNGLSAQHERIAPARHECVYTECYSEENVWKLCEHIKTQKRCPLGDVYAVFISNERKMVPIWKQKSGRGEEPVIWDYKVILLHDTHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKTEAAAKEAAAKEAAAKMNGLSAQHERIAPARHECVYTECYSEENVWKLCEHIKTQKRCPLGDVYAVFISNERKMVPIWKQKSGRGEEPVIWDYKVILLHDTHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKT

SEQ ID NO: 116 - protein, 411 aa, synthetic construct
MNGLSAQHERIAPARHECVYTECYSEENVWKLCEHIKTQKRCPLGDVYAVFISNERKMVPIWKQKSGRGEEPVIWDYKVILLHDTHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKTEEEKRKREEEEMNGLSAQHERIAPARHECVYTECYSEENVWKLCEHIKTQKRCPLGDVYAVFISNERKMVPIWKQKSGRGEEPVIWDYKVILLHDTHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKT

SEQ ID NO: 117 - protein, 415 aa, synthetic construct
MNGLSAQHERIAPARHECVYTECYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKTEAAAKEAAAKEAAAKMNGLSAQHERIAPARHECVYTECYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKT

SEQ ID NO: 118 - protein, 411 aa, synthetic construct
MNGLSAQHERIAPARHECVYTECYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKTEEEKRKREEEEMNGLSAQHERIAPARHECVYTECYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKT

SEQ ID NO: 119 - protein, 415 aa, synthetic construct
MNGLSAQHERIAPARHECVYTECYSEENVWKLCEHIKTSKRCPLGDVYAVFISNERKMVPIWKQKSGRGEEPVIWDYKVILLHDTHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKTEAAAKEAAAKEAAAKMNGLSAQHERIAPARHECVYTECYSEENVWKLCEHIKTSKRCPLGDVYAVFISNERKMVPIWKQKSGRGEEPVIWDYKVILLHDTHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKT

SEQ ID NO: 120 - protein, 411 aa, synthetic construct
MNGLSAQHERIAPARHECVYTECYSEENVWKLCEHIKTSKRCPLGDVYAVFISNERKMVPIWKQKSGRGEEPVIWDYKVILLHDTHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKTEEEKRKREEEEMNGLSAQHERIAPARHECVYTECYSEENVWKLCEHIKTSKRCPLGDVYAVFISNERKMVPIWKQKSGRGEEPVIWDYKVILLHDTHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKT

SEQ ID NO: 121 - protein, 415 aa, synthetic construct
MNGLSAQHERIAPARHECVYTECYSEENVWKLCEHIKTQKRCPLGDVYAVFISNERKMVPIWKQKSGRGEEPVIWDYKVILLHDCHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKTEAAAKEAAAKEAAAKMNGLSAQHERIAPARHECVYTECYSEENVWKLCEHIKTQKRCPLGDVYAVFISNERKMVPIWKQKSGRGEEPVIWDYKVILLHDCHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKT

SEQ ID NO: 122 - protein, 411 aa, synthetic construct
MNGLSAQHERIAPARHECVYTECYSEENVWKLCEHIKTQKRCPLGDVYAVFISNERKMVPIWKQKSGRGEEPVIWDYKVILLHDCHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKTEEEKRKREEEEMNGLSAQHERIAPARHECVYTECYSEENVWKLCEHIKTQKRCPLGDVYAVFISNERKMVPIWKQKSGRGEEPVIWDYKVILLHDCHKEQTFIYDLDTTLPFPCPFDTYVKEAFKSDNYIRPAFWRKLRVVPADVFLQNFASDRSHMKDASGGWRMPPPPYPCIETAESRMNLDDFISMNPSVGWGHVYTLEEFVQHFGKT

SEQ ID NO: 123 - protein, 415 aa, synthetic construct
MNGLSAQHERIAPARHECVYTECYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYIRPAFWRKLRVVPADVFLQNFASDRSHMKDVSGGWRMPPPPYPCIETAESRMNLDDFISMNPSVGWGHVYTLEEFVQHFGKTEAAAKEAAAKEAAAKMNGLSAQHERIAPARHECVYTECYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYIRPAFWRKLRVVPADVFLQNFASDRSHMKDVSGGWRMPPPPYPCIETAESRMNLDDFISMNPSVGWGHVYTLEEFVQHFGKT

SEQ ID NO: 124 - protein, 411 aa, synthetic construct
MNGLSAQHERIAPARHECVYTECYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYIRPAFWRKLRVVPADVFLQNFASDRSHMKDVSGGWRMPPPPYPCIETAESRMNLDDFISMNPSVGWGHVYTLEEFVQHFGKTEEEKRKREEEEMNGLSAQHERIAPARHECVYTECYSEENVWKLCEHIKTSKRCPLGDVYAVFISNERKMVPIWKQKSGRGEEPVIWDYKVILLHDCHKEQTFIYDLDTTLPFPCPFDTYVKEAFKSDNYIRPAFWRKLRVVPADVFLQNFASDRSHMKDVSGGWRMPPPPYPCIETAESRMNLDDFISMNPSVGWGHVYTLEEFVQHFGKT

SEQ ID NO: 125 - protein, 413 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKTGGGSGGGSGGGSGMNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 126 - protein, 412 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKTGSAGSAAGSGEFMNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 127 - protein, 436 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKTGSAGSAAGSGEFGSAGSAAGSGEFGSAGSAAGSGEFMNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 128 - protein, 407 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKTQGLQNEEMNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 129 - protein, 410 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKTDPGGGPSSRLMNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 130 - protein, 429 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKTGNDGLCQKLSVPCMSSKPQKPWEAKDAWEMNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 131 - protein, 412 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKTFSFGFSFGFSFGMNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 132 - protein, 424 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKTFSFGFSFGFSFGFSFGFSFGFSFGMNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 133 - protein, 415 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKTEAAAKEAAAKEAAAKMNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 134 - protein, 411 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKTEEEKRKREEEEMNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 135 - protein, 407 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKTLPSPDVHMNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 136 - protein, 416 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKTAKLKQKTEQLQDRIAGMNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 137 - protein, 416 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKTWRIRPRPPRLPRPRPRMNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 138 - protein, 420 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKTAPAPAPAPAPAPAPAPAPAPMNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 139 - protein, 422 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKTAEAAAKEAAAKEAAAKEAAAKAMNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 140 - protein, 427 aa, synthetic construct
MNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKTAEAAAKEAAAKEAAAKEAAAKEAAAKAMNGLSAQHERILPARHECVYTPGYGEENVWKLCEHIKTSKRCPLGDVYAVFISNERKMVPIWKQRSGRGEEPLIWDYRVILLHDCHKEQTFIYDLRTTLPFPCPFDTYVKEAFRSDNYINPRFWRKLRVVPADVFLQNFASDRSHMKDSRGGWRMPPPPYPCIETAESRMNLDDFISMNPSVGWGHVYTLEEFVQHFGKT

SEQ ID NO: 141 - protein, 414 aa, synthetic construct
MNGLSAQHERIAPARHECVYTVGYSEENVWKLCEHIKTVKRCPLGDVYAVFISNERKMVPIWKQKSGRGEEWVWWDYKVILLHDAHKEQTFIYDLGTTLPFPCPFDTYVKEAFKSDNYIHPAFWRKLRVVPADVFLQNFASDRSHMKDSSGGWRMPPPPYPCIETAESRMNLDDFISMNPSVGWGHVYTLEEFVQHFGKTEAAAKEAAAKEAAAKNGLSAQHERIAPARHECVYTVGYSEENVWKLCEHIKTVKRCPLGDVYAVFISNERKMVPIWKQKSGRGEEWVWWDYKVILLHDAHKEQTFIYDLGTTLPFPCPFDTYVKEAFKSDNYIHPAFWRKLRVVPADVFLQNFASDRSHMKDSSGGWRMPPPPYPCIETAESRMNLDDFISMNPSVGWGHVYTLEEFVQHFGKT

SEQ ID NO: 142 - protein, 410 aa, synthetic construct
MNGLSAQHERIAPARHECVYTVGYSEENVWKLCEHIKTVKRCPLGDVYAVFISNERKMVPIWKQKSGRGEEWVWWDYKVILLHDAHKEQTFIYDLGTTLPFPCPFDTYVKEAFKSDNYIHPAFWRKLRVVPADVFLQNFASDRSHMKDSSGGWRMPPPPYPCIETAESRMNLDDFISMNPSVGWGHVYTLEEFVQHFGKTEEEKRKREEEENGLSAQHERIAPARHECVYTVGYSEENVWKLCEHIKTVKRCPLGDVYAVFISNERKMVPIWKQKSGRGEEWVWWDYKVILLHDAHKEQTFIYDLGTTLPFPCPFDTYVKEAFKSDNYIHPAFWRKLRVVPADVFLQNFASDRSHMKDSSGGWRMPPPPYPCIETAESRMNLDDFISMNPSVGWGHVYTLEEFVQHFGKT

SEQ ID NO: 143 - protein, 421 aa, synthetic construct
MNGLSAQHERIAPARHECVYTVGYSEENVWKLCEHIKTVKRCPLGDVYAVFISNERKMVPIWKQKSGRGEEWVWWDYKVILLHDAHKEQTFIYDLGTTLPFPCPFDTYVKEAFKSDNYIHPAFWRKLRVVPADVFLQNFASDRSHMKDSSGGWRMPPPPYPCIETAESRMNLDDFISMNPSVGWGHVYTLEEFVQHFGKTAEAAAKEAAAKEAAAKEAAAKANGLSAQHERIAPARHECVYTVGYSEENVWKLCEHIKTVKRCPLGDVYAVFISNERKMVPIWKQKSGRGEEWVWWDYKVILLHDAHKEQTFIYDLGTTLPFPCPFDTYVKEAFKSDNYIHPAFWRKLRVVPADVFLQNFASDRSHMKDSSGGWRMPPPPYPCIETAESRMNLDDFISMNPSVGWGHVYTLEEFVQHFGKT

SEQ ID NO: 144 - protein, 426 aa, synthetic construct
MNGLSAQHERIAPARHECVYTVGYSEENVWKLCEHIKTVKRCPLGDVYAVFISNERKMVPIWKQKSGRGEEWVWWDYKVILLHDAHKEQTFIYDLGTTLPFPCPFDTYVKEAFKSDNYIHPAFWRKLRVVPADVFLQNFASDRSHMKDSSGGWRMPPPPYPCIETAESRMNLDDFISMNPSVGWGHVYTLEEFVQHFGKTAEAAAKEAAAKEAAAKEAAAKEAAAKANGLSAQHERIAPARHECVYTVGYSEENVWKLCEHIKTVKRCPLGDVYAVFISNERKMVPIWKQKSGRGEEWVWWDYKVILLHDAHKEQTFIYDLGTTLPFPCPFDTYVKEAFKSDNYIHPAFWRKLRVVPADVFLQNFASDRSHMKDSSGGWRMPPPPYPCIETAESRMNLDDFISMNPSVGWGHVYTLEEFVQHFGKT

SEQ ID NO: 145 - protein, 83 aa, synthetic construct
MHSKFSHAGRICGAKFKVGEPIYRCRECSFDPTCVLCVNCFNPKDHLGHHVYTTICTEFNNGECDCGDKTAWNHTLFCKAEEG

SEQ ID NO: 146 - protein, 83 aa, synthetic construct
MHSKFSHAGRICGAKFKVGEPIYRCHECSFDPTCVLCVNCFNPKDHLGHHVYTTICTEFNNGECDCGDKTAWNHTLFCKAEEG

SEQ ID NO: 147 - protein, 83 aa, synthetic construct
MHSKFSHAGRICGAKFKVGEPIYRCPECSFDPTCVLCVNCFNPKDHLGHHVYTTICTEFNNGECDCGDKTAWNHTLFCKAEEG

SEQ ID NO: 148 - protein, 83 aa, synthetic construct
MHSKFSHAGRICGAKFKVGEPIYRCRECSFDPTCVLCVNCFNPKDHLGHHVYTTICTQKNNGECDCGDKTAWNHTLFCKAEEG

SEQ ID NO: 149 - protein, 83 aa, synthetic construct
MHSKFSHAGRICGAKFKVGEPIYRCKECSFDDTCVLCVNCFNPKDHVGHHVYTTICTEFNNGECDCGDKTAWNHTLFCKAEEG

SEQ ID NO: 150 - protein, 83 aa, synthetic construct
MHSKFSHAGRICGAKFKVGEPIYRCKECSFDDTCVLCVNCFNPKDHRGHHVYTTICTEFNNGECDCGDKTAWNHTLFCKAEEG

SEQ ID NO: 151 - protein, 83 aa, synthetic construct
MHSKFSHAGRICGAKFKVGEPIYRCKECSFDDTCVLCVNCFNPKDHIGHHVYTTICTEFNNGECDCGDKTAWNHTLFCKAEEG

SEQ ID NO: 152 - protein, 83 aa, synthetic construct
MHSKFSHAGRICGAKFKVGEPIYRCKECSFDDTCVLCVNCFNPKDHLGHHVYVTICTEFNNGECDCGDKTAWNHTLFCKAEEG

SEQ ID NO: 153 - protein, 83 aa, synthetic construct
MHSKFSHAGRICGAKFKVGEPIYRCKECSFDDTCVLCVNCFNPKDHRGHHVYTTICTERNNGECDCGDKTAWNHTLFCKAEEG

SEQ ID NO: 154 - protein, 83 aa, synthetic construct
MHSKFSHAGRICGAKFKVGEPIYRCKECSFDDTCVLCVNCFNPKDHRGHHVYTTICTERNNGECDCGDKTAWNHELFCKAEEG

SEQ ID NO: 155 - protein, 83 aa, synthetic construct
MHSKFSHAGRICGAKFKVGEPIYRCKECSFDDTCVLCVNCFNPKDHRGHHVYTTICTERNNGECDCGDKTAWNHDLFCKAEEG

SEQ ID NO: 156 - protein, 83 aa, synthetic construct
MHSKFSHAGRICGAKFKVGEPIYRCKECSFDDTCVLCVNCFNPKDHIGHHVYTTICTERNNGECDCGDKTAWNHDLFCKAEEG

SEQ ID NO: 157 - protein, 83 aa, synthetic construct
MHSKFSHAGRICGAKFKVGEPIYRCKECSFDDTCVLCVNCFNPKDHIGHHVYTTICTERNNGECDCGDKTAWNHELFCKAEEG

SEQ ID NO: 158 - protein, 83 aa, synthetic construct
MHSKFSHAGRICGAKFKVGEPIYRCKECSFDDTCVLCVNCFNPKDHRGHHVYVTICTERNNGECDCGDKTAWNHELFCKAEEG

SEQ ID NO: 159 - protein, 83 aa, synthetic construct
MHSKFSHAGRICGAKFKVGEPIYRCRECSFDRTCVLCVNCFNPKDHRGHHVYVTICTERNNGECDCGDKTAWNHELFCKAEEG

SEQ ID NO: 160 - protein, 83 aa, synthetic construct
MHSKFSHAGRICGAKFKVGEPIYRCRECSFDPTCVLCVNCFNPKDHRGHHVYVTICTERNNGECDCGDKTAWNHELFCKAEEG

SEQ ID NO: 161 - protein, 83 aa, synthetic construct
MHSKFSHAGRICGAKFKVGEPIYRCRECSFDKTCVLCVNCFNPKDHLGHHVYTTICTQKNNGECDCGDKTAWNHTLFCKAEEG

SEQ ID NO: 162 - protein, 83 aa, synthetic construct
MHSKFSHAGRICGAKFKVGEPIYGCRECSFDRTCVLCVNCFNPNDHIGHHVYTTICTEKLNGECDCGDKTAWNHTLFCKAEEG

SEQ ID NO: 163 - protein, 177 aa, synthetic construct
MHSKFSHAGRICGAKFKVGEPIYRCRECSFDKTCVLCVNCFNPKDHLGHHVYTTICTQKNNGECDCGDKTAWNHTLFCKAEEGSAGSAAGSGEFMHSKFSHAGRICGAKFKVGEPIYRCRECSFDKTCVLCVNCFNPKDHLGHHVYTTICTQKNNGECDCGDKTAWNHTLFCKAEEG

SEQ ID NO: 164 - protein, 201 aa, synthetic construct
MHSKFSHAGRICGAKFKVGEPIYRCRECSFDKTCVLCVNCFNPKDHLGHHVYTTICTQKNNGECDCGDKTAWNHTLFCKAEEGSAGSAAGSGEFGSAGSAAGSGEFGSAGSAAGSGEFMHSKFSHAGRICGAKFKVGEPIYRCRECSFDKTCVLCVNCFNPKDHLGHHVYTTICTQKNNGECDCGDKTAWNHTLFCKAEEG

SEQ ID NO: 165 - protein, 177 aa, synthetic construct
MHSKFSHAGRICGAKFKVGEPIYGCRECSFDRTCVLCVNCFNPNDHIGHHVYTTICTEKLNGECDCGDKTAWNHTLFCKAEEGSAGSAAGSGEFMHSKFSHAGRICGAKFKVGEPIYGCRECSFDRTCVLCVNCFNPNDHIGHHVYTTICTEKLNGECDCGDKTAWNHTLFCKAEEG

SEQ ID NO: 166 - protein, 201 aa, synthetic construct
MHSKFSHAGRICGAKFKVGEPIYGCRECSFDRTCVLCVNCFNPNDHIGHHVYTTICTEKLNGECDCGDKTAWNHTLFCKAEEGSAGSAAGSGEFGSAGSAAGSGEFGSAGSAAGSGEFMHSKFSHAGRICGAKFKVGEPIYGCRECSFDRTCVLCVNCFNPNDHIGHHVYTTICTEKLNGECDCGDKTAWNHTLFCKAEEG

SEQ ID NO: 167 - protein, 177 aa, synthetic construct
MHSKFSHAGRICGAKFKVGEPIYRCKECSFDDTCVLCVNCFNPKDHRGHHVYVTICTERNNGECDCGDKTAWNHELFCKAEEGSAGSAAGSGEFMHSKFSHAGRICGAKFKVGEPIYRCKECSFDDTCVLCVNCFNPKDHRGHHVYVTICTERNNGECDCGDKTAWNHELFCKAEEG

SEQ ID NO: 168 - protein, 201 aa, synthetic construct
MHSKFSHAGRICGAKFKVGEPIYRCKECSFDDTCVLCVNCFNPKDHRGHHVYVTICTERNNGECDCGDKTAWNHELFCKAEEGSAGSAAGSGEFGSAGSAAGSGEFGSAGSAAGSGEFMHSKFSHAGRICGAKFKVGEPIYRCKECSFDDTCVLCVNCFNPKDHRGHHVYVTICTERNNGECDCGDKTAWNHELFCKAEEG

SEQ ID NO: 169 - protein, 173 aa, synthetic construct
MHSKFSHAGRICGAKFKVGEPIYRCKECSFDDTCVLCVNCFNPKDHRGHHVYVTICTERNNGECDCGDKTAWNHELFCKAEEGQGLQNEEMHSKFSHAGRICGAKFKVGEPIYRCKECSFDDTCVLCVNCFNPKDHRGHHVYVTICTERNNGECDCGDKTAWNHELFCKAEEG

SEQ ID NO: 170 - protein, 176 aa, synthetic construct
MHSKFSHAGRICGAKFKVGEPIYRCKECSFDDTCVLCVNCFNPKDHRGHHVYVTICTERNNGECDCGDKTAWNHELFCKAEEGDPGGGPSSRLMHSKFSHAGRICGAKFKVGEPIYRCKECSFDDTCVLCVNCFNPKDHRGHHVYVTICTERNNGECDCGDKTAWNHELFCKAEEG

SEQ ID NO: 171 - protein, 195 aa, synthetic construct
MHSKFSHAGRICGAKFKVGEPIYRCKECSFDDTCVLCVNCFNPKDHRGHHVYVTICTERNNGECDCGDKTAWNHELFCKAEEGGNDGLCQKLSVPCMSSKPQKPWEAKDAWEMHSKFSHAGRICGAKFKVGEPIYRCKECSFDDTCVLCVNCFNPKDHRGHHVYVTICTERNNGECDCGDKTAWNHELFCKAEEG

SEQ ID NO: 172 - protein, 178 aa, synthetic construct
MHSKFSHAGRICGAKFKVGEPIYRCKECSFDDTCVLCVNCFNPKDHRGHHVYVTICTERNNGECDCGDKTAWNHELFCKAEEGFSFGFSFGFSFGMHSKFSHAGRICGAKFKVGEPIYRCKECSFDDTCVLCVNCFNPKDHRGHHVYVTICTERNNGECDCGDKTAWNHELFCKAEEG

SEQ ID NO: 173 - protein, 190 aa, synthetic construct
MHSKFSHAGRICGAKFKVGEPIYRCKECSFDDTCVLCVNCFNPKDHRGHHVYVTICTERNNGECDCGDKTAWNHELFCKAEEGFSFGFSFGFSFGFSFGFSFGFSFGMHSKFSHAGRICGAKFKVGEPIYRCKECSFDDTCVLCVNCFNPKDHRGHHVYVTICTERNNGECDCGDKTAWNHELFCKAEEG

SEQ ID NO: 174 - protein, 181 aa, synthetic construct
MHSKFSHAGRICGAKFKVGEPIYRCKECSFDDTCVLCVNCFNPKDHRGHHVYVTICTERNNGECDCGDKTAWNHELFCKAEEGEAAAKEAAAKEAAAKMHSKFSHAGRICGAKFKVGEPIYRCKECSFDDTCVLCVNCFNPKDHRGHHVYVTICTERNNGECDCGDKTAWNHELFCKAEEG

SEQ ID NO: 175 - protein, 177 aa, synthetic construct
MHSKFSHAGRICGAKFKVGEPIYRCKECSFDDTCVLCVNCFNPKDHRGHHVYVTICTERNNGECDCGDKTAWNHELFCKAEEGEEEKRKREEEEMHSKFSHAGRICGAKFKVGEPIYRCKECSFDDTCVLCVNCFNPKDHRGHHVYVTICTERNNGECDCGDKTAWNHELFCKAEEG

SEQ ID NO: 176 - protein, 173 aa, synthetic construct
MHSKFSHAGRICGAKFKVGEPIYRCKECSFDDTCVLCVNCFNPKDHRGHHVYVTICTERNNGECDCGDKTAWNHELFCKAEEGLPSPDVHMHSKFSHAGRICGAKFKVGEPIYRCKECSFDDTCVLCVNCFNPKDHRGHHVYVTICTERNNGECDCGDKTAWNHELFCKAEEG

SEQ ID NO: 177 - protein, 182 aa, synthetic construct
MHSKFSHAGRICGAKFKVGEPIYRCKECSFDDTCVLCVNCFNPKDHRGHHVYVTICTERNNGECDCGDKTAWNHELFCKAEEGAKLKQKTEQLQDRIAGMHSKFSHAGRICGAKFKVGEPIYRCKECSFDDTCVLCVNCFNPKDHRGHHVYVTICTERNNGECDCGDKTAWNHELFCKAEEG

SEQ ID NO: 178 - protein, 182 aa, synthetic construct
MHSKFSHAGRICGAKFKVGEPIYRCKECSFDDTCVLCVNCFNPKDHRGHHVYVTICTERNNGECDCGDKTAWNHELFCKAEEGWRIRPRPPRLPRPRPRMHSKFSHAGRICGAKFKVGEPIYRCKECSFDDTCVLCVNCFNPKDHRGHHVYVTICTERNNGECDCGDKTAWNHELFCKAEEG

SEQ ID NO: 179 - protein, 186 aa, synthetic construct
MHSKFSHAGRICGAKFKVGEPIYRCKECSFDDTCVLCVNCFNPKDHRGHHVYVTICTERNNGECDCGDKTAWNHELFCKAEEGAPAPAPAPAPAPAPAPAPAPMHSKFSHAGRICGAKFKVGEPIYRCKECSFDDTCVLCVNCFNPKDHRGHHVYVTICTERNNGECDCGDKTAWNHELFCKAEEG

SEQ ID NO: 180 - protein, 188 aa, synthetic construct
MHSKFSHAGRICGAKFKVGEPIYRCKECSFDDTCVLCVNCFNPKDHRGHHVYVTICTERNNGECDCGDKTAWNHELFCKAEEGAEAAAKEAAAKEAAAKEAAAKAMHSKFSHAGRICGAKFKVGEPIYRCKECSFDDTCVLCVNCFNPKDHRGHHVYVTICTERNNGECDCGDKTAWNHELFCKAEEG

SEQ ID NO: 181 - protein, 193 aa, synthetic construct
MHSKFSHAGRICGAKFKVGEPIYRCKECSFDDTCVLCVNCFNPKDHRGHHVYVTICTERNNGECDCGDKTAWNHELFCKAEEGAEAAAKEAAAKEAAAKEAAAKEAAAKAMHSKFSHAGRICGAKFKVGEPIYRCKECSFDDTCVLCVNCFNPKDHRGHHVYVTICTERNNGECDCGDKTAWNHELFCKAEEG

SEQ ID NO: 182 - protein, 218 aa, synthetic construct
MSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEEGSAGSAAGSGEFMSDSPVDLKPKPKVKPKLERPKLYKVMLLNDDYTPMSFVTVVLKAVFRMSEDTGRRVMMTAHRFGSAVVVVCERDIAETKAKEATDLGKEAGFPLMFTTEPEE

SEQ ID NO: 183 - protein, 244 aa, synthetic construct
MGLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSEAAAKEAAAKEAAAKMGLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSG

SEQ ID NO: 184 - protein, 200 aa, synthetic construct
MNGLSAQHERIAPARHECVYTSCYSEENVWKLCEHIKTSKRCPLGDVYAVFISNERKMVPIWKQKSGRGERPVIWDYQVILLHDCHKEQTFIYDLDTTLPFPCPFDTYVKEAFKSDNYINPAFWRKLRVVPADVFLQNFASDRSHMKDASGGWRMPPPPYPCIETAESRMNLDDFISMNPSVGWGHVYTLEEFVQHFGKT

SEQ ID NO: 185 - protein, 119 aa, synthetic construct
MPTAASATESAIEDTPAPARPEVDGRTKPKRQPRYHVVLWDDDDHTYQYVVVMLRSLFGHPPERGYRMAKEMDTQGRVIVLTTTREHAELKRDQIHAFGRDRLLARSKGSMKASIEAEE

SEQ ID NO: 186 - protein, 27 aa, synthetic construct
GGGSGGGSGGGSGLNDFFEAQKIEWHE

SEQ ID NO: 187 - protein, 54 aa, synthetic construct
GGGSGGGSGGGSGLNDFFEAQKIEWHEGGGSGGGSGGGSGLNDFFEAQKIEWHE

SEQ ID NO: 188 - protein, 56 aa, synthetic construct
GSGGGSGGGSGGGSGLNDFFEAQKIEWHEGGGSGGGSGGGSGLNDFFEAQKIEWHE

SEQ ID NO: 189 - protein, 38 aa, synthetic construct
GHHHHHHHHHHGGGSGGGSGGGSGLNDFFEAQKIEWHE

SEQ ID NO: 190 - protein, 65 aa, synthetic construct
GHHHHHHHHHHGGGSGGGSGGGSGLNDFFEAQKIEWHEGGGSGGGSGGGSGLNDFFEAQKIEWHE

SEQ ID NO: 191 - protein, 67 aa, synthetic construct
GGSHHHHHHHHHHGGGSGGGSGGGSGLNDFFEAQKIEWHEGGGSGGGSGGGSGLNDFFEAQKIEWHE

SEQ ID NO: 192 - protein, 66 aa, synthetic construct
GSHHHHHHHHHHGGGSGGGSGGGSGLNDFFEAQKIEWHEGGGSGGGSGGGSGLNDFFEAQKIEWHE

SEQ ID NO: 193 - protein, 61 aa, synthetic construct
GGGSGGGSGGGSGLNDFFEAQKIEWHEGGGSGGGSGGGSGLNDFFEAQKIEWHEGHHHHHH

SEQ ID NO: 194 - protein, 13 aa, synthetic construct
GGGSGGGSGGGSG

SEQ ID NO: 195 - protein, 12 aa, synthetic construct
GSAGSAAGSGEF

SEQ ID NO: 196 - protein, 36 aa, synthetic construct
GSAGSAAGSGEFGSAGSAAGSGEFGSAGSAAGSGEF

SEQ ID NO: 197 - protein, 15 aa, synthetic construct
EAAAKEAAAKEAAAK

SEQ ID NO: 198 - protein, 22 aa, synthetic construct
AEAAAKEAAAKEAAAKEAAAKA

SEQ ID NO: 199 - protein, 27 aa, synthetic construct
AEAAAKEAAAKEAAAKEAAAKEAAAKA

SEQ ID NO: 200 - protein, 11 aa, synthetic construct
EEEKRKREEEE

SEQ ID NO: 201 - protein, 7 aa, synthetic construct
QGLQNEE

SEQ ID NO: 202 - protein, 10 aa, synthetic construct
DPGGGPSSRL

SEQ ID NO: 203 - protein, 29 aa, synthetic construct
GNDGLCQKLSVPCMSSKPQKPWEAKDAWE

SEQ ID NO: 204 - protein, 12 aa, synthetic construct
FSFGFSFGFSFG

SEQ ID NO: 205 - protein, 24 aa, synthetic construct
FSFGFSFGFSFGFSFGFSFGFSFG

SEQ ID NO: 206 - protein, 7 aa, synthetic construct
LPSPDVH

SEQ ID NO: 207 - protein, 16 aa, synthetic construct
AKLKQKTEQLQDRIAG

SEQ ID NO: 208 - protein, 16 aa, synthetic construct
WRIRPRPPRLPRPRPR

SEQ ID NO: 209 - protein, 20 aa, synthetic construct
APAPAPAPAPAPAPAPAPAP

SEQ ID NO: 210 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDTHKEQTFIYDLDTTLPFPCPFDTYVKEAFKSDNYINPAFWRKLRVVPADVFLQNFASDRSHMKMASGGWRMPPPPYPCIETAESRMNLDDFISMNPSVGWGHVYTLEEFVQHFGKT

SEQ ID NO: 211 - protein, 200 aa, synthetic construct
MNGLSAQHERIAPARHECVYTTCYSEENVWKLCEHIKTNKRCPLGDVYAVFISNERKMVPIWKQKSGRGEEPVYWDYQVILLHDCHKEQTFIYDLDTTLPFPCPFDTYVKEAFKSDNYINPAFWRKLRVVPADVFLQNFASDRSHMKYASGGWRMPPPPYPCIETAESRMNLDDFISMNPSVGWGHVYTLEEFVQHFGKT

SEQ ID NO: 212 - protein, 200 aa, synthetic construct
MNGLSAQHERIAPARHECVYTSCYSEENVWKLCEHIKTSKRCPLGDVYAVFISNERKMVPIWKQKSGRGEEPVIWDYQVILLHDCHKEQTFIYDLDTTLPFPCPFDTYVKEAFKSDNYINPAFWRKLRVVPADVFLQNFASDRSHMKFASGGWRMPPPPYPCIETAESRMNLDDFISMNPSVGWGHVYTLEEFVQHFGKT

SEQ ID NO: 213 - protein, 200 aa, synthetic construct
MNGLSAQHERILPARHECVYTECYSEENVWKLCEHIKTSKRCPLGDVYAVFISNERKMVPIWKQKVGRGEEPVIWDYQVILLHDCHKEQTFIYDLDTTLPFPCPFDTYVKEAFKSDNYIRPRFWRKLRVVPADVFLQNFASDRSHMKDAVGGWRMPPPPYPCIETAESRMNLDDFISMNPSVGWGHVYTLEEFVQHFGKT

SEQ ID NO: 214 - protein, 200 aa, synthetic construct
MNGLSAQHERILPARHECVYTECYGEENVYKLCEHIKTSKRCPLGDVYAVFISNERKMVPIWKQKVGRGEEPIIWDYQVILLHDCHKEQTFIYDLDTTLPFPCPFDTYVKEAFKSDNYIRPAFWRKLRVVPADVFLQNFASDRSHMKDGVGGWQMSPPPYPCIETAESRMNLDDFISMNPSVGWGHVYTLEEFVQHFGKT

SEQ ID NO: 215 - protein, 200 aa, synthetic construct
MNGLSAQHERILPARHECVYTECYSEENVWKLCEHIKTSKRCPLGDVYAVFISNERKMVPIWKQKVGRGERPVIWDYQVILLHDCHKEQTFIYDLDTTLPFPCPFDTYVKEAFKSDNYINPRFWRKLRVVPADVFLQNFASDRSHMKDLVGGWLMPPPPYPCIETAESRMNLDDFISMNPSVGWGHVYTLEEFVQHFGKT

SEQ ID NO: 216 - protein, 200 aa, synthetic construct
MNGLSAQHERILPARHECVYTECYSEENVWKLCEHIKTSKRCPLGDVYAVFISNERKMVPIWKQKVGRGERPVIWDYQVILLHDCHKEQTFIYDLDTTLPFPCPFDTYVKEAFKSDNYINPDFWRKLRVIPADVFLQNFASDRSHMKDLVGGWLMPPPPYPCIETAESRMNLDDFISMNPSVGWGHVYTLEEFVQHFGKT

SEQ ID NO: 217 - protein, 200 aa, synthetic construct
MNGLSAQHERILPARHECVYTSCYSEENVWKLCEHIKTSKRCPLGDVYAVFISNERKMVPIWKQKIGRGKRPIIWDYQVVLLHDRHKEQTFIYDLDTTLPFPCPFDTYVKEAFKSDNYINPAFWRKLRVVPADVFLQNFASDRSHMKDVGGGWLMPPPPYPCIETAESRMNLDDFISMNPSVGWGHVYTLEEFVQHFGKT

SEQ ID NO: 218 - protein, 200 aa, synthetic construct
MNGLSAQHERILPARHECVYTECYSEENVWKLCEHIKTSKRCPLGDVYAVFISNERKMVPIWKQKSGRGERPIIWDYQVVLLHDCHKEQTFIYDLDTTLPFPCPFDTYVKEAFKSDNYINPRFWRKLRVVPADVFLQNFASDRSHMRDNGGGWLMPPPPYPCIETAESRMNLDDFISMNPSVGWGHVYTLEEFVQHFGKT

SEQ ID NO: 219 - protein, 200 aa, synthetic construct
MNGLSAQHERILPARHECVYTSCYSEENVWKLCEHIKTSKRCPLGDVYAVFISNERKMVPIWKQKSGRGERPIIWDYQVVLLHDCHKEQTFIYDLDTTLPFPCPFDTYVKEAFKSDNYISPRFWRKLRVVPADVFLQNFASDRSHMKDDVGGWQMPPPPYPCIETAESRMNLDDFISMNPSVGWGHVYTLEEFVQHFGKT

SEQ ID NO: 220 - protein, 200 aa, synthetic construct
MNGLSAQHERITPARHECVYTECYSEENVYKLCEHIKTSKRCPLGDVYAVFISNERKMVPIWKQKSGRGEKPVIWDYQVILLHDCHKEQTFIYDLDTTLPFPCPFDTYVKEAFKSDNYIRPRFWRKLRVVPADVFLQNFASDRSHMKDDVGGWLMPPPPYPCIETAESRMNLDDFISMNPSVGWGHVYTLEEFVQHFGKT

SEQ ID NO: 221 - protein, 200 aa, synthetic construct
MNGLSAQHERILPARHECVYTEYYGWENVWKLCEHIKTSKRCPLGDVYAVFISNERKMVPIWKQKSGRGEEPIIWDYQVVLLHDCHKEQTFIYDLDTTLPFPCPFDTYVKEAFKSDNYINPDFWRKLRVVPADVFLQNFASDRSHMKDGCGGWQMPPPPYPCIETAESRMNLDDFISMNPSVGWGHVYTLEEFVQHFGKT

SEQ ID NO: 222 - protein, 200 aa, synthetic construct
MNGLSAQHERILPARHECVYTRCYSEENVWKLCEHIKTSKRCPLGDVYAVFISNERKMVPIWKQKVGRGERPVIWDYQVILLHDCHKEQTFIYDLDTTLPFPCPFDTYVKEAFKSDNYINPRFWRKLRVVPADVFLQNFASDRSHMKDLVGGWLMPPPPYPCIETAESRMNLDDFISMNPSVGWGHVYTLEEFVQHFGKT

SEQ ID NO: 223 - protein, 200 aa, synthetic construct
MNGLSAQHERILPARHECVYTECYSEENVWKLCEHIKTSKRCPLGDVYAVFISNERKMVPIWKQKSGRGERPVIWDYQVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKT

SEQ ID NO: 224 - protein, 200 aa, synthetic construct
MNGLCAQHERILPARHECVYTSCYSEENVWKLCEHIKTSKRCPLGDVYAVFISNERKMVPIWKQKSGRGERPIIWDYQVVLLHDCHKEQTFIYDLDTTLPFPCPFDTYVKEAFKSDNYISPRFWRKLRVVPADVFLQNFASDRSHMKDDVGGWQMPPPPYPCIETAESRMNLDDFISMNPSVGWGHVYTLEEFVQHFGKT

SEQ ID NO: 225 - protein, 200 aa, synthetic construct
MNGLSAQHERILPARHECVYTECYSEENVWKLCEHIKTSKRCPLGDVYAVFISNERKMVPIWKQRVGRGERPVIWDYQVILLHDCHKEQTFIYDLDTTLPFPCPFDTYVKEAFKSDNYINPRFWRKLRVVPADVFLQNFASDRSHMKDAVGGWLMPPPPYPCIETAESRMNLDDFISMNPSVGWGHVYTLEEFVQHFGKT

SEQ ID NO: 226 - protein, 200 aa, synthetic construct
MNGLSAQHERILPARHECVYTECYQEENVWKLCEHIKTSKRCPLGDVYAVFISNERKMVPIWKQKVGRGEEPVIWDYHVILLHDCHKEQTFIYDLDTTLPFPCPFDTYVKEAFKSDNYIRPRFWRKLRVVPADVFLQNFASDRSHMKDAVGGWRMPPPPYPCIETAESRMNLDDFISMNPSVGWGHVYTLEEFVQHFGKT

SEQ ID NO: 227 - protein, 200 aa, synthetic construct
MNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYINPRFWRKLRVVPADVFLQNFASDRSHMKDAVGGWLMPPPPYPCIETAESRMNLDDFISMNPSVGWGHVYTLEEFVQHFGKT

SEQ ID NO: 228 - protein, 200 aa, synthetic construct
MNGLSAQHERILPARHECVYTECYQEENVWKLCEHIKTSKRCPLGDVYAVFISNERKMVPIWKQKSGRGERPIIWDYHVVLLHDCHKEQTFIYDLDTTLPFPCPFDTYVKEAFKSDNYINPRFWRKLRVVPADVFLQNFASDRSHMRDNGGGWLMPPPPYPCIETAESRMNLDDFISMNPSVGWGHVYTLEEFVQHFGKT

SEQ ID NO: 229 - protein, 200 aa, synthetic construct
MNGLSAQHERILPARHECVYTSCYQEENVWKLCEHIKTSKRCPLGDVYAVFISNERKMVPIWKQKSGRGERPIIWDYHVVLLHDCHKEQTFIYDLDTTLPFPCPFDTYVKEAFKSDNYISPRFWRKLRVVPADVFLQNFASDRSHMKDDVGGWQMPPPPYPCIETAESRMNLDDFISMNPSVGWGHVYTLEEFVQHFGKT

SEQ ID NO: 230 - protein, 200 aa, synthetic construct
MNGLSAQHERITPARHECVYTECYQEENVYKLCEHIKTSKRCPLGDVYAVFISNERKMVPIWKQKSGRGEKPVIWDYHVILLHDCHKEQTFIYDLDTTLPFPCPFDTYVKEAFKSDNYIRPRFWRKLRVVPADVFLQNFASDRSHMKDDVGGWLMPPPPYPCIETAESRMNLDDFISMNPSVGWGHVYTLEEFVQHFGKT

SEQ ID NO: 231 - protein, 200 aa, synthetic construct
MNGLSAQHERILPARHECVYTRCYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYINPRFWRKLRVVPADVFLQNFASDRSHMKDLVGGWLMPPPPYPCIETAESRMNLDDFISMNPSVGWGHVYTLEEFVQHFGKT

SEQ ID NO: 232 - protein, 200 aa, synthetic construct
MNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKT

SEQ ID NO: 233 - protein, 200 aa, synthetic construct
MNGLCAQHERILPARHECVYTSCYQEENVWKLCEHIKTSKRCPLGDVYAVFISNERKMVPIWKQKSGRGERPIIWDYHVVLLHDCHKEQTFIYDLDTTLPFPCPFDTYVKEAFKSDNYISPRFWRKLRVVPADVFLQNFASDRSHMKDDVGGWQMPPPPYPCIETAESRMNLDDFISMNPSVGWGHVYTLEEFVQHFGKT

SEQ ID NO: 234 - protein, 200 aa, synthetic construct
MNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKT

SEQ ID NO: 235 - protein, 200 aa, synthetic construct
MNGLSAQHERILPARHECVYTECYQEENVWKLCEHIKTSKRCPLGDVYAVFISNERKMVPIWKQRVGRGERPVIWDYHVILLHDCHKEQTFIYDLDTTLPFPCPFDTYVKEAFKSDNYINPRFWRKLRVVPADVFLQNFASDRSHMKDAVGGWLMPPPPYPCIETAESRMNLDDFISMNPSVGWGHVYTLEEFVQHFGKT

SEQ ID NO: 236 - protein, 200 aa, synthetic construct
MNGLSAQHERILPARHECVYTECYQEENVWKLCEHIKTSKRCPLGDVYAVFISNERKMVPIWKQKVGRGERPLIWDYHVILLHDCHKEQTFIYDLDTTLPFPCPFDTYVKEAFKSDNYINPRFWRKLRVVPADVFLQNFASDRSHMKDAVGGWLMPPPPYPCIETAESRMNLDDFISMNPSVGWGHVYTLEEFVQHFGKT

SEQ ID NO: 237 - protein, 200 aa, synthetic construct
MNGLSAQHERILPARHECVYTECYQEENVWKLCEHIKTSKRCPLGDVYAVFISNERKMVPIWKQKVGRGERPVIWDYHVVLLHDCHKEQTFIYDLDTTLPFPCPFDTYVKEAFKSDNYINPRFWRKLRVVPADVFLQNFASDRSHMKDAVGGWLMPPPPYPCIETAESRMNLDDFISMNPSVGWGHVYTLEEFVQHFGKT

SEQ ID NO: 238 - protein, 200 aa, synthetic construct
MNGLSAQHERILPARHECVYTECYQEENVWKLCEHIKTSKRCPLGDVYAVFISNERKMVPIWKQKVGRGERPIIWDYHVVLLHDCHKEQTFIYDLDTTLPFPCPFDTYVKEAFKSDNYINPRFWRKLRVVPADVFLQNFASDRSHMKDAVGGWLMPPPPYPCIETAESRMNLDDFISMNPSVGWGHVYTLEEFVQHFGKT

SEQ ID NO: 239 - protein, 200 aa, synthetic construct
MNGLSAQHERILPARHECVYTECYQEENVWKLCEHIKTSKRCPLGDVYAVFISNERKMVPIWKQKVGRGERPVIWDYQVILLHDCHKEQTFIYDLDTTLPFPCPFDTYVKEAFKSDNYINPRFWRKLRVVPADVFLQNFASDRSHMKDAVGGWLMPPPPYPCIETAESRMNLDDFISMNPSVGWGHVYTLEEFVQHFGKT

SEQ ID NO: 240 - protein, 200 aa, synthetic construct
MNGLSAQHERILPARHECVYTECYSEENVWKLCEHIKTSKRCPLGDVYAVFISNERKMVPIWKQKVGRGERPVIWDYHVILLHDCHKEQTFIYDLDTTLPFPCPFDTYVKEAFKSDNYINPRFWRKLRVVPADVFLQNFASDRSHMKDAVGGWLMPPPPYPCIETAESRMNLDDFISMNPSVGWGHVYTLEEFVQHFGKT

SEQ ID NO: 241 - protein, 200 aa, synthetic construct
MNGLSAQHERILPARHECVYTECYNEENVWKLCEHIKTSKRCPLGDVYAVFISNERKMVPIWKQKVGRGERPVIWDYHVILLHDCHKEQTFIYDLDTTLPFPCPFDTYVKEAFKSDNYINPRFWRKLRVVPADVFLQNFASDRSHMKDAVGGWLMPPPPYPCIETAESRMNLDDFISMNPSVGWGHVYTLEEFVQHFGKT

SEQ ID NO: 242 - protein, 200 aa, synthetic construct
MNGLSAQHERILPARHECVYTECYQEENVWKLCEHIKTSKRCPLGDVYAVFISNERKMVPIWKQKVGRGERPVIWDYLVILLHDCHKEQTFIYDLDTTLPFPCPFDTYVKEAFKSDNYINPRFWRKLRVVPADVFLQNFASDRSHMKDAVGGWLMPPPPYPCIETAESRMNLDDFISMNPSVGWGHVYTLEEFVQHFGKT

SEQ ID NO: 243 - protein, 200 aa, synthetic construct
MNGLSAQHERILPARHECVYTSCYQEENVWKLCEHIKTSKRCPLGDVYAVFISNERKMVPIWKQKSGRGERPVIWDYHVILLHDCHKEQTFIYDLDTTLPFPCPFDTYVKEAFKSDNYINPRFWRKLRVVPADVFLQNFASDRSHMKDASGGWRMPPPPYPCIETAESRMNLDDFISMNPSVGWGHVYTLEEFVQHFGKT

SEQ ID NO: 244 - protein, 200 aa, synthetic construct
MNGLSAQHERILPARHECVYTECYQEENVWKLCEHIKTSKRCPLGDVYAVFISNERKMVPIWKQKVGRGERPVIWDYMVILLHDCHKEQTFIYDLDTTLPFPCPFDTYVKEAFKSDNYINPRFWRKLRVVPADVFLQNFASDRSHMKDAVGGWLMPPPPYPCIETAESRMNLDDFISMNPSVGWGHVYTLEEFVQHFGKT

SEQ ID NO: 245 - protein, 200 aa, synthetic construct
MNGLSAQHERILPARHECVYTEYYGEENVWKLCEHIKTSKRCPLGDVYAVFISNERKMVPIWKQKSGRGERPIIWDYQVILLHDCHKEQTFIYDLDTTLPFPCPFDTYVKEAFKSDNYINPRFWRKLRVVPADVFLQNFASDRSHMKDIVGGWLMPPPPYPCIETAESRMNLDDFISMNPSVGWGHVYTLEEFVQHFGKT

SEQ ID NO: 246 - protein, 200 aa, synthetic construct
MNGLSAQHERILPARHECVYTECYGEENVWKLCEHIKTSKRCPLGDVYAVFISNERKMVPIWKQKVGRGERPVIWDYQVILLHDCHKEQTFIYDLDTTLPFPCPFDTYVKEAFKSDNYINPRFWRKLRVVPADVFLQNFASDRSHMKDASGGWLMPPPPYPCIETAESRMNLDDFISMNPSVGWGHVYTLEEFVQHFGKT

SEQ ID NO: 247 - protein, 200 aa, synthetic construct
MNGLSAQHERILPARHECVYTECYQEENVWKLCEHIKTSKRCPLGDVYAVFISNERKMVPIWKQKVGRGERPVIWDYNVILLHDCHKEQTFIYDLDTTLPFPCPFDTYVKEAFKSDNYINPRFWRKLRVVPADVFLQNFASDRSHMKDAVGGWLMPPPPYPCIETAESRMNLDDFISMNPSVGWGHVYTLEEFVQHFGKT

SEQ ID NO: 248 - protein, 412 aa, synthetic construct
MNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKTGGGSGGGSGGGSGNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKT

SEQ ID NO: 249 - protein, 411 aa, synthetic construct
MNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKTGSAGSAAGSGEFNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKT

SEQ ID NO: 250 - protein, 435 aa, synthetic construct
MNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKTGSAGSAAGSGEFGSAGSAAGSGEFGSAGSAAGSGEFNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKT

SEQ ID NO: 251 - protein, 406 aa, synthetic construct
MNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKTQGLQNEENGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKT

SEQ ID NO: 252 - protein, 409 aa, synthetic construct
MNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKTDPGGGPSSRLNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKT

SEQ ID NO: 253 - protein, 428 aa, synthetic construct
MNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKTGNDGLCQKLSVPCMSSKPQKPWEAKDAWENGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKT

SEQ ID NO: 254 - protein, 411 aa, synthetic construct
MNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKTFSFGFSFGFSFGNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKT

SEQ ID NO: 255 - protein, 423 aa, synthetic construct
MNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKTFSFGFSFGFSFGFSFGFSFGFSFGNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKT

SEQ ID NO: 256 - protein, 414 aa, synthetic construct
MNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKTEAAAKEAAAKEAAAKNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKT

SEQ ID NO: 257 - protein, 410 aa, synthetic construct
MNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKTEEEKRKREEEENGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKT

SEQ ID NO: 258 - protein, 406 aa, synthetic construct
MNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKTLPSPDVHNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKT

SEQ ID NO: 259 - protein, 415 aa, synthetic construct
MNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKTAKLKQKTEQLQDRIAGNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKT

SEQ ID NO: 260 - protein, 415 aa, synthetic construct
MNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKTWRIRPRPPRLPRPRPRNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKT

SEQ ID NO: 261 - protein, 419 aa, synthetic construct
MNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKTAPAPAPAPAPAPAPAPAPAPNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKT

SEQ ID NO: 262 - protein, 421 aa, synthetic construct
MNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKTAEAAAKEAAAKEAAAKEAAAKANGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKT

SEQ ID NO: 263 - protein, 426 aa, synthetic construct
MNGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKTAEAAAKEAAAKEAAAKEAAAKEAAAKANGLSAQHERILPARHECVYTECYQEENVWKLCEHIKTSKRCPLGDVYAVFISNERKMVPIWKQKSGRGERPVIWDYHVVLLHDRHKEQTFIYDLDTTLPFPCPFDTYVKEAFKSDNYIRPRFWRKLRVVPADVFLQNFASDRSHMKDVSGGWRMPPPPYPCIETAESRMNLDDFISMNPSVGWGHVYTLEEFVQHFGKT

SEQ ID NO: 264 - protein, 412 aa, synthetic construct
MNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKTGGGSGGGSGGGSGNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKT

SEQ ID NO: 265 - protein, 411 aa, synthetic construct
MNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKTGSAGSAAGSGEFNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKT

SEQ ID NO: 266 - protein, 435 aa, synthetic construct
MNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKTGSAGSAAGSGEFGSAGSAAGSGEFGSAGSAAGSGEFNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKT

SEQ ID NO: 267 - protein, 406 aa, synthetic construct
MNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKTQGLQNEENGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKT

SEQ ID NO: 268 - protein, 409 aa, synthetic construct
MNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKTDPGGGPSSRLNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKT

SEQ ID NO: 269 - protein, 428 aa, synthetic construct
MNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKTGNDGLCQKLSVPCMSSKPQKPWEAKDAWENGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKT

SEQ ID NO: 270 - protein, 411 aa, synthetic construct
MNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKTFSFGFSFGFSFGNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKT

SEQ ID NO: 271 - protein, 423 aa, synthetic construct
MNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKTFSFGFSFGFSFGFSFGFSFGFSFGNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKT

SEQ ID NO: 272 - protein, 414 aa, synthetic construct
MNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKTEAAAKEAAAKEAAAKNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKT

SEQ ID NO: 273 - protein, 410 aa, synthetic construct
MNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKTEEEKRKREEEENGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKT

SEQ ID NO: 274 - protein, 406 aa, synthetic construct
MNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKTLPSPDVHNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKT

SEQ ID NO: 275 - protein, 415 aa, synthetic construct
MNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKTAKLKQKTEQLQDRIAGNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKT

SEQ ID NO: 276 - protein, 415 aa, synthetic construct
MNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKTWRIRPRPPRLPRPRPRNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKT

SEQ ID NO: 277 - protein, 419 aa, synthetic construct
MNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKTAPAPAPAPAPAPAPAPAPAPNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKT

SEQ ID NO: 278 - protein, 421 aa, synthetic construct
MNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKTAEAAAKEAAAKEAAAKEAAAKANGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKT

SEQ ID NO: 279 - protein, 426 aa, synthetic construct
MNGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKTAEAAAKEAAAKEAAAKEAAAKEAAAKANGLSAQHERILPARHECVYTECYQEENVWKLCEHIKTSKRCPLGDVYAVFISNERKMVPIWKQKVGRGERPVIWDYHVILLHDCHKEQTFIYDLDTTLPFPCPFDTYVKEAFKSDNYIRPRFWRKLRVVPADVFLQNFASDRSHMKDAVGGWLMPPPPYPCIETAESRMNLDDFISMNPSVGWGHVYTLEEFVQHFGKT

SEQ ID NO: 280 - protein, 67 aa, synthetic construct
GSGGGSGGGSGGGSGLNDIFEAQKIEWHEGGGSGGGSGGGSGLNDIFEAQKIEWHEGGGGSHHHHHH

SEQ ID NO: 281 - protein, 12 aa, synthetic construct
DAKLDEESILKQ

SEQ ID NO: 282 - protein, 4 aa, synthetic construct
DAKL

SEQ ID NO: 283 - protein, 4 aa, synthetic construct
EAKL

SEQ ID NO: 284 - protein, 4 aa, synthetic construct
EVFR

SEQ ID NO: 285 - protein, 4 aa, synthetic construct
DVFR

SEQ ID NO: 286 - protein, 4 aa, synthetic construct
QAKL

SEQ ID NO: 287 - protein, 4 aa, synthetic construct
NAKL

SEQ ID NO: 288 - protein, 4 aa, synthetic construct
RAKL

SEQ ID NO: 289 - protein, 13 aa, synthetic construct
  VARIANT 1: note = X can be an N, Q, E, or D
XAKLDEESILKQK

SEQ ID NO: 290 - protein, 17 aa, synthetic construct
  VARIANT 1: note = X can be an N, Q, E, or D
  SITE 5: note = Fluorescein isothiocyanate label is attached to K
XHGSKFITCDEESILKQ

SEQ ID NO: 291 - protein, 13 aa, synthetic construct
  VARIANT 1: note = X can be an N, Q, E, or D
XVFRDEESILKQK

SEQ ID NO: 292 - protein, 10 aa, synthetic construct
RLIFAYPDDD

SEQ ID NO: 293 - protein, 5 aa, synthetic construct
RLIFA

SEQ ID NO: 294 - protein, 4 aa, synthetic construct
LIFA

(19) United States
(12) Patent Application Publication
(10) Pub. No.: US 2026/0016481 A1
(43) Pub. Date: Jan. 15, 2026

(54) POLYPEPTIDE SEQUENCING REAGENTS AND METHODS OF USE

(71) Applicant: Quantum-Si Incorporated, Branford, CT (US)

(72) Inventor: Brian Reed, Madison, CT (US)

(73) Assignee: Quantum-Si Incorporated, Branford, CT (US)

(21) Appl. No.: 19/264,736

(22) Filed: Jul. 9, 2025

Related U.S. Application Data
(60) Provisional application No. 63/669,047, filed on Jul. 9, 2024.
(60) Provisional application No. 63/767,976, filed on Mar. 6, 2025.

(51) Int. Cl. G01N 33/68; C12N 9/10; C12N 9/80; C12Q 1/37; G01N 21/64

(52) U.S. Cl. CPC G01N 33/6824; C12N 9/104; C12N 9/80; C12Q 1/37; G01N 21/6428; C07K 2319/20; C12Y 203/02; C12Y 305/01; G01N 2021/6441; G01N 2201/12; G01N 2333/95

(57) ABSTRACT

Provided herein are novel amino acid binding proteins that recognize aspartate and/or glutamate in protein sequencing reactions; novel amino acid binding proteins that recognize arginine in protein sequencing reactions; and novel amino acid binding proteins that recognize glycine, alanine, and/or serine in protein sequencing reactions.

### CROSS-REFERENCE TO RELATED APPLICATIONS

This application claims priority under 35 U.S.C. § 119(e) to U.S. Provisional Application No. 63/669,047, filed Jul. 9, 2024, and U.S. Provisional Application No. 63/767,976, filed Mar. 6, 2025, each of which is hereby incorporated by reference in its entirety.

### REFERENCE TO AN ELECTRONIC SEQUENCE LISTING

The contents of the electronic sequence listing (R070870176US02-SEQ-JIB.xml; Size: 318,334 bytes; and Date of Creation: Jul. 8, 2025) is herein incorporated by reference in its entirety.

### BACKGROUND

Measurements of the proteome provide deep and valuable insight into biological processes. However, the complex nature of the proteome and the chemical properties of proteins present fundamental challenges to achieving sensitivity, throughput, cost, and adoption on par with DNA sequencing technologies. Methods to directly sequence single protein molecules offer the maximum possible detection sensitivity, with the potential to enable single-cell inputs, digital quantification based on read counts, detection of posttranslational modifications (PTMs) and low-abundance or aberrant proteoforms, and cost and throughput levels that favor broad adoption.

### SUMMARY

Provided herein are novel amino acid binding proteins that recognize specific amino acids in peptide ligands during protein sequencing reactions. In some embodiments, the amino acid binding proteins described herein bind to (and thereby recognize) aspartate, glutamate, and/or glutamine residues. In some embodiments, the amino acid binding proteins described herein bind to (and thereby recognize) arginine, histidine, and/or lysine residues. In some embodiments, the amino acid binding proteins described herein bind to (and thereby recognize) glycine, alanine, and/or serine residues.

Some aspects of the disclosure provide a recombinant amino acid binding protein comprising an amino acid sequence that is at least 80% identical to SEQ ID NO: 1 (PS1259), wherein the amino acid sequence comprises amino acid substitutions at positions corresponding to Q78 and one or more selected from A12, P43, K57, K65, S66, E71, E111, A122, P131, and F193 of SEQ ID NO: 1.

In some embodiments, the amino acid substitutions relative to SEQ ID NO: 1 comprise Q78R, Q78K, or Q78H. In some embodiments, the amino acid substitutions comprise A12L, A12V, or A12I. In some embodiments, the amino acid substitutions comprise K65R or K65H. In some embodiments, the amino acid substitutions comprise A122R, A122K, or A122H.

In some embodiments, the amino acid substitutions relative to SEQ ID NO: 1 comprise Q78K or Q78R and one or more substitutions selected from A12L, P43L, K57R, K65R, S66V, E71R, E111K, A122R, P131R, and F193L.

In some embodiments, the amino acid substitutions relative to SEQ ID NO: 1 comprises an amino acid substitution at one or more positions corresponding to S22, C23, S25, W30, E34, S39, C42, D46, P72, V73, I80, L81, T90, D96, K114, N120, A149, and S150 of SEQ ID NO: 1.

In some embodiments, the amino acid substitutions relative to SEQ ID NO: 1 comprise S22P. In some embodiments, the amino acid substitutions comprise C23G, C23A, C23V, C23I, or C23L. In some embodiments, the amino acid substitutions comprise S25G, S25A, S25V, S25I, or S25L. In some embodiments, the amino acid substitutions comprise V73L, V73I, or V73A. In some embodiments, the amino acid substitutions comprise D96R, D96K, or D96H. In some embodiments, the amino acid substitutions comprise K114R or K114H. In some embodiments, the amino acid substitutions comprise A149S or A149T. In some embodiments, the amino acid substitutions comprise S150R, S150K, or S150H.

In some embodiments, the amino acid substitutions relative to SEQ ID NO: 1 are selected from S22P, S22E, C23F, C23G, C23Q, S25G, W30Y, E34Q, S39Q, C42F, D46G, D46V, P72F, P72L, P72V, V73I, V73L, I80F, I80Q, L81M, T90S, D96R, K114R, N120R, A149S, A149D, A149E, S150L, and S150R.

In some embodiments, the amino acid sequence comprises amino acid substitutions at two or more positions corresponding to A12, S22, C23, S25, K65, V73, D96, K114, A122, A149, and S150 of SEQ ID NO: 1. In some embodiments, the amino acid substitutions relative to SEQ ID NO: 1 comprise Q78R and two or more substitutions selected from A12L, S22P, C23G, S25G, K65R, V73L, D96R, K114R, A122R, A149S, and S150R.

In some embodiments, the amino acid substitutions relative to SEQ ID NO: 1 comprise amino acid substitutions at positions corresponding to each of A12, S22, C23, S25, K65, V73, D96, K114, A122, A149, and S150 of SEQ ID NO: 1. In some embodiments, the amino acid substitutions relative to SEQ ID NO: 1 comprise A12L, S22P, C23G, S25G, K65R, V73L, Q78R, D96R, K114R, A122R, A149S, and S150R.

The amino acid binding protein may comprise an amino acid sequence that is at least 90%, at least 95%, at least 98%, at least 99%, 90-100%, 95-100%, 98-100%, or 100% identical to a sequence selected from any one of PS2150-2171, PS2195-2205, PS2244-2265, PS2278-2299, PS2392-2402, and PS2428-2449 (SEQ ID NOs: 3-112). In some embodiments, the amino acid sequence is at least 90%, at least 95%, at least 98%, at least 99%, 90-100%, 95-100%, 98-100%, or 100% identical to PS2195 (SEQ ID NO: 25).

In some embodiments, the amino acid substitutions relative to SEQ ID NO: 1 comprise Q78H. In some embodiments, the amino acid substitutions comprise one or more substitutions selected from A12L, K65R, S66V, E71R, and A122R.

In some embodiments, the amino acid substitutions relative to SEQ ID NO: 1 comprise an amino acid substitution at one or more positions corresponding to S5, S22, S25, V73, I80, C85, N120, K147, A149, S150, and R154. In some embodiments, the amino acid substitutions comprise one or more substitutions selected from S5C, S22E, V73I, V73L, Q78H, I80V, C85R, N120R, N120S, K147R, A149D, A149N, A149V, S150G, S150V, R154L, and R154Q.

In some embodiments, the amino acid substitutions relative to SEQ ID NO: 1 comprise S25Q. In some embodiments, the amino acid substitutions relative to SEQ ID NO: 1 comprise amino acid substitutions at positions corresponding to each of S66, S150, and R154. In some embodiments, the amino acid substitutions comprise S66V, Q78H, S150V, and R154L.

In some embodiments, the amino acid substitutions relative to SEQ ID NO: 1 comprise amino acid substitutions at positions corresponding to each of A12, S22, S66, E71, N120, A122, S150, and R154. In some embodiments, the amino acid substitutions comprise A12L, S22E, S66V, E71R, Q78H, N120R, A122R, S150V, and R154L.

The amino acid binding protein may comprise an amino acid sequence that is at least 90%, at least 95%, at least 98%, at least 99%, 90-100%, 95-100%, 98-100%, or 100% identical to a sequence selected from any one of PS2300-2314 and 2450-2472 (SEQ ID NOs: 210-247). In some embodiments, the amino acid sequence is at least 90%, at least 95%, at least 98%, at least 99%, 90-100%, 95-100%, 98-100%, or 100% identical to PS2459 (SEQ ID NO: 234).

Some aspects of the disclosure provide a recombinant amino acid binding protein comprising an amino acid sequence that is at least 80% identical to SEQ ID NO: 1 (PS1259), wherein the amino acid sequence comprises amino acid substitutions at positions corresponding to S22, C23, and S25 of SEQ ID NO: 1.

In some embodiments, the amino acid substitutions relative to SEQ ID NO: 1 comprise S22P. In some embodiments, the amino acid substitutions relative to SEQ ID NO: 1 comprise C23G, C23A, C23V, C23I, or C23L. In some embodiments, the amino acid substitutions relative to SEQ ID NO: 1 comprise S25G, S25A, S25V, S25I, or S25L. In some embodiments, the amino acid substitutions relative to SEQ ID NO: 1 are selected from S22E, S22P, C23F, C23G, C23Q, and S25G. In some embodiments, the amino acid substitutions relative to SEQ ID NO: 1 comprise C23G and/or S25G. In some embodiments, the amino acid substitutions relative to SEQ ID NO: 1 comprise S22P, C23G, and S25G.

In some embodiments, the amino acid substitutions relative to SEQ ID NO: 1 further comprise an amino acid substitution at a position corresponding to Q78 or D96 of SEQ ID NO: 1. In some embodiments, the amino acid substitution comprises Q78R, Q78K, or Q78H. In some embodiments, the amino acid substitution comprises D96R, D96K, or D96H.

In some embodiments, the amino acid sequence comprises amino acid substitutions at positions corresponding to Q78 and D96 of SEQ ID NO: 1. In some embodiments, the amino acid substitutions comprise Q78R and D96R. In some embodiments, the amino acid sequence comprises an amino acid substitution at one or more positions corresponding to A12, K65, V73, K114, A122, A149, and S150 of SEQ ID NO: 1. In some embodiments, the amino acid substitution is selected from A12L, K65R, V73L, K114R, A122R, A149S, and S150R.

In some embodiments, the amino acid sequence is at least 90%, at least 95%, at least 98%, at least 99%, 90-100%, 95-100%, 98-100%, or 100% identical to PS2195 (SEQ ID NO: 25).

Some aspects of the disclosure provide a recombinant amino acid binding protein comprising an amino acid sequence that is at least 80% identical to SEQ ID NO: 1 (PS1259), wherein the amino acid sequence comprises an arginine residue or a lysine residue at a position corresponding to D96 of SEQ ID NO: 1. In some embodiments, the amino acid sequence comprises an arginine residue at the position corresponding to D96 of SEQ ID NO: 1. In some embodiments, the amino acid sequence comprises an amino acid substitution at a position corresponding to Q78 of SEQ ID NO: 1. In some embodiments, the amino acid substitution is Q78R, Q78K, or Q78H. In some embodiments, the amino acid sequence comprises an arginine residue at one or more positions corresponding to K65, Q78, K114, A122, and S150 of SEQ ID NO: 1. In some embodiments, the amino acid sequence comprises an arginine residue at a position corresponding to Q78 and at one or more positions corresponding to K65, K114, A122, and S150 of SEQ ID NO: 1. In some embodiments, the amino acid sequence comprises an amino acid substitution at one or more positions corresponding to S22, C23, and S25 of SEQ ID NO: 1. In some embodiments, the amino acid substitution is selected from S22E, S22P, C23F, C23G, C23Q, C23A, C23V, C23I, C23L, S25G, S25A, S25V, S25I, or S25L. In some embodiments, the amino acid substitution is selected from S22E, S22P, C23F, C23G, C23Q, and S25G. In some embodiments, the amino acid sequence comprises amino acid substitutions at positions corresponding to each of S22, C23, and S25 of SEQ ID NO: 1. In some embodiments, the amino acid substitutions comprise S22P. In some embodiments, the amino acid substitutions comprise C23G and/or S25G. In some embodiments, the amino acid substitutions comprise S22P, C23G, and S25G. In some embodiments, the amino acid sequence comprises an amino acid substitution at one or more positions corresponding to A12, K65, V73, K114, A122, A149, and S150 of SEQ ID NO: 1. In some embodiments, the amino acid substitution is selected from A12L, K65R, V73L, K114R, A122R, A149S, and S150R. In some embodiments, the amino acid sequence is at least 85%, at least 90%, at least 95%, at least 98%, 80-98%, 80-95%, 80-90%, 85-95%, or 90-98% identical to SEQ ID NO: 1. In some embodiments, the amino acid sequence is at least 90%, at least 95%, at least 98%, at least 99%, 90-100%, 95-100%, 98-100%, or 100% identical to PS2195 (SEQ ID NO: 25).

Some aspects of the disclosure provide a recombinant amino acid binding protein comprising an amino acid sequence that is at least 80%, at least 85%, at least 90%, at least 95%, at least 98%, at least 99%, 90-100%, 95-100%, 98-100%, or 100% identical to a sequence selected from any one of PS2150-2171, PS2195-2205, PS2244-2265, PS2278-2299, PS2392-2402, and PS2428-2449 (SEQ ID NOs: 3-112).

Some aspects of the disclosure provide a recombinant amino acid binding protein comprising an amino acid sequence that is at least 80%, at least 85%, at least 90%, at least 95%, at least 98%, at least 99%, 90-100%, 95-100%, 98-100%, or 100% identical to a sequence selected from any one of PS2088-2089, PS2234-2243, PS2366-2379, PS2408-2409, and PS2424-2427 (SEQ ID NOs: 113-144).

Some aspects of the disclosure provide a recombinant amino acid binding protein comprising an amino acid sequence that is at least 80% identical to SEQ ID NO: 1 (PS1259), wherein the amino acid sequence comprises a glutamine residue at a position corresponding to S25 of SEQ ID NO: 1.

In some embodiments, the amino acid sequence comprises an amino acid substitution at one or more positions corresponding to S5, A12, S22, S25, K65, S66, E71, V73, Q78, I80, C85, N120, A122, K147, A149, S150, and R154 of SEQ ID NO: 1. In some embodiments, the amino acid substitution is selected from SSC, A12L, S22E, K65R, S66V, E71R, V73I, V73L, Q78H, I80V, C85R, N120R, N120S, A122R, K147R, A149D, A149N, A149V, S150G, S150V, R154L, and R154Q.

In some embodiments, the amino acid sequence comprises an amino acid substitution at one or more positions corresponding to S66, Q78, S150, and R154 of SEQ ID NO: 1. In some embodiments, the amino acid substitution is selected from S66V, Q78H, S150G, S150V, R154L, and R154Q.

In some embodiments, the amino acid sequence comprises a histidine residue at a position corresponding to Q78 of SEQ ID NO: 1. In some embodiments, the amino acid sequence comprises a valine residue at a position corresponding to S150 of SEQ ID NO: 1.

In some embodiments, the amino acid sequence comprises amino acid substitutions at positions corresponding to each of S66, Q78, 5150, and R154 of SEQ ID NO: 1. In some embodiments, the amino acid substitutions comprise S66V, Q78H, S150V, and R154L.

In some embodiments, the amino acid sequence comprises amino acid substitutions at positions corresponding to each of A12, S22, S66, E71, Q78, N120, A122, S150, and R154 of SEQ ID NO: 1. In some embodiments, the amino acid substitutions comprise A12L, S22E, S66V, E71R, Q78H, N120R, A122R, S150V, and R154L. In some embodiments, the amino acid sequence is at least 90%, at least 95%, at least 98%, at least 99%, 90-100%, 95-100%, 98-100%, or 100% identical to PS2459 (SEQ ID NO: 234).

Some aspects of the disclosure provide a recombinant amino acid binding protein comprising an amino acid sequence that is at least 80%, at least 85%, at least 90%, at least 95%, at least 98%, at least 99%, 90-100%, 95-100%, 98-100%, or 100% identical to a sequence selected from any one of PS2300-2314 and PS2450-2472 (SEQ ID NOs: 210-247).

Some aspects of the disclosure provide a recombinant amino acid binding protein comprising an amino acid sequence that is at least 80%, at least 85%, at least 90%, at least 95%, at least 98%, at least 99%, 90-100%, 95-100%, 98-100%, or 100% identical to a sequence selected from any one of PS2304, PS2305, PS2308, PS2310, PS2313, PS2451-2454, PS2457-2463, and PS2468 (SEQ ID NOs: 214-215, 218, 220, 223, 226-229, 232-238, and 243).

Some aspects of the disclosure provide a recombinant amino acid binding protein comprising an amino acid sequence that is at least 80%, at least 85%, at least 90%, at least 95%, at least 98%, at least 99%, 90-100%, 95-100%, 98-100%, or 100% identical to a sequence selected from any one of PS2604-2619 and PS2687-2702 (SEQ ID NOs: 248-279).

Some aspects of the disclosure provide a recombinant amino acid binding protein comprising an amino acid sequence that is at least 80% identical to SEQ ID NO: 2 (PS1122), wherein the amino acid sequence comprises amino acid substitutions at positions corresponding to T53 and one or more selected from K26, D32, L47, F59, and T75 of SEQ ID NO: 2.

In some embodiments, the amino acid substitutions relative to SEQ ID NO: 2 comprise T53V, T53A, T53I, or T53L. In some embodiments, the amino acid substitutions relative to SEQ ID NO: 2 comprise K26R or K26H. In some embodiments, the amino acid substitutions relative to SEQ ID NO: 2 comprise D32R, D32P, D32K, or D32H. In some embodiments, the amino acid substitutions relative to SEQ ID NO: 2 comprise L47R, L47K, or L47H. In some embodiments, the amino acid substitutions relative to SEQ ID NO: 2 comprise F59R, F59K, or F59H. In some embodiments, the amino acid substitutions relative to SEQ ID NO: 2 comprise T75D or T75E. In some embodiments, the amino acid substitutions relative to SEQ ID NO: 2 are at positions corresponding to each of L47, F59, and T75. In some embodiments, the amino acid substitutions relative to SEQ ID NO: 2 comprise L47R, T53V, F59R, and T75E.

In some embodiments, the amino acid substitutions relative to SEQ ID NO: 2 are at positions corresponding to each of K26 and D32. In some embodiments, the amino acid substitutions relative to SEQ ID NO: 2 comprise K26R and D32R or D32P.

In some embodiments, the amino acid sequence is at least 85%, at least 90%, at least 95%, at least 98%, 80-98%, 80-95%, 80-90%, 85-95%, or 90-98% identical to SEQ ID NO: 2.

In some embodiments, the amino acid sequence is at least 90%, at least 95%, at least 98%, at least 99%, 90-100%, 95-100%, 98-100%, or 100% identical to a sequence selected from any one of PS1936-PS1938 (SEQ ID NOs: 158-160). In some embodiments, the amino acid sequence is at least 90%, at least 95%, at least 98%, at least 99%, 90-100%, 95-100%, 98-100%, or 100% identical to PS1936 (SEQ ID NO: 158).

Some aspects of the disclosure provide a recombinant amino acid binding protein comprising an amino acid sequence that is at least 80%, at least 85%, at least 90%, at least 95%, at least 98%, at least 99%, 90-100%, 95-100%, 98-100%, or 100% identical to a sequence selected from any one of PS1923-PS1938, PS1659, and PS1715 (SEQ ID NOs: 145-162).

Some aspects of the disclosure provide a recombinant amino acid binding protein comprising an amino acid sequence that is at least 80%, at least 85%, at least 90%, at least 95%, at least 98%, at least 99%, 90-100%, 95-100%, 98-100%, or 100% identical to a sequence selected from any one of PS2080-2085, PS2173-2183, and PS2406-2407 (SEQ ID NOs: 163-181).

In some embodiments, an amino acid binding protein described herein further comprises one or more labels. The one or more labels may comprise a luminescent label, optionally wherein the luminescent label comprises at least one fluorophore dye molecule. The luminescent label may comprise 20 or fewer fluorophore dye molecules. In some embodiments, the luminescent label comprises at least one FRET pair comprising a donor label and an acceptor label. In some embodiments, the one or more labels comprise a tag peptide. In some embodiments, the tag peptide comprises one or more of a purification tag, a cleavage site, and a biotinylation sequence (e.g., comprising at least one biotin ligase recognition sequence or two biotin ligase recognition sequences oriented in tandem). In some embodiments, the one or more labels comprise a biotin moiety. The biotin moiety may comprise at least one biotin molecule (e.g., a bis-biotin moiety). In some embodiments, the label comprises at least one biotin ligase recognition sequence having the at least one biotin molecule attached thereto. In some embodiments, the biotin moiety is bound to a first biotin binding site of an avidin protein. In some embodiments, the avidin protein comprises a label component. In some embodiments, the label component comprises a luminescently labeled oligonucleotide comprising a second biotin moiety bound to a second biotin binding site of the avidin protein.

Some aspects of the disclosure provide an amino acid recognizer comprising: an amino acid binding protein comprising a first amino acid sequence that is at least 80% identical (e.g., at least 85%, at least 90%, at least 95%, at least 98%, 80-100%, 85-100%, 90-100%, 95-100%, or 100% identical) to a sequence selected from any one of SEQ ID NOs: 1-185 and 210-279; and a tag peptide comprising a second amino acid sequence that is at least 80% identical (e.g., at least 85%, at least 90%, at least 95%, at least 98%, 80-100%, 85-100%, 90-100%, 95-100%, or 100% identical) to a sequence selected from any one of SEQ ID NOs: 186-193 and 280.

In some embodiments, a terminal amino acid of the first amino acid sequence is attached to a terminal amino acid of the second amino acid sequence (e.g., thereby forming a fusion polypeptide comprising the amino acid binding protein and the tag peptide). In some embodiments, the terminal amino acid of the first amino acid sequence is attached (e.g., directly) to the terminal amino acid of the second amino acid sequence through a single peptide bond. In some embodiments, the terminal amino acid of the first amino acid sequence is attached to the terminal amino acid of the second amino acid sequence through a peptide linker (e.g., a linker comprising at least 2, at least 5, at least 8, at least 10, at least 15, at least 25, 2-100, 2-50, 2-30, 2-25, 5-60, 5-30, 10-50, or 20-50 amino acids). In some embodiments, the C-terminal amino acid of the first amino acid sequence is attached to the N-terminal amino acid of the second amino acid sequence. In some embodiments, the N-terminal amino acid of the first amino acid sequence is attached to the C-terminal amino acid of the second amino acid sequence.

In some embodiments, the first amino acid sequence is at least 80% identical (e.g., at least 85%, at least 90%, at least 95%, at least 98%, 80-100%, 85-100%, 90-100%, 95-100%, or 100% identical) to PS2195 (SEQ ID NO: 25). In some embodiments, the first amino acid sequence is at least 80% identical (e.g., at least 85%, at least 90%, at least 95%, at least 98%, 80-100%, 85-100%, 90-100%, 95-100%, or 100% identical) to PS2459 (SEQ ID NO: 234). In some embodiments, the first amino acid sequence is at least 80% identical (e.g., at least 85%, at least 90%, at least 95%, at least 98%, 80-100%, 85-100%, 90-100%, 95-100%, or 100% identical) to PS1936 (SEQ ID NO: 158).

In some embodiments, the first amino acid sequence is at least 80% identical (e.g., at least 85%, at least 90%, at least 95%, at least 98%, 80-100%, 85-100%, 90-100%, 95-100%, or 100% identical) to PS2459 (SEQ ID NO: 234), and the second amino acid sequence is at least 80% identical (e.g., at least 85%, at least 90%, at least 95%, at least 98%, 80-100%, 85-100%, 90-100%, 95-100%, or 100% identical) to SEQ ID NO: 280. In some embodiments, the first amino acid sequence comprises PS2459 (SEQ ID NO: 234), and the second amino acid sequence comprises SEQ ID NO: 280.

Other aspects of the disclosure provide a kit comprising one or more amino acid recognizers, wherein at least one amino acid recognizer comprises an amino acid binding protein or an amino acid recognizer as described herein. In some embodiments, a kit comprises an amino acid recognizer comprising a ClpS protein, a UBR protein, an Ntaq1 protein, a BIR3 domain protein, or a homolog or variant thereof. In some embodiments, a kit comprises one or more amino acid recognizers comprising an amino acid binding protein that comprises an amino acid sequence that is at least 80%, at least 85%, at least 90%, at least 95%, at least 98%, at least 99%, 90-100%, 95-100%, 98-100%, or 100% identical to a sequence selected from Table 1. In some embodiments, a kit further comprises one or more cleaving reagents (e.g., an aminopeptidase). In some embodiments, a kit further comprises instructions for using the kit in a method of polypeptide analysis.

Some aspects of the disclosure provide a composition comprising two or more amino acid recognizers, wherein at least one amino acid recognizer is an amino acid binding protein or an amino acid recognizer as described herein. In some embodiments, the composition comprises one or more cleaving reagents (e.g., an aminopeptidase).

Some aspects of the disclosure provide a method of determining at least one chemical characteristic of a polypeptide, the method comprising: contacting a polypeptide with an amino acid binding protein, an amino acid recognizer, or a composition as described herein; monitoring a signal for signal pulses corresponding to interactions between one or more amino acid recognizers and the polypeptide; and determining at least one chemical characteristic of the polypeptide based on a characteristic pattern in the signal.

The details of certain embodiments of the disclosure are set forth in the Detailed Description. Other features, objects, and advantages of the disclosure will be apparent from the Examples, Drawings, and Claims.

### BRIEF DESCRIPTION OF THE DRAWINGS

The accompanying Drawings, which constitute a part of this specification, illustrate several embodiments of the disclosure and together with the accompanying description, serve to explain the principles of the disclosure.

FIG. 1A shows an example overview of real-time dynamic protein sequencing. Protein samples are digested into peptide fragments, immobilized in nanoscale reaction chambers, and incubated with a mixture of freely diffusing N-terminal amino acid (NAA) recognizers and aminopeptidases that carry out the sequencing process. The labeled recognizers bind on and off to the peptide when one of their cognate NAAs is exposed at the N-terminus, thereby producing characteristic pulsing patterns. The NAA is cleaved by an aminopeptidase, exposing the next amino acid for recognition. The temporal order of NAA recognition and the kinetics of binding enable peptide identification and are sensitive to features that modulate binding kinetics, such as post-translational modifications (PTMs).

FIG. 1B shows an example schematic of a pixel of an integrated device.

FIGS. 2A-2C show example Octet binding analysis results from the design of Ntaq1-homologous variant recognizers. FIG. 2A shows the improved binding ability of PS2195 (an Ntaq-1 homologous variant) for aspartic acid-containing peptides (DA peptides) relative to a control Ntaq1-homologous variant. FIG. 2B shows the improved binding ability of PS2195 (an Ntaq-1 homologous variant) for glutamic acid-containing peptides (EA peptides) relative to a control Ntaq1-homologous variant. FIG. 2C shows the reduced binding ability of PS2195 (an Ntaq-1 homologous variant) for glutamine-containing peptides (QA peptides) relative to a control Ntaq1-homologous variant.

FIGS. 3A-3F show example single point fluorescent polarization binding analysis results from the development of aspartate/glutamate recognizers. FIG. 3A shows binding assay data (polarization response) for Ntaq1-homologous variants (2 μM) binding to glutamic acid-containing peptides (EA peptides). FIG. 3B shows binding assay data (polarization response) for Ntaq1-homologous variants (2 μM) binding to aspartic acid-containing peptides (DA peptides). FIG. 3C shows binding assay data (polarization response) for Ntaq1-homologous variants (2 μM) background binding to glutamine-containing peptides (QA peptides). FIG. 3D shows binding assay data (polarization response) for Ntaq1-homologous variants (2 μM) background binding to asparagine-containing peptides (NA peptides). FIG. 3E shows binding assay data (polarization response) for Ntaq1-homologous variants (0.5 μM) binding to glutamine-containing peptides (QA peptides). FIG. 3F shows binding assay data (polarization response) for Ntaq1-homologous variants (0.5 μM) background binding to asparagine-containing peptides (NA peptides).

FIG. 4 shows an example of multiplexed dynamic chip analysis using multiple recognizers including a control Ntaq1-homologous variant (PS2132) that demonstrated lack of aspartate recognition. Dotted box indicates location of aspartate in the CDNF protein library peptide sequence.

FIG. 5 shows an example of multiplexed dynamic chip analysis using multiple recognizers including a novel Ntaq1-homologous variant (PS2195) that demonstrates aspartate recognition. Dotted box indicates location of aspartate in the CDNF protein library peptide sequence.

FIG. 6 shows an example of multiplexed dynamic chip analysis using multiple recognizers including a control Ntaq1-homologous variant (PS2132) that demonstrates glutamate recognition. Dotted box indicates location of glutamate in the CDNF protein library peptide sequence.

FIG. 7 shows an example of multiplexed dynamic chip analysis using multiple recognizers including a novel Ntaq1-homologous variant (PS2195) that demonstrates improved glutamate recognition. Dotted box indicates location of glutamate in the CDNF protein library peptide sequence.

FIGS. 8A-8F show examples of the structural images of PS2195 based on experimentally determined crystal structures. FIG. 8A shows the binding pocket of PS2195 when complexed to a DAKLDEESILKQ (SEQ ID NO: 281) peptide. FIG. 8B shows residues in the binding pocket of PS2195 that interact with the aspartate side chain of a DAKL (SEQ ID NO: 282) peptide. FIG. 8C shows the recognition sites of PS2195 complexed to a glutamic acid-containing peptide. FIG. 8D shows the recognition sites of PS2195 complexed to an aspartic acid-containing peptide. FIG. 8E shows a full image of the crystal structure of PS2195 bound to a DAKL (SEQ ID NO: 282) peptide, with a disulfide linkage formed between C42 and C85 highlighted. FIG. 8F shows the disulfide linkage formed between C42 and C85 in PS2195, resulting in an alternate conformation of the H83-T90 loop, relative to PS1259.

FIGS. 9A-9C show example results from the development of arginine recognizers. FIG. 9A shows fluorescence polarization response for UBR variants (100 nM) binding to arginine-containing peptides (RA peptides). FIG. 9B shows fluorescence polarization response for UBR variants (2 μM) binding to histidine-containing peptides (HA peptides). FIG. 9C shows fluorescence polarization response for UBR variants (2 μM) binding to lysine-containing peptides (KA peptides). Asterisks indicate control UBR variants.

FIG. 10 shows an example of multiplexed dynamic chip analysis using a combination of recognizers (including a UBR variant) to demonstrate improved pulse width of PS1936 relative to PS1122 and uniformity in pulse width relative to an example control variant (PS1381).

FIG. 11 shows an example of multiplexed dynamic chip analysis using multiple recognizers including a control UBR that demonstrates arginine recognition (PS1122). Dotted box indicates location of arginine in the CDNF protein library peptide sequence.

FIG. 12 shows an example of multiplexed dynamic chip analysis of using multiple recognizers including a novel UBR variant (PS1936) that demonstrates improved arginine recognition performance and faster pulsing. Dotted box indicates location of arginine in the CDNF protein library peptide sequence.

FIGS. 13A-13B show example results of the crystal structure of PS1122 and the structure-based modeling of PS1936. FIG. 13A shows a full image of the crystal structure of PS1122 bound to a RAKL (SEQ ID NO: 288) peptide. FIG. 13B shows an image of a model of PS1936 complexed with a RAKL (SEQ ID NO: 288) peptide based on PS1122 crystal structure.

FIG. 14 shows SDS PAGE gel showing a HTP protein batch of Ntaq variants proteins conjugated with streptavidin.

FIGS. 15A-15C show example results from Octet binding assay for the design of PS1259 variant recognizers. FIG. 15A shows the improved binding ability of PS1259 variants, including PS2308, PS2313, and PS2310, for N-terminal glycine-containing peptides (GA peptides) relative to PS1259. FIG. 15B shows the binding ability of PS1259 variants for N-terminal serine-containing peptides (SA peptides). FIG. 15C shows the binding ability of PS1259 variants for N-terminal glutamine-containing peptides (QA peptides).

FIGS. 16A-16D show example results from single point fluorescent polarization binding assays for the development of PS1259 variant recognizers. FIG. 16A shows binding assay data (polarization response) for PS1259 variants (2 μM) binding to N-terminal glutamine-containing peptides (QA peptides). FIG. 16B shows binding assay data (polarization response) for PS1259 variants (2 μM) binding to N-terminal glycine-containing peptides (GA peptides). FIG. 16C shows binding assay data (polarization response) for PS1259 variants (2 μM) binding to N-terminal asparagine-containing peptides (NA peptides). FIG. 16D shows binding assay data (polarization response) for PS1259 variants (2 μM) binding to N-terminal serine-containing peptides (SA peptides).

FIG. 17 shows combined binding assay data (fluorescence polarization response) for PS1259 variants (2 μM) binding to N-terminal glutamine-containing peptides (QA peptides), N-terminal asparagine-containing peptides (NA peptides), N-terminal glycine-containing peptides (GA peptides), and N-terminal serine-containing peptides (SA peptides).

FIG. 18 shows binding assay data (fluorescence polarization response) for PS1259 variants (PS2308, PS2310, and PS2313) with peptides having different N-terminal dipeptide sequences. Data is shown for N-terminal benzyl-cysteine peptide (“CysBenzyl”) and other peptides having N-terminal dipeptide sequences labeled according to the N-terminal (position 1) and penultimate (position 2) residues (e.g., “DA” refers to a peptide having aspartate (D) at the N-terminal position (position 1) and alanine (A) at the penultimate position (position 2)).

FIG. 19 shows example results from dynamic chip analysis polypeptide sequencing reactions using multiple recognizers including a novel Ntaq1-homologous variant (PS2310) that demonstrates glycine as well as alanine recognition on a synthetic peptide.

FIGS. 20A-20H show example results from single point polarization binding assays for the development of PS1259 variant recognizers. FIG. 20A shows binding assay data (florescence polarization response) for PS1259 variants (2 μM) binding to N-terminal glycine-containing peptides (GA peptides). FIG. 20B shows binding assay data (florescence polarization response) for PS1259 variants (2 μM) binding to N-terminal serine-containing peptides (SA peptides). FIG. 20C shows binding assay data (florescence polarization response) for PS1259 variants (2 μM) binding to N-terminal glutamine-containing peptides (QA peptides). FIG. 20D shows binding assay data (florescence polarization response) for PS1259 variants (2 μM) binding to N-terminal threonine-containing peptides (TA peptides). FIG. 20E shows binding assay data (florescence polarization response) for PS1259 variants (2 μM) binding to N-terminal alanine-containing peptides (AA peptides). FIG. 20F shows binding assay data (florescence polarization response) for PS1259 variants (2 μM) binding to N-terminal methionine-containing peptides (MA peptides). FIG. 20G shows binding assay data (florescence polarization response) for PS1259 variants (2 μM) binding to N-terminal asparagine-containing peptides (NA peptides). FIG. 20H shows binding assay data (florescence polarization response) for PS1259 variants (2 μM) binding to N-terminal valine-containing peptides (VA peptides).

FIG. 21 shows binding assay data (fluorescence polarization response) for PS1259 variants (2 μM) binding to N-terminal glutamine-containing peptides (QA peptides), N-terminal glycine-containing peptides (GA peptides), and N-terminal serine-containing peptides (SA peptides).

FIG. 22 shows combined binding assay data (fluorescence polarization response) for PS1259 variants (2 μM) binding to N-terminal methionine-containing peptides (MA peptides), N-terminal asparagine-containing peptides (NA peptides), N-terminal valine-containing peptides (VA peptides), N-terminal threonine-containing peptides (TA peptides), and N-terminal alanine-containing peptides (AA peptides).

FIGS. 23A-23C show example Octet binding analysis results from the design of PS1259 variant recognizers. FIG. 23A shows the improved binding ability of PS2457 and PS2459 (PS1259 variants) for N-terminal glycine-containing peptides (GA peptides) relative to PS1259. FIG. 23B shows the improved binding ability of PS2457 and PS2459 (PS1259 variants) for N-terminal serine-containing peptides (SA peptides) relative to PS1259. FIG. 23C shows the decreased binding ability of PS2457 and PS2459 (PS1259 variants) for N-terminal glutamine-containing peptides (QA peptides) relative to PS1259.

FIGS. 24A-24D show binding assay data (fluorescence polarization response) for PS1259 variants PS2453 (FIG. 24A), PS2463 (FIG. 24B), PS2457 (FIG. 24C), and PS2459 (FIG. 24D) with peptides having different N-terminal dipeptide sequences. Labeling of dipeptide sequences is as described for FIG. 18.

FIGS. 25A-25F show example results from polypeptide sequencing reactions using multiple recognizers, including a novel PS1259 variant (PS2459) that demonstrates glycine, alanine, and serine recognition. A library mixture of human proteins comprising CDNF, PDL1, MAPK3, NGAL, IL18R, IL20, LMNB1, SFN, RAB11B, and VIME peptides were sequenced with a mixture of recognizers (PS610, PS1936, PS2225, PS1751, and PS2195) in addition to a novel PS1259 variant (“PS2459”) compared to “Control” (a recognizer mixture comprising of PS1587 the tandem BIR A/S recognizer, PS610, PS1936, PS2225, PS1751, and PS2195). FIGS. 25A-25B show serine and glycine recognition in a CDNF library peptide by PS2459. FIGS. 25C-25D show alanine recognition in a MAPK3 library peptide by PS2459. FIGS. 25E-25F show serine recognition in a RAB11B library peptide by PS2459.

FIGS. 26A-26E show sequencing statistics from polypeptide sequencing reactions as described for FIGS. 25A-25F. FIG. 26A shows a ratio of alignments to CDNF, IL18R, IL20, LMNB1, MAPK3, NGAL, PDL1, RAB11B, SFN, and VIME library peptides using a mixture of recognizers comprising PS2459 compared to “Control” (a recognizer mixture comprising PS1587 (tandem BIR A/S recognizer), PS610, PS1936, PS2225, PS1751, and PS2195). FIG. 26B shows a ratio of alignments to identified peptides using a mixture of recognizers comprising PS2459 compared to “Control” (a recognizer mixture comprising PS1587 (tandem BIR A/S recognizer), PS610, PS1936, PS2225, PS1751, and PS2195). FIG. 26C shows recognition sequence duration (top left), pulse duration (top right), number of pulses (bottom left), and interpulse duration (bottom right) using a mixture of recognizers comprising PS2459 compared to “Control” (a recognizer mixture comprising PS1587 (tandem BIR A/S recognizer), PS610, PS1936, PS2225, PS1751, and PS2195). FIG. 26D shows recognition sequence duration (top left), pulse duration (top right), number of pulses (bottom left), and interpulse duration (bottom right) using a mixture of recognizers comprising PS2459. FIG. 26E shows recognition site duration (top left), pulse duration (top right), number of pulses (bottom left), and interpulse duration (bottom right) using “Control” (a recognizer mixture comprising PS1587 (tandem BIR A/S recognizer), PS610, PS1936, PS2225, PS1751, and PS2195).

FIG. 27 shows a model image of a PS1259/glutamine complex crystal structure superimposed with a PS2457/glycine complex crystal structure.

FIG. 28 shows an image of the sidechain recognition of PS2457 complexed with glycine (white), alanine (green), and serine (blue) derived from the superposition of their respective determined crystal structure.

FIG. 29 shows an image of PS2457 experimentally determined crystal structure (white) superimposed with PS2459 experimentally determined crystal structure (green) bound to a glycine peptide.

### DETAILED DESCRIPTION

Aspects of the disclosure relate to compositions and methods for determining chemical characteristics of a polypeptide based on single-molecule binding interactions between the polypeptide and one or more reagents described herein. In some embodiments, the disclosure provides amino acid recognizers having improved performance in polypeptide sequencing reactions. In some embodiments, the disclosure provides an approach for polypeptide structure analysis based on kinetic information derived from single-molecule binding interactions between a polypeptide and one or more amino acid recognizers described herein.

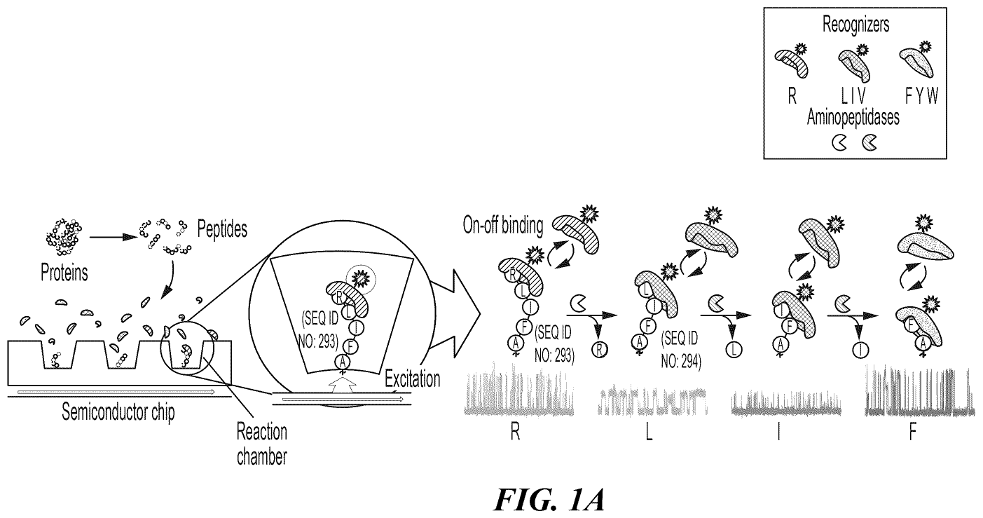

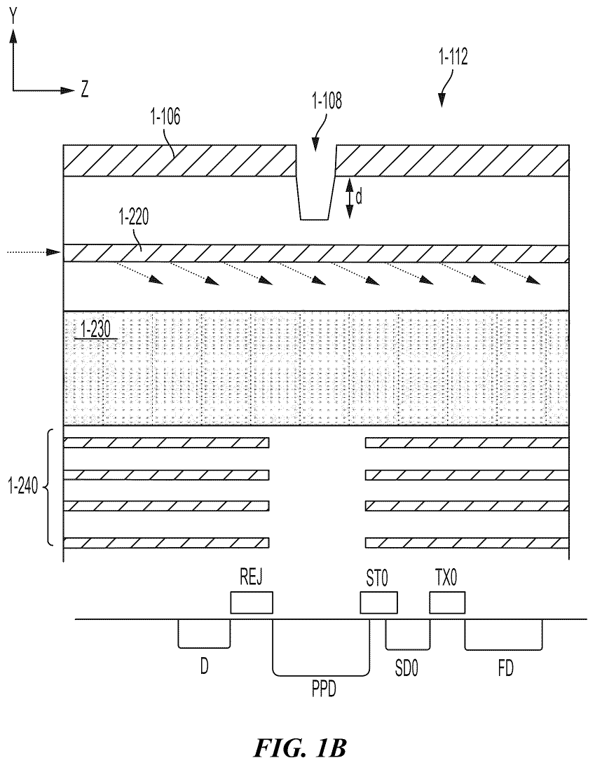

FIG. 1A shows an example of a dynamic peptide sequencing reaction in which individual on-off binding events give rise to signal pulses of a signal output. As shown at left, a protein sample may be fragmented into peptides, which are immobilized in reaction chambers of an array, where the immobilized peptides are exposed to one or more amino acid recognizers and one or more cleaving reagents (e.g., aminopeptidases). As shown at right, amino acid recognizers reversibly bind to the peptide, producing a series of changes in signal output (e.g., signal pulses) as amino acids are progressively cleaved from the peptide terminus. The temporal order of recognition and the kinetics of binding and/or cleaving can be used to determine structural information for the peptide.

Compositions and methods for performing dynamic polypeptide sequencing and analyzing data obtained therefrom are described more fully in PCT International Publication No. WO2020102741A1, filed Nov. 15, 2019, PCT International Publication No. WO2021236983A2, filed May 20, 2021, PCT International Publication No. WO2023122769A2, filed Dec. 22, 2022, PCT International Publication No. WO2024031031A2, filed Aug. 3, 2023, and PCT International Publication No. WO2024086832A1, filed Oct. 20, 2023, each of which is incorporated by reference in its entirety.

In some aspects, the disclosure provides amino acid recognizers with improved binding properties, which allow for more structural information to be obtained from polypeptides based on the kinetics of on-off binding between recognizer and polypeptide. In some embodiments, an amino acid recognizer comprises an amino acid binding protein with an engineered binding pocket having one or more modifications relative to a homologous protein. In some embodiments, the modified binding pocket increases the number of interactions (e.g., hydrogen bonding interactions, van der Waals interactions) formed between the binding pocket and an amino acid ligand as compared to an unmodified binding pocket of the homologous protein. In some embodiments, the modified binding pocket increases the number of types of amino acid ligands capable of being detectably bound as compared to an unmodified binding pocket of the homologous protein. In some embodiments, the modified binding pocket improves the kinetics of binding (e.g., K_{D}, k_{off}, kₒₙ) toward one or more types of amino acid ligands, which advantageously increases the amount of, or confidence in, structural information that may be derived from polypeptide analysis as described herein.

### I. Amino Acid Recognizers

In some aspects, the disclosure provides an amino acid recognizer comprising an amino acid binding protein having an amino acid sequence selected from Table 1. Table 1 herein provides a list of example sequences of amino acid binding proteins. It should be appreciated that these sequences and other examples described herein are meant to be non-limiting, and amino acid recognizers in accordance with the disclosure can include any homologs, variants, or fragments thereof minimally containing domains or subdomains responsible for amino acid recognition.

In some embodiments, the disclosure provides an amino acid binding protein having an amino acid sequence that is at least 80% identical to an amino acid sequence selected from Table 1. In some embodiments, an amino acid binding protein has at least 40%, at least 50%, at least 60%, at least 70%, at least 80%, at least 85%, at least 90%, at least 95%, at least 98%, or higher, amino acid sequence identity to an amino acid sequence selected from Table 1. In some embodiments, an amino acid binding protein has 25-50%, 50-60%, 60-70%, 70-80%, 80-90%, 90-95%, 95-99%, 40-100%, 50-100%, 60-100%, 70-100%, 80-100%, 90-100%, or 95-100% amino acid sequence identity to an amino acid sequence selected from Table 1. In some embodiments, the amino acid binding protein further comprises a tag sequence that provides one or more functions other than amino acid binding. For example, in some embodiments, an amino acid binding protein having an amino acid sequence that is at least 80% identical to a sequence selected from Table 1 is fused (e.g., at its N- or C-terminus) to a tag peptide having an amino acid sequence that is at least 80% identical to a sequence selected from Table 2A.

In some embodiments, an amino acid recognizer of the disclosure comprises a modified amino acid binding protein and includes one or more amino acid deletions, additions, or mutations relative to a sequence set forth in Table 1. In some embodiments, a modified amino acid binding protein includes a deletion, addition, or mutation of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acids (which may or may not be consecutive amino acids) relative to a sequence set forth in Table 1.

### A. Ntaq1-Homologous Recognizers

In some embodiments, an amino acid recognizer of the disclosure binds an amino acid ligand (e.g., a polypeptide) comprising an N-terminal amino acid selected from glutamine, asparagine, glutamate, aspartate, cysteine-S-acetamide, or a modified variant thereof (e.g., a post-translationally modified variant thereof, an oxidized variant thereof). In some embodiments, an amino acid recognizer of the disclosure binds an amino acid ligand (e.g., a polypeptide) comprising an N-terminal amino acid selected from glycine, alanine, serine, or a modified variant thereof (e.g., a post-translationally modified variant thereof, an oxidized variant thereof). In some embodiments, the amino acid recognizer comprises an amino acid binding protein derived from a variant of an Ntaq1 protein, such as Scleropages formosus Ntaq1 protein. For example, in some embodiments, the amino acid binding protein is an engineered variant comprising one or more modifications relative to an Ntaq1 protein variant referred to herein as “PS1259” (SEQ ID NO: 1).

In some embodiments, the amino acid binding protein binds glutamine (e.g., N-terminal glutamine) with a dissociation constant (K_{D}) of less than 3,000 nM, 2,500 nM, 2,000 nM, less than 1,500 nM, less than 1,000 nM, less than 750 nM, less than 500 nM, less than 250 nM, less than 150 nM, less than 100 nM, less than 50 nM, 50-3,000 nM, 50-2,500 nM, 100-3,000 nM, 10-2,000 nM, 25-1,000 nM, 50-500 nM, 10-100 nM, 25-250 nM, or 50-150 nM. In some embodiments, the amino acid binding protein binds glutamate (e.g., N-terminal glutamate) with a dissociation constant (K_{D}) of less than 2,000 nM, less than 1,500 nM, less than 1,000 nM, less than 750 nM, less than 500 nM, less than 250 nM, less than 150 nM, less than 100 nM, less than 50 nM, 10-2,000 nM, 25-1,000 nM, 50-500 nM, 10-100 nM, 25-250 nM, or 50-150 nM. In some embodiments, the amino acid binding protein binds aspartate (e.g., N-terminal aspartate) with a dissociation constant (K_{D}) of less than 2,000 nM, less than 1,500 nM, less than 1,000 nM, less than 750 nM, less than 500 nM, less than 250 nM, less than 150 nM, less than 100 nM, less than 50 nM, 10-2,000 nM, 25-1,000 nM, 50-500 nM, 10-100 nM, 25-250 nM, or 50-150 nM.

In some embodiments, the amino acid binding protein binds one or more types of N-terminal amino acids (e.g., glutamine, asparagine, glutamate, aspartate, cysteine-S-acetamide, or a modified variant thereof), where each type of binding interaction is characterized by a dissociation rate (k_{off}) of at least 0.1 s⁻¹. In some embodiments, the dissociation rate is between about 0.1 s⁻¹ and about 1,000 s⁻¹ (e.g., between about 0.5 s⁻¹ and about 500 s⁻¹, between about 0.1 s⁻¹ and about 100 s⁻¹, between about 1 s⁻¹ and about 100 s⁻¹, between about 5 s⁻¹ and about 50 s⁻¹, between about 10 s⁻¹ and about 40 s⁻¹, or between about 0.5 s⁻¹ and about 50 s⁻¹). In some embodiments, the dissociation rate is between about 0.5 s⁻¹ and about 20 s⁻¹. In some embodiments, the dissociation rate is between about 2 s⁻¹ and about 20 s⁻¹. In some embodiments, the dissociation rate is between about 0.5 s⁻¹ and about 2 s⁻¹

In some embodiments, the amino acid binding protein binds glycine (e.g., N-terminal glycine) with a dissociation constant (K_{D}) of less than 2,000 nM, less than 1,500 nM, less than 1,000 nM, less than 750 nM, less than 500 nM, less than 250 nM, less than 150 nM, less than 100 nM, less than 50 nM, 10-2,000 nM, 25-1,000 nM, 50-500 nM, 10-100 nM, 25-250 nM, or 50-150 nM. In some embodiments, the amino acid binding protein binds alanine (e.g., N-terminal alanine) with a dissociation constant (K_{D}) of less than 2,000 nM, less than 1,500 nM, less than 1,000 nM, less than 750 nM, less than 500 nM, less than 250 nM, less than 150 nM, less than 100 nM, less than 50 nM, 10-2,000 nM, 25-1,000 nM, 50-500 nM, 10-100 nM, 25-250 nM, or 50-150 nM. In some embodiments, the amino acid binding protein binds serine (e.g., N-terminal serine) with a dissociation constant (K_{D}) of less than 2,000 nM, less than 1,500 nM, less than 1,000 nM, less than 750 nM, less than 500 nM, less than 250 nM, less than 150 nM, less than 100 nM, less than 50 nM, 10-2,000 nM, 25-1,000 nM, 50-500 nM, 10-100 nM, 25-250 nM, or 50-150 nM.

In some embodiments, the amino acid binding protein binds one or more types of N-terminal amino acids (e.g., glycine, serine, alanine, or a modified variant thereof), where each type of binding interaction is characterized by a dissociation rate (k_{off}) of at least 0.1 s⁻¹. In some embodiments, the dissociation rate is between about 0.1 s⁻¹ and about 1,000 s⁻¹ (e.g., between about 0.5 s⁻¹ and about 500 s⁻¹, between about 0.1 s⁻¹ and about 100 s⁻¹, between about 1 s⁻¹ and about 100 s⁻¹, between about 5 s⁻¹ and about 50 s⁻¹, between about 10 s⁻¹ and about 40 s⁻¹, or between about 0.5 s⁻¹ and about 50 s⁻¹). In some embodiments, the dissociation rate is between about 0.5 s⁻¹ and about 20 s⁻¹. In some embodiments, the dissociation rate is between about 2 s⁻¹ and about 20 s⁻¹. In some embodiments, the dissociation rate is between about 0.5 s⁻¹ and about 2 s⁻¹

In some aspects, the disclosure provides a recombinant or synthetic amino acid binding protein having an amino acid sequence that is at least 80% identical (e.g., at least 85%, at least 90%, at least 95%, at least 98%, 80-98%, 80-95%, 80-90%, 85-95%, or 90-98% identical) to SEQ ID NO: 1, where the amino acid sequence comprises amino acid substitutions at positions corresponding to Q78 and one or more selected from A12, P43, K57, K65, S66, E71, E111, A122, P131, and F193 of SEQ ID NO: 1. In some embodiments, the amino acid sequence is at least 85%, at least 90%, at least 95%, at least 98%, 80-98%, 80-95%, 80-90%, 85-90%, 85-95%, 90-95%, or 90-98% identical to SEQ ID NO: 1.

In some embodiments, the amino acid substitutions comprise Q78R, Q78K, or Q78H. In some embodiments, the amino acid substitutions comprise A12L, A12V, or A12I. In some embodiments, the amino acid substitutions comprise K65R or K65H. In some embodiments, the amino acid substitutions comprise A122R, A122K, or A122H. In some embodiments, the amino acid substitutions comprise Q78K or Q78R and one or more substitutions selected from A12L, P43L, K57R, K65R, S66V, E71R, E111K, A122R, P131R, and F193L.

In some embodiments, the amino acid sequence comprises an amino acid substitution at one or more positions corresponding to S22, C23, S25, W30, E34, 539, C42, D46, P72, V73, 180, L81, T90, D96, K114, N120, A149, and 5150 of SEQ ID NO: 1. In some embodiments, the amino acid substitutions comprise S22P. In some embodiments, the amino acid substitutions comprise C23G, C23A, C23V, C23I, or C23L. In some embodiments, the amino acid substitutions comprise S25G, S25A, S25V, S25I, or S25L. In some embodiments, the amino acid substitutions comprise V73L, V73I, or V73A. In some embodiments, the amino acid substitutions comprise D96R, D96K, or D96H. In some embodiments, the amino acid substitutions comprise K114R or K114H. In some embodiments, the amino acid substitutions comprise A149S or A149T. In some embodiments, the amino acid substitutions comprise S150R, S150K, or S150H. In some embodiments, the amino acid substitution is selected from S22P, S22E, C23F, C23G, C23Q, S25G, W30Y, E34Q, S39Q, C42F, D46G, D46V, P72F, P72L, P72V, V73I, V73L, I80F, I80Q, L81M, T90S, D96R, K114R, N120R, A149S, A149D, A149E, S150L, and S150R.

In some embodiments, the amino acid sequence comprises amino acid substitutions at two or more positions corresponding to A12, S22, C23, S25, K65, V73, D96, K114, A122, A149, and S150 of SEQ ID NO: 1. In some embodiments, the amino acid substitutions comprise Q78R and two or more substitutions selected from A12L, S22P, C23G, S25G, K65R, V73L, D96R, K114R, A122R, A149S, and S150R. In some embodiments, the amino acid sequence comprises amino acid substitutions at positions corresponding to each of A12, S22, C23, S25, K65, V73, D96, K114, A122, A149, and S150 of SEQ ID NO: 1. In some embodiments, the amino acid substitutions comprise A12L, S22P, C23G, S25G, K65R, V73L, Q78R, D96R, K114R, A122R, A149S, and S150R.

In some embodiments, the amino acid sequence is at least 80%, at least 85%, at least 90%, at least 95%, at least 98%, at least 99%, 90-100%, 95-100%, 98-100%, or 100% identical to a sequence selected from any one of PS2150-2171, PS2195-2205, PS2244-2265, PS2278-2299, PS2392-2402, and PS2428-2449 (SEQ ID NOs: 3-112). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to a sequence selected from any one of PS2150-2171, PS2195-2205, PS2244-2265, PS2278-2299, PS2392-2402, and PS2428-2449 (SEQ ID NOs: 3-112). In some embodiments, the amino acid sequence is at least 80%, at least 85%, at least 90%, at least 95%, at least 98%, at least 99%, 90-100%, 95-100%, 98-100%, or 100% identical to PS2195 (SEQ ID NO: 25). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to PS2195 (SEQ ID NO: 25).

In some embodiments, the amino acid substitutions comprise Q78H. In some embodiments, the amino acid substitutions comprise one or more substitutions selected from A12L, K65R, S66V, E71R, and A122R. In some embodiments, the amino acid sequence comprises an amino acid substitution at one or more positions corresponding to S5, S22, S25, V73, I80, C85, N120, K147, A149, S150, and R154 of SEQ ID NO: 1. In some embodiments, the amino acid substitutions comprise one or more substitutions selected from S5C, S22E, V73I, V73L, Q78H, I80V, C85R, N120R, N120S, K147R, A149D, A149N, A149V, S150G, S150V, R154L, and R154Q. In some embodiments, the amino acid substitutions comprise S25Q.

In some embodiments, the amino acid sequence comprises amino acid substitutions at positions corresponding to each of S66, S150, and R154 of SEQ ID NO: 1. In some embodiments, the amino acid substitutions comprise S66V, Q78H, S150V, and R154L. In some embodiments, the amino acid sequence comprises amino acid substitutions at positions corresponding to each of A12, S22, S66, E71, N120, A122, S150, and R154 of SEQ ID NO: 1. In some embodiments, wherein the amino acid substitutions comprise A12L, S22E, S66V, E71R, Q78H, N120R, A122R, S150V, and R154L.

In some embodiments, the amino acid sequence is at least 80%, at least 85%, at least 90%, at least 95%, at least 98%, at least 99%, 90-100%, 95-100%, 98-100%, or 100% identical to PS2459 (SEQ ID NO: 234). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to PS2459 (SEQ ID NO: 234).

In some aspects, the disclosure provides a recombinant or synthetic amino acid binding protein having an amino acid sequence that is at least 80% identical (e.g., at least 85%, at least 90%, at least 95%, at least 98%, 80-98%, 80-95%, 80-90%, 85-95%, or 90-98% identical) to SEQ ID NO: 1, where the amino acid sequence comprises amino acid substitutions at positions corresponding to S22, C23, and S25 of SEQ ID NO: 1. In some embodiments, the amino acid sequence is at least 85%, at least 90%, at least 95%, at least 98%, 80-98%, 80-95%, 80-90%, 85-90%, 85-95%, 90-95%, or 90-98% identical to SEQ ID NO: 1.

In some embodiments, the amino acid substitutions comprise S22P. In some embodiments, the amino acid substitutions comprise C23G, C23A, C23V, C23I, or C23L. In some embodiments, the amino acid substitutions comprise S25G, S25A, S25V, S25I, or S25L. In some embodiments, the amino acid substitutions are selected from S22E, S22P, C23F, C23G, C23Q, and S25G. In some embodiments, the amino acid substitutions comprise C23G and/or S25G. In some embodiments, the amino acid substitutions comprise S22P, C23G, and S25G.

In some embodiments, the amino acid sequence comprises an amino acid substitution at a position corresponding to Q78 or D96 of SEQ ID NO: 1. In some embodiments, the amino acid substitution comprises Q78R, Q78K, or Q78H. In some embodiments, the amino acid substitution comprises D96R, D96K, or D96H. In some embodiments, the amino acid sequence comprises amino acid substitutions at positions corresponding to Q78 and D96 of SEQ ID NO: 1. In some embodiments, the amino acid substitutions comprise Q78R and D96R.

In some embodiments, the amino acid sequence comprises an amino acid substitution at one or more positions corresponding to A12, K65, V73, K114, A122, A149, and S150 of SEQ ID NO: 1. In some embodiments, the amino acid substitution is selected from A12L, K65R, V73L, K114R, A122R, A149S, and S150R.

In some embodiments, the amino acid sequence is at least 80%, at least 85%, at least 90%, at least 95%, at least 98%, at least 99%, 90-100%, 95-100%, 98-100%, or 100% identical to a sequence selected from any one of PS2150-2171, PS2195-2205, PS2244-2265, PS2278-2299, PS2392-2402, and PS2428-2449 (SEQ ID NOs: 3-112). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to a sequence selected from any one of PS2150-2171, PS2195-2205, PS2244-2265, PS2278-2299, PS2392-2402, and PS2428-2449 (SEQ ID NOs: 3-112). In some embodiments, the amino acid sequence is at least 80%, at least 85%, at least 90%, at least 95%, at least 98%, at least 99%, 90-100%, 95-100%, 98-100%, or 100% identical to PS2195 (SEQ ID NO: 25). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to PS2195 (SEQ ID NO: 25).

In some aspects, the disclosure provides a recombinant or synthetic amino acid binding protein having an amino acid sequence that is at least 80% identical (e.g., at least 85%, at least 90%, at least 95%, at least 98%, 80-98%, 80-95%, 80-90%, 85-95%, or 90-98% identical) to SEQ ID NO: 1, where the amino acid sequence comprises an arginine residue or a lysine residue at a position corresponding to D96 of SEQ ID NO: 1. In some embodiments, the amino acid sequence is at least 85%, at least 90%, at least 95%, at least 98%, 80-98%, 80-95%, 80-90%, 85-90%, 85-95%, 90-95%, or 90-98% identical to SEQ ID NO: 1.

In some embodiments, the amino acid sequence comprises an arginine residue at the position corresponding to D96 of SEQ ID NO: 1. In some embodiments, the amino acid sequence comprises an amino acid substitution at a position corresponding to Q78 of SEQ ID NO: 1. In some embodiments, the amino acid substitution is Q78R, Q78K, or Q78H.

In some embodiments, the amino acid sequence comprises an arginine residue at one or more positions corresponding to K65, Q78, K114, A122, and S150 of SEQ ID NO: 1. In some embodiments, the amino acid sequence comprises an arginine residue at a position corresponding to Q78 and at one or more positions corresponding to K65, K114, A122, and S150 of SEQ ID NO: 1.

In some embodiments, the amino acid sequence comprises an amino acid substitution at one or more positions corresponding to S22, C23, and S25 of SEQ ID NO: 1. In some embodiments, the amino acid substitution is selected from S22E, S22P, C23F, C23G, C23Q, C23A, C23V, C23I, C23L, S25G, S25A, S25V, S25I, or S25L. In some embodiments, the amino acid substitution is selected from S22E, S22P, C23F, C23G, C23Q, and S25G.

In some embodiments, the amino acid sequence comprises amino acid substitutions at positions corresponding to each of S22, C23, and S25 of SEQ ID NO: 1. In some embodiments, the amino acid substitutions comprise S22P. In some embodiments, the amino acid substitutions comprise C23G and/or S25G. In some embodiments, the amino acid substitutions comprise S22P, C23G, and S25G.

In some embodiments, the amino acid sequence comprises an amino acid substitution at one or more positions corresponding to A12, K65, V73, K114, A122, A149, and S150 of SEQ ID NO: 1. In some embodiments, the amino acid substitution is selected from A12L, K65R, V73L, K114R, A122R, A149S, and S150R.

In some embodiments, the amino acid sequence is at least 80%, at least 85%, at least 90%, at least 95%, at least 98%, at least 99%, 90-100%, 95-100%, 98-100%, or 100% identical to a sequence selected from any one of PS2150-2171, PS2195-2205, PS2244-2265, PS2278-2299, PS2392-2402, and PS2428-2449 (SEQ ID NOs: 3-112). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to a sequence selected from any one of PS2150-2171, PS2195-2205, PS2244-2265, PS2278-2299, PS2392-2402, and PS2428-2449 (SEQ ID NOs: 3-112). In some embodiments, the amino acid sequence is at least 80%, at least 85%, at least 90%, at least 95%, at least 98%, at least 99%, 90-100%, 95-100%, 98-100%, or 100% identical to PS2195 (SEQ ID NO: 25). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to PS2195 (SEQ ID NO: 25).

In some aspects, the disclosure provides a recombinant or synthetic amino acid binding protein having an amino acid sequence that is at least 80% identical (e.g., at least 85%, at least 90%, at least 95%, at least 98%, at least 99%, 80-98%, 80-95%, 80-90%, 85-95%, 90-98%, 90-100%, 95-100%, 98-100%, or 100% identical) to a sequence selected from any one of PS2150-2171, PS2195-2205, PS2244-2265, PS2278-2299, PS2392-2402, and PS2428-2449 (SEQ ID NOs: 3-112). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to a sequence selected from any one of PS2150-2171, PS2195-2205, PS2244-2265, PS2278-2299, PS2392-2402, and PS2428-2449 (SEQ ID NOs: 3-112).

In some aspects, the disclosure provides a recombinant or synthetic amino acid binding protein having an amino acid sequence that is at least 80% identical (e.g., at least 85%, at least 90%, at least 95%, at least 98%, at least 99%, 80-98%, 80-95%, 80-90%, 85-95%, 90-98%, 90-100%, 95-100%, 98-100%, or 100% identical) to a sequence selected from any one of PS2088-2089, PS2234-2243, PS2366-2379, PS2408-2409, and PS2424-2427 (SEQ ID NOs: 113-144). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to a sequence selected from any one of PS2088-2089, PS2234-2243, PS2366-2379, PS2408-2409, and PS2424-2427 (SEQ ID NOs: 113-144).

In some aspects, the disclosure provides a recombinant or synthetic amino acid binding protein having an amino acid sequence that is at least 80% identical (e.g., at least 85%, at least 90%, at least 95%, at least 98%, 80-98%, 80-95%, 80-90%, 85-95%, or 90-98% identical) to SEQ ID NO: 1, where the amino acid sequence comprises a glutamine residue at a position corresponding to S25 of SEQ ID NO: 1. In some embodiments, the amino acid sequence is at least 85%, at least 90%, at least 95%, at least 98%, 80-98%, 80-95%, 80-90%, 85-90%, 85-95%, 90-95%, or 90-98% identical to SEQ ID NO: 1.

In some embodiments, the amino acid sequence comprises an amino acid substitution at one or more positions corresponding to S5, A12, S22, S25, K65, S66, E71, V73, Q78, I80, C85, N120, A122, K147, A149, S150, and R154 of SEQ ID NO: 1. In some embodiments, the amino acid substitution is selected from SSC, A12L, S22E, K65R, S66V, E71R, V73I, V73L, Q78H, I80V, C85R, N120R, N120S, A122R, K147R, A149D, A149N, A149V, S150G, S150V, R154L, and R154Q.

In some embodiments, the amino acid sequence comprises an amino acid substitution at one or more positions corresponding to S66, Q78, S150, and R154 of SEQ ID NO: 1. In some embodiments, the amino acid substitution is selected from S66V, Q78H, S150G, S150V, R154L, and R154Q.

In some embodiments, the amino acid sequence comprises a histidine residue at a position corresponding to Q78 of SEQ ID NO: 1. In some embodiments, the amino acid sequence comprises a valine residue at a position corresponding to S150 of SEQ ID NO: 1.

In some embodiments, the amino acid sequence comprises amino acid substitutions at positions corresponding to each of S66, Q78, S150, and R154 of SEQ ID NO: 1. In some embodiments, the amino acid substitutions comprise S66V, Q78H, S150V, and R154L.

In some embodiments, the amino acid sequence comprises amino acid substitutions at positions corresponding to each of A12, S22, S66, E71, Q78, N120, A122, S150, and R154 of SEQ ID NO: 1. In some embodiments, the amino acid substitutions comprise A12L, S22E, S66V, E71R, Q78H, N120R, A122R, S150V, and R154L.

In some embodiments, the amino acid sequence is at least 80%, at least 85%, at least 90%, at least 95%, at least 98%, at least 99%, 90-100%, 95-100%, 98-100%, or 100% identical to PS2459 (SEQ ID NO: 234). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to PS2459 (SEQ ID NO: 234).

In some aspects, the disclosure provides a recombinant or synthetic amino acid binding protein having an amino acid sequence that is at least 80%, at least 85%, at least 90%, at least 95%, at least 98%, at least 99%, 90-100%, 95-100%, 98-100%, or 100% identical to a sequence selected from any one of PS2300-2314 and PS2450-2472 (SEQ ID NOs: 210-247). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to a sequence selected from any one of PS2300-2314 and PS2450-2472 (SEQ ID NOs: 210-247).

In some aspects, the disclosure provides a recombinant or synthetic amino acid binding protein having an amino acid sequence that is at least 80%, at least 85%, at least 90%, at least 95%, at least 98%, at least 99%, 90-100%, 95-100%, 98-100%, or 100% identical to a sequence selected from any one of PS2304, PS2305, PS2308, PS2310, PS2313, PS2451-2454, PS2457-2463, and PS2468 (SEQ ID NOs: 214-215, 218, 220, 223, 226-229, 232-238, and 243). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to a sequence selected from any one of PS2304, PS2305, PS2308, PS2310, PS2313, PS2451-2454, PS2457-2463, and PS2468 (SEQ ID NOs: 214-215, 218, 220, 223, 226-229, 232-238, and 243).

In some aspects, the disclosure provides a recombinant or synthetic amino acid binding protein having an amino acid sequence that is at least 80%, at least 85%, at least 90%, at least 95%, at least 98%, at least 99%, 90-100%, 95-100%, 98-100%, or 100% identical to a sequence selected from any one of PS2604-2619 and PS2687-2702 (SEQ ID NOs: 248-279). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to a sequence selected from any one of PS2604-2619 and PS2687-2702 (SEQ ID NOs: 248-279).

### B. UBR-Homologous Recognizers

In some embodiments, an amino acid recognizer of the disclosure binds an amino acid ligand (e.g., a polypeptide) comprising an N-terminal amino acid selected from arginine, histidine, lysine, or a modified variant thereof (e.g., a post-translationally modified variant thereof, an oxidized variant thereof). In some embodiments, the amino acid recognizer comprises an amino acid binding protein derived from a variant of a UBR protein, such as Kluyveromyces marxianus UBR protein. For example, in some embodiments, the amino acid binding protein is an engineered variant comprising one or more modifications relative to a UBR protein variant referred to herein as “PS1122” (SEQ ID NO: 2).

In some embodiments, the amino acid binding protein binds arginine (e.g., N-terminal arginine) with a dissociation constant (K_{D}) of less than 2,000 nM, less than 1,500 nM, less than 1,000 nM, less than 750 nM, less than 500 nM, less than 400 nM, less than 200 nM, less than 100 nM, less than 50 nM, 5-50 nM, 10-50 nM, 10-40 nM, 20-40 nM, 30-40 nM, 10-2,000 nM, 25-1,000 nM, 50-500 nM, 10-400 nM, 25-75 nM, or 50-80 nM. In some embodiments, the amino acid binding protein binds histidine (e.g., N-terminal histidine) with a dissociation constant (K_{D}) of less than 2,000 nM, less than 1,500 nM, less than 1,000 nM, less than 900 nM, less than 750 nM, less than 500 nM, less than 400 nM, less than 200 nM, less than 100 nM, 10-2,000 nM, 50-1,000 nM, 500-1,000 nM, 750-1,000 nM, 25-1,000 nM, 50-500 nM, 10-400 nM, 25-75 nM, or 50-80 nM. In some embodiments, the amino acid binding protein binds N-terminal lysine with a dissociation constant (K_{D}) of less than 2,000 nM, less than 1,500 nM, less than 1,000 nM, less than 750 nM, less than 500 nM, less than 400 nM, less than 200 nM, less than 100 nM, 10-2,000 nM, 25-1,000 nM, 50-500 nM, 10-400 nM, 25-75 nM, or 50-80 nM.

In some embodiments, the amino acid binding protein binds one or more types of N-terminal amino acids (e.g., arginine, histidine, lysine, or a modified variant thereof), where each type of binding interaction is characterized by a dissociation rate (k_{off}) of at least 0.1 s⁻¹. In some embodiments, the dissociation rate is between about 0.1 s⁻¹ and about 1,000 s⁻¹ (e.g., between about 0.5 s⁻¹ and about 500 s⁻¹, between about 0.1 s⁻¹ and about 100 s⁻¹, between about 1 s⁻¹ and about 100 s⁻¹, between about 5 s⁻¹ and about 100 s⁻¹, between about 5 s⁻¹ and about 50 s⁻¹, between about 10 s⁻¹ and about 25 s⁻¹, or between about 0.5 s⁻¹ and about 50 s⁻¹). In some embodiments, the dissociation rate is between about 0.5 s⁻¹ and about 20 s⁻¹. In some embodiments, the dissociation rate is between about 2 s⁻¹ and about 20 s⁻¹. In some embodiments, the dissociation rate is between about 0.5 s⁻¹ and about 2 s⁻¹

In some aspects, the disclosure provides a recombinant or synthetic amino acid binding protein having an amino acid sequence that is at least 80% identical (e.g., at least 85%, at least 90%, at least 95%, at least 98%, 80-98%, 80-95%, 80-90%, 85-95%, or 90-98% identical) to SEQ ID NO: 2, where the amino acid sequence comprises amino acid substitutions at positions corresponding to T53 and one or more selected from K26, D32, L47, F59, and T75 of SEQ ID NO: 2. In some embodiments, the amino acid sequence is at least 85%, at least 90%, at least 95%, at least 98%, 80-98%, 80-95%, 80-90%, 85-90%, 85-95%, 90-95%, or 90-98% identical to SEQ ID NO: 2.

In some embodiments, the amino acid substitutions comprise T53V, T53A, T53I, or T53L. In some embodiments, the amino acid substitutions comprise K26R or K26H. In some embodiments, the amino acid substitutions comprise D32R, D32P, D32K, or D32H. In some embodiments, the amino acid substitutions comprise L47R, L47K, or L47H. In some embodiments, the amino acid substitutions comprise F59R, F59K, or F59H. In some embodiments, the amino acid substitutions comprise T75D or T75E.

In some embodiments, the amino acid sequence comprises amino acid substitutions at positions corresponding to each of L47, F59, and T75. In some embodiments, the amino acid substitutions comprise L47R, T53V, F59R, and T75E. In some embodiments, the amino acid sequence comprises amino acid substitutions at positions corresponding to each of K26 and D32. In some embodiments, the amino acid substitutions comprise K26R and D32R or D32P.

In some embodiments, the amino acid sequence is at least 80%, at least 85%, at least 90%, at least 95%, at least 98%, at least 99%, 90-100%, 95-100%, 98-100%, or 100% identical to a sequence selected from any one of PS1936-PS1938 (SEQ ID NOs: 158-160). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to a sequence selected from any one of PS1936-PS1938 (SEQ ID NOs: 158-160). In some embodiments, the amino acid sequence is at least 80%, at least 85%, at least 90%, at least 95%, at least 98%, at least 99%, 90-100%, 95-100%, 98-100%, or 100% identical to PS1936 (SEQ ID NO: 158). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to PS1936 (SEQ ID NO: 158).

In some aspects, the disclosure provides a recombinant or synthetic amino acid binding protein having an amino acid sequence that is at least 80% identical (e.g., at least 85%, at least 90%, at least 95%, at least 98%, at least 99%, 80-98%, 80-95%, 80-90%, 85-95%, 90-98%, 90-100%, 95-100%, 98-100%, or 100% identical) to a sequence selected from any one of PS1923-PS1938, PS1659, and PS1715 (SEQ ID NOs: 145-162). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to a sequence selected from any one of PS1923-PS1938, PS1659, and PS1715 (SEQ ID NOs: 145-162).

In some aspects, the disclosure provides a recombinant or synthetic amino acid binding protein having an amino acid sequence that is at least 80% identical (e.g., at least 85%, at least 90%, at least 95%, at least 98%, at least 99%, 80-98%, 80-95%, 80-90%, 85-95%, 90-98%, 90-100%, 95-100%, 98-100%, or 100% identical) to a sequence selected from any one of PS2080-2085, PS2173-2183, and PS2406-2407 (SEQ ID NOs: 163-181). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to a sequence selected from any one of PS2080-2085, PS2173-2183, and PS2406-2407 (SEQ ID NOs: 163-181).

### C. Tandem Recognizers

In some embodiments, an amino acid recognizer comprises a single polypeptide having tandem copies of two or more amino acid binding proteins, where at least one of the two or more amino acid binding proteins is an amino acid binding protein of the disclosure. As used herein, in some embodiments, a tandem arrangement or orientation of elements in a molecule refers to an end-to-end joining of each element to the next element in a linear fashion such that the elements are fused in series. For example, in some embodiments, a polypeptide having tandem copies of two amino acid binding proteins refers to a fusion polypeptide in which the C-terminus of one protein is fused to the N-terminus of the other protein. Similarly, a polypeptide having tandem copies of two or more amino acid binding proteins refers to a fusion polypeptide in which the C-terminus of a first protein is fused to the N-terminus of a second protein, the C-terminus of the second protein is fused to the N-terminus of a third protein, and so forth. Such fusion polypeptides can comprise multiple copies of the same amino acid binding protein or multiple copies of different amino acid binding proteins. In some embodiments, a fusion polypeptide of the disclosure has at least two and up to ten amino acid binding proteins (e.g., at least 2 binders and up to eight, six, five, four, or three binders). In some embodiments, a fusion polypeptide of the disclosure has five or fewer amino acid binding proteins (e.g., two, three, four, or five amino acid binding proteins).

In some embodiments, a fusion polypeptide is provided by expression of a single coding sequence containing segments encoding monomeric amino acid binding protein subunits separated by segments encoding flexible linkers, where expression of the single coding sequence produces a single full-length polypeptide having two or more independent binding sites. In some embodiments, one or more of the monomeric subunits are Ntaq1-homologous proteins, UBR-homologous proteins, ClpS-homologous proteins, or BIR3 domain-homologous proteins. In some embodiments, the monomeric subunits may be identical or non-identical. Where non-identical, the monomeric subunits may be distinct variants of the same parent-homologous protein, or they may be derived from different parent-homologous proteins. In some embodiments, a fusion polypeptide comprises two or more Ntaq1-homologous monomers, two or more UBR-homologous monomers, two or more ClpS-homologous monomers, or two or more BIR3 domain-homologous monomers.

In some embodiments, at least one amino acid binding protein of a fusion polypeptide has an amino acid sequence selected from Table 1 (or having an amino acid sequence that has at least 50%, at least 60%, at least 70%, at least 80%, 80-90%, 90-95%, 95-99%, or higher, amino acid sequence identity to an amino acid sequence selected from Table 1). In some embodiments, each amino acid binding protein of a fusion polypeptide has an amino acid sequence that is at least 80% (e.g., 80-90%, 90-95%, 95-99%, or higher) identical to an amino acid sequence selected from Table 1 (or having an amino acid sequence that has at least 50%, at least 60%, at least 70%, at least 80%, 80-90%, 90-95%, 95-99%, or higher, amino acid sequence identity to an amino acid sequence selected from Table 1). In some embodiments, an amino acid binding protein of a fusion polypeptide is modified and includes one or more amino acid deletions, additions, or mutations relative to a sequence set forth in Table 1. In some embodiments, an amino acid binding protein of a fusion polypeptide includes a deletion, addition, or mutation of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acids (which may or may not be consecutive amino acids) relative to a sequence set forth in Table 1. In some embodiments, the linker of a fusion polypeptide has an amino acid sequence selected from Table 2B (or having an amino acid sequence that has at least 50%, at least 60%, at least 70%, at least 80%, 80-90%, 90-95%, 95-99%, or higher, amino acid sequence identity to an amino acid sequence selected from Table 2B).

In some embodiments, amino acid binding proteins of a fusion polypeptide recognize the same set of one or more amino acids. In some embodiments, amino acid binding proteins of a fusion polypeptide recognize a distinct set of one or more amino acids. In some embodiments, amino acid binding proteins of a fusion polypeptide recognize an overlapping set of amino acids. In some embodiments, where the amino acid binding proteins of a fusion polypeptide recognize the same amino acid, they may recognize the amino acid with the same characteristic pulsing pattern or with different characteristic pulsing patterns.

In some embodiments, amino acid binding proteins of a fusion polypeptide are joined end-to-end, either by a covalent bond or a linker that covalently joins the C-terminus of one protein to the N-terminus of another protein. In the context of fusion polypeptides of the disclosure, a linker refers to one or more amino acids within a fusion polypeptide that joins two amino acid binding proteins and that does not form part of the polypeptide sequence corresponding to either of the two proteins. In some embodiments, a linker comprises at least two amino acids (e.g., at least 2, at least 3, at least 4, at least 5, at least 6, at least 8, at least 10, at least 15, at least 25, at least 50, at least 100, or more, amino acids). In some embodiments, a linker comprises up to 5, up to 10, up to 15, up to 25, up to 50, or up to 100, amino acids. In some embodiments a linker comprises between about 2 and about 200 amino acids (e.g., between about 2 and about 100, between about 2 and about 50, between about 5 and about 50, between about 10 and about 40, between about 2 and about 20, between about 5 and about 20, or between about 2 and about 30, amino acids).

Accordingly, in some aspects, the disclosure provides an amino acid recognizer comprising a polypeptide having a first amino acid binding protein and a second amino acid binding protein joined end-to-end, where the first and second amino acid binding proteins are separated by a linker comprising at least two amino acids.

In some embodiments, each of the first and second amino acid binding proteins independently has an amino acid sequence that is at least 80% identical (e.g., at least 85%, at least 90%, at least 95%, at least 98%, 80-100%, 85-100%, 90-100%, 95-100%, or 100% identical) to a sequence selected from any one of PS2150-2171, PS2195-2205, PS2244-2265, PS2278-2299, PS2392-2402, and PS2428-2449 (SEQ ID NOs: 3-112). In some embodiments, the first and second amino acid binding proteins are separated by a linker having an amino acid sequence that is at least 80% identical (e.g., at least 85%, at least 90%, at least 95%, at least 98%, 80-100%, 85-100%, 90-100%, 95-100%, or 100% identical) to a sequence selected from Table 2B (SEQ ID NOs: 194-209). In some embodiments, the amino acid recognizer comprises a polypeptide having an amino acid sequence that is at least 80%, at least 85%, at least 90%, at least 95%, at least 98%, 80-100%, 85-100%, 90-100%, 95-100%, or 100% identical to a sequence selected from any one of PS2088-2089, PS2234-2243, PS2366-2379, PS2408-2409, and PS2424-2427 (SEQ ID NOs: 113-144).

In some embodiments, each of the first and second amino acid binding proteins independently has an amino acid sequence that is at least 80% identical (e.g., at least 85%, at least 90%, at least 95%, at least 98%, 80-100%, 85-100%, 90-100%, 95-100%, or 100% identical) to PS2195 (SEQ ID NO: 25). In some embodiments, the first and second amino acid binding proteins are separated by a linker having an amino acid sequence that is at least 80% identical (e.g., at least 85%, at least 90%, at least 95%, at least 98%, 80-100%, 85-100%, 90-100%, 95-100%, or 100% identical) to a sequence selected from Table 2B (SEQ ID NOs: 194-209). In some embodiments, the amino acid recognizer comprises a polypeptide having an amino acid sequence that is at least 80%, at least 85%, at least 90%, at least 95%, at least 98%, 80-100%, 85-100%, 90-100%, 95-100%, or 100% identical to a sequence selected from any one of PS2366-2379 and PS2408-2409 (SEQ ID NOs: 125-140).

In some embodiments, each of the first and second amino acid binding proteins independently has an amino acid sequence that is at least 80% identical (e.g., at least 85%, at least 90%, at least 95%, at least 98%, 80-100%, 85-100%, 90-100%, 95-100%, or 100% identical) to a sequence selected from any one of PS2300-2314 and PS2450-2472 (SEQ ID NOs: 210-247). In some embodiments, the first and second amino acid binding proteins are separated by a linker having an amino acid sequence that is at least 80% identical (e.g., at least 85%, at least 90%, at least 95%, at least 98%, 80-100%, 85-100%, 90-100%, 95-100%, or 100% identical) to a sequence selected from Table 2B (SEQ ID NOs: 194-209).

In some embodiments, each of the first and second amino acid binding proteins independently has an amino acid sequence that is at least 80% identical (e.g., at least 85%, at least 90%, at least 95%, at least 98%, 80-100%, 85-100%, 90-100%, 95-100%, or 100% identical) to PS2457 (SEQ ID NO: 232). In some embodiments, each of the first and second amino acid binding proteins independently has an amino acid sequence that is at least 80% identical (e.g., at least 85%, at least 90%, at least 95%, at least 98%, 80-100%, 85-100%, 90-100%, 95-100%, or 100% identical) to PS2459 (SEQ ID NO: 234). In some embodiments, the first and second amino acid binding proteins are separated by a linker having an amino acid sequence that is at least 80% identical (e.g., at least 85%, at least 90%, at least 95%, at least 98%, 80-100%, 85-100%, 90-100%, 95-100%, or 100% identical) to a sequence selected from Table 2B (SEQ ID NOs: 194-209). In some embodiments, the amino acid recognizer comprises a polypeptide having an amino acid sequence that is at least 80%, at least 85%, at least 90%, at least 95%, at least 98%, 80-100%, 85-100%, 90-100%, 95-100%, or 100% identical to a sequence selected from any one of PS2604-2619 and PS2687-2702 (SEQ ID NOs: 248-279).

In some embodiments, each of the first and second amino acid binding proteins independently has an amino acid sequence that is at least 80% identical (e.g., at least 85%, at least 90%, at least 95%, at least 98%, 80-100%, 85-100%, 90-100%, 95-100%, or 100% identical) to a sequence selected from any one of PS1923-PS1938, PS1659, and PS1715 (SEQ ID NOs: 145-162). In some embodiments, the first and second amino acid binding proteins are separated by a linker having an amino acid sequence that is at least 80% identical (e.g., at least 85%, at least 90%, at least 95%, at least 98%, 80-100%, 85-100%, 90-100%, 95-100%, or 100% identical) to a sequence selected from Table 2B (SEQ ID NOs: 194-209). In some embodiments, the amino acid recognizer comprises a polypeptide having an amino acid sequence that is at least 80%, at least 85%, at least 90%, at least 95%, at least 98%, 80-100%, 85-100%, 90-100%, 95-100%, or 100% identical to a sequence selected from any one of PS2080-2085, PS2173-2183, and PS2406-2407 (SEQ ID NOs: 163-181).

In some embodiments, each of the first and second amino acid binding proteins independently has an amino acid sequence that is at least 80% identical (e.g., at least 85%, at least 90%, at least 95%, at least 98%, 80-100%, 85-100%, 90-100%, 95-100%, or 100% identical) to PS1936 (SEQ ID NO: 158). In some embodiments, the first and second amino acid binding proteins are separated by a linker having an amino acid sequence that is at least 80% identical (e.g., at least 85%, at least 90%, at least 95%, at least 98%, 80-100%, 85-100%, 90-100%, 95-100%, or 100% identical) to a sequence selected from Table 2B (SEQ ID NOs: 194-209). In some embodiments, the amino acid recognizer comprises a polypeptide having an amino acid sequence that is at least 80%, at least 85%, at least 90%, at least 95%, at least 98%, 80-100%, 85-100%, 90-100%, 95-100%, or 100% identical to a sequence selected from any one of PS2084-2085, PS2173-2183, and PS2406-2407 (SEQ ID NOs: 167-181).

In some aspects, the disclosure provides a nucleic acid encoding a single polypeptide having tandem copies of two or more amino acid binding proteins. In some embodiments, the nucleic acid is an expression construct encoding a fusion polypeptide of the disclosure. In some embodiments, an expression construct encodes a fusion polypeptide having at least two and up to ten amino acid binding proteins (e.g., at least two and up to three, four, five, six, seven, eight, nine, or ten amino acid binding proteins). In some embodiments, an expression construct encodes a fusion polypeptide having five or fewer amino acid binding proteins (e.g., two, three, four, or five amino acid binding proteins).

### D. Shielded Recognizers

In accordance with embodiments described herein, single-molecule polypeptide sequencing methods can be carried out by illuminating a surface-immobilized polypeptide with excitation light, and detecting luminescence produced by a label attached to an amino acid recognizer. In some cases, radiative and/or non-radiative decay produced by the label can result in photodamage to the polypeptide, and the inventors have found that photodamage can be mitigated and recognition times extended by incorporation of a shielding element into an amino acid recognizer. See, for example, PCT International Publication No. WO2020102741A1, filed Nov. 15, 2019, and PCT International Publication No. WO2021236983A2, filed May 20, 2021, which describe shielded recognition molecules in detail, the relevant content of which is incorporated by reference in its entirety.

Accordingly, in some aspects, the disclosure provides shielded recognizers comprising at least one amino acid recognizer (e.g., amino acid binding protein) described herein, at least one detectable label, and a shielding element (e.g., a “shield”) that forms a covalent or non-covalent linkage group between the recognizer and label. In some embodiments, a shield forms a covalent or non-covalent linkage group between one or more amino acid binding proteins and one or more labels.

In some embodiments, a shielded recognizer comprises a fusion polypeptide having an amino acid binding protein of the disclosure and a protein shield joined end-to-end (e.g., in a C-terminal to N-terminal fashion). In some embodiments, the protein shield comprises a labeled protein, such as a fluorescent protein or a non-fluorescent protein that comprises a luminescent label.

In some embodiments, the amino acid binding protein and the protein shield are joined end-to-end, either by a covalent bond or a linker that covalently joins the C-terminus of one protein to the N-terminus of the other protein. In some embodiments, a linker in the context of a fusion polypeptide refers to one or more amino acids within the fusion polypeptide that joins the amino acid binding protein and the protein shield and that does not form part of the polypeptide sequence corresponding to either the amino acid binding protein or the protein shield. In some embodiments, a linker comprises at least two amino acids (e.g., at least 2, 3, 4, 5, 6, 8, 10, 15, 25, 50, 100, or more, amino acids). In some embodiments, a linker comprises up to 5, up to 10, up to 15, up to 25, up to 50, or up to 100, amino acids. In some embodiments a linker comprises between about 2 and about 200 amino acids (e.g., between about 2 and about 100, between about 5 and about 50, between about 2 and about 20, between about 5 and about 20, or between about 2 and about 30, amino acids).

In some embodiments, a protein shield of a fusion polypeptide is a protein having a molecular weight of at least 10 kDa. For example, in some embodiments, a protein shield is a protein having a molecular weight of at least 10 kDa and up to 500 kDa (e.g., between about 10 kDa and about 250 kDa, between about 10 kDa and about 150 kDa, between about 10 kDa and about 100 kDa, between about 20 kDa and about 80 kDa, between about 15 kDa and about 100 kDa, or between about 15 kDa and about 50 kDa). In some embodiments, a protein shield of a fusion polypeptide is a protein comprising at least 25 amino acids. For example, in some embodiments, a protein shield is a protein comprising at least 25 and up to 1,000 amino acids (e.g., between about 100 and about 1,000 amino acids, between about 100 and about 750 amino acids, between about 500 and about 1,000 amino acids, between about 250 and about 750 amino acids, between about 50 and about 500 amino acids, between about 100 and about 400 amino acids, or between about 50 and about 250 amino acids).

In some embodiments, a protein shield is a polypeptide comprising one or more tag proteins. In some embodiments, a protein shield is a polypeptide comprising at least two tag proteins. In some embodiments, the at least two tag proteins are the same (e.g., the polypeptide comprises at least two copies of a tag protein sequence). In some embodiments, the at least two tag proteins are different (e.g., the polypeptide comprises at least two different tag protein sequences). Examples of tag proteins include, without limitation, Fasciola hepatica 8-kDa antigen (Fh8), Maltose-binding protein (MBP), N-utilization substance (NusA), Thioredoxin (Trx), Small ubiquitin-like modifier (SUMO), Glutathione-S-transferase (GST), Solubility-enhancer peptide sequences (SET), IgG domain B1 of Protein G (GB1), IgG repeat domain ZZ of Protein A (ZZ), Mutated dehalogenase (HaloTag), Solubility eNhancing Ubiquitous Tag (SNUT), Seventeen kilodalton protein (Skp), Phage T7 protein kinase (T7PK), E. coli secreted protein A (EspA), Monomeric bacteriophage T7 0.3 protein (Orc protein; Mocr), E. coli trypsin inhibitor (Ecotin), Calcium-binding protein (CaBP), Stress-responsive arsenate reductase (ArsC), N-terminal fragment of translation initiation factor IF2 (IF2-domain I), Stress-responsive proteins (e.g., RpoA, SlyD, Tsf, RpoS, PotD, Crr), and E. coli acidic proteins (e.g., msyB, yjgD, rpoD). See, e.g., Costa, S., et al. “Fusion tags for protein solubility, purification and immunogenicity in Escherichia coli: the novel Fh8 system.” Front Microbiol. 2014 Feb. 19; 5:63, the relevant content of which is incorporated herein by reference.

A shielding element of the disclosure can advantageously absorb, deflect, or otherwise block radiative and/or non-radiative decay emitted by a label of an amino acid recognizer. Thus, it should be appreciated that a suitable protein shield of a fusion polypeptide can be readily selected by those skilled in the art. For example, the inventors have demonstrated the use of a variety of types of protein shields in the context of a fusion polypeptide, including polypeptides having an amino acid binding protein fused to an enzyme (e.g., DNA polymerase, glutathione S-transferase), a transport protein (e.g., maltose-binding protein), a fluorescent protein (e.g., GFP), and a commercially available tag protein (e.g., SNAP-Tag®). The inventors have further demonstrated the use of fusion polypeptides having multiple copies of a protein shield oriented in tandem. See, for example, PCT International Publication No. WO2021236983A2, filed May 20, 2021.

Accordingly, in some embodiments, the disclosure provides a fusion polypeptide having one or more tandemly-oriented amino acid binding proteins fused to one or more tandemly-oriented protein shields. In some embodiments, where a fusion polypeptide comprises two or more tandemly-oriented binders and/or two or more tandemly-oriented shields, a terminal end of one of the two or more binders is joined end-to-end with a terminal end of one of the two or more shields. Fusion polypeptides having tandem copies of two or more binders are described elsewhere herein, and in some embodiments, such fusions can further comprise a protein shield joined end-to-end with one of the two or more binders.

Additional example configurations of shielded recognizers and shielding elements (e.g., oligonucleotide shields, avidin protein shields) have been described and are contemplated for use in accordance with the present disclosure. See, for example, PCT International Publication No. WO2020102741A1, filed Nov. 15, 2019, and PCT International Publication No. WO2021236983A2, filed May 20, 2021, the relevant contents of each of which are incorporated herein.

### E. Labels

In some embodiments, an amino acid recognizer of the disclosure comprises one or more labels. In some embodiments, the one or more labels comprise a detectable label, such as a luminescent label or a conductivity label. As described herein, in some embodiments, one or more chemical characteristics of a polypeptide can be determined by monitoring a signal for changes in the signal (e.g., signal pulses) corresponding to binding events between one or more amino acid recognizers and the polypeptide. In some embodiments, an amino acid recognizer comprises a detectable label that produces a change in the signal during a binding event between the amino acid recognizer and the polypeptide. Accordingly, as used herein, a detectable label of an amino acid recognizer can refer to any label capable of producing a detectable change in signal during a binding event between the amino acid recognizer and a polypeptide.

In some embodiments, the one or more labels of an amino acid recognizer comprise a luminescent label. In some embodiments, a luminescent label comprises at least one fluorophore dye molecule (e.g., at least 2, at least 3, at least 4, at least 5, 20 or fewer, 15 or fewer, 10 or fewer fluorophore dye molecules). In some embodiments, a luminescent label comprises at least one FRET pair comprising a donor label and an accepter label. Examples of luminescent labels and their use in accordance with the disclosure are described in detail elsewhere herein.

In some embodiments, the one or more labels of an amino acid recognizer comprise a conductivity label. In some embodiments, the conductivity label is a charge label, such as a charged polymer. Examples of charge labels include dendrimers, nanoparticles, nucleic acids and other polymers having multiple charged groups. In some embodiments, a conductivity label is uniquely identifiable by its net charge (e.g., a net positive charge or a net negative charge), by its charge density, and/or by its number of charged groups.

In some embodiments, the one or more labels of an amino acid recognizer comprise a tag peptide. For example, in some embodiments, an amino acid recognizer comprises a tag peptide that provides one or more functions other than amino acid binding. In some embodiments, a tag peptide comprises at least one biotin ligase recognition sequence that permits biotinylation of the recognizer (e.g., incorporation of one or more biotin molecules, including biotin and bis-biotin moieties). In some embodiments, a tag peptide comprises two biotin ligase recognition sequences oriented in tandem. In some embodiments, a biotin ligase recognition sequence refers to an amino acid sequence that is recognized by a biotin ligase, which catalyzes a covalent linkage between the sequence and a biotin molecule. Each biotin ligase recognition sequence of a tag peptide can be covalently linked to a biotin moiety, such that a tag peptide having multiple biotin ligase recognition sequences can be covalently linked to multiple biotin molecules. A region of a tag peptide having one or more biotin ligase recognition sequences can be generally referred to as a biotinylation tag or a biotinylation sequence. In some embodiments, a bis-biotin or bis-biotin moiety can refer to two biotins bound to two biotin ligase recognition sequences oriented in tandem.

Additional examples of functional sequences in a tag peptide include purification tags, cleavage sites, and other moieties useful for purification and/or modification of recognizers. Table 2A provides a list of non-limiting sequences of tag peptides, any one or more of which may be used in combination with any one of the amino acid recognizers of the disclosure (e.g., in combination with a sequence set forth in Table 1). It should be appreciated that the tag peptides shown in Table 2A are meant to be non-limiting, and recognizers in accordance with the disclosure can include any one or more of the tag peptides (e.g., His-tags and/or biotinylation tags) at the N- or C-terminus of a recognizer polypeptide or at an internal position, split between the N- and C-terminus, or otherwise rearranged as practiced in the art.

In some embodiments, the disclosure provides amino acid recognizers comprising an amino acid binding protein described herein (e.g., a sequence selected from Table 1) and a tag peptide described herein (e.g., a sequence selected from Table 2A). In some embodiments, a terminal amino acid of the amino acid binding protein is attached to a terminal amino acid of the tag peptide, thereby forming a fusion polypeptide. In some embodiments, a fusion polypeptide comprises: (i) a first amino acid sequence (e.g., an amino acid binding protein) that is at least 80%, at least 85%, at least 90%, at least 95%, at least 98%, 80-100%, 85-100%, 90-100%, 95-100%, or 100% identical to a sequence selected from any one of SEQ ID NOs: 1-185 and 210-279; and (ii) a second amino acid sequence (e.g., a tag peptide) that is at least 80%, at least 85%, at least 90%, at least 95%, at least 98%, 80-100%, 85-100%, 90-100%, 95-100%, or 100% identical to a sequence selected from any one of SEQ ID NOs: 186-193 and 280.

In some embodiments, the fusion polypeptide comprises, in an N-terminal to C-terminal direction: (i) the first amino acid sequence (e.g., the amino acid binding protein); and (ii) the second amino acid sequence (e.g., the tag peptide). In some embodiments, the C-terminal amino acid of the first amino acid sequence is attached (e.g., fused) to the N-terminal amino acid of the second amino acid sequence through a peptide bond, such that the fusion polypeptide forms a contiguous amino acid sequence having, in an N-terminal to C-terminal direction: the first amino acid sequence and the second amino acid sequence.

In some embodiments, the first amino acid sequence is at least 80%, at least 85%, at least 90%, at least 95%, at least 98%, 80-100%, 85-100%, 90-100%, 95-100%, or 100% identical to PS2195 (SEQ ID NO: 25), and the second amino acid sequence is at least 80%, at least 85%, at least 90%, at least 95%, at least 98%, 80-100%, 85-100%, 90-100%, 95-100%, or 100% identical to a sequence selected from any one of SEQ ID NOs: 186-193 and 280.

In some embodiments, the first amino acid sequence is at least 80%, at least 85%, at least 90%, at least 95%, at least 98%, 80-100%, 85-100%, 90-100%, 95-100%, or 100% identical to PS2459 (SEQ ID NO: 234), and the second amino acid sequence is at least 80%, at least 85%, at least 90%, at least 95%, at least 98%, 80-100%, 85-100%, 90-100%, 95-100%, or 100% identical to a sequence selected from any one of SEQ ID NOs: 186-193 and 280. In some embodiments, the first amino acid sequence comprises PS2459 (SEQ ID NO: 234), and the second amino acid sequence comprises SEQ ID NO: 280. In some embodiments, the fusion polypeptide comprises, in an N-terminal to C-terminal direction: (i) SEQ ID NO: 234; and (ii) SEQ ID NO: 280, where the C-terminal amino acid of SEQ ID NO: 234 is attached to the N-terminal amino acid of SEQ ID NO: 280 through a peptide bond.

In some embodiments, the first amino acid sequence is at least 80%, at least 85%, at least 90%, at least 95%, at least 98%, 80-100%, 85-100%, 90-100%, 95-100%, or 100% identical to PS1936 (SEQ ID NO: 158), and the second amino acid sequence is at least 80%, at least 85%, at least 90%, at least 95%, at least 98%, 80-100%, 85-100%, 90-100%, 95-100%, or 100% identical to a sequence selected from any one of SEQ ID NOs: 186-193 and 280.

In some embodiments, the one or more labels of an amino acid recognizer comprise a biotin moiety. In some embodiments, the biotin moiety comprises at least one biotin molecule (e.g., 1, 2, 3, 4, or more biotin molecules). In some embodiments, the biotin moiety is a bis-biotin moiety. In some embodiments, the biotin moiety comprises at least one biotin molecule attached to at least one biotin ligase recognition sequence. For example, in some embodiments, the one or more labels comprise a tag peptide comprising two biotin ligase recognition sequences oriented in tandem, each biotin ligase recognition sequence having a biotin molecule attached thereto. In some embodiments, the biotin moiety comprises at least one biotin molecule attached to the amino acid recognizer through means other than a tag peptide. For example, in some embodiments, the at least one biotin molecule is chemically conjugated to an amino acid (e.g., an unnatural amino acid) of an amino acid binding protein.

In some embodiments, the biotin moiety is bound to a first biotin binding site of an avidin protein (e.g., streptavidin). In some embodiments, the avidin protein comprises a label component. In some embodiments, the label component comprises a luminescently labeled oligonucleotide comprising a second biotin moiety bound to a second biotin binding site of the avidin protein (e.g., thereby forming a shielded recognizer described herein).

In some embodiments, the one or more labels of an amino acid recognizer comprise one or more polyol moieties (e.g., one or more moieties selected from dextran, polyvinylpyrrolidone, polyethylene glycol, polypropylene glycol, polyoxyethylene glycol, and polyvinyl alcohol). For example, in some embodiments, an amino acid recognizer is PEGylated. In some embodiments, polyol modification (e.g., PEGylation) can limit the extent of non-specific sticking to a substrate (e.g., sequencing chip) surface. In some embodiments, polyol modification can limit the extent of aggregation or interaction between an amino acid recognizer with other recognizers, with a cleaving reagent, or with other species present in a sequencing reaction mixture. PEGylation can be performed by incubating a recognizer (e.g., an amino acid binding protein, such as a ClpS protein) with mPEG4-NHS ester, which labels primary amines such as surface-exposed lysine side chains. Other types of PEG and other methods of polyol modification are known in the art.

It should be appreciated that, in some embodiments, an amino acid recognizer of the disclosure can comprise one or more different types of labels described herein. For example, in some embodiments, an amino acid recognizer comprises one or more labels selected from a detectable label (e.g., a luminescent label, a conductivity label), a tag peptide (e.g., a purification tag, a cleavage site, a biotinylation sequence), a biotin moiety, and a polyol moiety. In some embodiments, an amino acid recognizer comprises a detectable label (e.g., a luminescent label, a conductivity label) and one or more labels selected from a tag peptide (e.g., a purification tag, a cleavage site, a biotinylation sequence), a biotin moiety, and a polyol moiety.

In some embodiments, the one or more labels of an amino acid recognizer comprise a luminescent label. As used herein, a luminescent label is a molecule that absorbs one or more photons and may subsequently emit one or more photons after one or more time durations. In some embodiments, the term is used interchangeably with “label,” “detectable label,” or “luminescent molecule” depending on context. A luminescent label in accordance with certain embodiments described herein may refer to a luminescent label of an amino acid recognizer, a luminescent label of a cleaving reagent (e.g., a peptidase, such as an aminopeptidase), or a luminescent label of another labeled composition described herein.

In some embodiments, a luminescent label comprises a first chromophore and a second chromophore. In some embodiments, an excited state of the first chromophore is capable of relaxation via an energy transfer to the second chromophore. In some embodiments, the energy transfer is a Förster resonance energy transfer (FRET). Such a FRET pair may be useful for providing a luminescent label with properties that make the label easier to differentiate from amongst a plurality of luminescent labels in a mixture, or for providing a binding-induced fluorescence that limits background fluorescence as described elsewhere herein. In yet other embodiments, a FRET pair comprises a first chromophore of a first luminescent label and a second chromophore of a second luminescent label. In certain embodiments, the FRET pair may absorb excitation energy in a first spectral range and emit luminescence in a second spectral range.

In some embodiments, a luminescent label refers to a fluorophore or a dye. Typically, a luminescent label comprises an aromatic or heteroaromatic compound and can be a pyrene, anthracene, naphthalene, naphthylamine, acridine, stilbene, indole, benzindole, oxazole, carbazole, thiazole, benzothiazole, benzoxazole, phenanthridine, phenoxazine, porphyrin, quinoline, ethidium, benzamide, cyanine, carbocyanine, salicylate, anthranilate, coumarin, fluorescein, rhodamine, xanthene, or other like compounds.

In some embodiments, a luminescent label comprises a dye selected from one or more of the following: 5/6-Carboxyrhodamine 6G, 5-Carboxyrhodamine 6G, 6-Carboxyrhodamine 6G, 6-TAMRA, Abberior® STAR 440SXP, Abberior® STAR 470SXP, Abberior® STAR 488, Abberior® STAR 512, Abberior® STAR 520SXP, Abberior® STAR 580, Abberior® STAR 600, Abberior® STAR 635, Abberior® STAR 635P, Abberior® STAR RED, Alexa Fluor®350, Alexa Fluor® 405, Alexa Fluor® 430, Alexa Fluor® 480, Alexa Fluor® 488, Alexa Fluor® 514, Alexa Fluor® 532, Alexa Fluor® 546, Alexa Fluor® 555, Alexa Fluor® 568, Alexa Fluor® 594, Alexa Fluor® 610-X, Alexa Fluor® 633, Alexa Fluor® 647, Alexa Fluor®660, Alexa Fluor® 680, Alexa Fluor® 700, Alexa Fluor® 750, Alexa Fluor® 790, AMCA, ATTO 390, ATTO 425, ATTO 465, ATTO 488, ATTO 495, ATTO 514, ATTO 520, ATTO 532, ATTO 542, ATTO 550, ATTO 565, ATTO 590, ATTO 610, ATTO 620, ATTO 633, ATTO 647, ATTO 647N, ATTO 655, ATTO 665, ATTO 680, ATTO 700, ATTO 725, ATTO 740, ATTO Oxa12, ATTO Rho101, ATTO Rho11, ATTO Rho12, ATTO Rho13, ATTO Rho14, ATTO Rho3B, ATTO Rho6G, ATTO Thio12, BD Horizon™ V450, BODIPY® 493/501, BODIPY® 530/550, BODIPY® 558/568, BODIPY® 564/570, BODIPY® 576/589, BODIPY® 581/591, BODIPY® 630/650, BODIPY® 650/665, BODIPY® FL, BODIPY® FL-X, BODIPY® R6G, BODIPY® TMR, BODIPY® TR, CAL Fluor® Gold 540, CAL Fluor® Green 510, CAL Fluor® Orange 560, CAL Fluor® Red 590, CAL Fluor® Red 610, CAL Fluor® Red 615, CAL Fluor® Red 635, Cascade® Blue, CF™ 350, CF™ 405M, CF™ 405S, CF™ 488A, CF™514, CF™ 532, CF™ 543, CF™ 546, CF™ 555, CF™ 568, CF™ 594, CF™ 620R, CF™ 633, CF™ 633-V1, CF™ 640R, CF™ 640R-V1, CF™ 640R-V2, CF™ 660C, CF™ 660R, CF™ 680, CF™ 680R, CF™ 680R-V1, CF™ 750, CF™ 770, CF™ 790, Chromeo™ 642, Chromis 425N, Chromis 500N, Chromis 515N, Chromis 530N, Chromis 550A, Chromis 550C, Chromis 550Z, Chromis 560N, Chromis 570N, Chromis 577N, Chromis 600N, Chromis 630N, Chromis 645A, Chromis 645C, Chromis 645Z, Chromis 678A, Chromis 678C, Chromis 678Z, Chromis 770A, Chromis 770C, Chromis 800A, Chromis 800C, Chromis 830A, Chromis 830C, Cy® 3, Cy® 3.5, Cy® 3B, Cy® 5, Cy® 5.5, Cy® 7, DyLight® 350, DyLight® 405, DyLight® 415-Co1, DyLight® 425Q, DyLight® 485-LS, DyLight® 488, DyLight® 504Q, DyLight® 510-LS, DyLight® 515-LS, DyLight® 521-LS, DyLight® 530-R2, DyLight® 543Q, DyLight® 550, DyLight® 554-R0, DyLight® 554-R1, DyLight® 590-R2, DyLight® 594, DyLight® 610-B1, DyLight® 615-B2, DyLight® 633, DyLight® 633-B1, DyLight® 633-B2, DyLight® 650, DyLight® 655-B1, DyLight® 655-B2, DyLight® 655-B3, DyLight® 655-B4, DyLight® 662Q, DyLight® 675-B1, DyLight® 675-B2, DyLight® 675-B3, DyLight® 675-B4, DyLight® 679-C5, DyLight® 680, DyLight® 683Q, DyLight® 690-B1, DyLight® 690-B2, DyLight® 696Q, DyLight® 700-B1, DyLight® 700-B1, DyLight® 730-B1, DyLight® 730-B2, DyLight® 730-B3, DyLight® 730-B4, DyLight® 747, DyLight® 747-B1, DyLight® 747-B2, DyLight® 747-B3, DyLight® 747-B4, DyLight® 755, DyLight® 766Q, DyLight® 775-B2, DyLight® 775-B3, DyLight® 775-B4, DyLight® 780-B1, DyLight® 780-B2, DyLight® 780-B3, DyLight® 800, DyLight® 830-B2, Dyomics-350, Dyomics-350XL, Dyomics-360XL, Dyomics-370XL, Dyomics-375XL, Dyomics-380XL, Dyomics-390XL, Dyomics-405, Dyomics-415, Dyomics-430, Dyomics-431, Dyomics-478, Dyomics-480XL, Dyomics-481XL, Dyomics-485XL, Dyomics-490, Dyomics-495, Dyomics-505, Dyomics-510XL, Dyomics-511XL, Dyomics-520XL, Dyomics-521XL, Dyomics-530, Dyomics-547, Dyomics-547P1, Dyomics-548, Dyomics-549, Dyomics-549P1, Dyomics-550, Dyomics-554, Dyomics-555, Dyomics-556, Dyomics-560, Dyomics-590, Dyomics-591, Dyomics-594, Dyomics-601XL, Dyomics-605, Dyomics-610, Dyomics-615, Dyomics-630, Dyomics-631, Dyomics-632, Dyomics-633, Dyomics-634, Dyomics-635, Dyomics-636, Dyomics-647, Dyomics-647P1, Dyomics-648, Dyomics-648P1, Dyomics-649, Dyomics-649P1, Dyomics-650, Dyomics-651, Dyomics-652, Dyomics-654, Dyomics-675, Dyomics-676, Dyomics-677, Dyomics-678, Dyomics-679P1, Dyomics-680, Dyomics-681, Dyomics-682, Dyomics-700, Dyomics-701, Dyomics-703, Dyomics-704, Dyomics-730, Dyomics-731, Dyomics-732, Dyomics-734, Dyomics-749, Dyomics-749P1, Dyomics-750, Dyomics-751, Dyomics-752, Dyomics-754, Dyomics-776, Dyomics-777, Dyomics-778, Dyomics-780, Dyomics-781, Dyomics-782, Dyomics-800, Dyomics-831, eFluor® 450, Eosin, FITC, Fluorescein, HiLyte™ Fluor 405, HiLyte™ Fluor 488, HiLyte™ Fluor 532, HiLyte™ Fluor 555, HiLyte™ Fluor 594, HiLyte™ Fluor 647, HiLyte™ Fluor 680, HiLyte™ Fluor 750, IRDye® 680LT, IRDye® 750, IRDye® 800CW, JOE, LightCycler® 640R, LightCycler® Red 610, LightCycler® Red 640, LightCycler® Red 670, LightCycler® Red 705, Lissamine Rhodamine B, Napthofluorescein, Oregon Green® 488, Oregon Green® 514, Pacific Blue™ Pacific Green™, Pacific Orange™, PET, PF350, PF405, PF415, PF488, PF505, PF532, PF546, PF555P, PF568, PF594, PF610, PF633P, PF647P, Quasar® 570, Quasar® 670, Quasar® 705, Rhodamine 123, Rhodamine 6G, Rhodamine B, Rhodamine Green, Rhodamine Green-X, Rhodamine Red, ROX, Seta™ 375, Seta™ 470, Seta™ 555, Seta™ 632, Seta™ 633, Seta™ 650, Seta™ 660, Seta™ 670, Seta™ 680, Seta™ 700, Seta™ 750, Seta™ 780, Seta™ APC-780, Seta™ PerCP-680, Seta™ R-PE-670, Seta™ 646, SeTau 380, SeTau 425, SeTau 647, SeTau 405, Square 635, Square 650, Square 660, Square 672, Square 680, Sulforhodamine 101, TAMRA, TET, Texas Red®, TMR, TRITC, Yakima Yellow™, Zenon®, Zy3, Zy5, Zy5.5, and Zy7.

In some aspects, the disclosure provides methods and compositions for polypeptide analysis (e.g., amino acid recognition) based on one or more luminescence properties of a luminescent label. In some embodiments, a luminescent label is identified based on luminescence lifetime, luminescence intensity, brightness, absorption spectra, emission spectra, luminescence quantum yield, or a combination of two or more thereof. In some embodiments, a plurality of types of luminescent labels can be distinguished from each other based on a difference in luminescence lifetime, luminescence intensity, brightness, absorption spectra, emission spectra, luminescence quantum yield, or combinations of two or more thereof.

In some embodiments, luminescence is detected by exposing a luminescent label to a series of separate light pulses and evaluating the timing or other properties of each photon that is emitted from the label. In some embodiments, information for a plurality of photons emitted sequentially from a label is aggregated and evaluated to identify the label and thereby identify an associated barcode site. In some embodiments, a luminescence lifetime of a label is determined from a plurality of photons that are emitted sequentially from the label, and the luminescence lifetime can be used to identify the label. In some embodiments, a luminescence intensity of a label is determined from a plurality of photons that are emitted sequentially from the label, and the luminescence intensity can be used to identify the label. In some embodiments, a luminescence lifetime and luminescence intensity of a label is determined from a plurality of photons that are emitted sequentially from the label, and the luminescence lifetime and luminescence intensity can be used to identify the label.

In some aspects of the disclosure, a single molecule is exposed to a plurality of separate light pulses and a series of emitted photons are detected and analyzed. In some embodiments, the series of emitted photons provides information about the single molecule that is present and that does not change in the mixture over the course of an experiment. However, in some embodiments, the series of emitted photons provides information about a series of different molecules that are present at different times in the mixture (e.g., as a reaction or process progresses).

In certain embodiments, a luminescent label absorbs one photon and emits one photon after a time duration. In some embodiments, the luminescence lifetime of a label can be determined or estimated by measuring the time duration. In some embodiments, the luminescence lifetime of a label can be determined or estimated by measuring a plurality of time durations for multiple pulse events and emission events. In some embodiments, the luminescence lifetime of a label can be differentiated amongst the luminescence lifetimes of a plurality of types of labels by measuring the time duration. In some embodiments, the luminescence lifetime of a label can be differentiated amongst the luminescence lifetimes of a plurality of types of labels by measuring a plurality of time durations for multiple pulse events and emission events. In certain embodiments, a label is identified or differentiated amongst a plurality of types of labels by determining or estimating the luminescence lifetime of the label. In certain embodiments, a label is identified or differentiated amongst a plurality of types of labels by differentiating the luminescence lifetime of the label amongst a plurality of the luminescence lifetimes of a plurality of types of labels.

Determination of a luminescence lifetime of a luminescent label can be performed using any suitable method (e.g., by measuring the lifetime using a suitable technique or by determining time-dependent characteristics of emission). In some embodiments, determining the luminescence lifetime of one label comprises determining the lifetime relative to another label. In some embodiments, determining the luminescence lifetime of a label comprises determining the lifetime relative to a reference. In some embodiments, determining the luminescence lifetime of a label comprises measuring the lifetime (e.g., fluorescence lifetime). In some embodiments, determining the luminescence lifetime of a label comprises determining one or more temporal characteristics that are indicative of lifetime. In some embodiments, the luminescence lifetime of a label can be determined based on a distribution of a plurality of emission events (e.g., 1, 2, 3, 4, 5, 6, 7, 8, 9, 10, 11, 12, 13, 14, 15, 16, 17, 18, 19, 20, 30, 40, 50, 60, 70, 80, 90, 100, or more emission events) occurring across one or more time-gated windows relative to an excitation pulse. For example, a luminescence lifetime of a label can be distinguished from a plurality of labels having different luminescence lifetimes based on the distribution of photon arrival times measured with respect to an excitation pulse.

It should be appreciated that a luminescence lifetime of a luminescent label is indicative of the timing of photons emitted after the label reaches an excited state and the label can be distinguished by information indicative of the timing of the photons. Some embodiments may include distinguishing a label from a plurality of labels based on the luminescence lifetime of the label by measuring times associated with photons emitted by the label. The distribution of times may provide an indication of the luminescence lifetime which may be determined from the distribution. In some embodiments, the label is distinguishable from the plurality of labels based on the distribution of times, such as by comparing the distribution of times to a reference distribution corresponding to a known label. In some embodiments, a value for the luminescence lifetime is determined from the distribution of times.

As used herein, in some embodiments, luminescence intensity refers to the number of emitted photons per unit time that are emitted by a luminescent label which is being excited by delivery of a pulsed excitation energy. In some embodiments, the luminescence intensity refers to the detected number of emitted photons per unit time that are emitted by a label which is being excited by delivery of a pulsed excitation energy, and are detected by a particular sensor or set of sensors.

As used herein, in some embodiments, brightness refers to a parameter that reports on the average emission intensity per luminescent label. Thus, in some embodiments, “emission intensity” may be used to generally refer to brightness of a composition comprising one or more labels. In some embodiments, brightness of a label is equal to the product of its quantum yield and extinction coefficient.

As used herein, in some embodiments, luminescence quantum yield refers to the fraction of excitation events at a given wavelength or within a given spectral range that lead to an emission event and is typically less than 1. In some embodiments, the luminescence quantum yield of a luminescent label described herein is between 0 and about 0.001, between about 0.001 and about 0.01, between about 0.01 and about 0.1, between about 0.1 and about 0.5, between about 0.5 and 0.9, or between about 0.9 and 1. In some embodiments, a label is identified by determining or estimating the luminescence quantum yield.

As used herein, in some embodiments, an excitation energy is a pulse of light from a light source. In some embodiments, an excitation energy is in the visible spectrum. In some embodiments, an excitation energy is in the ultraviolet spectrum. In some embodiments, an excitation energy is in the infrared spectrum. In some embodiments, an excitation energy is at or near the absorption maximum of a luminescent label from which a plurality of emitted photons are to be detected. In certain embodiments, the excitation energy is between about 500 nm and about 700 nm (e.g., between about 500 nm and about 600 nm, between about 600 nm and about 700 nm, between about 500 nm and about 550 nm, between about 550 nm and about 600 nm, between about 600 nm and about 650 nm, or between about 650 nm and about 700 nm). In certain embodiments, an excitation energy may be monochromatic or confined to a spectral range. In some embodiments, a spectral range has a range of between about 0.1 nm and about 1 nm, between about 1 nm and about 2 nm, or between about 2 nm and about 5 nm. In some embodiments, a spectral range has a range of between about 5 nm and about 10 nm, between about 10 nm and about 50 nm, or between about 50 nm and about 100 nm.

### II. Kits and Compositions

In some aspects, the disclosure provides a kit comprising one or more amino acid recognizers, where at least one amino acid recognizer comprises an amino acid binding protein described herein.

In some embodiments, the kit comprises at least one Ntaq1-homologous protein described herein. For example, in some embodiments, the kit comprises a recombinant or synthetic amino acid binding protein having an amino acid sequence that is at least 80% identical (e.g., at least 85%, at least 90%, at least 95%, at least 98%, at least 99%, 80-98%, 80-95%, 80-90%, 85-95%, 90-98%, 90-100%, 95-100%, 98-100%, or 100% identical) to a sequence selected from any one of PS2150-2171, PS2195-2205, PS2244-2265, PS2278-2299, PS2392-2402, PS2428-2449, PS2088-2089, PS2234-2243, PS2366-2379, PS2408-2409, and PS2424-2427 (SEQ ID NOs: 3-144). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to a sequence selected from any one of PS2150-2171, PS2195-2205, PS2244-2265, PS2278-2299, PS2392-2402, PS2428-2449, PS2088-2089, PS2234-2243, PS2366-2379, PS2408-2409, and PS2424-2427 (SEQ ID NOs: 3-144). In some embodiments, the kit comprises a recombinant or synthetic amino acid binding protein having an amino acid sequence that is at least 80% identical (e.g., at least 85%, at least 90%, at least 95%, at least 98%, at least 99%, 80-98%, 80-95%, 80-90%, 85-95%, 90-98%, 90-100%, 95-100%, 98-100%, or 100% identical) to a sequence selected from any one of PS2300-2314 and 2450-2472 (SEQ ID NOs: 210-247). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to a sequence selected from any one of PS2300-2314 and 2450-2472 (SEQ ID NOs: 210-247). In some embodiments, the kit comprises a recombinant or synthetic amino acid binding protein having an amino acid sequence that is at least 80% identical (e.g., at least 85%, at least 90%, at least 95%, at least 98%, at least 99%, 80-98%, 80-95%, 80-90%, 85-95%, 90-98%, 90-100%, 95-100%, 98-100%, or 100% identical) to a sequence selected from any one of PS2604-2619 and PS2687-2702 (SEQ ID NOs: 248-279). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to a sequence selected from any one of PS2604-2619 and PS2687-2702 (SEQ ID NOs: 248-279).

In some embodiments, the kit comprises at least one UBR-homologous protein described herein. For example, in some embodiments, the kit comprises a recombinant or synthetic amino acid binding protein having an amino acid sequence that is at least 80% identical (e.g., at least 85%, at least 90%, at least 95%, at least 98%, at least 99%, 80-98%, 80-95%, 80-90%, 85-95%, 90-98%, 90-100%, 95-100%, 98-100%, or 100% identical) to a sequence selected from any one of PS1923-PS1938, PS1659, PS1715, PS2080-2085, PS2173-2183, and PS2406-2407 (SEQ ID NOs: 145-181). In some embodiments, the amino acid sequence is between about 40% and about 100% (e.g., 80-98%, 80-95%, 80-90%, 85-95%, 90-98%, 50-100%, 60-100%, 70-100%, 80-100%, 90-100%, or 95-100%) identical to a sequence selected from any one of PS1923-PS1938, PS1659, PS1715, PS2080-2085, PS2173-2183, and PS2406-2407 (SEQ ID NOs: 145-181).

In some embodiments, a kit comprises a first amino acid recognizer comprising an Ntaq1-homologous amino acid binding protein described herein, and a second amino acid recognizer comprising a UBR-homologous amino acid binding protein described herein. In some embodiments, the kit further comprises at least a third amino acid recognizer. In some embodiments, the third amino acid recognizer comprises a ClpS protein, a UBR protein, an Ntaq1 protein, a BIR3 domain protein, or a homolog or variant thereof. In some embodiments, a kit comprises a first amino acid recognizer comprising an Ntaq1-homologous amino acid binding protein described herein, a second amino acid recognizer comprising a UBR-homologous amino acid binding protein described herein, and one or more amino acid recognizers comprising an amino acid binding protein having an amino acid sequence that is at least 80%, at least 85%, at least 90%, at least 95%, at least 98%, at least 99%, 90-100%, 95-100%, 98-100%, or 100% identical to a sequence selected from Table 1.

In some embodiments, the first amino acid recognizer comprises an amino acid binding protein having an amino acid sequence that is at least 80%, at least 85%, at least 90%, at least 95%, at least 98%, at least 99%, 90-100%, 95-100%, 98-100%, or 100% identical to PS2195 (SEQ ID NO: 25). In some embodiments, the second amino acid recognizer comprises an amino acid binding protein having an amino acid sequence that is at least 80%, at least 85%, at least 90%, at least 95%, at least 98%, at least 99%, 90-100%, 95-100%, 98-100%, or 100% identical to PS1936 (SEQ ID NO: 158). In some embodiments, the one or more amino acid recognizers comprise an amino acid binding protein having an amino acid sequence that is at least 80%, at least 85%, at least 90%, at least 95%, at least 98%, at least 99%, 90-100%, 95-100%, 98-100%, or 100% identical to a sequence selected from any one of PS610, PS1587, PS1751, and PS2225 (SEQ ID NOs: 182-185).

In some embodiments, a kit comprises a first amino acid recognizer comprising an Ntaq1-homologous amino acid binding protein described herein, a second amino acid recognizer comprising a UBR-homologous amino acid binding protein described herein, a third amino acid recognizer comprising an Ntaq-1 homologous amino acid binding protein described herein, and one or more amino acid recognizers comprising an amino acid binding protein having an amino acid sequence that is at least 80%, at least 85%, at least 90%, at least 95%, at least 98%, at least 99%, 90-100%, 95-100%, 98-100%, or 100% identical to a sequence selected from Table 1.

In some embodiments, the first amino acid recognizer comprises an amino acid binding protein having an amino acid sequence that is at least 80%, at least 85%, at least 90%, at least 95%, at least 98%, at least 99%, 90-100%, 95-100%, 98-100%, or 100% identical to PS2195 (SEQ ID NO: 25). In some embodiments, the second amino acid recognizer comprises an amino acid binding protein having an amino acid sequence that is at least 80%, at least 85%, at least 90%, at least 95%, at least 98%, at least 99%, 90-100%, 95-100%, 98-100%, or 100% identical to PS1936 (SEQ ID NO: 158). In some embodiments, the third amino acid recognizer comprises an amino acid binding protein having an amino acid sequence that is at least 80%, at least 85%, at least 90%, at least 95%, at least 98%, at least 99%, 90-100%, 95-100%, 98-100%, or 100% identical to PS2459 (SEQ ID NO: 234). In some embodiments, the one or more amino acid recognizers comprise an amino acid binding protein having an amino acid sequence that is at least 80%, at least 85%, at least 90%, at least 95%, at least 98%, at least 99%, 90-100%, 95-100%, 98-100%, or 100% identical to a sequence selected from any one of PS610, PS1587, PS1751, and PS2225 (SEQ ID NOs: 182-185).

In some embodiments, the kit comprises one or more cleaving reagents described herein or known in the art. In some embodiments, at least one cleaving reagent comprises an aminopeptidase. In some embodiments, the kit comprises instructions for using the kit in a method of polypeptide analysis described herein or known in the art. See, for example, PCT International Publication No. WO2020102741A1, filed Nov. 15, 2019, PCT International Publication No. WO2021236983A2, filed May 20, 2021, and PCT International Publication No. WO2024086832A1, filed Oct. 20, 2023, the relevant contents of each of which are incorporated herein.

In some aspects, the disclosure provides compositions comprising two or more amino acid recognizers, where at least one amino acid recognizer comprises an amino acid binding protein described herein. In some embodiments, the composition comprises at least one Ntaq1-homologous protein. In some embodiments, the composition comprises at least one UBR-homologous protein. In some embodiments, the composition comprises at least one ClpS-homologous protein. In some embodiments, the composition comprises at least one BIR3 domain-homologous protein. In some embodiments, each of the two or more amino acid recognizers of the composition comprises an amino acid binding protein described herein.

In some embodiments, the composition further comprises at least one type of cleaving reagent. Compositions comprising amino acid recognizer and cleaving reagent may be referred to herein as a reaction mixture (e.g., a polypeptide sequencing reaction mixture). A peptidase, also referred to as a protease or proteinase, is an enzyme that catalyzes the hydrolysis of a peptide bond. Peptidases digest polypeptides into shorter fragments and may be generally classified into endopeptidases and exopeptidases, which cleave a polypeptide chain internally and terminally, respectively. In some embodiments, a cleaving reagent comprises an exopeptidase (e.g., an aminopeptidase). Examples of suitable peptidases have been described and are contemplated for use in accordance with the present disclosure. See, for example, PCT International Publication No. WO2020102741A1, filed Nov. 15, 2019, PCT International Publication No. WO2021236983A2, filed May 20, 2021, and PCT International Publication No. WO2024086832A1, filed Oct. 20, 2023, the relevant contents of each of which are incorporated herein.

As described herein, compositions of the disclosure can be used to determine at least one chemical characteristic of a polypeptide based on a characteristic pattern. In some embodiments, polypeptide sequencing reaction conditions can be configured to achieve a time interval that allows for sufficient association events which provide a desired confidence level with a characteristic pattern. This can be achieved, for example, by configuring the reaction conditions based on various properties, including: reagent concentration, molar ratio of one reagent to another (e.g., ratio of amino acid recognition molecule to cleaving reagent, ratio of one recognizer to another, ratio of one cleaving reagent to another), number of different reagent types (e.g., the number of different types of recognizers and/or cleaving reagents, the number of recognizer types relative to the number of cleaving reagent types), cleavage activity (e.g., peptidase activity), binding properties (e.g., kinetic and/or thermodynamic binding parameters for recognition molecule binding), reagent modification (e.g., polyol and other recognizer modifications which can alter interaction dynamics), reaction mixture components (e.g., one or more components, such as pH, buffering agent, salt, divalent cation, surfactant, and other reaction mixture components described herein), temperature of the reaction, and various other parameters apparent to those skilled in the art, and combinations thereof. The reaction conditions can be configured based on one or more aspects described herein, including, for example, signal pulse information (e.g., pulse duration, interpulse duration, change in magnitude), labeling strategies (e.g., number and/or type of fluorophore, linkers with or without shielding element), surface modification (e.g., modification of sample well surface, including polypeptide immobilization), sample preparation (e.g., polypeptide fragment size, polypeptide modification for immobilization), and other aspects described herein.

In some embodiments, a polypeptide sequencing reaction in accordance with the disclosure is performed under conditions in which recognition and cleavage of amino acids can occur simultaneously in a single reaction mixture. For example, in some embodiments, a polypeptide sequencing reaction is performed in a reaction mixture having a pH at which association events and cleavage events can occur. Accordingly, in some embodiments, a reaction mixture has a pH of between about 6.5 and about 9.0. In some embodiments, a reaction mixture has a pH of between about 7.0 and about 8.5 (e.g., between about 7.0 and about 8.0, between about 7.5 and about 8.5, between about 7.5 and about 8.0, or between about 8.0 and about 8.5).

In some embodiments, a polypeptide sequencing reaction is performed in a reaction mixture comprising one or more buffering agents. In some embodiments, a reaction mixture comprises a buffering agent in a concentration of at least 10 mM (e.g., at least 20 mM and up to 250 mM, at least 50 mM, 10-250 mM, 10-100 mM, 20-100 mM, 50-100 mM, or 100-200 mM). In some embodiments, a reaction mixture comprises a buffering agent in a concentration of between about 10 mM and about 50 mM (e.g., between about 10 mM and about 25 mM, between about 25 mM and about 50 mM, or between about 20 mM and about 40 mM). Examples of buffering agents include, without limitation, HEPES (4-(2-hydroxyethyl)-1-piperazineethanesulfonic acid), Tris (tris(hydroxymethyl)aminomethane), and MOPS (3-(N-morpholino) propane sulfonic acid).

In some embodiments, a polypeptide sequencing reaction is performed in a reaction mixture comprising salt in a concentration of at least 10 mM. In some embodiments, a reaction mixture comprises salt in a concentration of at least 10 mM (e.g., at least 20 mM, at least 50 mM, at least 100 mM, or more). In some embodiments, a reaction mixture comprises salt in a concentration of between about 10 mM and about 250 mM (e.g., between about 20 mM and about 200 mM, between about 50 mM and about 150 mM, between about 10 mM and about 50 mM, or between about 10 mM and about 100 mM). Examples of salts include, without limitation, sodium salts, potassium salts, and acetates, such as sodium chloride (NaCl), sodium acetate (NaOAc), and potassium acetate (KOAc).

Additional examples of components for use in a reaction mixture include divalent cations (e.g., Mg²⁺, Co²⁺) and surfactants (e.g., polysorbate 20). In some embodiments, a reaction mixture comprises a divalent cation in a concentration of between about 0.1 mM and about 50 mM (e.g., between about 10 mM and about 50 mM, between about 0.1 mM and about 10 mM, or between about 1 mM and about 20 mM). In some embodiments, a reaction mixture comprises a surfactant in a concentration of at least 0.01% (e.g., between about 0.01% and about 0.10%). In some embodiments, a reaction mixture comprises one or more components useful in single-molecule analysis, such as an oxygen-scavenging system (e.g., a PCA/PCD system or a Pyranose oxidase/Catalase/glucose system) and/or one or more triplet state quenchers (e.g., Trolox, COT, and NBA).

In some embodiments, a polypeptide sequencing reaction is performed at a temperature at which association events and cleavage events can occur. In some embodiments, a polypeptide sequencing reaction is performed at a temperature of at least 10° C. In some embodiments, a polypeptide sequencing reaction is performed at a temperature of between about 10° C. and about 50° C. (e.g., 15-45° C., 20-40° C., at or around 25° C., at or around 30° C., at or around 35° C., at or around 37° C.). In some embodiments, a polypeptide sequencing reaction is performed at or around room temperature.

As detailed above, a real-time sequencing process as illustrated by FIG. 1A can generally involve cycles of amino acid recognition and terminal amino acid cleavage. In some embodiments, the relative occurrence of recognition and cleavage can be controlled by a concentration differential between one or more amino acid recognizers and at least one cleaving reagent. In some embodiments, the concentration differential can be optimized such that the number of signal pulses detected during recognition of an individual amino acid provides a desired confidence interval for identification. For example, if an initial sequencing reaction provides signal data with too few signal pulses between cleavage events to permit determination of characteristic patterns with a desired confidence interval, the sequencing reaction can be repeated using a decreased concentration of non-specific exopeptidase relative to recognition molecule.

In some embodiments, polypeptide analysis in accordance with the disclosure may be carried out by contacting a polypeptide with a reaction mixture comprising one or more amino acid recognizers and one or more cleaving reagents (e.g., peptidases). In some embodiments, a reaction mixture comprises an amino acid recognizer at a concentration of between about 10 nM and about 10 μM. In some embodiments, a reaction mixture comprises a cleaving reagent at a concentration of between about 500 nM and about 500 μM.

In some embodiments, a reaction mixture comprises an amino acid recognizer at a concentration of between about 100 nM and about 10 μM, between about 250 nM and about 10 μM, between about 100 nM and about 1 μM, between about 250 nM and about 1 μM, between about 250 nM and about 750 nM, or between about 500 nM and about 1 μM. In some embodiments, a reaction mixture comprises an amino acid recognizer at a concentration of about 100 nM, about 250 nM, about 500 nM, about 750 nM, or about 1 μM. In some embodiments, a reaction mixture comprises a cleaving reagent at a concentration of between about 500 nM and about 250 μM, between about 500 nM and about 100 μM, between about 1 μM and about 100 μM, between about 500 nM and about 50 μM, between about 1 μM and about 100 μM, between about 10 μM and about 200 μM, or between about 10 μM and about 100 μM. In some embodiments, a reaction mixture comprises a cleaving reagent at a concentration of about 1 μM, about 5 μM, about 10 μM, about 30 μM, about 50 μM, about 70 μM, or about 100 μM.

In some embodiments, a reaction mixture comprises an amino acid recognizer at a concentration of between about 10 nM and about 10 μM, and a cleaving reagent at a concentration of between about 500 nM and about 500 μM. In some embodiments, a reaction mixture comprises an amino acid recognizer at a concentration of between about 100 nM and about 1 μM, and a cleaving reagent at a concentration of between about 1 μM and about 100 μM. In some embodiments, a reaction mixture comprises an amino acid recognizer at a concentration of between about 250 nM and about 1 μM, and a cleaving reagent at a concentration of between about 10 μM and about 100 μM. In some embodiments, a reaction mixture comprises an amino acid recognizer at a concentration of about 500 nM, and a cleaving reagent at a concentration of between about 25 μM and about 75 μM. In some embodiments, the concentration of an amino acid recognizer and/or the concentration of a cleaving reagent in a reaction mixture is as described elsewhere herein.

In some embodiments, a reaction mixture comprises one or more amino acid recognizer and one or more cleaving reagents. In some embodiments, a reaction mixture comprises at least three amino acid recognizers and at least one cleaving reagent. In some embodiments, the reaction mixture comprises two or more cleaving reagents. In some embodiments, the reaction mixture comprises at least one and up to ten cleaving reagents (e.g., 1-3 cleaving reagents, 2-10 cleaving reagents, 1-5 cleaving reagents, 3-10 cleaving reagents). In some embodiments, the reaction mixture comprises at least three and up to thirty amino acid recognizers (e.g., between 3 and 25, between 3 and 20, between 3 and 10, between 3 and 5, between 5 and 30, between 5 and 20, between 5 and 10, or between 10 and 20, amino acid recognizers). In some embodiments, the one or more amino acid recognizers include at least one amino acid binding protein selected from Table 1.

In some embodiments, a reaction mixture comprises more than one amino acid recognizer and/or more than one cleaving reagent. In some embodiments, a reaction mixture described as comprising more than one amino acid recognizer (or cleaving reagent) refers to the mixture as having more than one type of amino acid recognizer (or cleaving reagent). For example, in some embodiments, a reaction mixture comprises two or more amino acid binding proteins, where the two or more amino acid binding proteins refer to two or more types of amino acid binding proteins. In some embodiments, one type of amino acid binding protein has an amino acid sequence that is different from another type of amino acid binding protein in the reaction mixture. In some embodiments, one type of amino acid binding protein has a label that is different from a label of another type of amino acid binding protein in the reaction mixture. In some embodiments, one type of amino acid binding protein associates with (e.g., binds to) an amino acid that is different from an amino acid with which another type of amino acid binding protein in the reaction mixture associates. In some embodiments, one type of amino acid binding protein associates with (e.g., binds to) a subset of amino acids that is different from a subset of amino acids with which another type of amino acid binding protein in the reaction mixture associates.

### III. Polypeptide Analysis

In some aspects, the disclosure provides methods of polypeptide analysis (e.g., polypeptide sequencing). In some embodiments, a method of polypeptide analysis comprises contacting a polypeptide with at least one amino acid recognizer described herein; monitoring a signal for signal pulses corresponding to interactions between the polypeptide and the at least one amino acid recognizer; and determining at least one chemical characteristic of the polypeptide based on a characteristic pattern in the signal.

A non-limiting example of polypeptide structure analysis by detecting single molecule binding interactions during a polypeptide degradation process is illustrated in FIG. 1A. An example signal trace is shown depicting different association (e.g., binding) events at times corresponding to changes in the signal. As shown, an association event between an amino acid recognizer and a terminal end of a polypeptide produces a change in magnitude of the signal that persists for a duration of time. Different association events are illustrated for different amino acids exposed at the terminal end of the polypeptide. As described herein, an amino acid that is “exposed” at the terminus of a polypeptide is an amino acid that is still attached to the polypeptide and that becomes the terminal amino acid upon removal of the prior terminal amino acid during degradation (e.g., either alone or along with one or more additional amino acids).

As generically depicted, the association events between amino acid recognizers and different types of amino acids at the terminal end of the polypeptide produce distinctive changes in the signal, referred to herein as a characteristic pattern, which may be used to determine chemical characteristics of the polypeptide. In some embodiments, a characteristic pattern corresponding to one type of terminal amino acid can be used to determine structural information for the terminal amino acid and one or more amino acids contiguous to the terminal amino acid. Accordingly, in some embodiments, a characteristic pattern corresponding to one type of terminal amino acid can be used to determine structural information for at least two (e.g., at least three, at least four, at least five, two, three, four, or between two and five) amino acids of a polypeptide.

In some embodiments, a transition from one characteristic pattern to another is indicative of amino acid cleavage. As used herein, in some embodiments, amino acid cleavage refers to the removal of at least one amino acid from a terminus of a polypeptide (e.g., the removal of at least one terminal amino acid from the polypeptide). In some embodiments, amino acid cleavage is determined by inference based on a time duration between characteristic patterns. In some embodiments, amino acid cleavage is determined by detecting a change in signal produced by association of a labeled cleaving reagent with an amino acid at the terminus of the polypeptide. As amino acids are sequentially cleaved from the terminus of the polypeptide during degradation, a series of changes in magnitude, or a series of signal pulses, is detected.

In some embodiments, signal data can be analyzed to extract signal pulse information by applying threshold levels to one or more parameters of the signal data. For example, in some embodiments, a threshold magnitude level may be applied to the signal data of a signal trace. In some embodiments, the threshold magnitude level is a minimum difference between a signal detected at a point in time and a baseline determined for a given set of data. In some embodiments, a signal pulse is assigned to each portion of the data that is indicative of a change in magnitude exceeding the threshold magnitude level and persisting for a duration of time. In some embodiments, a threshold time duration may be applied to a portion of the data that satisfies the threshold magnitude level to determine whether a signal pulse is assigned to that portion. For example, experimental artifacts may give rise to a change in magnitude exceeding the threshold magnitude level but that does not persist for a duration of time sufficient to assign a signal pulse with a desired confidence (e.g., transient association events which could be non-discriminatory for amino acid type, non-specific detection events such as diffusion into an observation region or reagent sticking within an observation region). Accordingly, in some embodiments, a signal pulse is extracted from signal data based on a threshold magnitude level and a threshold time duration.

In some embodiments, a peak in magnitude of a signal pulse is determined by averaging the magnitude detected over a duration of time that persists above the threshold magnitude level. It should be appreciated that, in some embodiments, a “signal pulse” as used herein can refer to a change in signal data that persists for a duration of time above a baseline (e.g., raw signal data), or to signal pulse information extracted therefrom (e.g., processed signal data).

In some embodiments, signal pulse information can be analyzed to identify different types of amino acids in a polypeptide based on different characteristic patterns in a series of signal pulses. For example, as shown in FIG. 1A, the signal pulse information is indicative of different types of amino acids at a terminal end of a polypeptide (e.g., arginine, leucine, isoleucine, phenylalanine). By way of example, the signal pulses detected at the earliest time points provide information indicative of (at least) arginine at the terminus of the polypeptide based on a first characteristic pattern, and the signal pulses detected at the latest time points provide information indicative of at least phenylalanine at the terminus of the polypeptide based on a second characteristic pattern.

In some embodiments, each signal pulse of a characteristic pattern comprises a pulse duration corresponding to an association event between an amino acid recognizer and an amino acid ligand. In some embodiments, the pulse duration is characteristic of a dissociation rate of binding. In some embodiments, each signal pulse of a characteristic pattern is separated from another signal pulse of the characteristic pattern by an interpulse duration. In some embodiments, the interpulse duration is characteristic of an association rate of binding. In some embodiments, a change in magnitude in a signal can be determined for a signal pulse based on a difference between baseline and the peak of a signal pulse. In some embodiments, a characteristic pattern is determined based on pulse duration. In some embodiments, a characteristic pattern is determined based on pulse duration and interpulse duration. In some embodiments, a characteristic pattern is determined based on any one or more of pulse duration, interpulse duration, and change in magnitude.

Accordingly, as illustrated by FIG. 1A, in some embodiments, polypeptide analysis is performed by detecting a series of signal pulses indicative of association of one or more amino acid recognizers with successive amino acids exposed at the terminus of a polypeptide in an ongoing degradation reaction. The series of signal pulses can be analyzed to determine characteristic patterns in the series of signal pulses, and the time course of characteristic patterns can be used to determine chemical characteristics throughout an amino acid sequence of the polypeptide.

As described herein, signal pulse information may be used to identify an amino acid based on a characteristic pattern in a series of signal pulses. In some embodiments, a characteristic pattern comprises a plurality of signal pulses, each signal pulse comprising a pulse duration. In some embodiments, the plurality of signal pulses may be characterized by a summary statistic (e.g., mean, median, time decay constant) of the distribution of pulse durations in a characteristic pattern. In some embodiments, the mean pulse duration of a characteristic pattern is between about 1 millisecond and about 10 seconds (e.g., between about 1 ms and about 1 s, between about 1 ms and about 100 ms, between about 1 ms and about 10 ms, between about 10 ms and about 10 s, between about 100 ms and about 10 s, between about 1 s and about 10 s, between about 10 ms and about 100 ms, or between about 100 ms and about 500 ms). In some embodiments, the mean pulse duration is between about 50 milliseconds and about 2 seconds, between about 50 milliseconds and about 500 milliseconds, or between about 500 milliseconds and about 2 seconds.

In some embodiments, different characteristic patterns corresponding to different types of amino acids in a single polypeptide may be distinguished from one another based on a statistically significant difference in the summary statistic. For example, in some embodiments, one characteristic pattern may be distinguishable from another characteristic pattern based on a difference in mean pulse duration of at least 10 milliseconds (e.g., between about 10 ms and about 10 s, between about 10 ms and about 1 s, between about 10 ms and about 100 ms, between about 100 ms and about 10 s, between about 1 s and about 10 s, or between about 100 ms and about 1 s). In some embodiments, the difference in mean pulse duration is at least 50 ms, at least 100 ms, at least 250 ms, at least 500 ms, or more. In some embodiments, the difference in mean pulse duration is between about 50 ms and about 1 s, between about 50 ms and about 500 ms, between about 50 ms and about 250 ms, between about 100 ms and about 500 ms, between about 250 ms and about 500 ms, or between about 500 ms and about 1 s. In some embodiments, the mean pulse duration of one characteristic pattern is different from the mean pulse duration of another characteristic pattern by about 10-25%, 25-50%, 50-75%, 75-100%, or more than 100%, for example by about 2-fold, 3-fold, 4-fold, 5-fold, or more. It should be appreciated that, in some embodiments, smaller differences in mean pulse duration between different characteristic patterns may require a greater number of pulse durations within each characteristic pattern to distinguish one from another with statistical confidence.

In some embodiments, a characteristic pattern generally refers to a plurality of association events between an amino acid of a polypeptide and a means for binding the amino acid (e.g., an amino acid recognition molecule). In some embodiments, a characteristic pattern comprises at least 10 association events (e.g., at least 25, at least 50, at least 75, at least 100, at least 250, at least 500, at least 1,000, or more, association events). In some embodiments, a characteristic pattern comprises between about 10 and about 1,000 association events (e.g., between about 10 and about 500 association events, between about 10 and about 250 association events, between about 10 and about 100 association events, or between about 50 and about 500 association events). In some embodiments, the plurality of association events is detected as a plurality of signal pulses.

In some embodiments, a characteristic pattern refers to a plurality of signal pulses which may be characterized by a summary statistic as described herein. In some embodiments, a characteristic pattern comprises at least 10 signal pulses (e.g., at least 25, at least 50, at least 75, at least 100, at least 250, at least 500, at least 1,000, or more, signal pulses). In some embodiments, a characteristic pattern comprises between about 10 and about 1,000 signal pulses (e.g., between about 10 and about 500 signal pulses, between about 10 and about 250 signal pulses, between about 10 and about 100 signal pulses, or between about 50 and about 500 signal pulses).

In some embodiments, a characteristic pattern refers to a plurality of association events between an amino acid recognition molecule and an amino acid of a polypeptide occurring over a time interval prior to removal of the amino acid (e.g., a cleavage event). In some embodiments, a characteristic pattern refers to a plurality of association events occurring over a time interval between two cleavage events (e.g., prior to removal of the amino acid and after removal of an amino acid previously exposed at the terminus). In some embodiments, the time interval of a characteristic pattern is between about 1 minute and about 30 minutes (e.g., between about 1 minute and about 20 minutes, between about 1 minute and 10 minutes, between about 5 minutes and about 20 minutes, between about 5 minutes and about 15 minutes, or between about 5 minutes and about 10 minutes).

In some embodiments, the series of signal pulses comprises a series of changes in magnitude of an optical signal over time. In some embodiments, the series of changes in the optical signal comprises a series of changes in luminescence produced during association events. In some embodiments, luminescence is produced by a detectable label associated with one or more reagents of a sequencing reaction. For example, in some embodiments, each of the one or more amino acid recognizers comprises a luminescent label. In some embodiments, a cleaving reagent comprises a luminescent label. Examples of luminescent labels and their use in accordance with the disclosure are provided herein.

In some embodiments, the series of signal pulses comprises a series of changes in magnitude of an electrical signal over time. In some embodiments, the series of changes in the electrical signal comprises a series of changes in conductance produced during association events. In some embodiments, conductivity is produced by a detectable label associated with one or more reagents of a sequencing reaction. For example, in some embodiments, each of the one or more amino acid recognizers comprises a conductivity label. Examples of conductivity labels and their use in accordance with the disclosure are provided elsewhere herein. Methods for identifying single molecules using conductivity labels have been described (see, e.g., U.S. Patent Publication No. 2017/0037462).

In some embodiments, the series of changes in conductance comprises a series of changes in conductance through a nanopore. For example, methods of evaluating receptor-ligand interactions using nanopores have been described (see, e.g., Thakur, A. K. & Movileanu, L. (2019) Nature Biotechnology 37(1)). The inventors have recognized and appreciated that such nanopores may be used to monitor polypeptide sequencing reactions in accordance with the disclosure. Accordingly, in some embodiments, the disclosure provides methods of polypeptide analysis comprising contacting a single polypeptide molecule with one or more amino acid recognizers described herein, where the single polypeptide molecule is immobilized to a nanopore. In some embodiments, the methods further comprise detecting a series of changes in conductance through the nanopore indicative of association of the one or more amino acid recognizers with successive amino acids exposed at a terminus of the single polypeptide while the single polypeptide is being degraded.

As described herein, in some embodiments, amino acid recognizers of the disclosure may be used to determine at least one chemical characteristic of a polypeptide. In some embodiments, determining at least one chemical characteristic comprises determining the type of amino acid that is present at a terminal end of a polypeptide and/or the types of amino acids that are present at one or more positions contiguous to the amino acid at the terminal end. In some embodiments, determining the type of amino acid comprises determining the actual amino acid identity, for example by determining which of the naturally-occurring 20 amino acids is present. In some embodiments, the type of amino acid is selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine.

In some embodiments, determining at least one chemical characteristic of a polypeptide comprises determining a subset of potential amino acids that can be present in the polypeptide. In some embodiments, this can be accomplished by determining that an amino acid is not one or more specific amino acids (and therefore could be any of the other amino acids). In some embodiments, this can be accomplished by determining which of a specified subset of amino acids (e.g., based on size, charge, hydrophobicity, post-translational modification, binding properties) could be in the polypeptide (e.g., using a recognizer that binds to a specified subset of two or more amino acids).

In some embodiments, determining at least one chemical characteristic of a polypeptide comprises determining that an amino acid comprises a post-translational modification. Non-limiting examples of post-translational modifications include acetylation (e.g., acetylated lysine), ADP-ribosylation, caspase cleavage, citrullination, formylation, N-linked glycosylation (e.g., glycosylated asparagine), O-linked glycosylation (e.g., glycosylated serine, glycosylated threonine), hydroxylation, methylation (e.g., methylated lysine, methylated arginine), myristoylation (e.g., myristoylated glycine), neddylation, nitration (e.g., nitrated tyrosine), chlorination (e.g., chlorinated tyrosine), oxidation/reduction (e.g., oxidized cysteine, oxidized methionine), palmitoylation (e.g., palmitoylated cysteine), phosphorylation, prenylation (e.g., prenylated cysteine), S-nitrosylation (e.g., S-nitrosylated cysteine, S-nitrosylated methionine), sulfation, sumoylation (e.g., sumoylated lysine), and ubiquitination (e.g., ubiquitinated lysine).

In some embodiments, determining at least one chemical characteristic of a polypeptide comprises determining that an amino acid comprises an arginine post-translational modification. For example, as described herein, amino acid recognizers of the disclosure are capable of distinguishing between different arginine modifications, including symmetric dimethylarginine (SDMA), asymmetric dimethylarginine (ADMA), and citrullinated arginine.

In some embodiments, determining at least one chemical characteristic of a polypeptide comprises determining that an amino acid comprises a phosphorylated side chain. For example, in some embodiments, determining at least one chemical characteristic of a polypeptide comprises determining that an amino acid comprises phosphorylated threonine (e.g., phospho-threonine). In some embodiments, determining at least one chemical characteristic of a polypeptide comprises determining that an amino acid comprises phosphorylated tyrosine (e.g., phospho-tyrosine). In some embodiments, determining at least one chemical characteristic of a polypeptide comprises determining that an amino acid comprises phosphorylated serine (e.g., phospho-serine).

In some embodiments, determining at least one chemical characteristic of a polypeptide comprises determining that an amino acid comprises a chemically modified variant, an unnatural amino acid, or a proteinogenic amino acid such as selenocysteine and pyrrolysine. Examples of unnatural amino acids include, without limitation, 2-naphthyl-alanine, statine, homoalanine, α-amino acid, β2-amino acid, β3-amino acid, 7-amino acid, 3-pyridyl-alanine, 4-fluorophenyl-alanine, cyclohexyl-alanine, N-alkyl amino acid, peptoid amino acid, homo-cysteine, penicillamine, 3-nitro-tyrosine, homo-phenyl-alanine, t-leucine, hydroxy-proline, 3-Abz, 5-F-tryptophan, and azabicyclo-[2.2.1]heptane.

In some embodiments, determining at least one chemical characteristic of a polypeptide comprises determining that an amino acid comprises an oxidative modification. For example, as described herein, amino acid recognizers of the disclosure are capable of distinguishing between oxidized methionine and its unmodified variant. In some embodiments, the oxidative modification comprises an oxidatively-damaged side chain of an amino acid. In some embodiments, the oxidatively-damaged side chain comprises a cysteine-derived product (e.g., disulfide, sulfinic acid, sulfonic acid, sulfenic acid, S-nitrosocysteine), a tyrosine-derived product (e.g., di-tyrosine, 3,4-dihydroxyphenylalanine, 3-chlorotyrosine, 3-nitrotyrosine), a histidine-derived product (e.g., 2-oxohistidine, 4-hydroxy-2-oxohistidine, di-histidine, asparagine, aspartic acid, urea), a methionine-derived product (e.g., sulfoxide, sulfone), a tryptophan-derived product (e.g., di-tryptophan, N-formylkynurenine, kynurenine, 2-oxo-tryptophan oxindolylalanine, 6-nitrotryptophan, hydroxytryptophan), a phenylalanine-derived product (e.g., meta-tyrosine, ortho-tyrosine), or a generic side-chain product (e.g., alcohol, hydroperoxide, aldehyde/ketone carbonyl). Examples of oxidatively damaged amino acids are known in the art, see, e.g., Hawkins, C. L., Davies, M. J. Detection, identification, and quantification of oxidative protein modifications. J Biol Chem. 2019 Dec. 20; 294(51):19683-19708.

In some embodiments, determining at least one chemical characteristic of a polypeptide comprises determining that an amino acid comprises a side chain characterized by one or more biochemical properties. For example, an amino acid may comprise a nonpolar aliphatic side chain, a positively charged side chain, a negatively charged side chain, a nonpolar aromatic side chain, or a polar uncharged side chain. Non-limiting examples of an amino acid comprising a nonpolar aliphatic side chain include alanine, glycine, valine, leucine, methionine, and isoleucine. Non-limiting examples of an amino acid comprising a positively charged side chain includes lysine, arginine, and histidine. Non-limiting examples of an amino acid comprising a negatively charged side chain include aspartate and glutamate. Non-limiting examples of an amino acid comprising a nonpolar, aromatic side chain include phenylalanine, tyrosine, and tryptophan. Non-limiting examples of an amino acid comprising a polar uncharged side chain include serine, threonine, cysteine, proline, asparagine, and glutamine.

In some embodiments, a protein or polypeptide can be digested into a plurality of smaller polypeptides and chemical characteristics can be determined for one or more of these smaller polypeptides. In some embodiments, a first terminus (e.g., N or C terminus) of a polypeptide is immobilized and the other terminus (e.g., the C or N terminus) is analyzed as described herein.

As used herein, sequencing a polypeptide refers to determining sequence information for a polypeptide. In some embodiments, this can involve determining the identity of each sequential amino acid for a portion (or all) of the polypeptide. However, in some embodiments, this can involve assessing the identity of a subset of amino acids within the polypeptide (e.g., and determining the relative position of one or more amino acid types without determining the identity of each amino acid in the polypeptide). However, in some embodiments, amino acid content information can be obtained from a polypeptide without directly determining the relative position of different types of amino acids in the polypeptide. The amino acid content alone may be used to infer the identity of the polypeptide that is present (e.g., by comparing the amino acid content to a database of polypeptide information and determining which polypeptide(s) have the same amino acid content).

In some embodiments, sequence information for a plurality of polypeptide products obtained from a longer polypeptide or protein (e.g., via enzymatic and/or chemical cleavage) can be analyzed to reconstruct or infer the sequence of the longer polypeptide or protein.

In some aspects, the polypeptide analysis described herein generates data indicating how a polypeptide interacts with a binding means while the polypeptide is being degraded by a cleaving means. As discussed above, the data can include a series of characteristic patterns corresponding to association events at a terminus of a polypeptide in between cleavage events at the terminus. In some embodiments, methods of polypeptide analysis described herein comprise contacting a single polypeptide molecule with a binding means and a cleaving means, where the binding means and the cleaving means are configured to achieve at least 10 association events prior to a cleavage event. In some embodiments, the means are configured to achieve the at least 10 association events between two cleavage events.

In some embodiments, a plurality of single-molecule sequencing reactions are performed in parallel in an array of sample wells. In some embodiments, an array comprises between about 10,000 and about 1,000,000 sample wells. The volume of a sample well may be between about 10⁻²¹ liters and about 10⁻¹⁵ liters, in some implementations. Because the sample well has a small volume, detection of single-molecule events may be possible as only about one polypeptide may be within a sample well at any given time. Statistically, some sample wells may not contain a single-molecule sequencing reaction and some may contain more than one single polypeptide molecule. However, an appreciable number of sample wells may each contain a single-molecule reaction (e.g., at least 30% in some embodiments), so that single-molecule analysis can be carried out in parallel for a large number of sample wells. In some embodiments, the binding means and the cleaving means are configured to achieve at least 10 association events prior to a cleavage event in at least 10% (e.g., 10-50%, more than 50%, 25-75%, at least 80%, or more) of the sample wells in which a single-molecule reaction is occurring. In some embodiments, the binding means and the cleaving means are configured to achieve at least 10 association events prior to a cleavage event for at least 50% (e.g., more than 50%, 50-75%, at least 80%, or more) of the amino acids of a polypeptide in a single-molecule reaction.

### IV. Devices and Systems

Methods in accordance with the disclosure, in some aspects, may be performed using a system that permits single-molecule analysis. The system may include an integrated device and an instrument configured to interface with the integrated device. The integrated device may include an array of pixels, where individual pixels include a sample well and at least one photodetector. The sample wells of the integrated device may be formed on or through a surface of the integrated device and be configured to receive a sample placed on the surface of the integrated device. Collectively, the sample wells may be considered as an array of sample wells. The plurality of sample wells may have a suitable size and shape such that at least a portion of the sample wells receive a single sample (e.g., a single molecule, such as a polypeptide). In some embodiments, the number of samples within a sample well may be distributed among the sample wells of the integrated device such that some sample wells contain one sample while others contain zero, two or more samples.

Excitation light is provided to the integrated device from one or more light source external to the integrated device. Optical components of the integrated device may receive the excitation light from the light source and direct the light towards the array of sample wells of the integrated device and illuminate an illumination region within the sample well. In some embodiments, a sample well may have a configuration that allows for the sample to be retained in proximity to a surface of the sample well, which may ease delivery of excitation light to the sample and detection of emission light from the sample. A sample positioned within the illumination region may emit emission light in response to being illuminated by the excitation light. For example, the sample may be labeled with a fluorescent label, which emits light in response to achieving an excited state through the illumination of excitation light. Emission light emitted by a sample may then be detected by one or more photodetectors within a pixel corresponding to the sample well with the sample being analyzed. When performed across the array of sample wells, which may range in number between approximately 10,000 pixels to 1,000,000 pixels according to some embodiments, multiple samples can be analyzed in parallel.

The integrated device may include an optical system for receiving excitation light and directing the excitation light among the sample well array. The optical system may include one or more grating couplers configured to couple excitation light to other optical components of the integrated device and direct the excitation light to the other optical components. For example, the optical system may include optical components that direct the excitation light from the grating coupler(s) towards the sample well array. Such optical components may include optical splitters, optical combiners, and waveguides. In some embodiments, one or more optical splitters may couple excitation light from a grating coupler and deliver excitation light to at least one of the waveguides. According to some embodiments, the optical splitter may have a configuration that allows for delivery of excitation light to be substantially uniform across all the waveguides such that each of the waveguides receives a substantially similar amount of excitation light. Such embodiments may improve performance of the integrated device by improving the uniformity of excitation light received by sample wells of the integrated device. Examples of suitable components, e.g., for coupling excitation light to a sample well and/or directing emission light to a photodetector, to include in an integrated device are described in U.S. patent application Ser. No. 14/821,688, filed Aug. 7, 2015, titled “INTEGRATED DEVICE FOR PROBING, DETECTING AND ANALYZING MOLECULES,” and U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES,” both of which are incorporated by reference in their entirety. Examples of suitable grating couplers and waveguides that may be implemented in the integrated device are described in U.S. patent application Ser. No. 15/844,403, filed Dec. 15, 2017, titled “OPTICAL COUPLER AND WAVEGUIDE SYSTEM,” which is incorporated by reference in its entirety.

Additional photonic structures may be positioned between the sample wells and the photodetectors and configured to reduce or prevent excitation light from reaching the photodetectors, which may otherwise contribute to signal noise in detecting emission light. In some embodiments, metal layers which may act as a circuitry for the integrated device, may also act as a spatial filter. Examples of suitable photonic structures may include spectral filters, polarization filters, and spatial filters and are described in U.S. patent application Ser. No. 16/042,968, filed Jul. 23, 2018, titled “OPTICAL REJECTION PHOTONIC STRUCTURES,” and U.S. Provisional Patent Application No. 63/124,655, filed Dec. 11, 2020, titled “INTEGRATED CIRCUIT WITH IMPROVED CHARGE TRANSFER EFFICIENCY AND ASSOCIATED TECHNIQUES,” both of which are incorporated by reference in their entirety.

Components located off of the integrated device may be used to position and align an excitation source to the integrated device. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application Ser. No. 15/161,088, filed May 20, 2016, titled “PULSED LASER AND SYSTEM,” which is incorporated by reference in its entirety. Another example of a beam-steering module is described in U.S. patent application Ser. No. 15/842,720, filed Dec. 14, 2017, titled “COMPACT BEAM SHAPING AND STEERING ASSEMBLY,” which is incorporated herein by reference. Additional examples of suitable excitation sources are described in U.S. patent application Ser. No. 14/821,688, filed Aug. 7, 2015, titled “INTEGRATED DEVICE FOR PROBING, DETECTING AND ANALYZING MOLECULES,” which is incorporated by reference in its entirety.

The photodetector(s) positioned with individual pixels of the integrated device may be configured and positioned to detect emission light from the pixel's corresponding sample well. Examples of suitable photodetectors are described in U.S. patent application Ser. No. 14/821,656, filed Aug. 7, 2015, titled “INTEGRATED DEVICE FOR TEMPORAL BINNING OF RECEIVED PHOTONS,” which is incorporated by reference in its entirety. In some embodiments, a sample well and its respective photodetector(s) may be aligned along a common axis. In this manner, the photodetector(s) may overlap with the sample well within the pixel.

Characteristics of the detected emission light may provide an indication for identifying the label associated with the emission light. Such characteristics may include any suitable type of characteristic, including an arrival time of photons detected by a photodetector, an amount of photons accumulated over time by a photodetector, and/or a distribution of photons across two or more photodetectors. In some embodiments, such characteristics can be any one or a combination of two or more of luminescence lifetime, luminescence intensity, brightness, absorption spectra, emission spectra, luminescence quantum yield, wavelength (e.g., peak wavelength), and signal characteristics (e.g., pulse duration, interpulse durations, change in signal magnitude).

In some embodiments, a photodetector may have a configuration that allows for the detection of one or more timing characteristics associated with a sample's emission light (e.g., luminescence lifetime). The photodetector may detect a distribution of photon arrival times after a pulse of excitation light propagates through the integrated device, and the distribution of arrival times may provide an indication of a timing characteristic of the sample's emission light (e.g., a proxy for luminescence lifetime). In some embodiments, the one or more photodetectors provide an indication of the probability of emission light emitted by the label (e.g., luminescence intensity). In some embodiments, a plurality of photodetectors may be sized and arranged to capture a spatial distribution of the emission light. Output signals from the one or more photodetectors may then be used to distinguish a label from among a plurality of labels, where the plurality of labels may be used to identify a sample within the sample. In some embodiments, a sample may be excited by multiple excitation energies, and emission light and/or timing characteristics of the emission light emitted by the sample in response to the multiple excitation energies may distinguish a label from a plurality of labels.

In operation, parallel analyses of samples within the sample wells are carried out by exciting some or all of the samples within the wells using excitation light and detecting signals from sample emission with the photodetectors. Emission light from a sample may be detected by a corresponding photodetector and converted to at least one electrical signal. The electrical signals may be transmitted along conducting lines in the circuitry of the integrated device, which may be connected to an instrument interfaced with the integrated device. The electrical signals may be subsequently processed and/or analyzed. Processing or analyzing of electrical signals may occur on a suitable computing device either located on or off the instrument.

The instrument may include a user interface for controlling operation of the instrument and/or the integrated device. The user interface may be configured to allow a user to input information into the instrument, such as commands and/or settings used to control the functioning of the instrument. In some embodiments, the user interface may include buttons, switches, dials, and a microphone for voice commands. The user interface may allow a user to receive feedback on the performance of the instrument and/or integrated device, such as proper alignment and/or information obtained by readout signals from the photodetectors on the integrated device. In some embodiments, the user interface may provide feedback using a speaker to provide audible feedback. In some embodiments, the user interface may include indicator lights and/or a display screen for providing visual feedback to a user.

In some embodiments, the instrument may include a computer interface configured to connect with a computing device. The computer interface may be a USB interface, a FireWire interface, or any other suitable computer interface. A computing device may be any general purpose computer, such as a laptop or desktop computer. In some embodiments, a computing device may be a server (e.g., cloud-based server) accessible over a wireless network via a suitable computer interface. The computer interface may facilitate communication of information between the instrument and the computing device. Input information for controlling and/or configuring the instrument may be provided to the computing device and transmitted to the instrument via the computer interface. Output information generated by the instrument may be received by the computing device via the computer interface. Output information may include feedback about performance of the instrument, performance of the integrated device, and/or data generated from the readout signals of the photodetector.

In some embodiments, the instrument may include a processing device configured to analyze data received from one or more photodetectors of the integrated device and/or transmit control signals to the excitation source(s). In some embodiments, the processing device may comprise a general purpose processor, a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof). In some embodiments, the processing of data from one or more photodetectors may be performed by both a processing device of the instrument and an external computing device. In other embodiments, an external computing device may be omitted and processing of data from one or more photodetectors may be performed solely by a processing device of the integrated device.

According to some embodiments, the instrument that is configured to analyze samples based on luminescence emission characteristics may detect differences in luminescence lifetimes and/or intensities between different luminescent molecules, and/or differences between lifetimes and/or intensities of the same luminescent molecules in different environments. The inventors have recognized and appreciated that differences in luminescence emission lifetimes can be used to discern between the presence or absence of different luminescent molecules and/or to discern between different environments or conditions to which a luminescent molecule is subjected. In some cases, discerning luminescent molecules based on lifetime (rather than emission wavelength, for example) can simplify aspects of the system. As an example, wavelength-discriminating optics (such as wavelength filters, dedicated detectors for each wavelength, dedicated pulsed optical sources at different wavelengths, and/or diffractive optics) may be reduced in number or eliminated when discerning luminescent molecules based on lifetime. In some cases, a single pulsed optical source operating at a single characteristic wavelength may be used to excite different luminescent molecules that emit within a same wavelength region of the optical spectrum but have measurably different lifetimes. An analytic system that uses a single pulsed optical source, rather than multiple sources operating at different wavelengths, to excite and discern different luminescent molecules emitting in a same wavelength region can be less complex to operate and maintain, more compact, and may be manufactured at lower cost.

Although analytic systems based on luminescence lifetime analysis may have certain benefits, the amount of information obtained by an analytic system and/or detection accuracy may be increased by allowing for additional detection techniques. For example, some embodiments of the systems may additionally be configured to discern one or more properties of a sample based on luminescence wavelength and/or luminescence intensity. In some implementations, luminescence intensity may be used additionally or alternatively to distinguish between different luminescent labels. For example, some luminescent labels may emit at significantly different intensities or have a significant difference in their probabilities of excitation (e.g., at least a difference of about 35%) even though their decay rates may be similar. By referencing binned signals to measured excitation light, it may be possible to distinguish different luminescent labels based on intensity levels.

According to some embodiments, different luminescence lifetimes may be distinguished with a photodetector that is configured to time-bin luminescence emission events following excitation of a luminescent label. The time binning may occur during a single charge-accumulation cycle for the photodetector. A charge-accumulation cycle is an interval between read-out events during which photo-generated carriers are accumulated in bins of the time-binning photodetector. Examples of a time-binning photodetector are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference. In some embodiments, a time-binning photodetector may generate charge carriers in a photon absorption/carrier generation region and directly transfer charge carriers to a charge carrier storage bin in a charge carrier storage region. In such embodiments, the time-binning photodetector may not include a carrier travel/capture region. Such a time-binning photodetector may be referred to as a “direct binning pixel.” Examples of time-binning photodetectors, including direct binning pixels, are described in U.S. patent application Ser. No. 15/852,571, filed Dec. 22, 2017, titled “INTEGRATED PHOTODETECTOR WITH DIRECT BINNING PIXEL,” which is incorporated herein by reference.

In some embodiments, different numbers of fluorophores of the same type may be linked to different reagents in a sample, so that each reagent may be identified based on luminescence intensity. For example, two fluorophores may be linked to a first labeled recognition molecule and four or more fluorophores may be linked to a second labeled recognition molecule. Because of the different numbers of fluorophores, there may be different excitation and fluorophore emission probabilities associated with the different recognition molecules. For example, there may be more emission events for the second labeled recognition molecule during a signal accumulation interval, so that the apparent intensity of the bins is significantly higher than for the first labeled recognition molecule.

The inventors have recognized and appreciated that distinguishing biological or chemical samples based on fluorophore decay rates and/or fluorophore intensities may enable a simplification of the optical excitation and detection systems. For example, optical excitation may be performed with a single-wavelength source (e.g., a source producing one characteristic wavelength rather than multiple sources or a source operating at multiple different characteristic wavelengths). Additionally, wavelength discriminating optics and filters may not be needed in the detection system. Also, a single photodetector may be used for each sample well to detect emission from different fluorophores. The phrase “characteristic wavelength” or “wavelength” is used to refer to a central or predominant wavelength within a limited bandwidth of radiation (e.g., a central or peak wavelength within a 20 nm bandwidth output by a pulsed optical source). In some cases, “characteristic wavelength” or “wavelength” may be used to refer to a peak wavelength within a total bandwidth of radiation output by a source.

According to an aspect of the present disclosure, an exemplary integrated device may be configured to perform single-molecule analysis in combination with an instrument as described above. It should be appreciated that the exemplary integrated device described herein is intended to be illustrative and that other integrated device configurations may be configured to perform any or all techniques described herein.

FIG. 1B illustrates a cross-sectional view of a pixel 1-112 of an integrated device 1-102. Pixel 1-112 includes a photodetection region, which may be a pinned photodiode (PPD), and a charge storage region, which may be a storage diode (SD0). In some embodiments, a photodetection region and charge storage regions may be formed in semiconductor material of a pixel by doping regions of the semiconductor material. For example, the photodetection region and charge storage regions can be formed using a same conductivity type (e.g., n-type doping or p-type doping).

During operation of pixel 1-112, excitation light may illuminate sample well 1-108 causing incident photons, including fluorescence emissions from a sample, to flow along the optical axis to photodetection region PPD. As shown in FIG. 1B, pixel 1-112 may include a waveguide 1-220 configured to optically (e.g., evanescently) couple excitation light from a grating coupler of the integrated device (not shown) to the sample well 1-108. In response, a sample in the sample well 1-108 may emit fluorescent light toward photodetection region PPD. In some embodiments, pixel 1-112 may also include one or more photonic structures 1-230, which may include one or more optical rejection structures such as a spectral filter, a polarization filter, and/or a spatial filter. For example, the photonic structures 1-230 may be configured to reduce the amount of excitation light that reaches the photodetection region PPD and/or increase the amount of fluorescent emissions that reach the photodetection region PPD. Also shown in pixel 1-112, pixel 1-112 may include one or more metal layers 1-240, which may be configured as a filter and/or may carry control signals from a control circuit configured to control transfer gates, as described further herein.

In some embodiments, pixel 1-112 may include one or more transfer gates configured to control operation of pixel 1-112 by applying an electrical bias to one or more semiconductor regions of pixel 1-112 in response to one or more control signals. For example, when transfer gate ST0 induces a first electrical bias at the semiconductor region between photodetection region PPD and storage region SD0, a transfer path (e.g., charge transfer channel) may be formed in the semiconductor region. Charge carriers (e.g., photo-electrons) generated in photodetection region PPD by the incident photons may flow along the transfer path to storage region SD0. In some embodiments, the first electrical bias may be applied during a collection period during which charge carriers from the sample are selectively directed to storage region SD0. Alternatively, when transfer gate ST0 provides a second electrical bias at the semiconductor region between photodetection region PPD and storage region SD0, charge carriers from photodetection region PPD may be blocked from reaching storage region SD0 along the transfer path. In some embodiments, drain gate REJ may provide a channel to drain D to draw noise charge carriers generated in photodetection region PPD by the excitation light away from photodetection region PPD and storage region SD0, such as during a rejection period before fluorescent emission photons from the sample reach photodetection region PPD. In some embodiments, during a readout period, transfer gate ST0 may provide the second electrical bias and transfer gate TX0 may provide an electrical bias to cause charge carriers stored in storage region SD0 to flow to the readout region, which may be a floating diffusion (FD) region, for processing.

It should be appreciated that, in accordance with various embodiments, transfer gates described herein may include semiconductor material(s) and/or metal, and may include a gate of a field effect transistor (FET), a base of a bipolar junction transistor (BJT), and/or the like.

In some embodiments, operation of pixel 1-112 may include one or more collection sequences, each collection sequence including one or more rejection (e.g., drain) periods and one or more collection periods. In one example, a collection sequence performed in accordance with one or more pulses of an excitation light source may begin with a rejection period, such as to discard charge carriers generated in pixel 1-112 (e.g., in photodetection region PD) responsive to excitation photons from the light source. For instance, the excitation photons may arrive at pixel 1-112 prior to the arrival of fluorescence emission photons from the sample well. Transfer gates for the charge storage regions may be biased to have low conductivity in the charge transfer channels coupling the charge storage regions to the photodetection region, blocking transfer and accumulation of charge carriers in the charge storage regions. A drain gate for the drain region may be biased to have high conductivity in a drain channel between the photodetection region and the drain region, facilitating draining of charge carriers from the photodetection region to the drain region. Transfer gates for any charge storage regions coupled to the photodetection region may be biased to have low conductivity between the photodetection region and the charge storage regions, such that charge carriers are not transferred to or accumulated in the charge storage regions during the rejection period.

Following the rejection period, a collection period may occur in which charge carriers generated responsive to the incident photons are transferred to one or more charge storage regions. During the collection period, the incident photons may include fluorescent emission photons, resulting in accumulation of fluorescent emission charge carriers in the charge storage region(s). For instance, a transfer gate for one of the charge storage regions may be biased to have high conductivity between the photodetection region and the charge storage region, facilitating accumulation of charge carriers in the charge storage region. Any drain gates coupled to the photodetection region may be biased to have low conductivity between the photodetection region and the drain region such that charge carriers are not discarded during the collection period.

Some embodiments may include multiple rejection and/or collection periods in a collection sequence, such as a second rejection period and second collection period following a first rejection period and a collection period, where each pair of rejection and collection periods is conducted in response to a pulse of excitation light. In one example, charge carriers generated in the photodetection region during each collection period of a collection sequence (e.g., in response to a plurality of pulses of excitation light) may be aggregated in a single charge storage region. In some embodiments, charge carriers aggregated in the charge storage region may be read out for processing prior to the next collection sequence. Alternatively or additionally, in some embodiments, charge carriers aggregated in a first charge storage region during a first collection sequence may be transferred to a second charge storage region sequentially coupled to the first charge storage region and read out simultaneously with the next collection sequence. In some embodiments, a processing circuit configured to read out charge carriers from one or more pixels may be configured to determine one or more of luminescence intensity information, luminescence lifetime information, luminescence spectral information, and/or any other mode of luminescence information associated with performing techniques described herein.

In some embodiments, a first collection sequence may include transferring, to a charge storage region at a first time following each excitation pulse, charge carriers generated in the photodetection response in response to the excitation pulse, and a second collection sequence may include transferring, to the charge storage region at a second time following each excitation pulse, charge carriers generated in the photodetection response in response to the excitation pulse. For example, the number of charge carriers aggregated after the first and second times may indicate luminance lifetime information of the received light.

As described further herein, pixels of an integrated device may be controlled to perform one or more collection sequences using one or more control signals from a control circuit of the integrated circuit, such as by providing the control signal(s) to drain and/or transfer gates of the pixel(s) of the integrated circuit. In some embodiments, charge carriers may be read out from the FD region of each pixel during a readout pixel associated with each pixel and/or a row or column of pixels for processing. In some embodiments, FD regions of the pixels may be read out using correlated double sampling (CDS) techniques.

### V. Sequence Information

As described herein, in some embodiments, an amino acid recognizer of the disclosure comprises an amino acid binding protein having an amino acid sequence that shares a percentage of sequence identity with an amino acid sequence selected from Table 1. In some embodiments, an amino acid recognizer comprises an amino acid binding protein described herein and a tag peptide having an amino acid sequence that shares a percentage of sequence identity with an amino acid sequence selected from Table 2A. For the purposes of comparing two or more amino acid sequences, the percentage of “sequence identity” between a first amino acid sequence and a second amino acid sequence (also referred to herein as “amino acid identity”) may be calculated by: dividing [the number of amino acid residues in the first amino acid sequence that are identical to the amino acid residues at the corresponding positions in the second amino acid sequence] by [the total number of amino acid residues in the first amino acid sequence] and multiplying by [100], in which each deletion, insertion, substitution or addition of an amino acid residue in the second amino acid sequence compared to the first amino acid sequence is considered as a difference at a single amino acid residue (position).

Alternatively, the degree of sequence identity between two amino acid sequences may be calculated using a known computer algorithm (e.g., by the local homology algorithm of Smith and Waterman (1970) Adv. Appl. Math. 2:482c, by the homology alignment algorithm of Needleman and Wunsch, J. Mol. Biol. (1970) 48:443, by the search for similarity method of Pearson and Lipman. Proc. Natl. Acad. Sci. USA (1998) 85:2444, or by computerized implementations of algorithms available as Blast, Clustal Omega, or other sequence alignment algorithms) and, for example, using standard settings. Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Additionally, or alternatively, two or more sequences may be assessed for the identity between the sequences. The terms “identical” or percent “identity” in the context of two or more amino acid sequences, refer to two or more sequences or subsequences that are the same. Two sequences are “substantially identical” if two sequences have a specified percentage of amino acid residues that are the same (e.g., at least 80%, 85%, 90%, 95%, 96%, 97%, 98%, 99%, 99.5%, 99.6%, 99.7%, 99.8%, or 99.9% identical) over a specified region or over the entire sequence, when compared and aligned for maximum correspondence over a comparison window, or designated region as measured using one of the above sequence comparison algorithms or by manual alignment and visual inspection. Optionally, the identity exists over a region that is at least about 25, 50, 75, or 100 amino acids in length, or over a region that is 100 to 150, 150 to 200, 100 to 200, or 200 or more, amino acids in length.

Additionally, or alternatively, two or more sequences may be assessed for the alignment between the sequences. The terms “alignment” or “percent alignment” in the context of two or more amino acid sequences, refer to two or more sequences or subsequences that are the same. Two sequences are “substantially aligned” if two sequences have a specified percentage of amino acid residues that are the same (e.g., at least 80%, 85%, 90%, 95%, 96%, 97%, 98%, 99%, 99.5%, 99.6%, 99.7%, 99.8% or 99.9% identical) over a specified region or over the entire sequence, when compared and aligned for maximum correspondence over a comparison window, or designated region as measured using one of the above sequence comparison algorithms or by manual alignment and visual inspection. Optionally, the alignment exists over a region that is at least about 25, 50, 75, or 100 amino acids in length, or over a region that is 100 to 150, 150 to 200, 100 to 200, or 200 or more amino acids in length.

TABLE 1
Non-limiting example sequences of amino acid binding proteins.

| | SEQ ID | |
|---|---|---|
| Name | NO. | Sequence |
| PS1259 | 1 | MNGLSAQHERIAPARHECVYTSCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKT |
| PS1122 | 2 | MHSKFSHAGRICGAKFKVGEPIYRCKECSFDDTCVLCVNCFNPKDHLGHHVYTTICTEFN |
| | | NGECDCGDKTAWNHTLFCKAEEG |
| PS2150 | 3 | MNGLSAQHERILPARHECVYTPCYSEENVYKLCEHIKTSKRCPLGDVYAVFISNERKMVP |
| | | IWKQKSGRGERVVIWDYQVILLHDCHKEQTFIYDLDTTLPFPCPFDTYVKEAFKSDNYIN |
| | | PRFWRKLRVVPADVFLQNFASDRSHMKDASGGWRMPPPPYPCIETAESRMNLDDFISMNP |
| | | SVGWGHVYTLEEFVQHFGKT |
| PS2151 | 4 | MNGLSAQHERILPARHECVYTPCYSEENVYKLCEHIKTSKRCPLGDVYAVFISNERKMVP |
| | | IWKQRSGRGERVVIWDYQVILLHDCHKEQTFIYDLDTTLPFPCPFDTYVKEAFKSDNYIN |
| | | PRFWRKLRVVRADVFLQNFASDRSHMKDASGGWRMPPPPYPCIETAESRMNLDDFISMNP |
| | | SVGWGHVYTLEEFVQHFGKT |
| PS2152 | 5 | MNGLSAQHERILPARHECVYTSCYSEENVWKLCEHIKTSKRCPLGDVYAVFISNERKMVP |
| | | IWKQKSGRGERLVIWDYQVILLHDCHKEQTFIYDLDTTLPFPCPFDTYVKEAFKSDNYIN |
| | | PRFWRKLRVVPADVFLQNFASDRSHMKDASGGWRMPPPPYPCIETAESRMNLDDFISMNP |
| | | SVGWGHVYTLEEFVQHFGKT |
| PS2153 | 6 | MNGLSAQHERILPARHECVYTEFYSEENVWKLCEHIKTSKRCPLGDVYAVFISNERKMVP |
| | | IWKQKSGRGERPVIWDYQVILLHDCHKEQTFIYDLDTTLPFPCPFDTYVKEAFKSDNYIN |
| | | PRFWRKLRVVPADVFLQNFASDRSHMKDASGGWRMPPPPYPCIETAESRMNLDDFISMNP |
| | | SVGWGHVYTLEEFVQHFGKT |
| PS2154 | 7 | MNGLSAQHERIAPARHECVYTPCYSEENVWKLCEHIKTSKRCPLGDVYAVFISNERKMVP |
| | | IWKQRSGRGERPVIWDYQVILLHDCHKEQTFIYDLDTTLPFPCPFDTYVKEAFKSDNYIN |
| | | PRFWRKLRVVPADVFLQNFASDRSHMKDASGGWRMPPPPYPCIETAESRMNLDDFISMNP |
| | | SVGWGHVYTLEEFVQHFGKT |
| PS2155 | 8 | MNGLSAQHERILPARHECVYTECYSEENVWKLCEHIKTSKRCPLGDVYAVFISNERKMVP |
| | | IWKQKSGRGERVVIWDYQVIMLHDCHKEQTFIYDLDTTLPFPCPFDTYVKEAFKSDNYIN |
| | | PRFWRKLRVVPADVFLQNFASDRSHMKDASGGWRMPPPPYPCIETAESRMNLDDFISMNP |
| | | SVGWGHVYTLEEFVQHFGKT |
| PS2156 | 9 | MNGLSAQHERILPARHECVYTSCYSEENVWKLCEHIKTSKRCPLGDVYAVFISNERKMVP |
| | | IWKQKSGRGERPVIWDYQVILLHDCHKEQTFIYDLDTTLPFPCPFDTYVKEAFKSDNYIN |
| | | PRFWRKLRVVPADVFLQNFASDRSHMKDASGGWRMPPPPYPCIETAESRMNLDDFISMNP |
| | | SVGWGHVYTLEEFVQHFGKT |
| PS2157 | 10 | MNGLSAQHERILPARHECVYTSCYSEENVWKLCEHIKTSKRCPLGDVYAVFISNERKMVP |
| | | IWKQRSGRGERVVIWDYQVILLHDCHKEQTFIYDLDTTLPFPCPFDTYVKEAFKSDNYIN |
| | | PRFWRKLRVVPADVFLQNFASDRSHMKDASGGWRMPPPPYPCIETAESRMNLDDFISMNP |
| | | SVGWGHVYTLEEFVQHFGKT |
| PS2158 | 11 | MNGLSAQHERILPARHECVYTECYSEENVWKLCEHIKTSKRCPLGDVYAVFISNERKMVP |
| | | IWKQKSGRGERPVIWDYQVILLHDCHKEQTFIYDLDTTLPFPCPFDTYVKEAFKSDNYIN |
| | | PRFWRKLRVVPADVFLQNFASDRSHMKDASGGWRMPPPPYPCIETAESRMNLDDFISMNP |
| | | SVGWGHVYTLEEFVQHFGKT |
| PS2159 | 12 | MNGLSAQHERILPARHECVYTEFYSEENVWKLCEHIKTSKRCPLGDVYAVFISNERKMVP |
| | | IWKQKSGRGERPVIWDYQVILLHDCHKEQTFIYDLDTTLPFPCPFDTYVKEAFKSDNYIR |
| | | PRFWRKLRVVPADVFLQNFASDRSHMKDASGGWRMPPPPYPCIETAESRMNLDDFISMNP |
| | | SVGWGHVYTLEEFVQHFGKT |
| PS2160 | 13 | MNGLSAQHERILPARHECVYTPCYSEENVWKLCEHIKTQKRCPLGDVYAVFISNERKMVP |
| | | IWKQKVGRGEEPVIWDYQVILLHDCHKEQTFIYDLDTTLPFPCPFDTYVKEAFKSDNYIN |
| | | PRFWRKLRVVPADVFLQNFASDRSHMKDASGGWRMPPPPYPCIETAESRMNLDDFISMNP |
| | | SVGWGHVYTLEEFVQHFGKT |
| PS2161 | 14 | MNGLSAQHERILPARHECVYTSCYSEENVYKLCEHIKTSKRCPLGDVYAVFISNERKMVP |
| | | IWKQRSGRGERVVIWDYQVILLHDCHKEQSFIYDLDTTLPFPCPFDTYVKEAFKSDNYIN |
| | | PRFWRKLRVVPADVFLQNFASDRSHMKDASGGWRMPPPPYPCIETAESRMNLDDFISMNP |
| | | SVGWGHVYTLEEFVQHFGKT |
| PS2162 | 15 | MNGLSAQHERILPARHECVYTPCYSEENVYKLCEHIKTSKRCPLGDVYAVFISNERKMVP |
| | | IWKQKSGRGERVVIWDYQVIMLHDCHKEQTFIYDLDTTLPFPCPFDTYVKEAFKSDNYIN |
| | | PRFWRKLRVVRADVFLQNFASDRSHMKDASGGWRMPPPPYPCIETAESRMNLDDFISMNP |
| | | SVGWGHVYTLEEFVQHFGKT |
| PS2163 | 16 | MNGLSAQHERILPARHECVYTPCYSEENVWKLCEHIKTSKRCPLGDVYAVFISNERKMVP |
| | | IWKQRSGRGERVVIWDYQVILLHDCHKEQTFIYDLDTTLPFPCPFDTYVKEAFKSDNYIN |
| | | PRFWRKLRVVPADVFLQNFASDRSHMKDASGGWRMPPPPYPCIETAESRMNLDDFISMNP |
| | | SVGWGHVYTLEEFVQHFGKT |
| PS2164 | 17 | MNGLSAQHERILPARHECVYTPCYSEENVWKLCEHIKTSKRCPLGDVYAVFISNERKMVP |
| | | IWKQKSGRGEEFVIWDYQVILLHDCHKEQTFIYDLDTTLPFPCPFDTYVKEAFKSDNYIN |
| | | PRFWRKLRVVPADVFLQNFASDRSHMKDASGGWRMPPPPYPCIETAESRMNLDDFISMNP |
| | | SVGWGHVYTLEEFVQHFGKT |
| PS2165 | 18 | MNGLSAQHERILPARHECVYTSCYSEENVWKLCEHIKTQKRCPLGDVYAVFISNERKMVP |
| | | IWKQKVGRGEEPVIWDYQVILLHDCHKEQTFIYDLDTTLPFPCPFDTYVKEAFKSDNYIR |
| | | PRFWRKLRVVPADVFLQNFASDRSHMKDASGGWRMPPPPYPCIETAESRMNLDDFISMNP |
| | | SVGWGHVYTLEEFVQHFGKT |
| PS2166 | 19 | MNGLSAQHERIAPARHECVYTPGYSEENVWKLCQHIKTSKRCPLGDVYAVFISNERKMVP |
| | | IWKQRSGRGEEPLIWDYKVILLHDCHKEQTFIYDLDTTLPFPCPFDTYVKEAFKSDNYIN |
| | | PRFWRKLRVVPADVFLQNFASDRSHMKDSSGGWRMPPPPYPCIETAESRMNLDDFISMNP |
| | | SVGWGHVYTLEEFVQHFGKT |
| PS2167 | 20 | MNGLSAQHERIAPARHECVYTPGYSEENVWKLCQHIKTSKRCPLGDVYAVFISNERKMVP |
| | | IWKQRSGRGEEPLIWDYKVILLHDCHKEQTFIYDLDTTLPFPCPFDTYVKEAFKSDNYIN |
| | | PRFWRKLRVVPADVFLQNFASDRSHMKDASGGWRMPPPPYPCIETAESRMNLDDFISMNP |
| | | SVGWGHVYTLEEFVQHFGKT |
| PS2168 | 21 | MNGLSAQHERILPARHECVYTSGYSEENVWKLCEHIKTSKRCPLGDVYAVFISNERKMVP |
| | | IWKQKSGRGEEPVIWDYKVQLLHDCHKEQTFIYDLDTTLPFPCPFDTYVKEAFKSDNYIN |
| | | PRFWRKLRVVPADVFLQNFASDRSHMKDDLGGWRMPPPPYPCIETAESRMNLDDFISMNP |
| | | SVGWGHVYTLEEFVQHFGKT |
| PS2169 | 22 | MNGLSAQHERIAPARHECVYTPGYGEENVWKLCQHIKTSKRCPLGDVYAVFISNERKMVP |
| | | IWKQRSGRGEEPLIWDYRVILLHDCHKEQTFIYDLDTTLPFPCPFDTYVKEAFRSDNYIN |
| | | PRFWRKLRVVPADVFLQNFASDRSHMKDESGGWRMPPPPYPCIETAESRMNLDDFISMNP |
| | | SVGWGHVYTLEEFVQHFGKT |
| PS2170 | 23 | MNGLSAQHERILPARHECVYTSGYSEENVWKLCEHIKTSKRCPLGDVYAVFISNERKMVP |
| | | IWKQKSGRGEEPLIWDYKVILLHDCHKEQTFIYDLDTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKT |
| PS2171 | 24 | MNGLSAQHERILPARHECVYTPCYSEENVWKLCEHIKTSKRCPLGDVYAVFISNERKMVP |
| | | IWKQKVGRGEEPVIWDYQVILLHDCHKEQTFIYDLDTTLPFPCPFDTYVKEAFKSDNYIR |
| | | PRFWRKLRVVPADVFLQNFASDRSHMKDASGGWRMPPPPYPCIETAESRMNLDDFISMNP |
| | | SVGWGHVYTLEEFVQHFGKT |
| PS2195 | 25 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKT |
| PS2196 | 26 | MNGLSAQHERILPARHECVYTPGYSEENVWKLCEHIKTSKRCPLGVVYAVFISNERKMVP |
| | | IWKQRSGRGEEPLIWDYQVILLHDCHKEQTFIYDLDTTLPFPCPFDTYVKEAFRSDNYIN |
| | | PRFWRKLRVVPADVFLQNFASDRSHMKDERGGWRMPPPPYPCIETAESRMNLDDFISMNP |
| | | SVGWGHVYTLEEFVQHFGKT |
| PS2197 | 27 | MNGLSAQHERILPARHECVYTPCYSEENVWKLCQHIKTSKRCPLGDVYAVFISNERKMVP |
| | | IWKQRSGRGEEPLIWDYQVILLHDCHKEQTFIYDLDTTLPFPCPFDTYVKEAFKSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKT |
| PS2198 | 28 | MNGLSAQHERILPARHECVYTPQYSEENVWKLCEHIKTSKRFPLGDVYAVFISNERKMVP |
| | | IWKQRSGRGEEPVIWDYKVILLHDCHKEQTFIYDLDTTLPFPCPFDTYVKEAFKSDNYIN |
| | | PRFWRKLRVVPADVFLQNFASDRSHMKDSSGGWRMPPPPYPCIETAESRMNLDDFISMNP |
| | | SVGWGHVYTLEEFVQHFGKT |
| PS2199 | 29 | MNGLSAQHERILPARHECVYTPQYSEENVWKLCEHIKTSKRCPLGDVYAVFISNERKMVP |
| | | IWKQRSGRGEEPIIWDYKVFLLHDCHKEQTFIYDLDTTLPFPCPFDTYVKEAFKSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKT |
| PS2200 | 30 | MNGLSAQHERILPARHECVYTPGYGEENVWKLCEHIKTSKRCPLGGVYAVFISNERKMVP |
| | | IWKQRSGRGEEPLIWDYRVILLHDCHKEQTFIYDLDTTLPFPCPFDTYVKKAFRSDNYIN |
| | | PRFWRKLRVVPADVFLQNFASDRSHMKDARGGWRMPPPPYPCIETAESRMNLDDFISMNP |
| | | SVGWGHVYTLEEFVQHFGKT |
| PS2201 | 31 | MNGLSAQHERILPARHECVYTPGYGEENVWKLCEHIKTSKRCPLGDVYAVFISNERRMVP |
| | | IWKQRSGRGEEPLIWDYRVFLLHDCHKEQTFIYDLRTTLPFPCPFDTYVKEAFKSDNYIN |
| | | PRFWRKLRVVPADVFLQNFASDRSHMKDARGGWRMPPPPYPCIETAESRMNLDDFISMNP |
| | | SVGWGHVYTLEEFVQHFGKT |
| PS2202 | 32 | MNGLSAQHERILPARHECVYTPGYSEENVWKLCQHIKTSKRCLLGDVYAVFISNERKMVP |
| | | IWKQKSGRGEEPLIWDYRVILLHDCHKEQTFIYDLRTTLPFPCPFDTYVKEAFRSDNYIN |
| | | PAFWRKLRVVPADVFLQNFASDRSHMKDARGGWRMPPPPYPCIETAESRMNLDDFISMNP |
| | | SVGWGHVYTLEEFVQHFGKT |
| PS2203 | 33 | MNGLSAQHERILPARHECVYTPGYGEENVWKLCEHIKTSKRCPLGDVYAVFISNERKMVP |
| | | IWKQRSGRGEEPLIWDYKVILLHDCHKEQTFIYDLDTTLPFPCPFDTYVKEAFKSDNYIN |
| | | PRFWRKLRVVPADVFLQNFASDRSHMKDSRGGWRMPPPPYPCIETAESRMNLDDFISMNP |
| | | SVGWGHVYTLEELVQHFGKT |
| PS2204 | 34 | MNGLSAQHERILPARHECVYTSGYGEENVWKLCQHIKTSKRCPLGDVYAVFISNERKMVP |
| | | IWKQRSGRGEEPLIWDYKVILLHDCHKEQTFIYDLDTTLPFPCPFDTYVKEAFRSDNYIN |
| | | PRFWRKLRVVPADVFLQNFASDRSHMKDSSGGWRMPPPPYPCIETAESRMNLDDFISMNP |
| | | SVGWGHVYTLEEFVQHFGKT |
| PS2205 | 35 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLDTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKT |
| PS2244 | 36 | MNGLSAQHERIAPARHECVYTVGYSEENVWKLCEHIKTVKRCPLGDVYAVFISNERKMVP |
| | | IWKQKSGRGEEWVWWDYKVILLHDAHKEQTFIYDLGTTLPFPCPFDTYVKEAFKSDNYIH |
| | | PAFWRKLRVVPADVFLQNFASDRSHMKDSSGGWRMPPPPYPCIETAESRMNLDDFISMNP |
| | | SVGWGHVYTLEEFVQHFGKT |
| PS2245 | 37 | MNGLSAQHERIAPARHECVYTQGYSEENVWKLCEHIKTYKRCPLGDVYAVFISNERKMVP |
| | | IWKQKSGRGEEYVVWDYKVILLHDCHKEQTFIYDLGTTLPFPCPFDTYVKEAFKSDNYIN |
| | | PAFWRKLRVVPADVFLQNFASDRSHMKDSSGGWRMPPPPYPCIETAESRMNLDDFISMNP |
| | | SVGWGHVYTLEEFVQHFGKT |
| PS2246 | 38 | MNGLSAQHERIAPARHECVYTPGYSEENVWKLCEHIKTSKRCPLGDVYAVFISNERKMVP |
| | | IWKQKSGRGEEHVLWDYKVILLHDCHKEQTFIYDLGTTLPFPCPFDTYVKEAFKSDNYIN |
| | | PAFWRKLRVVPADVFLQNFASDRSHMKDRSGGWRMPPPPYPCIETAESRMNLDDFISMNP |
| | | SVGWGHVYTLEEFVQHFGKT |
| PS2247 | 39 | MNGLSAQHERIAPARHECVYTQGYSEENVWKLCEHIKTQKRCPLGDVYAVFISNERKMVP |
| | | IWKQKSGRGEEYVLWDYKVILLHDGHKEQTFIYDLGTTLPFPCPFDTYVKEAFKSDNYIN |
| | | PAFWRKLRVVPADVFLQNFASDRSHMKLASGGWRMPPPPYPCIETAESRMNLDDFISMNP |
| | | SVGWGHVYTLEEFVQHFGKT |
| PS2248 | 40 | MNGLSAQHERIAPARHECVYTLGYSEENVWKLCEHIKTGKRCPLGDVYAVFISNERKMVP |
| | | IWKQKSGRGEEHVLWDYKVILLHDCHKEQTFIYDLGTTLPFPCPFDTYVKEAFKSDNYIN |
| | | PAFWRKLRVVPADVFLQNFASDRSHMKLASGGWRMPPPPYPCIETAESRMNLDDFISMNP |
| | | SVGWGHVYTLEEFVQHFGKT |
| PS2249 | 41 | MNGLSAQHERIAPARHECVYTQGYSEENVWKLCEHIKTQKRCPLGDVYAVFISNERKMVP |
| | | IWKQKSGRGEEYVLWDYKVILLHDGHKEQTFIYDLGTTLPFPCPFDTYVKEAFKSDNYIN |
| | | PAFWRKLRVVPADVFLQNFASDRSHMKDKSGGWRMPPPPYPCIETAESRMNLDDFISMNP |
| | | SVGWGHVYTLEEFVQHFGKT |
| PS2250 | 42 | MNGLSAQHERIAPARHECVYTVGYSEENVWKLCEHIKTRKRCPLGDVYAVFISNERKMVP |
| | | IWKQKSGRGEEHVLWDYKVILLHDCHKEQTFIYDLGTTLPFPCPFDTYVKEAFKSDNYIN |
| | | PAFWRKLRVVPADVFLQNFASDRSHMKTASGGWRMPPPPYPCIETAESRMNLDDFISMNP |
| | | SVGWGHVYTLEEFVQHFGKT |
| PS2251 | 43 | MNGLSAQHERIAPARHECVYTPGYSEENVWKLCEHIKTNKRCPLGDVYAVFISNERKMVP |
| | | IWKQKSGRGEEWVLWDYKVILLHDRHKEQTFIYDLGTTLPFPCPFDTYVKEAFKSDNYIN |
| | | PAFWRKLRVVPADVFLQNFASDRSHMKDLSGGWRMPPPPYPCIETAESRMNLDDFISMNP |
| | | SVGWGHVYTLEEFVQHFGKT |
| PS2252 | 44 | MNGLSAQHERIAPARHECVYTEGYSEENVWKLCEHIKTRKRCPLGDVYAVFISNERKMVP |
| | | IWKQKSGRGEEHVVWDYKVILLHDFHKEQTFIYDLGTTLPFPCPFDTYVKEAFKSDNYIN |
| | | PAFWRKLRVVPADVFLQNFASDRSHMKDRSGGWRMPPPPYPCIETAESRMNLDDFISMNP |
| | | SVGWGHVYTLEEFVQHFGKT |
| PS2253 | 45 | MNGLSAQHERIAPARHECVYTSGYSEENVWKLCEHIKTKKRCPLGDVYAVFISNERKMVP |
| | | IWKQKSGRGEEHVLWDYKVILLHDCHKEQTFIYDLGTTLPFPCPFDTYVKEAFKSDNYIN |
| | | PAFWRKLRVVPADVFLQNFASDRSHMKDSSGGWRMPPPPYPCIETAESRMNLDDFISMNP |
| | | SVGWGHVYTLEEFVQHFGKT |
| PS2254 | 46 | MNGLSAQHERIAPARHECVYTQGYSEENVWKLCEHIKTYKRCPLGDVYAVFISNERKMVP |
| | | IWKQKSGRGEEYVVWDYKVILLHDCHKEQTFIYDLGTTLPFPCPFDTYVKEAFKSDNYIN |
| | | PAFWRKLRVVPADVFLQNFASDRSHMKLASGGWRMPPPPYPCIETAESRMNLDDFISMNP |
| | | SVGWGHVYTLEEFVQHFGKT |
| PS2255 | 47 | MNGLSAQHERIAPARHECVYTSGYSEENVWKLCEHIKTVKRCPLGDVYAVFISNERKMVP |
| | | IWKQKSGRGEEWVLWDYKVILLHDCHKEQTFIYDLGTTLPFPCPFDTYVKEAFKSDNYIN |
| | | PAFWRKLRVVPADVFLQNFASDRSHMKLASGGWRMPPPPYPCIETAESRMNLDDFISMNP |
| | | SVGWGHVYTLEEFVQHFGKT |
| PS2256 | 48 | MNGLSAQHERIAPARHECVYTTGYSEENVWKLCEHIKTMKRCPLGDVYAVFISNERKMVP |
| | | IWKQKSGRGEEHVIWDYKVILLHDCHKEQTFIYDLGTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKT |
| PS2257 | 49 | MNGLSAQHERIAPARHECVYTAGYSEENVWKLCEHIKTFKRCPLGDVYAVFISNERKMVP |
| | | IWKQKSGRGEEHVIWDYKVILLHDIHKEQTFIYDLGTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKT |
| PS2258 | 50 | MNGLSAQHERIAPARHECVYTEGYSEENVWKLCEHIKTSKRCPLGDVYAVFISNERKMVP |
| | | IWKQKSGRGEEHVLWDYKVILLHDCHKEQTFIYDLGTTLPFPCPFDTYVKEAFKSDNYIN |
| | | PAFWRKLRVVPADVFLQNFASDRSHMKDRSGGWRMPPPPYPCIETAESRMNLDDFISMNP |
| | | SVGWGHVYTLEEFVQHFGKT |
| PS2259 | 51 | MNGLSAQHERIAPARHECVYTSGYSEENVWKLCEHIKTKKRCPLGDVYAVFISNERKMVP |
| | | IWKQKSGRGEEHVLWDYKVILLHDCHKEQTFIYDLGTTLPFPCPFDTYVKEAFKSDNYIN |
| | | PAFWRKLRVVPADVFLQNFASDRSHMKDPSGGWRMPPPPYPCIETAESRMNLDDFISMNP |
| | | SVGWGHVYTLEEFVQHFGKT |
| PS2260 | 52 | MNGLSAQHERIAPARHECVYTPGYSEENVWKLCEHIKTSKRCPLGDVYAVFISNERKMVP |
| | | IWKQKSGRGEEHVLWDYKVILLHDCHKEQTFIYDLGTTLPFPCPFDTYVKEAFKSDNYIN |
| | | PAFWRKLRVVPADVFLQNFASDRSHMKLASGGWRMPPPPYPCIETAESRMNLDDFISMNP |
| | | SVGWGHVYTLEEFVQHFGKT |
| PS2261 | 53 | MNGLSAQHERIAPARHECVYTSGYSEENVWKLCEHIKTKKRCPLGDVYAVFISNERKMVP |
| | | IWKQKSGRGEEHVLWDYKVILLHDCHKEQTFIYDLGTTLPFPCPFDTYVKEAFKSDNYIN |
| | | PAFWRKLRVVPADVFLQNFASDRSHMKLASGGWRMPPPPYPCIETAESRMNLDDFISMNP |
| | | SVGWGHVYTLEEFVQHFGKT |
| PS2262 | 54 | MNGLSAQHERIAPARHECVYTEGYSEENVWKLCEHIKTRKRCPLGDVYAVFISNERKMVP |
| | | IWKQKSGRGEEWVLWDYKVILLHDCHKEQTFIYDLGTTLPFPCPFDTYVKEAFKSDNYIN |
| | | PAFWRKLRVVPADVFLQNFASDRSHMKDTSGGWRMPPPPYPCIETAESRMNLDDFISMNP |
| | | SVGWGHVYTLEEFVQHFGKT |
| PS2263 | 55 | MNGLSAQHERIAPARHECVYTSGYSEENVWKLCEHIKTKKRCPLGDVYAVFISNERKMVP |
| | | IWKQKSGRGEEHVLWDYKVILLHDCHKEQTFIYDLGTTLPFPCPFDTYVKEAFKSDNYIN |
| | | PAFWRKLRVVPADVFLQNFASDRSHMKDNSGGWRMPPPPYPCIETAESRMNLDDFISMNP |
| | | SVGWGHVYTLEEFVQHFGKT |
| PS2264 | 56 | MNGLSAQHERIAPARHECVYTQGYSEENVWKLCEHIKTVKRCPLGDVYAVFISNERKMVP |
| | | IWKQKSGRGEEHVLWDYKVILLHDCHKEQTFIYDLGTTLPFPCPFDTYVKEAFKSDNYIN |
| | | PAFWRKLRVVPADVFLQNFASDRSHMKDRSGGWRMPPPPYPCIETAESRMNLDDFISMNP |
| | | SVGWGHVYTLEEFVQHFGKT |
| PS2265 | 57 | MNGLSAQHERIAPARHECVYTQGYSEENVWKLCEHIKTQKRCPLGDVYAVFISNERKMVP |
| | | IWKQKSGRGEEYVLWDYKVILLHDGHKEQTFIYDLGTTLPFPCPFDTYVKEAFKSDNYIN |
| | | PAFWRKLRVVPADVFLQNFASDRSHMKTASGGWRMPPPPYPCIETAESRMNLDDFISMNP |
| | | SVGWGHVYTLEEFVQHFGKT |
| PS2278 | 58 | MNGLSAQHERIAPARHECVYTSCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKDKSGGWRMPPPPYPCIETAESRMNLDDFISMNP |
| | | SVGWGHVYTLEEFVQHFGKT |
| PS2279 | 59 | MNGLSAQHERIAPARHECVYTSCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKDMSGGWRMPPPPYPCIETAESRMNLDDFISMNP |
| | | SVGWGHVYTLEEFVQHFGKT |
| PS2280 | 60 | MNGLSAQHERIAPARHECVYTSCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKDTSGGWRMPPPPYPCIETAESRMNLDDFISMNP |
| | | SVGWGHVYTLEEFVQHFGKT |
| PS2281 | 61 | MNGLSAQHERIAPARHECVYTSCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKDPSGGWRMPPPPYPCIETAESRMNLDDFISMNP |
| | | SVGWGHVYTLEEFVQHFGKT |
| PS2282 | 62 | MNGLSAQHERIAPARHECVYTSCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKTASGGWRMPPPPYPCIETAESRMNLDDFISMNP |
| | | SVGWGHVYTLEEFVQHFGKT |
| PS2283 | 63 | MNGLSAQHERIAPARHECVYTSCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKDDSGGWRMPPPPYPCIETAESRMNLDDFISMNP |
| | | SVGWGHVYTLEEFVQHFGKT |
| PS2284 | 64 | MNGLSAQHERIAPARHECVYTSCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKSASGGWRMPPPPYPCIETAESRMNLDDFISMNP |
| | | SVGWGHVYTLEEFVQHFGKT |
| PS2285 | 65 | MNGLSAQHERIAPARHECVYTSCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKDISGGWRMPPPPYPCIETAESRMNLDDFISMNP |
| | | SVGWGHVYTLEEFVQHFGKT |
| PS2286 | 66 | MNGLSAQHERIAPARHECVYTDCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKT |
| PS2287 | 67 | MNGLSAQHERIAPARHECVYTSCYSEENVWKLCEHIKTGKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKT |
| PS2288 | 68 | MNGLSAQHERIAPARHECVYTDSYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKT |
| PS2289 | 69 | MNGLSAQHERIAPARHECVYTSCYSEENVWKLCEHIKTRKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKT |
| PS2290 | 70 | MNGLSAQHERIAPARHECVYTSSYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKT |
| PS2291 | 71 | MNGLSAQHERIAPARHECVYTSCYSEENVWKLCEHIKTSKRCPLGDVYAVFISNERKMVP |
| | | IWKQKSGRGEEPVIWDYQVILLHDSHKEQTFIYDLDTTLPFPCPFDTYVKEAFKSDNYIN |
| | | PAFWRKLRVVPADVFLQNFASDRSHMKDRSGGWRMPPPPYPCIETAESRMNLDDFISMNP |
| | | SVGWGHVYTLEEFVQHFGKT |
| PS2292 | 72 | MNGLSAQHERIAPARHECVYTSCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKEASGGWRMPPPPYPCIETAESRMNLDDFISMNP |
| | | SVGWGHVYTLEEFVQHFGKT |
| PS2293 | 73 | MNGLSAQHERIAPARHECVYTSCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKYASGGWRMPPPPYPCIETAESRMNLDDFISMNP |
| | | SVGWGHVYTLEEFVQHFGKT |
| PS2294 | 74 | MNGLSAQHERIAPARHECVYTSCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKDLSGGWRMPPPPYPCIETAESRMNLDDFISMNP |
| | | SVGWGHVYTLEEFVQHFGKT |
| PS2295 | 75 | MNGLSAQHERIAPARHECVYTWCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKT |
| PS2296 | 76 | MNGLSAQHERIAPARHECVYTSCYSEENVWKLCEHIKTSKRCPLGDVYAVFISNERKMVP |
| | | IWKQKSGRGEEMVIWDYQVILLHDCHKEQTFIYDLDTTLPFPCPFDTYVKEAFKSDNYIN |
| | | PAFWRKLRVVPADVFLQNFASDRSHMKDTSGGWRMPPPPYPCIETAESRMNLDDFISMNP |
| | | SVGWGHVYTLEEFVQHFGKT |
| PS2297 | 77 | MNGLSAQHERIAPARHECVYTSCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKNASGGWRMPPPPYPCIETAESRMNLDDFISMNP |
| | | SVGWGHVYTLEEFVQHFGKT |
| PS2298 | 78 | MNGLSAQHERIAPARHECVYTSGYSEENVWKLCEHIKTGKRCPLGDVYAVFISNERKMVP |
| | | IWKQKSGRGEELVIWDYQVILLHDCHKEQTFIYDLDTTLPFPCPFDTYVKEAFKSDNYIN |
| | | PAFWRKLRVVPADVFLQNFASDRSHMKDLSGGWRMPPPPYPCIETAESRMNLDDFISMNP |
| | | SVGWGHVYTLEEFVQHFGKT |
| PS2299 | 79 | MNGLSAQHERIAPARHECVYTSGYSEENVWKLCEHIKTRKRCPLGDVYAVFISNERKMVP |
| | | IWKQKSGRGEEMVIWDYQVILLHDCHKEQTFIYDLDTTLPFPCPFDTYVKEAFKSDNYIN |
| | | PAFWRKLRVVPADVFLQNFASDRSHMKDRSGGWRMPPPPYPCIETAESRMNLDDFISMNP |
| | | SVGWGHVYTLEEFVQHFGKT |
| PS2392 | 80 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIR |
| | | PRFWRKLRVVPADVFLQNFASDRSHMKDSRGGWRMPPPPYPCIETAESRMNLDDFISMNP |
| | | SVGWGHVYTLEEFVQHFGKT |
| PS2393 | 81 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDARGGWRMPPPPYPCIETAESRMNLDDFISMNP |
| | | SVGWGHVYTLEEFVQHFGKT |
| PS2394 | 82 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSSGGWRMPPPPYPCIETAESRMNLDDFISMNP |
| | | SVGWGHVYTLEEFVQHFGKT |
| PS2395 | 83 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDASGGWRMPPPPYPCIETAESRMNLDDFISMNP |
| | | SVGWGHVYTLEEFVQHFGKT |
| PS2396 | 84 | MNGLSAQHERILPARHECVYTPGYSEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKT |
| PS2397 | 85 | MNGLSAQHERILPARHECVYTPCYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKT |
| PS2398 | 86 | MNGLSAQHERILPARHECVYTSGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKT |
| PS2399 | 87 | MNGLSAQHERILPARHECVYTPGYGEENVWKLCEHIKTSKRCPLGDVYAVFISNERKMVP |
| | | IWKQKSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKT |
| PS2400 | 88 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIR |
| | | PRFWRKLRVVPADVFLQNFASDRSHMKDARGGWRMPPPPYPCIETAESRMNLDDFISMNP |
| | | SVGWGHVYTLEEFVQHFGKT |
| PS2401 | 89 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIR |
| | | PRFWRKLRVVPADVFLQNFASDRSHMKDSRGGWLMPPPPYPCIETAESRMNLDDFISMNP |
| | | SVGWGHVYTLEEFVQHFGKT |
| PS2402 | 90 | MNGLSAQHERILPARHECVYTPGYGEENVWKLCEHIKTSKRCPLGDVYAVFISNERKMVP |
| | | IWKQRSGRGERPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKT |
| PS2428 | 91 | MNGLSAQHERILPARHECVYTEGYGEENVWKLCEHIKTSKRCPLGDVYAVFISNERKMVP |
| | | IWKQKSGRGEEPVIWDYRVILLHDTHKEQTFIYDLRTTLSFPCPFDTYVKEAFKSDNYIR |
| | | PRFWRKLRVVPADVFLQNFASDRSHMKDASGGWQMPPPPYPCIETAESRMNLDDFISMNP |
| | | SVGWGHVYTLEEFVQHFGKT |
| PS2429 | 92 | MNGLSAQHERILPARHECVYTEGYGEENVWKLCEHIKTSKRCPLGDVYAVFISNERKMVP |
| | | IWKQKSGRGERPLIWDYRVILLHDCHKEQTFIYDLRTTLPFPCPFDTYVKEAFKSDNYIN |
| | | PRFWRKLRVVPADVFLQNFASDRSHMKDSRGGWPMPPPPYPCIETAESRMNLDDFISMNP |
| | | SVGWGHVYTLEEFVQHFGKT |
| PS2430 | 93 | MNGLSAQHERILPARHECVYTEGYGEENVWKLCEHIKTSKRCPLGDVYAVFISNERKMVP |
| | | IWKQKSGRGEEPVIWDYRVILLHDTHKEQTFIYDLRTTLSFPCPFDTYVKEAFKSDNYIR |
| | | PRFWRKLRVVPADVFLQNFASDRSHMKGASGGWQMPPPPYPCIETAESRMNLDDFISMNP |
| | | SVGWGHVYTLEEFVQHFGKT |
| PS2431 | 94 | MNGLSAQHERILPARHECVYTEGYGEENVWKLCEHIKTSKRCPLGDVYAVFISNERKMVP |
| | | IWKQKSGRGEEPVIWDYRVILLHDPHKEQTFIYDLRTTLSFPCPFDTYVKEAFKSDNYIR |
| | | PRFWRKLRVVPADVFLQNFASDRSHMKDASGGWQMPPPPYPCIETAESRMNLDDFISMNP |
| | | SVGWGHVYTLEEFVQHFGKT |
| PS2432 | 95 | MNGLSAQHERILPARHECVYTECYSEENVWKLCEHIKTSKRCPLGDVYAVFISNERKMVP |
| | | IWKQKSGRGERPVIWDYKVILLHDTHKEQTFIHDLRTTLPFPCPFDTYVKEAFRSDNYIN |
| | | PRFWRKLRVVPADVFLQNFASDRSHMKDSRGGWLMPPPPYPCIETAESRMNLDDFISMNP |
| | | SVGWGHVYTLEEFVQHFGKT |
| PS2433 | 96 | MNGLSAQHERILPARHECVYTPGYSEENVWILCEHIKTSKRCPLGDVYAVFISNERKMVP |
| | | IWKQKSGRGERPVIWDYRVILLHDTHKEQTFIYDLKTTLPFSCPFDTYVKEAFRSDNYIR |
| | | PRFWRKLRVVPADVFLQNFASDRSHMKDSSGGWQMPPPPYPCIETAESRMNLDDFISMNP |
| | | SVGWGHVYTLEEFVQHFGKT |
| PS2434 | 97 | MNGLSAQHERILPARHECVYTPCYGEENVWKLCEHIKTSKRCPLGDVYAVFISNERKMVP |
| | | IWKQKSGRGERPLIWDYRVILLHDTHKEQTFIYDLRTTLPFPCPFDTYVKEAFKSDNYIN |
| | | PRFWRKLRVVPADVFLQNFASDRSHMKDSRGGWLMPPPPYPCIETAESRMNLDDFISMNP |
| | | SVGWGHVYTLEEFVQHFGKT |
| PS2435 | 98 | MNGLSAQHERILPARHECVYTPGYSEENVWKLCEHIKTSKRCPLGDVYAVFISNERKMVP |
| | | IWKQKSGRGEEPLIWDYRVILLHDCHKEQTFIYDLRTTLPFPCPFDTYVKEAFKSDNYIR |
| | | PRFWRKLRVVPADVFLQNFASDRSHMKDARGGWRMPPPPYPCIETAESRMNLDDFISMNP |
| | | SVGWGHVYTLEEFVQHFGKT |
| PS2436 | 99 | MNGLSAQHERILPARHECVYTEGYGEENVWKLCEHIETSKRCPLGDVYAVFISNERKMVP |
| | | IWKQKSGRGEEPVIWDYRVILLHDTHKEQTFIYDLRTTLSFPCPFDTYVKEAFKSDNYIR |
| | | PRFWRKLRVVPADVFLQNFASDRSHMKDASGGWQMPPPPYPCIETAESRMNLDDFISMNP |
| | | SVGWGHVYTLEEFVQHFGKT |
| PS2437 | 100 | MNGLSAQHERILPARHECVYTPCYSEENVWKLCEHIKTSKRCPLGDVYAVFISNERKMVP |
| | | IWKQKSGRGERPVIWDYRVILLHDCHKEQTFIHDLDTTLPFPCPFDTYVEEAFRSDNYIR |
| | | PRFWRKLRVVPADVFLQNFASDRSHMKDARGGWPMPPPPYPCIETAESRMNLDDFISMNP |
| | | SVGWGHVYTLEEFVQHFGKT |
| PS2438 | 101 | MNGLSAQHERILPARHECVYTEGYGEENVWKLCEHIKTSKRCPLGDVYAVFISNERKMVP |
| | | IWKQKSGRGGRPLIWDYRVILLHDCHKEQTFIYDLRTTLPFPCPFDTYVKEAFKSDNYIN |
| | | PRFWRKLRVVPADVFLQNFASDRSHMKDSRGGWPMPPPPYPCIETAESRMNLDDFISMNP |
| | | SVGWGHVYTLEEFVQHFGKT |
| PS2439 | 102 | MNGLSAQHERILPARHECVYTEGYGEENVWKLCEHIKTSKRCPLGDVYAVFISNERKMVP |
| | | IWKQKSGRGEEPLIWDYKVILLHDCHKEQTFIYDLDTTLPFPCPFDTYVKEAFRSDNYIR |
| | | PRFWRKLRVVPADVFLQNFASDRSHMKDASGGWPMPPPPYPCIETAESRMNLDDFISMNP |
| | | SVGWGHVYTLEEFVQHFGKT |
| PS2440 | 103 | MNGLSAQHERILPARHECVYTPGYGEENVWKLCEHIKTSKRCPLGDVYAVFISNERKMVP |
| | | IWKQKSGRGEEPLIWDYRVILLHDCHKEQTFIYDLRTTLPFPCPFDTYVKEAFKSDNYIR |
| | | PRFWRKLRVVPADVFLQNFASDRSHMKDARGGWLMPPPPYPCIETAESRMNLDDFISMNP |
| | | SVGWGHVYTLEEFVQHFGKT |
| PS2441 | 104 | MNGLSAQHERILPARHECVYTPGYGEENVWKLCEHIKTSKRCPLGDVYAVFISNERKMVP |
| | | IWKQKSGRGERPVIWDYRVILLHDTHKEQTFIYDLRTTLPFPCPFDTYVKEAFRSDNYIR |
| | | PRFWRKLRVVPADVFLQNFASDRSHMKDSRGGWRMPPPPYPCIETAESRMNLDDFISMNP |
| | | SVGWGHVYTLEEFVQHFGKT |
| PS2442 | 105 | MNGLSAQHERILPARHECVYTPGYGEENVWKLCEHIKTSKRCPLGDVYAVFISNERKMVP |
| | | IWKQKSGRGEEPLIWDYRVILLHDCHKEQTFIYDLRTTLPFPCPFDTYVKEAFRSDNYIR |
| | | PRFWRKLRVVPADVFLQNFASDRSHMKDSRGGWRMPPPPYPCIETAESRMNLDDFISMNP |
| | | SVGWGHVYTLEEFVQHFGKT |
| PS2443 | 106 | MNGLSAQHERILPARHECVYTPGYGEENVWKLCEHIKTSKRCPLGDVYAVFISNERKMVP |
| | | IWKQKSGRGERPLIWDYRVILLHDCHKEQTFIYDLRTTLPFPCPFDTYVKEAFRSDNYIR |
| | | PRFWRKLRVVPADVFLQNFASDRSHMKDSRGGWRMPPPPYPCIETAESRMNLDDFISMNP |
| | | SVGWGHVYTLEEFVQHFGKT |
| PS2444 | 107 | MNGLSAQHERILPARHECVYTPGYGEENVWKLCEHIKTSKRCPLGDVYAVFISNERKMVP |
| | | IWKQKSGRGERPLIWDYRVILLHDTHKEQTFIYDLRTTLPFPCPFDTYVKEAFRSDNYIR |
| | | PRFWRKLRVVPADVFLQNFASDRSHMKDSRGGWRMPPPPYPCIETAESRMNLDDFISMNP |
| | | SVGWGHVYTLEEFVQHFGKT |
| PS2445 | 108 | MNGLSAQHERILPARHECVYTPGYGEENVWKLCEHIKTSKRCPLGDVYAVFISNERKMVP |
| | | IWKQKSGRGEEPLIWDYRVILLHDCHKEQTFIYDLRTTLPFPCPFDTYVKEAFRSDNYIN |
| | | PRFWRKLRVVPADVFLQNFASDRSHMKDARGGWRMPPPPYPCIETAESRMNLDDFISMNP |
| | | SVGWGHVYTLEEFVQHFGKT |
| PS2446 | 109 | MNGLSAQHERILPARHECVYTPGYGEENVWKLCEHIKTSKRCPLGDVYAVFISNERKMVP |
| | | IWKQKSGRGEEPLIWDYRVILLHDCHKEQTFIYDLRTTLPFPCPFDTYVKEAFRSDNYIR |
| | | PRFWRKLRVVPADVFLQNFASDRSHMKDARGGWRMPPPPYPCIETAESRMNLDDFISMNP |
| | | SVGWGHVYTLEEFVQHFGKT |
| PS2447 | 110 | MNGLSAQHERILPARHECVYTPGYGEENVWKLCEHIKTSKRCPLGDVYAVFISNERKMVP |
| | | IWKQKSGRGERPLIWDYRVILLHDCHKEQTFIYDLRTTLPFPCPFDTYVKEAFRSDNYIR |
| | | PRFWRKLRVVPADVFLQNFASDRSHMKDARGGWRMPPPPYPCIETAESRMNLDDFISMNP |
| | | SVGWGHVYTLEEFVQHFGKT |
| PS2448 | 111 | MNGLSAQHERILPARHECVYTPGYGEENVWKLCEHIKTSKRCPLGDVYAVFISNERKMVP |
| | | IWKQKSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWLMPPPPYPCIETAESRMNLDDFISMNP |
| | | SVGWGHVYTLEEFVQHFGKT |
| PS2449 | 112 | MNGLSAQHERILPARHECVYTPGYGEENVWKLCEHIKTSKRCPLGDVYAVFISNERKMVP |
| | | IWKQKSGRGERPLIWDYRVILLHDCHKEQTFIYDLRTTLPFPCPFDTYVKEAFRSDNYIR |
| | | PRFWRKLRVVPADVFLQNFASDRSHMKDARGGWLMPPPPYPCIETAESRMNLDDFISMNP |
| | | SVGWGHVYTLEEFVQHFGKT |
| PS2088 | 113 | MNGLSAQHERIAPARHECVYTSCYSEENVWKLCEHIKTSKRCPLGDVYAVFISNERKMVP |
| | | IWKQKSGRGEEPVIWDYKVILLHDCHKEQTFIYDLDTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKTEAAAKEAAAKEAAAKMNGLSAQHERIAPARHECVYTSCYS |
| | | EENVWKLCEHIKTSKRCPLGDVYAVFISNERKMVPIWKQKSGRGEEPVIWDYKVILLHDC |
| | | HKEQTFIYDLDTTLPFPCPFDTYVKEAFKSDNYINPAFWRKLRVVPADVFLQNFASDRSH |
| | | MKDASGGWRMPPPPYPCIETAESRMNLDDFISMNPSVGWGHVYTLEEFVQHFGKT |
| PS2089 | 114 | MNGLSAQHERIAPARHECVYTSCYSEENVWKLCEHIKTSKRCPLGDVYAVFISNERKMVP |
| | | IWKQKSGRGEEPVIWDYKVILLHDCHKEQTFIYDLDTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKTEEEKRKREEEEMNGLSAQHERIAPARHECVYTSCYSEENV |
| | | WKLCEHIKTSKRCPLGDVYAVFISNERKMVPIWKQKSGRGEEPVIWDYKVILLHDCHKEQ |
| | | TFIYDLDTTLPFPCPFDTYVKEAFKSDNYINPAFWRKLRVVPADVFLQNFASDRSHMKDA |
| | | SGGWRMPPPPYPCIETAESRMNLDDFISMNPSVGWGHVYTLEEFVQHFGKT |
| PS2234 | 115 | MNGLSAQHERIAPARHECVYTECYSEENVWKLCEHIKTQKRCPLGDVYAVFISNERKMVP |
| | | IWKQKSGRGEEPVIWDYKVILLHDTHKEQTFIYDLDTTLPFPCPFDTYVKEAFKSDNYIR |
| | | PAFWRKLRVVPADVFLQNFASDRSHMKDASGGWRMPPPPYPCIETAESRMNLDDFISMNP |
| | | SVGWGHVYTLEEFVQHFGKTEAAAKEAAAKEAAAKMNGLSAQHERIAPARHECVYTECYS |
| | | EENVWKLCEHIKTQKRCPLGDVYAVFISNERKMVPIWKQKSGRGEEPVIWDYKVILLHDT |
| | | HKEQTFIYDLDTTLPFPCPFDTYVKEAFKSDNYIRPAFWRKLRVVPADVFLQNFASDRSH |
| | | MKDASGGWRMPPPPYPCIETAESRMNLDDFISMNPSVGWGHVYTLEEFVQHFGKT |
| PS2235 | 116 | MNGLSAQHERIAPARHECVYTECYSEENVWKLCEHIKTQKRCPLGDVYAVFISNERKMVP |
| | | IWKQKSGRGEEPVIWDYKVILLHDTHKEQTFIYDLDTTLPFPCPFDTYVKEAFKSDNYIR |
| | | PAFWRKLRVVPADVFLQNFASDRSHMKDASGGWRMPPPPYPCIETAESRMNLDDFISMNP |
| | | SVGWGHVYTLEEFVQHFGKTEEEKRKREEEEMNGLSAQHERIAPARHECVYTECYSEENV |
| | | WKLCEHIKTQKRCPLGDVYAVFISNERKMVPIWKQKSGRGEEPVIWDYKVILLHDTHKEQ |
| | | TFIYDLDTTLPFPCPFDTYVKEAFKSDNYIRPAFWRKLRVVPADVFLQNFASDRSHMKDA |
| | | SGGWRMPPPPYPCIETAESRMNLDDFISMNPSVGWGHVYTLEEFVQHFGKT |
| PS2236 | 117 | MNGLSAQHERIAPARHECVYTECYSEENVWKLCEHIKTSKRCPLGDVYAVFISNERKMVP |
| | | IWKQKSGRGEEPVIWDYKVILLHDCHKEQTFIYDLDTTLPFPCPFDTYVKEAFKSDNYIR |
| | | PAFWRKLRVVPADVFLQNFASDRSHMKDASGGWRMPPPPYPCIETAESRMNLDDFISMNP |
| | | SVGWGHVYTLEEFVQHFGKTEAAAKEAAAKEAAAKMNGLSAQHERIAPARHECVYTECYS |
| | | EENVWKLCEHIKTSKRCPLGDVYAVFISNERKMVPIWKQKSGRGEEPVIWDYKVILLHDC |
| | | HKEQTFIYDLDTTLPFPCPFDTYVKEAFKSDNYIRPAFWRKLRVVPADVFLQNFASDRSH |
| | | MKDASGGWRMPPPPYPCIETAESRMNLDDFISMNPSVGWGHVYTLEEFVQHFGKT |
| PS2237 | 118 | MNGLSAQHERIAPARHECVYTECYSEENVWKLCEHIKTSKRCPLGDVYAVFISNERKMVP |
| | | IWKQKSGRGEEPVIWDYKVILLHDCHKEQTFIYDLDTTLPFPCPFDTYVKEAFKSDNYIR |
| | | PAFWRKLRVVPADVFLQNFASDRSHMKDASGGWRMPPPPYPCIETAESRMNLDDFISMNP |
| | | SVGWGHVYTLEEFVQHFGKTEEEKRKREEEEMNGLSAQHERIAPARHECVYTECYSEENV |
| | | WKLCEHIKTSKRCPLGDVYAVFISNERKMVPIWKQKSGRGEEPVIWDYKVILLHDCHKEQ |
| | | TFIYDLDTTLPFPCPFDTYVKEAFKSDNYIRPAFWRKLRVVPADVFLQNFASDRSHMKDA |
| | | SGGWRMPPPPYPCIETAESRMNLDDFISMNPSVGWGHVYTLEEFVQHFGKT |
| PS2238 | 119 | MNGLSAQHERIAPARHECVYTECYSEENVWKLCEHIKTSKRCPLGDVYAVFISNERKMVP |
| | | IWKQKSGRGEEPVIWDYKVILLHDTHKEQTFIYDLDTTLPFPCPFDTYVKEAFKSDNYIR |
| | | PAFWRKLRVVPADVFLQNFASDRSHMKDASGGWRMPPPPYPCIETAESRMNLDDFISMNP |
| | | SVGWGHVYTLEEFVQHFGKTEAAAKEAAAKEAAAKMNGLSAQHERIAPARHECVYTECYS |
| | | EENVWKLCEHIKTSKRCPLGDVYAVFISNERKMVPIWKQKSGRGEEPVIWDYKVILLHDT |
| | | HKEQTFIYDLDTTLPFPCPFDTYVKEAFKSDNYIRPAFWRKLRVVPADVFLQNFASDRSH |
| | | MKDASGGWRMPPPPYPCIETAESRMNLDDFISMNPSVGWGHVYTLEEFVQHFGKT |
| PS2239 | 120 | MNGLSAQHERIAPARHECVYTECYSEENVWKLCEHIKTSKRCPLGDVYAVFISNERKMVP |
| | | IWKQKSGRGEEPVIWDYKVILLHDTHKEQTFIYDLDTTLPFPCPFDTYVKEAFKSDNYIR |
| | | PAFWRKLRVVPADVFLQNFASDRSHMKDASGGWRMPPPPYPCIETAESRMNLDDFISMNP |
| | | SVGWGHVYTLEEFVQHFGKTEEEKRKREEEEMNGLSAQHERIAPARHECVYTECYSEENV |
| | | WKLCEHIKTSKRCPLGDVYAVFISNERKMVPIWKQKSGRGEEPVIWDYKVILLHDTHKEQ |
| | | TFIYDLDTTLPFPCPFDTYVKEAFKSDNYIRPAFWRKLRVVPADVFLQNFASDRSHMKDA |
| | | SGGWRMPPPPYPCIETAESRMNLDDFISMNPSVGWGHVYTLEEFVQHFGKT |
| PS2240 | 121 | MNGLSAQHERIAPARHECVYTECYSEENVWKLCEHIKTQKRCPLGDVYAVFISNERKMVP |
| | | IWKQKSGRGEEPVIWDYKVILLHDCHKEQTFIYDLDTTLPFPCPFDTYVKEAFKSDNYIR |
| | | PAFWRKLRVVPADVFLQNFASDRSHMKDASGGWRMPPPPYPCIETAESRMNLDDFISMNP |
| | | SVGWGHVYTLEEFVQHFGKTEAAAKEAAAKEAAAKMNGLSAQHERIAPARHECVYTECYS |
| | | EENVWKLCEHIKTQKRCPLGDVYAVFISNERKMVPIWKQKSGRGEEPVIWDYKVILLHDC |
| | | HKEQTFIYDLDTTLPFPCPFDTYVKEAFKSDNYIRPAFWRKLRVVPADVFLQNFASDRSH |
| | | MKDASGGWRMPPPPYPCIETAESRMNLDDFISMNPSVGWGHVYTLEEFVQHFGKT |
| PS2241 | 122 | MNGLSAQHERIAPARHECVYTECYSEENVWKLCEHIKTQKRCPLGDVYAVFISNERKMVP |
| | | IWKQKSGRGEEPVIWDYKVILLHDCHKEQTFIYDLDTTLPFPCPFDTYVKEAFKSDNYIR |
| | | PAFWRKLRVVPADVFLQNFASDRSHMKDASGGWRMPPPPYPCIETAESRMNLDDFISMNP |
| | | SVGWGHVYTLEEFVQHFGKTEEEKRKREEEEMNGLSAQHERIAPARHECVYTECYSEENV |
| | | WKLCEHIKTQKRCPLGDVYAVFISNERKMVPIWKQKSGRGEEPVIWDYKVILLHDCHKEQ |
| | | TFIYDLDTTLPFPCPFDTYVKEAFKSDNYIRPAFWRKLRVVPADVFLQNFASDRSHMKDA |
| | | SGGWRMPPPPYPCIETAESRMNLDDFISMNPSVGWGHVYTLEEFVQHFGKT |
| PS2242 | 123 | MNGLSAQHERIAPARHECVYTECYSEENVWKLCEHIKTSKRCPLGDVYAVFISNERKMVP |
| | | IWKQKSGRGEEPVIWDYKVILLHDCHKEQTFIYDLDTTLPFPCPFDTYVKEAFKSDNYIR |
| | | PAFWRKLRVVPADVFLQNFASDRSHMKDVSGGWRMPPPPYPCIETAESRMNLDDFISMNP |
| | | SVGWGHVYTLEEFVQHFGKTEAAAKEAAAKEAAAKMNGLSAQHERIAPARHECVYTECYS |
| | | EENVWKLCEHIKTSKRCPLGDVYAVFISNERKMVPIWKQKSGRGEEPVIWDYKVILLHDC |
| | | HKEQTFIYDLDTTLPFPCPFDTYVKEAFKSDNYIRPAFWRKLRVVPADVFLQNFASDRSH |
| | | MKDVSGGWRMPPPPYPCIETAESRMNLDDFISMNPSVGWGHVYTLEEFVQHFGKT |
| PS2243 | 124 | MNGLSAQHERIAPARHECVYTECYSEENVWKLCEHIKTSKRCPLGDVYAVFISNERKMVP |
| | | IWKQKSGRGEEPVIWDYKVILLHDCHKEQTFIYDLDTTLPFPCPFDTYVKEAFKSDNYIR |
| | | PAFWRKLRVVPADVFLQNFASDRSHMKDVSGGWRMPPPPYPCIETAESRMNLDDFISMNP |
| | | SVGWGHVYTLEEFVQHFGKTEEEKRKREEEEMNGLSAQHERIAPARHECVYTECYSEENV |
| | | WKLCEHIKTSKRCPLGDVYAVFISNERKMVPIWKQKSGRGEEPVIWDYKVILLHDCHKEQ |
| | | TFIYDLDTTLPFPCPFDTYVKEAFKSDNYIRPAFWRKLRVVPADVFLQNFASDRSHMKDV |
| | | SGGWRMPPPPYPCIETAESRMNLDDFISMNPSVGWGHVYTLEEFVQHFGKT |
| PS2366 | 125 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKTGGGSGGGSGGGSGMNGLSAQHERILPARHECVYTPGYGEE |
| | | NVWKLCEHIKTSKRCPLGDVYAVFISNERKMVPIWKQRSGRGEEPLIWDYRVILLHDCHK |
| | | EQTFIYDLRTTLPFPCPFDTYVKEAFRSDNYINPRFWRKLRVVPADVFLQNFASDRSHMK |
| | | DSRGGWRMPPPPYPCIETAESRMNLDDFISMNPSVGWGHVYTLEEFVQHFGKT |
| PS2367 | 126 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKTGSAGSAAGSGEFMNGLSAQHERILPARHECVYTPGYGEEN |
| | | VWKLCEHIKTSKRCPLGDVYAVFISNERKMVPIWKQRSGRGEEPLIWDYRVILLHDCHKE |
| | | QTFIYDLRTTLPFPCPFDTYVKEAFRSDNYINPRFWRKLRVVPADVFLQNFASDRSHMKD |
| | | SRGGWRMPPPPYPCIETAESRMNLDDFISMNPSVGWGHVYTLEEFVQHFGKT |
| PS2368 | 127 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKTGSAGSAAGSGEFGSAGSAAGSGEFGSAGSAAGSGEFMNGL |
| | | SAQHERILPARHECVYTPGYGEENVWKLCEHIKTSKRCPLGDVYAVFISNERKMVPIWKQ |
| | | RSGRGEEPLIWDYRVILLHDCHKEQTFIYDLRTTLPFPCPFDTYVKEAFRSDNYINPRFW |
| | | RKLRVVPADVFLQNFASDRSHMKDSRGGWRMPPPPYPCIETAESRMNLDDFISMNPSVGW |
| | | GHVYTLEEFVQHFGKT |
| PS2369 | 128 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKTQGLQNEEMNGLSAQHERILPARHECVYTPGYGEENVWKLC |
| | | EHIKTSKRCPLGDVYAVFISNERKMVPIWKQRSGRGEEPLIWDYRVILLHDCHKEQTFIY |
| | | DLRTTLPFPCPFDTYVKEAFRSDNYINPRFWRKLRVVPADVFLQNFASDRSHMKDSRGGW |
| | | RMPPPPYPCIETAESRMNLDDFISMNPSVGWGHVYTLEEFVQHFGKT |
| PS2370 | 129 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKTDPGGGPSSRLMNGLSAQHERILPARHECVYTPGYGEENVW |
| | | KLCEHIKTSKRCPLGDVYAVFISNERKMVPIWKQRSGRGEEPLIWDYRVILLHDCHKEQT |
| | | FIYDLRTTLPFPCPFDTYVKEAFRSDNYINPRFWRKLRVVPADVFLQNFASDRSHMKDSR |
| | | GGWRMPPPPYPCIETAESRMNLDDFISMNPSVGWGHVYTLEEFVQHFGKT |
| PS2371 | 130 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKTGNDGLCQKLSVPCMSSKPQKPWEAKDAWEMNGLSAQHERI |
| | | LPARHECVYTPGYGEENVWKLCEHIKTSKRCPLGDVYAVFISNERKMVPIWKQRSGRGEE |
| | | PLIWDYRVILLHDCHKEQTFIYDLRTTLPFPCPFDTYVKEAFRSDNYINPRFWRKLRVVP |
| | | ADVFLQNFASDRSHMKDSRGGWRMPPPPYPCIETAESRMNLDDFISMNPSVGWGHVYTLE |
| | | EFVQHFGKT |
| PS2372 | 131 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKTFSFGFSFGFSFGMNGLSAQHERILPARHECVYTPGYGEEN |
| | | VWKLCEHIKTSKRCPLGDVYAVFISNERKMVPIWKQRSGRGEEPLIWDYRVILLHDCHKE |
| | | QTFIYDLRTTLPFPCPFDTYVKEAFRSDNYINPRFWRKLRVVPADVFLQNFASDRSHMKD |
| | | SRGGWRMPPPPYPCIETAESRMNLDDFISMNPSVGWGHVYTLEEFVQHFGKT |
| PS2373 | 132 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKTFSFGFSFGFSFGFSFGFSFGFSFGMNGLSAQHERILPARH |
| | | ECVYTPGYGEENVWKLCEHIKTSKRCPLGDVYAVFISNERKMVPIWKQRSGRGEEPLIWD |
| | | YRVILLHDCHKEQTFIYDLRTTLPFPCPFDTYVKEAFRSDNYINPRFWRKLRVVPADVFL |
| | | QNFASDRSHMKDSRGGWRMPPPPYPCIETAESRMNLDDFISMNPSVGWGHVYTLEEFVQH |
| | | FGKT |
| PS2374 | 133 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKTEAAAKEAAAKEAAAKMNGLSAQHERILPARHECVYTPGYG |
| | | EENVWKLCEHIKTSKRCPLGDVYAVFISNERKMVPIWKQRSGRGEEPLIWDYRVILLHDC |
| | | HKEQTFIYDLRTTLPFPCPFDTYVKEAFRSDNYINPRFWRKLRVVPADVFLQNFASDRSH |
| | | MKDSRGGWRMPPPPYPCIETAESRMNLDDFISMNPSVGWGHVYTLEEFVQHFGKT |
| PS2375 | 134 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKTEEEKRKREEEEMNGLSAQHERILPARHECVYTPGYGEENV |
| | | WKLCEHIKTSKRCPLGDVYAVFISNERKMVPIWKQRSGRGEEPLIWDYRVILLHDCHKEQ |
| | | TFIYDLRTTLPFPCPFDTYVKEAFRSDNYINPRFWRKLRVVPADVFLQNFASDRSHMKDS |
| | | RGGWRMPPPPYPCIETAESRMNLDDFISMNPSVGWGHVYTLEEFVQHFGKT |
| PS2376 | 135 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKTLPSPDVHMNGLSAQHERILPARHECVYTPGYGEENVWKLC |
| | | EHIKTSKRCPLGDVYAVFISNERKMVPIWKQRSGRGEEPLIWDYRVILLHDCHKEQTFIY |
| | | DLRTTLPFPCPFDTYVKEAFRSDNYINPRFWRKLRVVPADVFLQNFASDRSHMKDSRGGW |
| | | RMPPPPYPCIETAESRMNLDDFISMNPSVGWGHVYTLEEFVQHFGKT |
| PS2377 | 136 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKTAKLKQKTEQLQDRIAGMNGLSAQHERILPARHECVYTPGY |
| | | GEENVWKLCEHIKTSKRCPLGDVYAVFISNERKMVPIWKQRSGRGEEPLIWDYRVILLHD |
| | | CHKEQTFIYDLRTTLPFPCPFDTYVKEAFRSDNYINPRFWRKLRVVPADVFLQNFASDRS |
| | | HMKDSRGGWRMPPPPYPCIETAESRMNLDDFISMNPSVGWGHVYTLEEFVQHFGKT |
| PS2378 | 137 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKTWRIRPRPPRLPRPRPRMNGLSAQHERILPARHECVYTPGY |
| | | GEENVWKLCEHIKTSKRCPLGDVYAVFISNERKMVPIWKQRSGRGEEPLIWDYRVILLHD |
| | | CHKEQTFIYDLRTTLPFPCPFDTYVKEAFRSDNYINPRFWRKLRVVPADVFLQNFASDRS |
| | | HMKDSRGGWRMPPPPYPCIETAESRMNLDDFISMNPSVGWGHVYTLEEFVQHFGKT |
| PS2379 | 138 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKTAPAPAPAPAPAPAPAPAPAPMNGLSAQHERILPARHECVY |
| | | TPGYGEENVWKLCEHIKTSKRCPLGDVYAVFISNERKMVPIWKQRSGRGEEPLIWDYRVI |
| | | LLHDCHKEQTFIYDLRTTLPFPCPFDTYVKEAFRSDNYINPRFWRKLRVVPADVFLQNFA |
| | | SDRSHMKDSRGGWRMPPPPYPCIETAESRMNLDDFISMNPSVGWGHVYTLEEFVQHFGKT |
| PS2408 | 139 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKTAEAAAKEAAAKEAAAKEAAAKAMNGLSAQHERILPARHEC |
| | | VYTPGYGEENVWKLCEHIKTSKRCPLGDVYAVFISNERKMVPIWKQRSGRGEEPLIWDYR |
| | | VILLHDCHKEQTFIYDLRTTLPFPCPFDTYVKEAFRSDNYINPRFWRKLRVVPADVFLQN |
| | | FASDRSHMKDSRGGWRMPPPPYPCIETAESRMNLDDFISMNPSVGWGHVYTLEEFVQHFG |
| | | KT |
| PS2409 | 140 | MNGLSAQHERILPARHECVYTPGYGEENVWKLCEHIKTSKRCPLGDVYAVFISNERKMVP |
| | | IWKQRSGRGEEPLIWDYRVILLHDCHKEQTFIYDLRTTLPFPCPFDTYVKEAFRSDNYIN |
| | | PRFWRKLRVVPADVFLQNFASDRSHMKDSRGGWRMPPPPYPCIETAESRMNLDDFISMNP |
| | | SVGWGHVYTLEEFVQHFGKTAEAAAKEAAAKEAAAKEAAAKEAAAKAMNGLSAQHERILP |
| | | ARHECVYTPGYGEENVWKLCEHIKTSKRCPLGDVYAVFISNERKMVPIWKQRSGRGEEPL |
| | | IWDYRVILLHDCHKEQTFIYDLRTTLPFPCPFDTYVKEAFRSDNYINPRFWRKLRVVPAD |
| | | VFLQNFASDRSHMKDSRGGWRMPPPPYPCIETAESRMNLDDFISMNPSVGWGHVYTLEEF |
| | | VQHFGKT |
| PS2424 | 141 | MNGLSAQHERIAPARHECVYTVGYSEENVWKLCEHIKTVKRCPLGDVYAVFISNERKMVP |
| | | IWKQKSGRGEEWVWWDYKVILLHDAHKEQTFIYDLGTTLPFPCPFDTYVKEAFKSDNYIH |
| | | PAFWRKLRVVPADVFLQNFASDRSHMKDSSGGWRMPPPPYPCIETAESRMNLDDFISMNP |
| | | SVGWGHVYTLEEFVQHFGKTEAAAKEAAAKEAAAKNGLSAQHERIAPARHECVYTVGYSE |
| | | ENVWKLCEHIKTVKRCPLGDVYAVFISNERKMVPIWKQKSGRGEEWVWWDYKVILLHDAH |
| | | KEQTFIYDLGTTLPFPCPFDTYVKEAFKSDNYIHPAFWRKLRVVPADVFLQNFASDRSHM |
| | | KDSSGGWRMPPPPYPCIETAESRMNLDDFISMNPSVGWGHVYTLEEFVQHFGKT |
| PS2425 | 142 | MNGLSAQHERIAPARHECVYTVGYSEENVWKLCEHIKTVKRCPLGDVYAVFISNERKMVP |
| | | IWKQKSGRGEEWVWWDYKVILLHDAHKEQTFIYDLGTTLPFPCPFDTYVKEAFKSDNYIH |
| | | PAFWRKLRVVPADVFLQNFASDRSHMKDSSGGWRMPPPPYPCIETAESRMNLDDFISMNP |
| | | SVGWGHVYTLEEFVQHFGKTEEEKRKREEEENGLSAQHERIAPARHECVYTVGYSEENVW |
| | | KLCEHIKTVKRCPLGDVYAVFISNERKMVPIWKQKSGRGEEWVWWDYKVILLHDAHKEQT |
| | | FIYDLGTTLPFPCPFDTYVKEAFKSDNYIHPAFWRKLRVVPADVFLQNFASDRSHMKDSS |
| | | GGWRMPPPPYPCIETAESRMNLDDFISMNPSVGWGHVYTLEEFVQHFGKT |
| PS2426 | 143 | MNGLSAQHERIAPARHECVYTVGYSEENVWKLCEHIKTVKRCPLGDVYAVFISNERKMVP |
| | | IWKQKSGRGEEWVWWDYKVILLHDAHKEQTFIYDLGTTLPFPCPFDTYVKEAFKSDNYIH |
| | | PAFWRKLRVVPADVFLQNFASDRSHMKDSSGGWRMPPPPYPCIETAESRMNLDDFISMNP |
| | | SVGWGHVYTLEEFVQHFGKTAEAAAKEAAAKEAAAKEAAAKANGLSAQHERIAPARHECV |
| | | YTVGYSEENVWKLCEHIKTVKRCPLGDVYAVFISNERKMVPIWKQKSGRGEEWVWWDYKV |
| | | ILLHDAHKEQTFIYDLGTTLPFPCPFDTYVKEAFKSDNYIHPAFWRKLRVVPADVFLQNF |
| | | ASDRSHMKDSSGGWRMPPPPYPCIETAESRMNLDDFISMNPSVGWGHVYTLEEFVQHFGK |
| | | T |
| PS2427 | 144 | MNGLSAQHERIAPARHECVYTVGYSEENVWKLCEHIKTVKRCPLGDVYAVFISNERKMVP |
| | | IWKQKSGRGEEWVWWDYKVILLHDAHKEQTFIYDLGTTLPFPCPFDTYVKEAFKSDNYIH |
| | | PAFWRKLRVVPADVFLQNFASDRSHMKDSSGGWRMPPPPYPCIETAESRMNLDDFISMNP |
| | | SVGWGHVYTLEEFVQHFGKTAEAAAKEAAAKEAAAKEAAAKEAAAKANGLSAQHERIAPA |
| | | RHECVYTVGYSEENVWKLCEHIKTVKRCPLGDVYAVFISNERKMVPIWKQKSGRGEEWVW |
| | | WDYKVILLHDAHKEQTFIYDLGTTLPFPCPFDTYVKEAFKSDNYIHPAFWRKLRVVPADV |
| | | FLQNFASDRSHMKDSSGGWRMPPPPYPCIETAESRMNLDDFISMNPSVGWGHVYTLEEFV |
| | | QHFGKT |
| PS1923 | 145 | MHSKFSHAGRICGAKFKVGEPIYRCRECSFDPTCVLCVNCFNPKDHLGHHVYTTICTEFN |
| | | NGECDCGDKTAWNHTLFCKAEEG |
| PS1924 | 146 | MHSKFSHAGRICGAKFKVGEPIYRCHECSFDPTCVLCVNCFNPKDHLGHHVYTTICTEFN |
| | | NGECDCGDKTAWNHTLFCKAEEG |
| PS1925 | 147 | MHSKFSHAGRICGAKFKVGEPIYRCPECSFDPTCVLCVNCFNPKDHLGHHVYTTICTEFN |
| | | NGECDCGDKTAWNHTLFCKAEEG |
| PS1926 | 148 | MHSKFSHAGRICGAKFKVGEPIYRCRECSFDPTCVLCVNCFNPKDHLGHHVYTTICTQKN |
| | | NGECDCGDKTAWNHTLFCKAEEG |
| PS1927 | 149 | MHSKFSHAGRICGAKFKVGEPIYRCKECSFDDTCVLCVNCFNPKDHVGHHVYTTICTEFN |
| | | NGECDCGDKTAWNHTLFCKAEEG |
| PS1928 | 150 | MHSKFSHAGRICGAKFKVGEPIYRCKECSFDDTCVLCVNCFNPKDHRGHHVYTTICTEFN |
| | | NGECDCGDKTAWNHTLFCKAEEG |
| PS1929 | 151 | MHSKFSHAGRICGAKFKVGEPIYRCKECSFDDTCVLCVNCFNPKDHIGHHVYTTICTEFN |
| | | NGECDCGDKTAWNHTLFCKAEEG |
| PS1930 | 152 | MHSKFSHAGRICGAKFKVGEPIYRCKECSFDDTCVLCVNCFNPKDHLGHHVYVTICTEFN |
| | | NGECDCGDKTAWNHTLFCKAEEG |
| PS1931 | 153 | MHSKFSHAGRICGAKFKVGEPIYRCKECSFDDTCVLCVNCFNPKDHRGHHVYTTICTERN |
| | | NGECDCGDKTAWNHTLFCKAEEG |
| PS1932 | 154 | MHSKFSHAGRICGAKFKVGEPIYRCKECSFDDTCVLCVNCFNPKDHRGHHVYTTICTERN |
| | | NGECDCGDKTAWNHELFCKAEEG |
| PS1933 | 155 | MHSKFSHAGRICGAKFKVGEPIYRCKECSFDDTCVLCVNCFNPKDHRGHHVYTTICTERN |
| | | NGECDCGDKTAWNHDLFCKAEEG |
| PS1934 | 156 | MHSKFSHAGRICGAKFKVGEPIYRCKECSFDDTCVLCVNCFNPKDHIGHHVYTTICTERN |
| | | NGECDCGDKTAWNHDLFCKAEEG |
| PS1935 | 157 | MHSKFSHAGRICGAKFKVGEPIYRCKECSFDDTCVLCVNCFNPKDHIGHHVYTTICTERN |
| | | NGECDCGDKTAWNHELFCKAEEG |
| PS1936 | 158 | MHSKFSHAGRICGAKFKVGEPIYRCKECSFDDTCVLCVNCFNPKDHRGHHVYVTICTERN |
| | | NGECDCGDKTAWNHELFCKAEEG |
| PS1937 | 159 | MHSKFSHAGRICGAKFKVGEPIYRCRECSFDRTCVLCVNCFNPKDHRGHHVYVTICTERN |
| | | NGECDCGDKTAWNHELFCKAEEG |
| PS1938 | 160 | MHSKFSHAGRICGAKFKVGEPIYRCRECSFDPTCVLCVNCFNPKDHRGHHVYVTICTERN |
| | | NGECDCGDKTAWNHELFCKAEEG |
| PS1659 | 161 | MHSKFSHAGRICGAKFKVGEPIYRCRECSFDKTCVLCVNCFNPKDHLGHHVYTTICTQKN |
| | | NGECDCGDKTAWNHTLFCKAEEG |
| PS1715 | 162 | MHSKFSHAGRICGAKFKVGEPIYGCRECSFDRTCVLCVNCFNPNDHIGHHVYTTICTEKL |
| | | NGECDCGDKTAWNHTLFCKAEEG |
| PS2080 | 163 | MHSKFSHAGRICGAKFKVGEPIYRCRECSFDKTCVLCVNCFNPKDHLGHHVYTTICTQKN |
| | | NGECDCGDKTAWNHTLFCKAEEGSAGSAAGSGEFMHSKFSHAGRICGAKFKVGEPIYRCR |
| | | ECSFDKTCVLCVNCFNPKDHLGHHVYTTICTQKNNGECDCGDKTAWNHTLFCKAEEG |
| PS2081 | 164 | MHSKFSHAGRICGAKFKVGEPIYRCRECSFDKTCVLCVNCFNPKDHLGHHVYTTICTQKN |
| | | NGECDCGDKTAWNHTLFCKAEEGSAGSAAGSGEFGSAGSAAGSGEFGSAGSAAGSGEFMH |
| | | SKFSHAGRICGAKFKVGEPIYRCRECSFDKTCVLCVNCFNPKDHLGHHVYTTICTQKNNG |
| | | ECDCGDKTAWNHTLFCKAEEG |
| PS2082 | 165 | MHSKFSHAGRICGAKFKVGEPIYGCRECSFDRTCVLCVNCFNPNDHIGHHVYTTICTEKL |
| | | NGECDCGDKTAWNHTLFCKAEEGSAGSAAGSGEFMHSKFSHAGRICGAKFKVGEPIYGCR |
| | | ECSFDRTCVLCVNCFNPNDHIGHHVYTTICTEKLNGECDCGDKTAWNHTLFCKAEEG |
| PS2083 | 166 | MHSKFSHAGRICGAKFKVGEPIYGCRECSFDRTCVLCVNCFNPNDHIGHHVYTTICTEKL |
| | | NGECDCGDKTAWNHTLFCKAEEGSAGSAAGSGEFGSAGSAAGSGEFGSAGSAAGSGEFMH |
| | | SKFSHAGRICGAKFKVGEPIYGCRECSFDRTCVLCVNCFNPNDHIGHHVYTTICTEKLNG |
| | | ECDCGDKTAWNHTLFCKAEEG |
| PS2084 | 167 | MHSKFSHAGRICGAKFKVGEPIYRCKECSFDDTCVLCVNCFNPKDHRGHHVYVTICTERN |
| | | NGECDCGDKTAWNHELFCKAEEGSAGSAAGSGEFMHSKFSHAGRICGAKFKVGEPIYRCK |
| | | ECSFDDTCVLCVNCFNPKDHRGHHVYVTICTERNNGECDCGDKTAWNHELFCKAEEG |
| PS2085 | 168 | MHSKFSHAGRICGAKFKVGEPIYRCKECSFDDTCVLCVNCFNPKDHRGHHVYVTICTERN |
| | | NGECDCGDKTAWNHELFCKAEEGSAGSAAGSGEFGSAGSAAGSGEFGSAGSAAGSGEFMH |
| | | SKFSHAGRICGAKFKVGEPIYRCKECSFDDTCVLCVNCFNPKDHRGHHVYVTICTERNNG |
| | | ECDCGDKTAWNHELFCKAEEG |
| PS2173 | 169 | MHSKFSHAGRICGAKFKVGEPIYRCKECSFDDTCVLCVNCFNPKDHRGHHVYVTICTERN |
| | | NGECDCGDKTAWNHELFCKAEEGQGLQNEEMHSKFSHAGRICGAKFKVGEPIYRCKECSF |
| | | DDTCVLCVNCFNPKDHRGHHVYVTICTERNNGECDCGDKTAWNHELFCKAEEG |
| PS2174 | 170 | MHSKFSHAGRICGAKFKVGEPIYRCKECSFDDTCVLCVNCFNPKDHRGHHVYVTICTERN |
| | | NGECDCGDKTAWNHELFCKAEEGDPGGGPSSRLMHSKFSHAGRICGAKFKVGEPIYRCKE |
| | | CSFDDTCVLCVNCFNPKDHRGHHVYVTICTERNNGECDCGDKTAWNHELFCKAEEG |
| PS2175 | 171 | MHSKFSHAGRICGAKFKVGEPIYRCKECSFDDTCVLCVNCFNPKDHRGHHVYVTICTERN |
| | | NGECDCGDKTAWNHELFCKAEEGGNDGLCQKLSVPCMSSKPQKPWEAKDAWEMHSKFSHA |
| | | GRICGAKFKVGEPIYRCKECSFDDTCVLCVNCFNPKDHRGHHVYVTICTERNNGECDCGD |
| | | KTAWNHELFCKAEEG |
| PS2176 | 172 | MHSKFSHAGRICGAKFKVGEPIYRCKECSFDDTCVLCVNCFNPKDHRGHHVYVTICTERN |
| | | NGECDCGDKTAWNHELFCKAEEGFSFGFSFGFSFGMHSKFSHAGRICGAKFKVGEPIYRC |
| | | KECSFDDTCVLCVNCFNPKDHRGHHVYVTICTERNNGECDCGDKTAWNHELFCKAEEG |
| PS2177 | 173 | MHSKFSHAGRICGAKFKVGEPIYRCKECSFDDTCVLCVNCFNPKDHRGHHVYVTICTERN |
| | | NGECDCGDKTAWNHELFCKAEEGFSFGFSFGFSFGFSFGFSFGFSFGMHSKFSHAGRICG |
| | | AKFKVGEPIYRCKECSFDDTCVLCVNCFNPKDHRGHHVYVTICTERNNGECDCGDKTAWN |
| | | HELFCKAEEG |
| PS2178 | 174 | MHSKFSHAGRICGAKFKVGEPIYRCKECSFDDTCVLCVNCFNPKDHRGHHVYVTICTERN |
| | | NGECDCGDKTAWNHELFCKAEEGEAAAKEAAAKEAAAKMHSKFSHAGRICGAKFKVGEPI |
| | | YRCKECSFDDTCVLCVNCFNPKDHRGHHVYVTICTERNNGECDCGDKTAWNHELFCKAEE |
| | | G |
| PS2179 | 175 | MHSKFSHAGRICGAKFKVGEPIYRCKECSFDDTCVLCVNCFNPKDHRGHHVYVTICTERN |
| | | NGECDCGDKTAWNHELFCKAEEGEEEKRKREEEEMHSKFSHAGRICGAKFKVGEPIYRCK |
| | | ECSFDDTCVLCVNCFNPKDHRGHHVYVTICTERNNGECDCGDKTAWNHELFCKAEEG |
| PS2180 | 176 | MHSKFSHAGRICGAKFKVGEPIYRCKECSFDDTCVLCVNCFNPKDHRGHHVYVTICTERN |
| | | NGECDCGDKTAWNHELFCKAEEGLPSPDVHMHSKFSHAGRICGAKFKVGEPIYRCKECSF |
| | | DDTCVLCVNCFNPKDHRGHHVYVTICTERNNGECDCGDKTAWNHELFCKAEEG |
| PS2181 | 177 | MHSKFSHAGRICGAKFKVGEPIYRCKECSFDDTCVLCVNCFNPKDHRGHHVYVTICTERN |
| | | NGECDCGDKTAWNHELFCKAEEGAKLKQKTEQLQDRIAGMHSKFSHAGRICGAKFKVGEP |
| | | IYRCKECSFDDTCVLCVNCFNPKDHRGHHVYVTICTERNNGECDCGDKTAWNHELFCKAE |
| | | EG |
| PS2182 | 178 | MHSKFSHAGRICGAKFKVGEPIYRCKECSFDDTCVLCVNCFNPKDHRGHHVYVTICTERN |
| | | NGECDCGDKTAWNHELFCKAEEGWRIRPRPPRLPRPRPRMHSKFSHAGRICGAKFKVGEP |
| | | IYRCKECSFDDTCVLCVNCFNPKDHRGHHVYVTICTERNNGECDCGDKTAWNHELFCKAE |
| | | EG |
| PS2183 | 179 | MHSKFSHAGRICGAKFKVGEPIYRCKECSFDDTCVLCVNCFNPKDHRGHHVYVTICTERN |
| | | NGECDCGDKTAWNHELFCKAEEGAPAPAPAPAPAPAPAPAPAPMHSKFSHAGRICGAKFK |
| | | VGEPIYRCKECSFDDTCVLCVNCFNPKDHRGHHVYVTICTERNNGECDCGDKTAWNHELF |
| | | CKAEEG |
| PS2406 | 180 | MHSKFSHAGRICGAKFKVGEPIYRCKECSFDDTCVLCVNCFNPKDHRGHHVYVTICTERN |
| | | NGECDCGDKTAWNHELFCKAEEGAEAAAKEAAAKEAAAKEAAAKAMHSKFSHAGRICGAK |
| | | FKVGEPIYRCKECSFDDTCVLCVNCFNPKDHRGHHVYVTICTERNNGECDCGDKTAWNHE |
| | | LFCKAEEG |
| PS2407 | 181 | MHSKFSHAGRICGAKFKVGEPIYRCKECSFDDTCVLCVNCFNPKDHRGHHVYVTICTERN |
| | | NGECDCGDKTAWNHELFCKAEEGAEAAAKEAAAKEAAAKEAAAKEAAAKAMHSKFSHAGR |
| | | ICGAKFKVGEPIYRCKECSFDDTCVLCVNCFNPKDHRGHHVYVTICTERNNGECDCGDKT |
| | | AWNHELFCKAEEG |
| PS610 | 182 | MSDSPVDLKPKPKVKPKLERPKLYKVMLLNDDYTPMSFVTVVLKAVFRMSEDTGRRVMMT |
| | | AHRFGSAVVVVCERDIAETKAKEATDLGKEAGFPLMFTTEPEEGSAGSAAGSGEFMSDSP |
| | | VDLKPKPKVKPKLERPKLYKVMLLNDDYTPMSFVTVVLKAVFRMSEDTGRRVMMTAHRFG |
| | | SAVVVVCERDIAETKAKEATDLGKEAGFPLMFTTEPEE |
| PS1587 | 183 | MGLENSLETLRFSISNLSMQTHAARMRTFMYWPSSVPVQPEQLASAGFYYVGRNDDVKCF |
| | | CCDGGLRCWESGDDPWVEHAKWFPRCEFLIRMKGQEFVDEIQGRYPHLLEQLLSEAAAKE |
| | | AAAKEAAAKMGLENSLETLRFSISNLSMQTHAARMRTFMYWPSSVPVQPEQLASAGFYYV |
| | | GRNDDVKCFCCDGGLRCWESGDDPWVEHAKWFPRCEFLIRMKGQEFVDEIQGRYPHLLEQ |
| | | LLSG |
| PS1751 | 184 | MNGLSAQHERIAPARHECVYTSCYSEENVWKLCEHIKTSKRCPLGDVYAVFISNERKMVP |
| | | IWKQKSGRGERPVIWDYQVILLHDCHKEQTFIYDLDTTLPFPCPFDTYVKEAFKSDNYIN |
| | | PAFWRKLRVVPADVFLQNFASDRSHMKDASGGWRMPPPPYPCIETAESRMNLDDFISMNP |
| | | SVGWGHVYTLEEFVQHFGKT |
| PS2225 | 185 | MPTAASATESAIEDTPAPARPEVDGRTKPKRQPRYHVVLWDDDDHTYQYVVVMLRSLFGH |
| | | PPERGYRMAKEMDTQGRVIVLTTTREHAELKRDQIHAFGRDRLLARSKGSMKASIEAEE |
| PS2300 | 210 | MNGLSAQHERIAPARHECVYTSCYSEENVWKLCEHIKTSKRCPLGDVYAVFISNERKMVP |
| | | IWKQKSGRGEEPVIWDYQVILLHDTHKEQTFIYDLDTTLPFPCPFDTYVKEAFKSDNYIN |
| | | PAFWRKLRVVPADVFLQNFASDRSHMKMASGGWRMPPPPYPCIETAESRMNLDDFISMNP |
| | | SVGWGHVYTLEEFVQHFGKT |
| PS2301 | 211 | MNGLSAQHERIAPARHECVYTTCYSEENVWKLCEHIKTNKRCPLGDVYAVFISNERKMVP |
| | | IWKQKSGRGEEPVYWDYQVILLHDCHKEQTFIYDLDTTLPFPCPFDTYVKEAFKSDNYIN |
| | | PAFWRKLRVVPADVFLQNFASDRSHMKYASGGWRMPPPPYPCIETAESRMNLDDFISMNP |
| | | SVGWGHVYTLEEFVQHFGKT |
| PS2302 | 212 | MNGLSAQHERIAPARHECVYTSCYSEENVWKLCEHIKTSKRCPLGDVYAVFISNERKMVP |
| | | IWKQKSGRGEEPVIWDYQVILLHDCHKEQTFIYDLDTTLPFPCPFDTYVKEAFKSDNYIN |
| | | PAFWRKLRVVPADVFLQNFASDRSHMKFASGGWRMPPPPYPCIETAESRMNLDDFISMNP |
| | | SVGWGHVYTLEEFVQHFGKT |
| PS2303 | 213 | MNGLSAQHERILPARHECVYTECYSEENVWKLCEHIKTSKRCPLGDVYAVFISNERKMVP |
| | | IWKQKVGRGEEPVIWDYQVILLHDCHKEQTFIYDLDTTLPFPCPFDTYVKEAFKSDNYIR |
| | | PRFWRKLRVVPADVFLQNFASDRSHMKDAVGGWRMPPPPYPCIETAESRMNLDDFISMNP |
| | | SVGWGHVYTLEEFVQHFGKT |
| PS2304 | 214 | MNGLSAQHERILPARHECVYTECYGEENVYKLCEHIKTSKRCPLGDVYAVFISNERKMVP |
| | | IWKQKVGRGEEPIIWDYQVILLHDCHKEQTFIYDLDTTLPFPCPFDTYVKEAFKSDNYIR |
| | | PAFWRKLRVVPADVFLQNFASDRSHMKDGVGGWQMSPPPYPCIETAESRMNLDDFISMNP |
| | | SVGWGHVYTLEEFVQHFGKT |
| PS2305 | 215 | MNGLSAQHERILPARHECVYTECYSEENVWKLCEHIKTSKRCPLGDVYAVFISNERKMVP |
| | | IWKQKVGRGERPVIWDYQVILLHDCHKEQTFIYDLDTTLPFPCPFDTYVKEAFKSDNYIN |
| | | PRFWRKLRVVPADVFLQNFASDRSHMKDLVGGWLMPPPPYPCIETAESRMNLDDFISMNP |
| | | SVGWGHVYTLEEFVQHFGKT |
| PS2306 | 216 | MNGLSAQHERILPARHECVYTECYSEENVWKLCEHIKTSKRCPLGDVYAVFISNERKMVP |
| | | IWKQKVGRGERPVIWDYQVILLHDCHKEQTFIYDLDTTLPFPCPFDTYVKEAFKSDNYIN |
| | | PDFWRKLRVIPADVFLQNFASDRSHMKDLVGGWLMPPPPYPCIETAESRMNLDDFISMNP |
| | | SVGWGHVYTLEEFVQHFGKT |
| PS2307 | 217 | MNGLSAQHERILPARHECVYTSCYSEENVWKLCEHIKTSKRCPLGDVYAVFISNERKMVP |
| | | IWKQKIGRGKRPIIWDYQVVLLHDRHKEQTFIYDLDTTLPFPCPFDTYVKEAFKSDNYIN |
| | | PAFWRKLRVVPADVFLQNFASDRSHMKDVGGGWLMPPPPYPCIETAESRMNLDDFISMNP |
| | | SVGWGHVYTLEEFVQHFGKT |
| PS2308 | 218 | MNGLSAQHERILPARHECVYTECYSEENVWKLCEHIKTSKRCPLGDVYAVFISNERKMVP |
| | | IWKQKSGRGERPIIWDYQVVLLHDCHKEQTFIYDLDTTLPFPCPFDTYVKEAFKSDNYIN |
| | | PRFWRKLRVVPADVFLQNFASDRSHMRDNGGGWLMPPPPYPCIETAESRMNLDDFISMNP |
| | | SVGWGHVYTLEEFVQHFGKT |
| PS2309 | 219 | MNGLSAQHERILPARHECVYTSCYSEENVWKLCEHIKTSKRCPLGDVYAVFISNERKMVP |
| | | IWKQKSGRGERPIIWDYQVVLLHDCHKEQTFIYDLDTTLPFPCPFDTYVKEAFKSDNYIS |
| | | PRFWRKLRVVPADVFLQNFASDRSHMKDDVGGWQMPPPPYPCIETAESRMNLDDFISMNP |
| | | SVGWGHVYTLEEFVQHFGKT |
| PS2310 | 220 | MNGLSAQHERITPARHECVYTECYSEENVYKLCEHIKTSKRCPLGDVYAVFISNERKMVP |
| | | IWKQKSGRGEKPVIWDYQVILLHDCHKEQTFIYDLDTTLPFPCPFDTYVKEAFKSDNYIR |
| | | PRFWRKLRVVPADVFLQNFASDRSHMKDDVGGWLMPPPPYPCIETAESRMNLDDFISMNP |
| | | SVGWGHVYTLEEFVQHFGKT |
| PS2311 | 221 | MNGLSAQHERILPARHECVYTEYYGWENVWKLCEHIKTSKRCPLGDVYAVFISNERKMVP |
| | | IWKQKSGRGEEPIIWDYQVVLLHDCHKEQTFIYDLDTTLPFPCPFDTYVKEAFKSDNYIN |
| | | PDFWRKLRVVPADVFLQNFASDRSHMKDGCGGWQMPPPPYPCIETAESRMNLDDFISMNP |
| | | SVGWGHVYTLEEFVQHFGKT |
| PS2312 | 222 | MNGLSAQHERILPARHECVYTRCYSEENVWKLCEHIKTSKRCPLGDVYAVFISNERKMVP |
| | | IWKQKVGRGERPVIWDYQVILLHDCHKEQTFIYDLDTTLPFPCPFDTYVKEAFKSDNYIN |
| | | PRFWRKLRVVPADVFLQNFASDRSHMKDLVGGWLMPPPPYPCIETAESRMNLDDFISMNP |
| | | SVGWGHVYTLEEFVQHFGKT |
| PS2313 | 223 | MNGLSAQHERILPARHECVYTECYSEENVWKLCEHIKTSKRCPLGDVYAVFISNERKMVP |
| | | IWKQKSGRGERPVIWDYQVVLLHDRHKEQTFIYDLDTTLPFPCPFDTYVKEAFKSDNYIR |
| | | PRFWRKLRVVPADVFLQNFASDRSHMKDVSGGWRMPPPPYPCIETAESRMNLDDFISMNP |
| | | SVGWGHVYTLEEFVQHFGKT |
| PS2314 | 224 | MNGLCAQHERILPARHECVYTSCYSEENVWKLCEHIKTSKRCPLGDVYAVFISNERKMVP |
| | | IWKQKSGRGERPIIWDYQVVLLHDCHKEQTFIYDLDTTLPFPCPFDTYVKEAFKSDNYIS |
| | | PRFWRKLRVVPADVFLQNFASDRSHMKDDVGGWQMPPPPYPCIETAESRMNLDDFISMNP |
| | | SVGWGHVYTLEEFVQHFGKT |
| PS2450 | 225 | MNGLSAQHERILPARHECVYTECYSEENVWKLCEHIKTSKRCPLGDVYAVFISNERKMVP |
| | | IWKQRVGRGERPVIWDYQVILLHDCHKEQTFIYDLDTTLPFPCPFDTYVKEAFKSDNYIN |
| | | PRFWRKLRVVPADVFLQNFASDRSHMKDAVGGWLMPPPPYPCIETAESRMNLDDFISMNP |
| | | SVGWGHVYTLEEFVQHFGKT |
| PS2451 | 226 | MNGLSAQHERILPARHECVYTECYQEENVWKLCEHIKTSKRCPLGDVYAVFISNERKMVP |
| | | IWKQKVGRGEEPVIWDYHVILLHDCHKEQTFIYDLDTTLPFPCPFDTYVKEAFKSDNYIR |
| | | PRFWRKLRVVPADVFLQNFASDRSHMKDAVGGWRMPPPPYPCIETAESRMNLDDFISMNP |
| | | SVGWGHVYTLEEFVQHFGKT |
| PS2452 | 227 | MNGLSAQHERILPARHECVYTECYQEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIN |
| | | PRFWRKLRVVPADVFLQNFASDRSHMKDAVGGWLMPPPPYPCIETAESRMNLDDFISMNP |
| | | SVGWGHVYTLEEFVQHFGKT |
| PS2453 | 228 | MNGLSAQHERILPARHECVYTECYQEENVWKLCEHIKTSKRCPLGDVYAVFISNERKMVP |
| | | IWKQKSGRGERPIIWDYHVVLLHDCHKEQTFIYDLDTTLPFPCPFDTYVKEAFKSDNYIN |
| | | PRFWRKLRVVPADVFLQNFASDRSHMRDNGGGWLMPPPPYPCIETAESRMNLDDFISMNP |
| | | SVGWGHVYTLEEFVQHFGKT |
| PS2454 | 229 | MNGLSAQHERILPARHECVYTSCYQEENVWKLCEHIKTSKRCPLGDVYAVFISNERKMVP |
| | | IWKQKSGRGERPIIWDYHVVLLHDCHKEQTFIYDLDTTLPFPCPFDTYVKEAFKSDNYIS |
| | | PRFWRKLRVVPADVFLQNFASDRSHMKDDVGGWQMPPPPYPCIETAESRMNLDDFISMNP |
| | | SVGWGHVYTLEEFVQHFGKT |
| PS2455 | 230 | MNGLSAQHERITPARHECVYTECYQEENVYKLCEHIKTSKRCPLGDVYAVFISNERKMVP |
| | | IWKQKSGRGEKPVIWDYHVILLHDCHKEQTFIYDLDTTLPFPCPFDTYVKEAFKSDNYIR |
| | | PRFWRKLRVVPADVFLQNFASDRSHMKDDVGGWLMPPPPYPCIETAESRMNLDDFISMNP |
| | | SVGWGHVYTLEEFVQHFGKT |
| PS2456 | 231 | MNGLSAQHERILPARHECVYTRCYQEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIN |
| | | PRFWRKLRVVPADVFLQNFASDRSHMKDLVGGWLMPPPPYPCIETAESRMNLDDFISMNP |
| | | SVGWGHVYTLEEFVQHFGKT |
| PS2457 | 232 | MNGLSAQHERILPARHECVYTECYQEENVWKLCEHIKTSKRCPLGDVYAVFISNERKMVP |
| | | IWKQKSGRGERPVIWDYHVVLLHDRHKEQTFIYDLDTTLPFPCPFDTYVKEAFKSDNYIR |
| | | PRFWRKLRVVPADVFLQNFASDRSHMKDVSGGWRMPPPPYPCIETAESRMNLDDFISMNP |
| | | SVGWGHVYTLEEFVQHFGKT |
| PS2458 | 233 | MNGLCAQHERILPARHECVYTSCYQEENVWKLCEHIKTSKRCPLGDVYAVFISNERKMVP |
| | | IWKQKSGRGERPIIWDYHVVLLHDCHKEQTFIYDLDTTLPFPCPFDTYVKEAFKSDNYIS |
| | | PRFWRKLRVVPADVFLQNFASDRSHMKDDVGGWQMPPPPYPCIETAESRMNLDDFISMNP |
| | | SVGWGHVYTLEEFVQHFGKT |
| PS2459 | 234 | MNGLSAQHERILPARHECVYTECYQEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIR |
| | | PRFWRKLRVVPADVFLQNFASDRSHMKDAVGGWLMPPPPYPCIETAESRMNLDDFISMNP |
| | | SVGWGHVYTLEEFVQHFGKT |
| PS2460 | 235 | MNGLSAQHERILPARHECVYTECYQEENVWKLCEHIKTSKRCPLGDVYAVFISNERKMVP |
| | | IWKQRVGRGERPVIWDYHVILLHDCHKEQTFIYDLDTTLPFPCPFDTYVKEAFKSDNYIN |
| | | PRFWRKLRVVPADVFLQNFASDRSHMKDAVGGWLMPPPPYPCIETAESRMNLDDFISMNP |
| | | SVGWGHVYTLEEFVQHFGKT |
| PS2461 | 236 | MNGLSAQHERILPARHECVYTECYQEENVWKLCEHIKTSKRCPLGDVYAVFISNERKMVP |
| | | IWKQKVGRGERPLIWDYHVILLHDCHKEQTFIYDLDTTLPFPCPFDTYVKEAFKSDNYIN |
| | | PRFWRKLRVVPADVFLQNFASDRSHMKDAVGGWLMPPPPYPCIETAESRMNLDDFISMNP |
| | | SVGWGHVYTLEEFVQHFGKT |
| PS2462 | 237 | MNGLSAQHERILPARHECVYTECYQEENVWKLCEHIKTSKRCPLGDVYAVFISNERKMVP |
| | | IWKQKVGRGERPVIWDYHVVLLHDCHKEQTFIYDLDTTLPFPCPFDTYVKEAFKSDNYIN |
| | | PRFWRKLRVVPADVFLQNFASDRSHMKDAVGGWLMPPPPYPCIETAESRMNLDDFISMNP |
| | | SVGWGHVYTLEEFVQHFGKT |
| PS2463 | 238 | MNGLSAQHERILPARHECVYTECYQEENVWKLCEHIKTSKRCPLGDVYAVFISNERKMVP |
| | | IWKQKVGRGERPIIWDYHVVLLHDCHKEQTFIYDLDTTLPFPCPFDTYVKEAFKSDNYIN |
| | | PRFWRKLRVVPADVFLQNFASDRSHMKDAVGGWLMPPPPYPCIETAESRMNLDDFISMNP |
| | | SVGWGHVYTLEEFVQHFGKT |
| PS2464 | 239 | MNGLSAQHERILPARHECVYTECYQEENVWKLCEHIKTSKRCPLGDVYAVFISNERKMVP |
| | | IWKQKVGRGERPVIWDYQVILLHDCHKEQTFIYDLDTTLPFPCPFDTYVKEAFKSDNYIN |
| | | PRFWRKLRVVPADVFLQNFASDRSHMKDAVGGWLMPPPPYPCIETAESRMNLDDFISMNP |
| | | SVGWGHVYTLEEFVQHFGKT |
| PS2465 | 240 | MNGLSAQHERILPARHECVYTECYSEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIN |
| | | PRFWRKLRVVPADVFLQNFASDRSHMKDAVGGWLMPPPPYPCIETAESRMNLDDFISMNP |
| | | SVGWGHVYTLEEFVQHFGKT |
| PS2466 | 241 | MNGLSAQHERILPARHECVYTECYNEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIN |
| | | PRFWRKLRVVPADVFLQNFASDRSHMKDAVGGWLMPPPPYPCIETAESRMNLDDFISMNP |
| | | SVGWGHVYTLEEFVQHFGKT |
| PS2467 | 242 | MNGLSAQHERILPARHECVYTECYQEENVWKLCEHIKTSKRCPLGDVYAVFISNERKMVP |
| | | IWKQKVGRGERPVIWDYLVILLHDCHKEQTFIYDLDTTLPFPCPFDTYVKEAFKSDNYIN |
| | | PRFWRKLRVVPADVFLQNFASDRSHMKDAVGGWLMPPPPYPCIETAESRMNLDDFISMNP |
| | | SVGWGHVYTLEEFVQHFGKT |
| PS2468 | 243 | MNGLSAQHERILPARHECVYTSCYQEENVWKLCEHIKTSKRCPLGDVYAVFISNERKMVP |
| | | IWKQKSGRGERPVIWDYHVILLHDCHKEQTFIYDLDTTLPFPCPFDTYVKEAFKSDNYIN |
| | | PRFWRKLRVVPADVFLQNFASDRSHMKDASGGWRMPPPPYPCIETAESRMNLDDFISMNP |
| | | SVGWGHVYTLEEFVQHFGKT |
| PS2469 | 244 | MNGLSAQHERILPARHECVYTECYQEENVWKLCEHIKTSKRCPLGDVYAVFISNERKMVP |
| | | IWKQKVGRGERPVIWDYMVILLHDCHKEQTFIYDLDTTLPFPCPFDTYVKEAFKSDNYIN |
| | | PRFWRKLRVVPADVFLQNFASDRSHMKDAVGGWLMPPPPYPCIETAESRMNLDDFISMNP |
| | | SVGWGHVYTLEEFVQHFGKT |
| PS2470 | 245 | MNGLSAQHERILPARHECVYTEYYGEENVWKLCEHIKTSKRCPLGDVYAVFISNERKMVP |
| | | IWKQKSGRGERPIIWDYQVILLHDCHKEQTFIYDLDTTLPFPCPFDTYVKEAFKSDNYIN |
| | | PRFWRKLRVVPADVFLQNFASDRSHMKDIVGGWLMPPPPYPCIETAESRMNLDDFISMNP |
| | | SVGWGHVYTLEEFVQHFGKT |
| PS2471 | 246 | MNGLSAQHERILPARHECVYTECYGEENVWKLCEHIKTSKRCPLGDVYAVFISNERKMVP |
| | | IWKQKVGRGERPVIWDYQVILLHDCHKEQTFIYDLDTTLPFPCPFDTYVKEAFKSDNYIN |
| | | PRFWRKLRVVPADVFLQNFASDRSHMKDASGGWLMPPPPYPCIETAESRMNLDDFISMNP |
| | | SVGWGHVYTLEEFVQHFGKT |
| PS2472 | 247 | MNGLSAQHERILPARHECVYTECYQEENVWKLCEHIKTSKRCPLGDVYAVFISNERKMVP |
| | | IWKQKVGRGERPVIWDYNVILLHDCHKEQTFIYDLDTTLPFPCPFDTYVKEAFKSDNYIN |
| | | PRFWRKLRVVPADVFLQNFASDRSHMKDAVGGWLMPPPPYPCIETAESRMNLDDFISMNP |
| | | SVGWGHVYTLEEFVQHFGKT |
| PS2604 | 248 | MNGLSAQHERILPARHECVYTECYQEENVWKLCEHIKTSKRCPLGDVYAVFISNERKMVP |
| | | IWKQKSGRGERPVIWDYHVVLLHDRHKEQTFIYDLDTTLPFPCPFDTYVKEAFKSDNYIR |
| | | PRFWRKLRVVPADVFLQNFASDRSHMKDVSGGWRMPPPPYPCIETAESRMNLDDFISMNP |
| | | SVGWGHVYTLEEFVQHFGKTGGGSGGGSGGGSGNGLSAQHERILPARHECVYTECYQEEN |
| | | VWKLCEHIKTSKRCPLGDVYAVFISNERKMVPIWKQKSGRGERPVIWDYHVVLLHDRHKE |
| | | QTFIYDLDTTLPFPCPFDTYVKEAFKSDNYIRPRFWRKLRVVPADVFLQNFASDRSHMKD |
| | | VSGGWRMPPPPYPCIETAESRMNLDDFISMNPSVGWGHVYTLEEFVQHFGKT |
| PS2605 | 249 | MNGLSAQHERILPARHECVYTECYQEENVWKLCEHIKTSKRCPLGDVYAVFISNERKMVP |
| | | IWKQKSGRGERPVIWDYHVVLLHDRHKEQTFIYDLDTTLPFPCPFDTYVKEAFKSDNYIR |
| | | PRFWRKLRVVPADVFLQNFASDRSHMKDVSGGWRMPPPPYPCIETAESRMNLDDFISMNP |
| | | SVGWGHVYTLEEFVQHFGKTGSAGSAAGSGEFNGLSAQHERILPARHECVYTECYQEENV |
| | | WKLCEHIKTSKRCPLGDVYAVFISNERKMVPIWKQKSGRGERPVIWDYHVVLLHDRHKEQ |
| | | TFIYDLDTTLPFPCPFDTYVKEAFKSDNYIRPRFWRKLRVVPADVFLQNFASDRSHMKDV |
| | | SGGWRMPPPPYPCIETAESRMNLDDFISMNPSVGWGHVYTLEEFVQHFGKT |
| PS2606 | 250 | MNGLSAQHERILPARHECVYTECYQEENVWKLCEHIKTSKRCPLGDVYAVFISNERKMVP |
| | | IWKQKSGRGERPVIWDYHVVLLHDRHKEQTFIYDLDTTLPFPCPFDTYVKEAFKSDNYIR |
| | | PRFWRKLRVVPADVFLQNFASDRSHMKDVSGGWRMPPPPYPCIETAESRMNLDDFISMNP |
| | | SVGWGHVYTLEEFVQHFGKTGSAGSAAGSGEFGSAGSAAGSGEFGSAGSAAGSGEFNGLS |
| | | AQHERILPARHECVYTECYQEENVWKLCEHIKTSKRCPLGDVYAVFISNERKMVPIWKQK |
| | | SGRGERPVIWDYHVVLLHDRHKEQTFIYDLDTTLPFPCPFDTYVKEAFKSDNYIRPRFWR |
| | | KLRVVPADVFLQNFASDRSHMKDVSGGWRMPPPPYPCIETAESRMNLDDFISMNPSVGWG |
| | | HVYTLEEFVQHFGKT |
| PS2607 | 251 | MNGLSAQHERILPARHECVYTECYQEENVWKLCEHIKTSKRCPLGDVYAVFISNERKMVP |
| | | IWKQKSGRGERPVIWDYHVVLLHDRHKEQTFIYDLDTTLPFPCPFDTYVKEAFKSDNYIR |
| | | PRFWRKLRVVPADVFLQNFASDRSHMKDVSGGWRMPPPPYPCIETAESRMNLDDFISMNP |
| | | SVGWGHVYTLEEFVQHFGKTQGLQNEENGLSAQHERILPARHECVYTECYQEENVWKLCE |
| | | HIKTSKRCPLGDVYAVFISNERKMVPIWKQKSGRGERPVIWDYHVVLLHDRHKEQTFIYD |
| | | LDTTLPFPCPFDTYVKEAFKSDNYIRPRFWRKLRVVPADVFLQNFASDRSHMKDVSGGWR |
| | | MPPPPYPCIETAESRMNLDDFISMNPSVGWGHVYTLEEFVQHFGKT |
| PS2608 | 252 | MNGLSAQHERILPARHECVYTECYQEENVWKLCEHIKTSKRCPLGDVYAVFISNERKMVP |
| | | IWKQKSGRGERPVIWDYHVVLLHDRHKEQTFIYDLDTTLPFPCPFDTYVKEAFKSDNYIR |
| | | PRFWRKLRVVPADVFLQNFASDRSHMKDVSGGWRMPPPPYPCIETAESRMNLDDFISMNP |
| | | SVGWGHVYTLEEFVQHFGKTDPGGGPSSRLNGLSAQHERILPARHECVYTECYQEENVWK |
| | | LCEHIKTSKRCPLGDVYAVFISNERKMVPIWKQKSGRGERPVIWDYHVVLLHDRHKEQTF |
| | | IYDLDTTLPFPCPFDTYVKEAFKSDNYIRPRFWRKLRVVPADVFLQNFASDRSHMKDVSG |
| | | GWRMPPPPYPCIETAESRMNLDDFISMNPSVGWGHVYTLEEFVQHFGKT |
| PS2609 | 253 | MNGLSAQHERILPARHECVYTECYQEENVWKLCEHIKTSKRCPLGDVYAVFISNERKMVP |
| | | IWKQKSGRGERPVIWDYHVVLLHDRHKEQTFIYDLDTTLPFPCPFDTYVKEAFKSDNYIR |
| | | PRFWRKLRVVPADVFLQNFASDRSHMKDVSGGWRMPPPPYPCIETAESRMNLDDFISMNP |
| | | SVGWGHVYTLEEFVQHFGKTGNDGLCQKLSVPCMSSKPQKPWEAKDAWENGLSAQHERIL |
| | | PARHECVYTECYQEENVWKLCEHIKTSKRCPLGDVYAVFISNERKMVPIWKQKSGRGERP |
| | | VIWDYHVVLLHDRHKEQTFIYDLDTTLPFPCPFDTYVKEAFKSDNYIRPRFWRKLRVVPA |
| | | DVFLQNFASDRSHMKDVSGGWRMPPPPYPCIETAESRMNLDDFISMNPSVGWGHVYTLEE |
| | | FVQHFGKT |
| PS2610 | 254 | MNGLSAQHERILPARHECVYTECYQEENVWKLCEHIKTSKRCPLGDVYAVFISNERKMVP |
| | | IWKQKSGRGERPVIWDYHVVLLHDRHKEQTFIYDLDTTLPFPCPFDTYVKEAFKSDNYIR |
| | | PRFWRKLRVVPADVFLQNFASDRSHMKDVSGGWRMPPPPYPCIETAESRMNLDDFISMNP |
| | | SVGWGHVYTLEEFVQHFGKTFSFGFSFGFSFGNGLSAQHERILPARHECVYTECYQEENV |
| | | WKLCEHIKTSKRCPLGDVYAVFISNERKMVPIWKQKSGRGERPVIWDYHVVLLHDRHKEQ |
| | | TFIYDLDTTLPFPCPFDTYVKEAFKSDNYIRPRFWRKLRVVPADVFLQNFASDRSHMKDV |
| | | SGGWRMPPPPYPCIETAESRMNLDDFISMNPSVGWGHVYTLEEFVQHFGKT |
| PS2611 | 255 | MNGLSAQHERILPARHECVYTECYQEENVWKLCEHIKTSKRCPLGDVYAVFISNERKMVP |
| | | IWKQKSGRGERPVIWDYHVVLLHDRHKEQTFIYDLDTTLPFPCPFDTYVKEAFKSDNYIR |
| | | PRFWRKLRVVPADVFLQNFASDRSHMKDVSGGWRMPPPPYPCIETAESRMNLDDFISMNP |
| | | SVGWGHVYTLEEFVQHFGKTFSFGFSFGFSFGFSFGFSFGFSFGNGLSAQHERILPARHE |
| | | CVYTECYQEENVWKLCEHIKTSKRCPLGDVYAVFISNERKMVPIWKQKSGRGERPVIWDY |
| | | HVVLLHDRHKEQTFIYDLDTTLPFPCPFDTYVKEAFKSDNYIRPRFWRKLRVVPADVFLQ |
| | | NFASDRSHMKDVSGGWRMPPPPYPCIETAESRMNLDDFISMNPSVGWGHVYTLEEFVQHF |
| | | GKT |
| PS2612 | 256 | MNGLSAQHERILPARHECVYTECYQEENVWKLCEHIKTSKRCPLGDVYAVFISNERKMVP |
| | | IWKQKSGRGERPVIWDYHVVLLHDRHKEQTFIYDLDTTLPFPCPFDTYVKEAFKSDNYIR |
| | | PRFWRKLRVVPADVFLQNFASDRSHMKDVSGGWRMPPPPYPCIETAESRMNLDDFISMNP |
| | | SVGWGHVYTLEEFVQHFGKTEAAAKEAAAKEAAAKNGLSAQHERILPARHECVYTECYQE |
| | | ENVWKLCEHIKTSKRCPLGDVYAVFISNERKMVPIWKQKSGRGERPVIWDYHVVLLHDRH |
| | | KEQTFIYDLDTTLPFPCPFDTYVKEAFKSDNYIRPRFWRKLRVVPADVFLQNFASDRSHM |
| | | KDVSGGWRMPPPPYPCIETAESRMNLDDFISMNPSVGWGHVYTLEEFVQHFGKT |
| PS2613 | 257 | MNGLSAQHERILPARHECVYTECYQEENVWKLCEHIKTSKRCPLGDVYAVFISNERKMVP |
| | | IWKQKSGRGERPVIWDYHVVLLHDRHKEQTFIYDLDTTLPFPCPFDTYVKEAFKSDNYIR |
| | | PRFWRKLRVVPADVFLQNFASDRSHMKDVSGGWRMPPPPYPCIETAESRMNLDDFISMNP |
| | | SVGWGHVYTLEEFVQHFGKTEEEKRKREEEENGLSAQHERILPARHECVYTECYQEENVW |
| | | KLCEHIKTSKRCPLGDVYAVFISNERKMVPIWKQKSGRGERPVIWDYHVVLLHDRHKEQT |
| | | FIYDLDTTLPFPCPFDTYVKEAFKSDNYIRPRFWRKLRVVPADVFLQNFASDRSHMKDVS |
| | | GGWRMPPPPYPCIETAESRMNLDDFISMNPSVGWGHVYTLEEFVQHFGKT |
| PS2614 | 258 | MNGLSAQHERILPARHECVYTECYQEENVWKLCEHIKTSKRCPLGDVYAVFISNERKMVP |
| | | IWKQKSGRGERPVIWDYHVVLLHDRHKEQTFIYDLDTTLPFPCPFDTYVKEAFKSDNYIR |
| | | PRFWRKLRVVPADVFLQNFASDRSHMKDVSGGWRMPPPPYPCIETAESRMNLDDFISMNP |
| | | SVGWGHVYTLEEFVQHFGKTLPSPDVHNGLSAQHERILPARHECVYTECYQEENVWKLCE |
| | | HIKTSKRCPLGDVYAVFISNERKMVPIWKQKSGRGERPVIWDYHVVLLHDRHKEQTFIYD |
| | | LDTTLPFPCPFDTYVKEAFKSDNYIRPRFWRKLRVVPADVFLQNFASDRSHMKDVSGGWR |
| | | MPPPPYPCIETAESRMNLDDFISMNPSVGWGHVYTLEEFVQHFGKT |
| PS2615 | 259 | MNGLSAQHERILPARHECVYTECYQEENVWKLCEHIKTSKRCPLGDVYAVFISNERKMVP |
| | | IWKQKSGRGERPVIWDYHVVLLHDRHKEQTFIYDLDTTLPFPCPFDTYVKEAFKSDNYIR |
| | | PRFWRKLRVVPADVFLQNFASDRSHMKDVSGGWRMPPPPYPCIETAESRMNLDDFISMNP |
| | | SVGWGHVYTLEEFVQHFGKTAKLKQKTEQLQDRIAGNGLSAQHERILPARHECVYTECYQ |
| | | EENVWKLCEHIKTSKRCPLGDVYAVFISNERKMVPIWKQKSGRGERPVIWDYHVVLLHDR |
| | | HKEQTFIYDLDTTLPFPCPFDTYVKEAFKSDNYIRPRFWRKLRVVPADVFLQNFASDRSH |
| | | MKDVSGGWRMPPPPYPCIETAESRMNLDDFISMNPSVGWGHVYTLEEFVQHFGKT |
| PS2616 | 260 | MNGLSAQHERILPARHECVYTECYQEENVWKLCEHIKTSKRCPLGDVYAVFISNERKMVP |
| | | IWKQKSGRGERPVIWDYHVVLLHDRHKEQTFIYDLDTTLPFPCPFDTYVKEAFKSDNYIR |
| | | PRFWRKLRVVPADVFLQNFASDRSHMKDVSGGWRMPPPPYPCIETAESRMNLDDFISMNP |
| | | SVGWGHVYTLEEFVQHFGKTWRIRPRPPRLPRPRPRNGLSAQHERILPARHECVYTECYQ |
| | | EENVWKLCEHIKTSKRCPLGDVYAVFISNERKMVPIWKQKSGRGERPVIWDYHVVLLHDR |
| | | HKEQTFIYDLDTTLPFPCPFDTYVKEAFKSDNYIRPRFWRKLRVVPADVFLQNFASDRSH |
| | | MKDVSGGWRMPPPPYPCIETAESRMNLDDFISMNPSVGWGHVYTLEEFVQHFGKT |
| PS2617 | 261 | MNGLSAQHERILPARHECVYTECYQEENVWKLCEHIKTSKRCPLGDVYAVFISNERKMVP |
| | | IWKQKSGRGERPVIWDYHVVLLHDRHKEQTFIYDLDTTLPFPCPFDTYVKEAFKSDNYIR |
| | | PRFWRKLRVVPADVFLQNFASDRSHMKDVSGGWRMPPPPYPCIETAESRMNLDDFISMNP |
| | | SVGWGHVYTLEEFVQHFGKTAPAPAPAPAPAPAPAPAPAPNGLSAQHERILPARHECVYT |
| | | ECYQEENVWKLCEHIKTSKRCPLGDVYAVFISNERKMVPIWKQKSGRGERPVIWDYHVVL |
| | | LHDRHKEQTFIYDLDTTLPFPCPFDTYVKEAFKSDNYIRPRFWRKLRVVPADVFLQNFAS |
| | | DRSHMKDVSGGWRMPPPPYPCIETAESRMNLDDFISMNPSVGWGHVYTLEEFVQHFGKT |
| PS2618 | 262 | MNGLSAQHERILPARHECVYTECYQEENVWKLCEHIKTSKRCPLGDVYAVFISNERKMVP |
| | | IWKQKSGRGERPVIWDYHVVLLHDRHKEQTFIYDLDTTLPFPCPFDTYVKEAFKSDNYIR |
| | | PRFWRKLRVVPADVFLQNFASDRSHMKDVSGGWRMPPPPYPCIETAESRMNLDDFISMNP |
| | | SVGWGHVYTLEEFVQHFGKTAEAAAKEAAAKEAAAKEAAAKANGLSAQHERILPARHECV |
| | | YTECYQEENVWKLCEHIKTSKRCPLGDVYAVFISNERKMVPIWKQKSGRGERPVIWDYHV |
| | | VLLHDRHKEQTFIYDLDTTLPFPCPFDTYVKEAFKSDNYIRPRFWRKLRVVPADVFLQNF |
| | | ASDRSHMKDVSGGWRMPPPPYPCIETAESRMNLDDFISMNPSVGWGHVYTLEEFVQHFGK |
| | | T |
| PS2619 | 263 | MNGLSAQHERILPARHECVYTECYQEENVWKLCEHIKTSKRCPLGDVYAVFISNERKMVP |
| | | IWKQKSGRGERPVIWDYHVVLLHDRHKEQTFIYDLDTTLPFPCPFDTYVKEAFKSDNYIR |
| | | PRFWRKLRVVPADVFLQNFASDRSHMKDVSGGWRMPPPPYPCIETAESRMNLDDFISMNP |
| | | SVGWGHVYTLEEFVQHFGKTAEAAAKEAAAKEAAAKEAAAKEAAAKANGLSAQHERILPA |
| | | RHECVYTECYQEENVWKLCEHIKTSKRCPLGDVYAVFISNERKMVPIWKQKSGRGERPVI |
| | | WDYHVVLLHDRHKEQTFIYDLDTTLPFPCPFDTYVKEAFKSDNYIRPRFWRKLRVVPADV |
| | | FLQNFASDRSHMKDVSGGWRMPPPPYPCIETAESRMNLDDFISMNPSVGWGHVYTLEEFV |
| | | QHFGKT |
| PS2687 | 264 | MNGLSAQHERILPARHECVYTECYQEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIR |
| | | PRFWRKLRVVPADVFLQNFASDRSHMKDAVGGWLMPPPPYPCIETAESRMNLDDFISMNP |
| | | SVGWGHVYTLEEFVQHFGKTGGGSGGGSGGGSGNGLSAQHERILPARHECVYTECYQEEN |
| | | VWKLCEHIKTSKRCPLGDVYAVFISNERKMVPIWKQKVGRGERPVIWDYHVILLHDCHKE |
| | | QTFIYDLDTTLPFPCPFDTYVKEAFKSDNYIRPRFWRKLRVVPADVFLQNFASDRSHMKD |
| | | AVGGWLMPPPPYPCIETAESRMNLDDFISMNPSVGWGHVYTLEEFVQHFGKT |
| PS2688 | 265 | MNGLSAQHERILPARHECVYTECYQEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIR |
| | | PRFWRKLRVVPADVFLQNFASDRSHMKDAVGGWLMPPPPYPCIETAESRMNLDDFISMNP |
| | | SVGWGHVYTLEEFVQHFGKTGSAGSAAGSGEFNGLSAQHERILPARHECVYTECYQEENV |
| | | WKLCEHIKTSKRCPLGDVYAVFISNERKMVPIWKQKVGRGERPVIWDYHVILLHDCHKEQ |
| | | TFIYDLDTTLPFPCPFDTYVKEAFKSDNYIRPRFWRKLRVVPADVFLQNFASDRSHMKDA |
| | | VGGWLMPPPPYPCIETAESRMNLDDFISMNPSVGWGHVYTLEEFVQHFGKT |
| PS2689 | 266 | MNGLSAQHERILPARHECVYTECYQEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIR |
| | | PRFWRKLRVVPADVFLQNFASDRSHMKDAVGGWLMPPPPYPCIETAESRMNLDDFISMNP |
| | | SVGWGHVYTLEEFVQHFGKTGSAGSAAGSGEFGSAGSAAGSGEFGSAGSAAGSGEFNGLS |
| | | AQHERILPARHECVYTECYQEENVWKLCEHIKTSKRCPLGDVYAVFISNERKMVPIWKQK |
| | | VGRGERPVIWDYHVILLHDCHKEQTFIYDLDTTLPFPCPFDTYVKEAFKSDNYIRPRFWR |
| | | KLRVVPADVFLQNFASDRSHMKDAVGGWLMPPPPYPCIETAESRMNLDDFISMNPSVGWG |
| | | HVYTLEEFVQHFGKT |
| PS2690 | 267 | MNGLSAQHERILPARHECVYTECYQEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIR |
| | | PRFWRKLRVVPADVFLQNFASDRSHMKDAVGGWLMPPPPYPCIETAESRMNLDDFISMNP |
| | | SVGWGHVYTLEEFVQHFGKTQGLQNEENGLSAQHERILPARHECVYTECYQEENVWKLCE |
| | | HIKTSKRCPLGDVYAVFISNERKMVPIWKQKVGRGERPVIWDYHVILLHDCHKEQTFIYD |
| | | LDTTLPFPCPFDTYVKEAFKSDNYIRPRFWRKLRVVPADVFLQNFASDRSHMKDAVGGWL |
| | | MPPPPYPCIETAESRMNLDDFISMNPSVGWGHVYTLEEFVQHFGKT |
| PS2691 | 268 | MNGLSAQHERILPARHECVYTECYQEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIR |
| | | PRFWRKLRVVPADVFLQNFASDRSHMKDAVGGWLMPPPPYPCIETAESRMNLDDFISMNP |
| | | SVGWGHVYTLEEFVQHFGKTDPGGGPSSRLNGLSAQHERILPARHECVYTECYQEENVWK |
| | | LCEHIKTSKRCPLGDVYAVFISNERKMVPIWKQKVGRGERPVIWDYHVILLHDCHKEQTF |
| | | IYDLDTTLPFPCPFDTYVKEAFKSDNYIRPRFWRKLRVVPADVFLQNFASDRSHMKDAVG |
| | | GWLMPPPPYPCIETAESRMNLDDFISMNPSVGWGHVYTLEEFVQHFGKT |
| PS2692 | 269 | MNGLSAQHERILPARHECVYTECYQEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIR |
| | | PRFWRKLRVVPADVFLQNFASDRSHMKDAVGGWLMPPPPYPCIETAESRMNLDDFISMNP |
| | | SVGWGHVYTLEEFVQHFGKTGNDGLCQKLSVPCMSSKPQKPWEAKDAWENGLSAQHERIL |
| | | PARHECVYTECYQEENVWKLCEHIKTSKRCPLGDVYAVFISNERKMVPIWKQKVGRGERP |
| | | VIWDYHVILLHDCHKEQTFIYDLDTTLPFPCPFDTYVKEAFKSDNYIRPRFWRKLRVVPA |
| | | DVFLQNFASDRSHMKDAVGGWLMPPPPYPCIETAESRMNLDDFISMNPSVGWGHVYTLEE |
| | | FVQHFGKT |
| PS2693 | 270 | MNGLSAQHERILPARHECVYTECYQEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIR |
| | | PRFWRKLRVVPADVFLQNFASDRSHMKDAVGGWLMPPPPYPCIETAESRMNLDDFISMNP |
| | | SVGWGHVYTLEEFVQHFGKTFSFGFSFGFSFGNGLSAQHERILPARHECVYTECYQEENV |
| | | WKLCEHIKTSKRCPLGDVYAVFISNERKMVPIWKQKVGRGERPVIWDYHVILLHDCHKEQ |
| | | TFIYDLDTTLPFPCPFDTYVKEAFKSDNYIRPRFWRKLRVVPADVFLQNFASDRSHMKDA |
| | | VGGWLMPPPPYPCIETAESRMNLDDFISMNPSVGWGHVYTLEEFVQHFGKT |
| PS2694 | 271 | MNGLSAQHERILPARHECVYTECYQEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIR |
| | | PRFWRKLRVVPADVFLQNFASDRSHMKDAVGGWLMPPPPYPCIETAESRMNLDDFISMNP |
| | | SVGWGHVYTLEEFVQHFGKTFSFGFSFGFSFGFSFGFSFGFSFGNGLSAQHERILPARHE |
| | | CVYTECYQEENVWKLCEHIKTSKRCPLGDVYAVFISNERKMVPIWKQKVGRGERPVIWDY |
| | | HVILLHDCHKEQTFIYDLDTTLPFPCPFDTYVKEAFKSDNYIRPRFWRKLRVVPADVFLQ |
| | | NFASDRSHMKDAVGGWLMPPPPYPCIETAESRMNLDDFISMNPSVGWGHVYTLEEFVQHF |
| | | GKT |
| PS2695 | 272 | MNGLSAQHERILPARHECVYTECYQEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIR |
| | | PRFWRKLRVVPADVFLQNFASDRSHMKDAVGGWLMPPPPYPCIETAESRMNLDDFISMNP |
| | | SVGWGHVYTLEEFVQHFGKTEAAAKEAAAKEAAAKNGLSAQHERILPARHECVYTECYQE |
| | | ENVWKLCEHIKTSKRCPLGDVYAVFISNERKMVPIWKQKVGRGERPVIWDYHVILLHDCH |
| | | KEQTFIYDLDTTLPFPCPFDTYVKEAFKSDNYIRPRFWRKLRVVPADVFLQNFASDRSHM |
| | | KDAVGGWLMPPPPYPCIETAESRMNLDDFISMNPSVGWGHVYTLEEFVQHFGKT |
| PS2696 | 273 | MNGLSAQHERILPARHECVYTECYQEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIR |
| | | PRFWRKLRVVPADVFLQNFASDRSHMKDAVGGWLMPPPPYPCIETAESRMNLDDFISMNP |
| | | SVGWGHVYTLEEFVQHFGKTEEEKRKREEEENGLSAQHERILPARHECVYTECYQEENVW |
| | | KLCEHIKTSKRCPLGDVYAVFISNERKMVPIWKQKVGRGERPVIWDYHVILLHDCHKEQT |
| | | FIYDLDTTLPFPCPFDTYVKEAFKSDNYIRPRFWRKLRVVPADVFLQNFASDRSHMKDAV |
| | | GGWLMPPPPYPCIETAESRMNLDDFISMNPSVGWGHVYTLEEFVQHFGKT |
| PS2697 | 274 | MNGLSAQHERILPARHECVYTECYQEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIR |
| | | PRFWRKLRVVPADVFLQNFASDRSHMKDAVGGWLMPPPPYPCIETAESRMNLDDFISMNP |
| | | SVGWGHVYTLEEFVQHFGKTLPSPDVHNGLSAQHERILPARHECVYTECYQEENVWKLCE |
| | | HIKTSKRCPLGDVYAVFISNERKMVPIWKQKVGRGERPVIWDYHVILLHDCHKEQTFIYD |
| | | LDTTLPFPCPFDTYVKEAFKSDNYIRPRFWRKLRVVPADVFLQNFASDRSHMKDAVGGWL |
| | | MPPPPYPCIETAESRMNLDDFISMNPSVGWGHVYTLEEFVQHFGKT |
| PS2698 | 275 | MNGLSAQHERILPARHECVYTECYQEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIR |
| | | PRFWRKLRVVPADVFLQNFASDRSHMKDAVGGWLMPPPPYPCIETAESRMNLDDFISMNP |
| | | SVGWGHVYTLEEFVQHFGKTAKLKQKTEQLQDRIAGNGLSAQHERILPARHECVYTECYQ |
| | | EENVWKLCEHIKTSKRCPLGDVYAVFISNERKMVPIWKQKVGRGERPVIWDYHVILLHDC |
| | | HKEQTFIYDLDTTLPFPCPFDTYVKEAFKSDNYIRPRFWRKLRVVPADVFLQNFASDRSH |
| | | MKDAVGGWLMPPPPYPCIETAESRMNLDDFISMNPSVGWGHVYTLEEFVQHFGKT |
| PS2699 | 276 | MNGLSAQHERILPARHECVYTECYQEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIR |
| | | PRFWRKLRVVPADVFLQNFASDRSHMKDAVGGWLMPPPPYPCIETAESRMNLDDFISMNP |
| | | SVGWGHVYTLEEFVQHFGKTWRIRPRPPRLPRPRPRNGLSAQHERILPARHECVYTECYQ |
| | | EENVWKLCEHIKTSKRCPLGDVYAVFISNERKMVPIWKQKVGRGERPVIWDYHVILLHDC |
| | | HKEQTFIYDLDTTLPFPCPFDTYVKEAFKSDNYIRPRFWRKLRVVPADVFLQNFASDRSH |
| | | MKDAVGGWLMPPPPYPCIETAESRMNLDDFISMNPSVGWGHVYTLEEFVQHFGKT |
| PS2700 | 277 | MNGLSAQHERILPARHECVYTECYQEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIR |
| | | PRFWRKLRVVPADVFLQNFASDRSHMKDAVGGWLMPPPPYPCIETAESRMNLDDFISMNP |
| | | SVGWGHVYTLEEFVQHFGKTAPAPAPAPAPAPAPAPAPAPNGLSAQHERILPARHECVYT |
| | | ECYQEENVWKLCEHIKTSKRCPLGDVYAVFISNERKMVPIWKQKVGRGERPVIWDYHVIL |
| | | LHDCHKEQTFIYDLDTTLPFPCPFDTYVKEAFKSDNYIRPRFWRKLRVVPADVFLQNFAS |
| | | DRSHMKDAVGGWLMPPPPYPCIETAESRMNLDDFISMNPSVGWGHVYTLEEFVQHFGKT |
| PS2701 | 278 | MNGLSAQHERILPARHECVYTECYQEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIR |
| | | PRFWRKLRVVPADVFLQNFASDRSHMKDAVGGWLMPPPPYPCIETAESRMNLDDFISMNP |
| | | SVGWGHVYTLEEFVQHFGKTAEAAAKEAAAKEAAAKEAAAKANGLSAQHERILPARHECV |
| | | YTECYQEENVWKLCEHIKTSKRCPLGDVYAVFISNERKMVPIWKQKVGRGERPVIWDYHV |
| | | ILLHDCHKEQTFIYDLDTTLPFPCPFDTYVKEAFKSDNYIRPRFWRKLRVVPADVFLQNF |
| | | ASDRSHMKDAVGGWLMPPPPYPCIETAESRMNLDDFISMNPSVGWGHVYTLEEFVQHFGK |
| | | T |
| PS2702 | 279 | MNGLSAQHERILPARHECVYTECYQEENVWKLCEHIKTSKRCPLGDVYAVFISNERKMVP |
| | | IWKQKVGRGERPVIWDYHVILLHDCHKEQTFIYDLDTTLPFPCPFDTYVKEAFKSDNYIR |
| | | PRFWRKLRVVPADVFLQNFASDRSHMKDAVGGWLMPPPPYPCIETAESRMNLDDFISMNP |
| | | SVGWGHVYTLEEFVQHFGKTAEAAAKEAAAKEAAAKEAAAKEAAAKANGLSAQHERILPA |
| | | RHECVYTECYQEENVWKLCEHIKTSKRCPLGDVYAVFISNERKMVPIWKQKVGRGERPVI |
| | | WDYHVILLHDCHKEQTFIYDLDTTLPFPCPFDTYVKEAFKSDNYIRPRFWRKLRVVPADV |
| | | FLQNFASDRSHMKDAVGGWLMPPPPYPCIETAESRMNLDDFISMNPSVGWGHVYTLEEFV |
| | | QHFGKT |

TABLE 2A
Non-limiting examples of tag peptides.

| | SEQ ID | |
|---|---|---|
| Name | NO: | Sequence |
| Biotinylation tag | 186 | GGGSGGGSGGGSGLNDFFEAQKIEWHE |
| Bis-biotinylation tag | 187 | GGGSGGGSGGGSGLNDFFEAQKIEWHEGGGSGGGSGGGSGLNDF |
| | | FEAQKIEWHE |
| Bis-biotinylation tag | 188 | GSGGGSGGGSGGGSGLNDFFEAQKIEWHEGGGSGGGSGGGSGLN |
| | | DFFEAQKIEWHE |
| His/biotinylation tag | 189 | GHHHHHHHHHHGGGSGGGSGGGSGLNDFFEAQKIEWHE |
| His/bis-biotinylation tag | 190 | GHHHHHHHHHHGGGSGGGSGGGSGLNDFFEAQKIEWHEGGGSGG |
| | | GSGGGSGLNDFFEAQKIEWHE |
| His/bis-biotinylation tag | 191 | GGSHHHHHHHHHHGGGSGGGSGGGSGLNDFFEAQKIEWHEGGGS |
| | | GGGSGGGSGLNDFFEAQKIEWHE |
| His/bis-biotinylation tag | 192 | GSHHHHHHHHHHGGGSGGGSGGGSGLNDFFEAQKIEWHEGGGSG |
| | | GGSGGGSGLNDFFEAQKIEWHE |
| Bis-biotinylation/His tag | 193 | GGGSGGGSGGGSGLNDFFEAQKIEWHEGGGSGGGSGGGSGLNDF |
| | | FEAQKIEWHEGHHHHHH |
| Bis-biotinylation/His tag | 280 | GSGGGSGGGSGGGSGLNDIFEAQKIEWHEGGGSGGGSGGGSGLN |
| | | DIFEAQKIEWHEGGGGSHHHHHH |

TABLE 2B
Non-limiting examples of tandem linkers.

| | SEQ ID | |
|---|---|---|
| Name | NO: | Sequence |
| Linker 1 | 194 | GGGSGGGSGGGSG |
| Linker 2 | 195 | GSAGSAAGSGEF |
| Linker 3 | 196 | GSAGSAAGSGEFGSAGSAAGSGEFGSAGSAAGSGEF |
| Linker 4 | 197 | EAAAKEAAAKEAAAK |
| Linker 5 | 198 | AEAAAKEAAAKEAAAKEAAAKA |
| Linker 6 | 199 | AEAAAKEAAAKEAAAKEAAAKEAAAKA |
| Linker 7 | 200 | EEEKRKREEEE |
| Linker 8 | 201 | QGLQNEE |
| Linker 9 | 202 | DPGGGPSSRL |
| Linker 10 | 203 | GNDGLCQKLSVPCMSSKPQKPWEAKDAWE |
| Linker 11 | 204 | FSFGFSFGFSFG |
| Linker 12 | 205 | FSFGFSFGFSFGFSFGFSFGFSFG |
| Linker 13 | 206 | LPSPDVH |
| Linker 14 | 207 | AKLKQKTEQLQDRIAG |
| Linker 15 | 208 | WRIRPRPPRLPRPRPR |
| Linker 16 | 209 | APAPAPAPAPAPAPAPAPAP |

### EXAMPLES

### Example 1. Development of Aspartate/Glutamate Recognizer PS2195

This Example describes the development of PS2195 (SEQ ID NO: 25), an engineered variant of an Ntaq1-homologous recognizer from Scleropages Formosus (PS1259) capable of recognizing aspartate. PS1259 is an engineered glutaminase variant with improved binding properties for recognizing glutamine and asparagine, and this was attributed in part to a mutation in the catalytic triad (H78Q). It was discovered that an alternative mutation at the same position (H78K) changed the homolog from an improved glutamine/asparagine recognizer to a glutamate recognizer in PS1875, which led to development of PS2132 via several rounds of development techniques including, e.g., directed evolution and protein engineering guided by protein ensemble and single molecule kinetic analysis. Through additional rounds of directed evolution, protein engineering, and subsequent evaluation, PS2195 was developed which changed the homolog to an aspartate/glutamate recognizer.

Ntaq1-homologous protein candidate recognizers were identified by development techniques including, e.g., directed evolution, expressed in E. coli and purified. The candidates were evaluated for binding to N-terminal amino acids on the Octet binding platform. The peptides used in the assay contained (i) a penultimate alanine and an N-terminal asparagine (NA); (ii) a penultimate alanine and an N-terminal glutamine (QA); (iii) a penultimate alanine and an N-terminal glutamate (EA); or (iv) a penultimate alanine and an N-terminal aspartate (DA). In the high-throughput assay, Octet sensors were coated with the peptide of interest and dipped in buffer containing the purified protein. The set of Octet response measurements is summarized in Table 3 (an empty cell indicates not measured or candidate did not express protein).

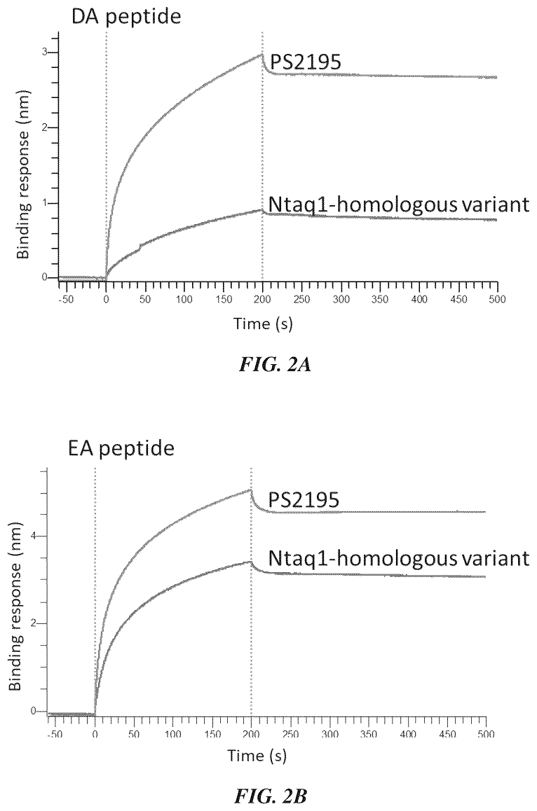

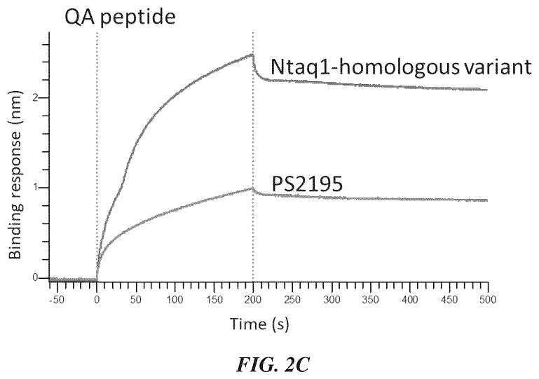

These results led to the identification of PS2195 (D and E recognizer). The binding data representative of the binding interaction between PS2195 and the DA, LA, and QA peptides are shown in FIGS. 2A-2C, respectively. A control Ntaq1-homologous variant is also shown. Improved binding can be illustrated by an increase in the response based on a shift in wavelength, given in nm, over time (association curves between 0 and 200 sec, dissociation curves between 200 and 500 sec).

TABLE 3
Octet response for Ntaq-1 homologous variants.

| Binders | NA | QA | EA | DA | Homologs/Mutations |
|---|---|---|---|---|---|
| PS1246 | 0.1 | 0 | 0 | 0.01 | hntaq1 |
| PS1259 | 1.7 | 3.6 | 0.1 | 0.03 | hntaq1 + C25S, H78Q |
| PS1875 | 0.1 | 0.8 | 1 | 0.2 | PS1259 + Q78K |
| PS2029 | 0.3 | 0.9 | 1.2 | 0.4 | PS1259 + K31H, E34Q, Q78K |
| PS2116 | | 0.9 | 2.5 | 0.8 | PS1259 + S22E, Q78K |
| PS2117 | | 3.5 | 3.2 | 0.9 | PS1259 + P72R, Q78K |
| PS2118 | | 1.4 | 2 | 0.8 | PS1259 + Q78K, A149Q |
| PS2119 | | 2.9 | 2.3 | 0.9 | PS1259 + Q78K, A149V |
| PS2120 | | 2.4 | 3.7 | 1.2 | PS1259 + S39Q, Q78K, C85T, N120R |
| PS2121 | | 2.5 | 3.5 | 1.3 | PS1259 + S22E, S39Q, Q78K, C85T, N120R |
| PS2122 | | 1.5 | 2.4 | 0.8 | PS1259 + S22E, Q78K, A149Q |
| PS2123 | | 2.4 | 2.2 | 1 | PS1259 + S22E, Q78K, N120R |
| PS2124 | | 1.2 | 2 | 0.6 | PS1259 + S22E, Q78K, C85T |
| PS2125 | | 1.2 | 2.1 | 0.8 | PS1259 + S22E, S39Q, Q78K |
| PS2126 | | 1.8 | 3.2 | 1.1 | PS1259 + Q78K, N120R, A149Q |
| PS2127 | | 1.3 | 2.2 | 0.8 | PS1259 + Q78K, C85T, A149Q |
| PS2128 | | 1.2 | 1.9 | 0.9 | PS1259 + S39Q, Q78K, A149Q |
| PS2129 | | 1.7 | 3.2 | 0.2 | PS1259 + S22E, Q78K, N120R, A149Q |
| PS2130 | | 1.6 | 2.4 | 0.9 | PS1259 + S22E, Q78K, C85T, A149Q |
| PS2131 | | 1.4 | 2.2 | 0.8 | PS1259 + S22E, S39Q, Q78K, A149Q |
| PS2132 | | 2.5 | 3.4 | 0.9 | PS1259 + S22E, Q78K, C85T, N120R |
| PS2133 | | 2.2 | 3.3 | 1.2 | PS1259 + S22E, S39Q, Q78K, N120R |
| PS2134 | | 2.1 | 3.5 | 0.4 | PS1259 + S22E, Q78K, N120R, A149V |
| PS2135 | | | | | PS1259 + S22E, S39Q, Q78K, C85T |
| PS2136 | | | | | PS1259 + S22E, Q78K, C85T, A149V |
| PS2137 | | | | | PS1259 + S22E, S39Q, Q78K, A149V |
| PS2150 | 4.9 | 4.5 | | 0.3 | PS1259 + A12L, S22P, W30Y, E71R, P72V, A122R |
| PS2151 | 5.7 | 6.6 | | 0.5 | PS1259 + A12L, S22P, W30Y, K65R, E71R, P72V, A122R, P131R |
| PS2152 | 5.2 | 6.2 | | 0.1 | PS1259 + A12L, E71R, P72L, A122R |
| PS2153 | 7.2 | 7.8 | | 0.8 | PS1259 + A12L, S22E, C23F, E71R, A122R |
| PS2154 | 5.9 | 6.8 | | 0.6 | PS1259 + S22P, K65R, E71R, A122R |
| PS2155 | 7.6 | 7.8 | | 0.1 | PS1259 + A12L, S22E, E71R, P72V, L81M, A122R |
| PS2156 | 5.7 | 7.1 | | 0.1 | PS1259 + A12L, E71R, A122R |
| PS2157 | 5.2 | 6.1 | | 0.1 | PS1259 + A12L, K65R, E71R, P72V, A122R |
| PS2158 | 6.4 | 6.9 | | 0.1 | PS1259 + A12L, S22E, E71R, A122R |
| PS2159 | 7.1 | 8.5 | 0 | 0 | PS1259 + A12L, S22E, C23F, E71R, N120R, A122R |
| PS2160 | 4.5 | 6 | 0.1 | 0.2 | PS1259 + A12L, S22P, S39Q, S66V, A122R |
| PS2161 | 4.8 | 5.9 | 0.1 | 0.1 | PS1259 + A12L, W30Y, K65R, E71R, P72V, T90S, A122R |
| PS2162 | | 6.5 | 0.1 | 0.1 | PS1259 + A12L, S22P, W30Y, E71R, P72V, L81M, A122R, P131R |
| PS2163 | | 5.1 | 0.1 | 0.1 | PS1259 + A12L, S22P, K65R, E71R, P72V, A122R |
| PS2164 | | 4.7 | 0.1 | 0 | PS1259 + A12L, S22P, P72F, A122R |
| PS2165 | | 8 | 0.1 | 0.1 | PS1259 + A12L, S39Q, S66V, N120R, A122R |
| PS2166 | | 0.9 | 2.7 | 1.5 | PS1259 + S22P, C23G, E34Q, K65R, V73L, Q78K, A122R, A149S |
| PS2167 | | 0.9 | 2.2 | 1.2 | PS1259 + S22P, C23G, E34Q, K65R, V73L, Q78K, A122R |
| PS2168 | | 0.9 | 4.9 | 3 | PS1259 + A12L, C23G, Q78K, I80Q, A122R, A149D, S150L |
| PS2169 | | 0 | 0.2 | 0 | PS1259 + S22P, C23G, S25G, E34Q, K65R, V73L, Q78R, K114R, A122R, |
| | | | | | A149E |
| PS2170 | | 0.4 | 0.5 | 0.4 | PS1259 + A12L, C23G, V73L, Q78K |
| PS2171 | | 6.9 | 0.1 | 0.1 | PS1259 + A12L, S22P, S66V, N120R, A122R |
| PS2195 | | 1 | 5.2 | 2.9 | PS1259 + A12L, S22P, C23G, S25G, K65R, V73L, Q78R, D96R, K114R, |
| | | | | | A122R, A149S, S150R |
| PS2196 | | 2.7 | 0 | 0.1 | PS1259 + A12L, S22P, C23G, D46V, K65R, V73L, K114R, A122R, |
| | | | | | A149E, S150R |
| PS2197 | | 5.6 | 0 | 0 | PS1259 + A12L, S22P, E34Q, K65R, V73L, A122R, A149S, S150R |
| PS2198 | | 1.5 | 3.8 | 0.8 | PS1259 + A12L, S22P, C23Q, C42F, K65R, Q78K, A122R, A149S |
| PS2199 | | 0.3 | 3.1 | 0.3 | PS1259 + A12L, S22P, C23Q, K65R, V73I, Q78K, I80F, A122R, A149S, |
| | | | | | S150R |
| PS2200 | | 0.8 | 2 | 0.8 | PS1259 + A12L, S22P, C23G, S25G, D46G, K65R, V73L, Q78R, E111K, |
| | | | | | K114R, A122R, S150R |
| PS2201 | | 1 | 1.7 | 1.4 | PS1259 + A12L, S22P, C23G, S25G, K57R, K65R, V73L, Q78R, I80F, |
| | | | | | D96R, A122R, S150R |
| PS2202 | | 1 | 1.4 | 1.1 | PS1259 + A12L, S22P, C23G, E34Q, P43L, V73L, Q78R, D96R, K114R, |
| | | | | | S150R |
| PS2203 | | 1 | 0.8 | 0.5 | PS1259 + A12L, S22P, C23G, S25G, K65R, V73L, Q78K, A122R, A149S, |
| | | | | | S150R, F193L |
| PS2204 | | 0.7 | 0.3 | 0.1 | PS1259 + A12L, C23G, S25G, E34Q, K65R, V73L, Q78K, K114R, |
| | | | | | A122R, A149S |
| PS2205 | | 0.7 | 1.2 | 0.7 | PS1259 + A12L, S22P, C23G, S25G, K65R, V73L, Q78R, K114R, A122R, |
| | | | | | A149S, S150R |
| PS2244 | | 0.9 | 4.3 | 2 | PS1259 + S22V, C23G, S39V, P72W, I74W, Q78K, C85A, D96G, N120H, |
| | | | | | A149S |
| PS2245 | | 1.8 | 1.9 | 0.9 | PS1259 + S22Q, C23G, S39Y, P72Y, I74V, Q78K, D96G, A149S |
| PS2246 | | 1.2 | 4.4 | 2 | PS1259 + S22P, C23G, P72H, I74L, Q78K, D96G, A149R |
| PS2247 | | 2.1 | 3.2 | 1.5 | PS1259 + S22Q, C23G, S39Q, P72Y, I74L, Q78K, C85G, D96G, D148L |
| PS2248 | | 1.1 | 4.5 | 1.8 | PS1259 + S22L, C23G, S39G, P72H, I74L, Q78K, D96G, D148L |
| PS2249 | | 0 | 2.9 | 2.2 | PS1259 + S22Q, C23G, S39Q, P72Y, I74L, Q78K, C85G, D96G, A149K |
| PS2250 | | 1.2 | 4.4 | 1.9 | PS1259 + S22V, C23G, S39R, P72H, I74L, Q78K, D96G, D148T |
| PS2251 | | 1 | 3.1 | 1.4 | PS1259 + S22P, C23G, S39N, P72W, I74L, Q78K, C85R, D96G, A149L |
| PS2252 | | 1.6 | 3.1 | 1.6 | PS1259 + S22E, C23G, S39R, P72H, I74V, Q78K, C85F, D96G, A149R |
| PS2253 | | 1.3 | 3.5 | 1.8 | PS1259 + C23G, S39K, P72H, I74L, Q78K, D96G, A149S |
| PS2254 | | 2.1 | 2.1 | 1.2 | PS1259 + S22Q, C23G, S39Y, P72Y, I74V, Q78K, D96G, D148L |
| PS2255 | | 1.3 | 3.8 | 1.8 | PS1259 + C23G, S39V, P72W, I74L, Q78K, D96G, D148L |
| PS2256 | | 1.1 | 1.6 | 1.2 | PS1259 + S22T, C23G, S39M, P72H, Q78K, D96G |
| PS2257 | | 1 | 1.8 | 1.2 | PS1259 + S22A, C23G, S39F, P72H, Q78K, C85I, D96G |
| PS2258 | | 1.1 | 4.8 | 2.7 | PS1259 + S22E, C23G, P72H, I74L, Q78K, D96G, A149R |
| PS2259 | | 1.1 | 4 | 1.9 | PS1259 + C23G, S39K, P72H, I74L, Q78K, D96G, A149P |
| PS2260 | | 1.1 | 4.5 | 1.8 | PS1259 + S22P, C23G, P72H, I74L, Q78K, D96G, D148L |
| PS2261 | | 1.3 | 4.4 | 2 | PS1259 + C23G, S39K, P72H, I74L, Q78K, D96G, D148L |
| PS2262 | | 1.3 | 3.6 | 2.1 | PS1259 + S22E, C23G, S39R, P72W, I74L, Q78K, D96G, A149T |
| PS2263 | | 1.1 | 3.5 | 1.8 | PS1259 + C23G, S39K, P72H, I74L, Q78K, D96G, A149N |
| PS2264 | | 1.1 | 4.9 | 2.5 | PS1259 + S22Q, C23G, S39V, P72H, I74L, Q78K, D96G, A149R |
| PS2265 | | 2 | 2.8 | 1.3 | PS1259 + S22Q, C23G, S39Q, P72Y, I74L, Q78K, C85G, D96G, D148T |
| PS2278 | | | | | PS1259 + A149K |
| PS2279 | | | | | PS1259 + A149M |
| PS2280 | 1.3 | 4.7 | | 0.1 | PS1259 + A149T |
| PS2281 | 3.7 | 5.5 | | 0.1 | PS1259 + A149P |
| PS2282 | 2.6 | 5 | | 0.1 | PS1259 + D148T |
| PS2283 | 2.3 | 4.1 | | 0.1 | PS1259 + A149D |
| PS2284 | 3.3 | 5.6 | | 0.1 | PS1259 + D148S |
| PS2285 | 1.7 | 3.9 | | 0.1 | PS1259 + A149I |
| PS2286 | 2.7 | 4 | | 0.1 | PS1259 + S22D |
| PS2287 | 3.6 | 5.5 | | 0.4 | PS1259 + S39G |
| PS2288 | 3.6 | 4.4 | | 0.4 | PS1259 + S22D, C23S |
| PS2289 | 3.3 | 5.5 | | 0.1 | PS1259 + S39R |
| PS2290 | 2.3 | 4.1 | | 0.4 | PS1259 + C23S |
| PS2291 | 2.4 | 4.7 | | 0.1 | PS1259 + C85S, A149R |
| PS2292 | 3.1 | 5.6 | | 0.3 | PS1259 + D148E |
| PS2293 | 1.5 | 4.6 | | 0.2 | PS1259 + D148Y |
| PS2294 | 1.6 | 3.8 | | 0.1 | PS1259 + A149L |
| PS2295 | 5.3 | 5.2 | | 0.4 | PS1259 + S22W |
| PS2296 | 3.1 | 5.2 | | 0.1 | PS1259 + P72M, A149T |
| PS2297 | 2.4 | 4.5 | | 0.1 | PS1259 + D148N |
| PS2298 | 2.3 | 2.7 | | 0.2 | PS1259 + C23G, S39G, P72L, A149L |
| PS2299 | 6.3 | 2.9 | | 0.3 | PS1259 + C23G, S39R, P72M, A149R |
| PS2392 | | 1.4 | 5 | 2.6 | PS2195 + A12L, S22P, C23G, S25G, K65R, V73L, Q78R, D96R, K114R, |
| | | | | | N120R, A122R, A149S, S150R |
| PS2393 | | 1.4 | 4.8 | 3 | PS2195 + A12L, S22P, C23G, S25G, K65R, V73L, Q78R, D96R, K114R, |
| | | | | | A122R, S150R |
| PS2394 | | 1.2 | 3.7 | 2.4 | PS2195 + A12L, S22P, C23G, S25G, K65R, V73L, Q78R, D96R, K114R, |
| | | | | | A122R, A149S |
| PS2395 | | 1.4 | 3.6 | 2.3 | PS2195 + A12L, S22P, C23G, S25G, K65R, V73L, Q78R, D96R, K114R, |
| | | | | | A122R |
| PS2396 | | 1.1 | 2.2 | 1.5 | PS2195 + A12L, S22P, C23G, K65R, V73L, Q78R, D96R, K114R, A122R, |
| | | | | | A149S, S150R |
| PS2397 | | 1.7 | 8.1 | 2.4 | PS2195 + A12L, S22P, S25G, K65R, V73L, Q78R, D96R, K114R, A122R, |
| | | | | | A149S, S150R |
| PS2398 | | 1.4 | 5.6 | 3.7 | PS2195 + A12L, C23G, S25G, K65R, V73L, Q78R, D96R, K114R, |
| | | | | | A122R, A149S, S150R |
| PS2399 | | 0.4 | 8.9 | 4.2 | PS2195 + A12L, S22P, C23G, S25G, V73L, Q78R, D96R, K114R, A122R, |
| | | | | | A149S, S150R |
| PS2400 | | 1.4 | 3.9 | 2.4 | PS2195 + A12L, S22P, C23G, S25G, K65R, V73L, Q78R, D96R, K114R, |
| | | | | | N120R, A122R, S150R |
| PS2401 | | 0.9 | 4 | 1.5 | PS2195 + A12L, S22P, C23G, S25G, K65R, V73L, Q78R, D96R, K114R, |
| | | | | | N120R, A122R, A149S, S150R, R154L |
| PS2402 | | 1.5 | 6.5 | 4.8 | PS2195 + A12L, S22P, C23G, S25G, K65R, E71R, V73L, Q78R, D96R, |
| | | | | | K114R, A122R, A149S, S150R |
| PS2428 | | 1.3 | 4.1 | 2.3 | PS1259 + A12L, S22E, C23G, S25G, Q78R, C85T, D96R, P100S, N120R, |
| | | | | | A122R, R154Q |
| PS2429 | | 1.1 | 4.1 | 2.3 | PS1259 + A12L, S22E, C23G, S25G, E71R, V73L, Q78R, D96R, A122R, |
| | | | | | A149S, S150R, R154P |
| PS2430 | | 0.4 | 9 | 5.6 | PS1259 + A12L, S22E, C23G, S25G, Q78R, C85T, D96R, P100S, N120R, |
| | | | | | A122R, D148G, R154Q |
| PS2431 | | 0.3 | 1.6 | 1.7 | PS1259 + A12L, S22E, C23G, S25G, Q78R, C85P, D96R, P100S, N120R, |
| | | | | | A122R, R154Q |
| PS2432 | | 5.1 | 0.9 | 0.5 | PS1259 + A12L, S22E, E71R, Q78K, C85T, Y93H, D96R, K114R, A122R, |
| | | | | | A149S, S150R, R154L |
| PS2433 | | 0.3 | 0.3 | 0.3 | PS1259 + A12L, S22P, C23G, K31I, E71R, Q78R, C85T, D96K, P102S, |
| | | | | | K114R, N120R, A122R, A149S, R154Q |
| PS2434 | | 1.7 | 5.4 | 1.6 | PS1259 + A12L, S22P, S25G, E71R, V73L, Q78R, C85T, D96R, A122R, |
| | | | | | A149S, S150R, R154L |
| PS2435 | | 1.4 | 1.3 | 1.4 | PS1259 + A12L, S22P, C23G, V73L, Q78R, D96R, N120R, A122R, |
| | | | | | S150R |
| PS2436 | | 1 | 3.9 | 1.5 | PS1259 + A12L, S22E, C23G, S25G, K37E, Q78R, C85T, D96R, P100S, |
| | | | | | N120R, A122R, R154Q |
| PS2437 | | 0.3 | 0.3 | 0.3 | PS1259 + A12L, S22P, E71R, Q78R, Y93H, K110E, K114R, N120R, |
| | | | | | A122R, S150R, R154P |
| PS2438 | | 0.8 | 4.5 | 2 | PS1259 + A12L, S22E, C23G, S25G, E70G, E71R, V73L, Q78R, D96R, |
| | | | | | A122R, A149S, S150R, R154P |

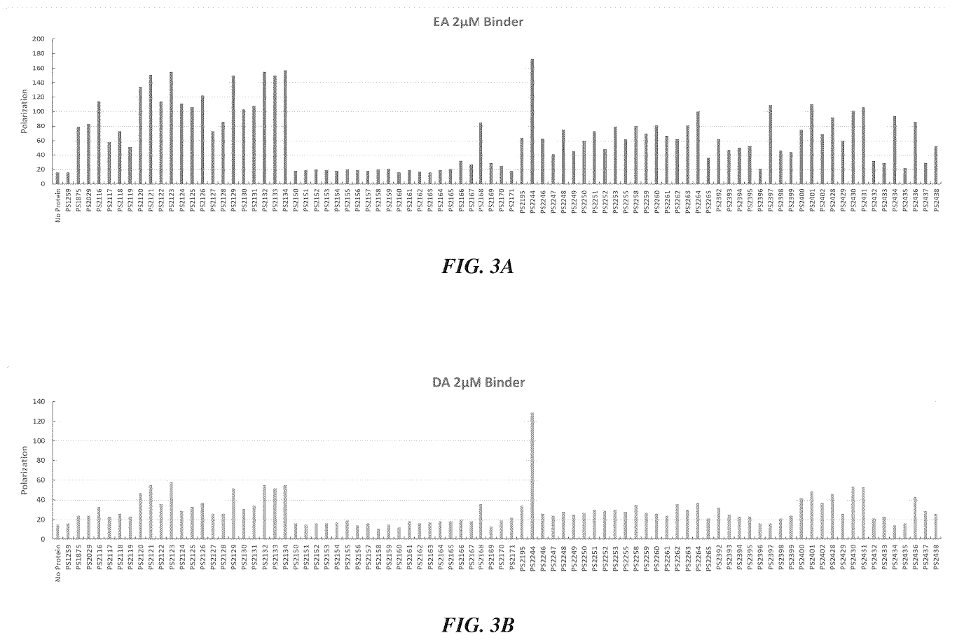

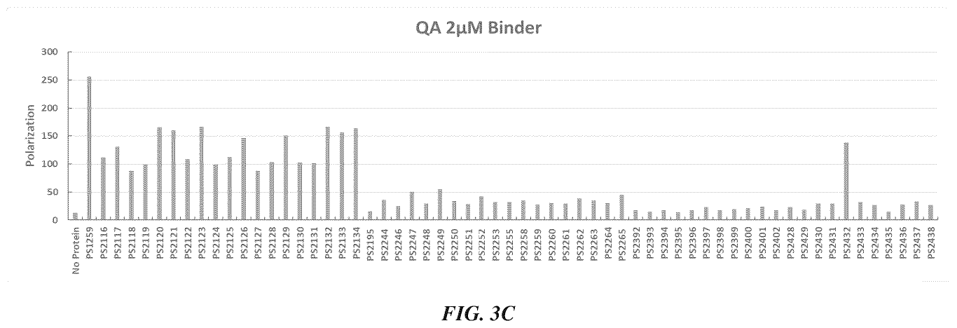

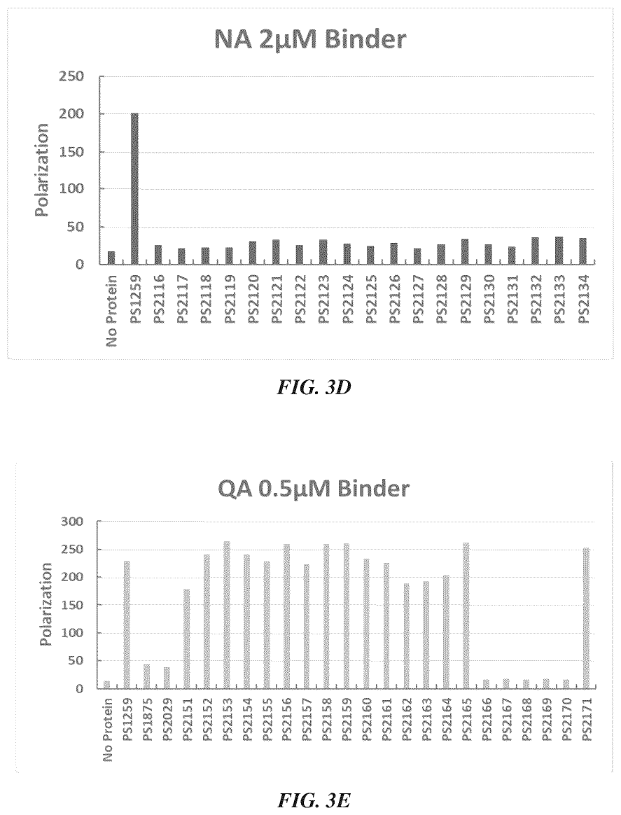

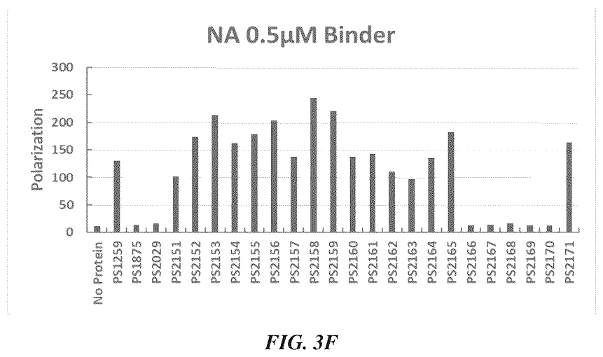

Fluorescence polarization assays were performed with a subset of candidates, and single point binding responses were measured at a fixed concentration of the binders (FIGS. 3A-3F). This assay measures the strength of the interaction between a binder and fluorescein labeled peptides (with XAKLDEESILKQK-FITC (SEQ ID NO: 289), XHGSK-FITC-DEESILKQ (SEQ ID NO: 290), or XVFRDEESILKQK-FITC (SEQ ID NO: 291)). In these sequences, the ‘X’ can be an N, Q, E, or D; and the ‘FITC’ represents fluorescein. Ensemble Rapid kinetics measurements were obtained for N-terminal N, Q, E, and D binding by select variants, with the highly pure unconjugated protein preps of top Ntaq1-homologous variants after high-throughput kinetics evaluation. Binding affinities (K_{d}) were determined by fluorescence polarization at 20° C. (results summarized in Tables 4-6: dash indicates not measured). The kₒₙ rate constants and k_{off} rates were derived by stopped-flow rapid kinetic analysis at 30° C. for NA, QA, EA, and DA peptides (results summarized in Table 4; dash indicates not measured)

TABLE 4
Kinetics Study: Ntaq1-homologous variants (EA/EH/DA/DH peptides).

| Variant | EA Kd ± std. error (nM) | EH Kd ± std. error (nM) | DA Kd ± std. error (nM) | DH Kd ± std. error (nM) |
|---|---|---|---|---|
| PS1875 | 1993 ± 174 | — | — | — |
| PS2120 | 1355 ± 123 | — | 15432 ± 2024 | — |
| PS2121 | 842 ± 49 | — | 9504 ± 1557 | — |
| PS2123 | 896 ± 77 | — | 10050 ± 970 | — |
| PS2129 | 1122 ± 106 | — | 16139 ± 1586 | — |
| PS2132 | 746 ± 27 | Very weak binding | 8876 ± 1787 | Very weak binding |
| PS2133 | 908 ± 172 | — | 11328 ± 1717 | — |
| PS2134 | 899 ± 99 | — | 5455 ± 570 | — |
| PS2167 | — | — | very weak binding | — |
| PS2168 | — | — | 32381 ± 7129 | — |
| PS2195 | ND | ND | 27630 ± 5289 | ND |
| PS2244 | 171 ± 11 | 4700 ± 2024 | 913 ± 85 | Very weak binding |
| PS2258 | 3296 ± 522 | Very weak binding | Very weak binding | Very weak binding |
| PS2264 | 1979 ± 139 | Very weak binding | Very weak binding | Very weak binding |

TABLE 5
Kinetics Study: Ntaq1-homologous variants EAKL (SEQ ID NO: 283)/DAKL (SEQ ID NO: 282)/EVFR (SEQ
ID NO: 284)/DVFR (SEQ ID NO: 285)/QAKL (SEQ ID NO: 286)/NAKL (SEQ ID NO: 287) peptides).

| | EAKL | DAKL | EVFR | DVFR | QAKL | NAKL |
|---|---|---|---|---|---|---|
| | (SEQ ID | (SEQ ID | (SEQ ID | (SEQ ID | (SEQ ID | (SEQ ID |
| | NO: 283) | NO: 282) | NO: 284) | NO: 285) | NO: 286) | NO: 287) |
| | Kd ± std. | Kd ± std. | Kd ± std. | Kd ± std. | Kd ± std. | Kd ± std. |
| Variant | error (nM) | error (nM) | error (nM) | error (nM) | error (nM) | error (nM) |
| PS1259 | No binding | No binding | — | — | 279 ± 20 | 1326 ± 44 |
| PS1875 | 11242 ± 717 | No binding | — | — | — | — |
| PS2121 | 2570 ± 117 | 21951 ± 1942 | — | — | 3995 ±137 | Very weak binding |
| PS2123 | 3025 ± 140 | 33054 ± 4895 | — | — | 4628 ± 167 | Very weak binding |
| PS2132 | 2171 ± 69 | 19167 ± 2025 | — | — | 2893 ± 91 | Very weak binding |
| PS2134 | 4109 ± 442 | 22904 ± 1427 | — | — | — | — |
| PS2195 | 19540 ± 1504 | 50709 ± 3209 | 1470 ± 14 | 2021 ± 49 | ND | ND |
| PS2244 | 1178 ± 155 | 4510 ± 434 | 12412 ± 2260 | Very weak binding | 24272 ± 3782 | Very weak binding |

TABLE 6
Kinetics Study: Ntaq1-homologous variants (EA/DA peptides).

| Variant | EA (kon/nM/s) | EA (koff/s) | DA (kon/nM/s) | DA (koff/s) |
|---|---|---|---|---|
| PS1875 | 0.0014 | 17.26 | — | — |
| PS2121 | 0.0019 | 10.28 | — | — |
| PS2123 | 0.0018 | 8.5 | — | — |
| PS2132 | 0.0028 | 10.27 | — | — |
| PS2134 | 0.0017 | 9.12 | — | — |
| PS2195 | ~0.012* fast | ~27.6* fast | very fast* | very fast* |
| PS2244 | 0.0021 | 5.06 | 0.0017 | 11.8 |

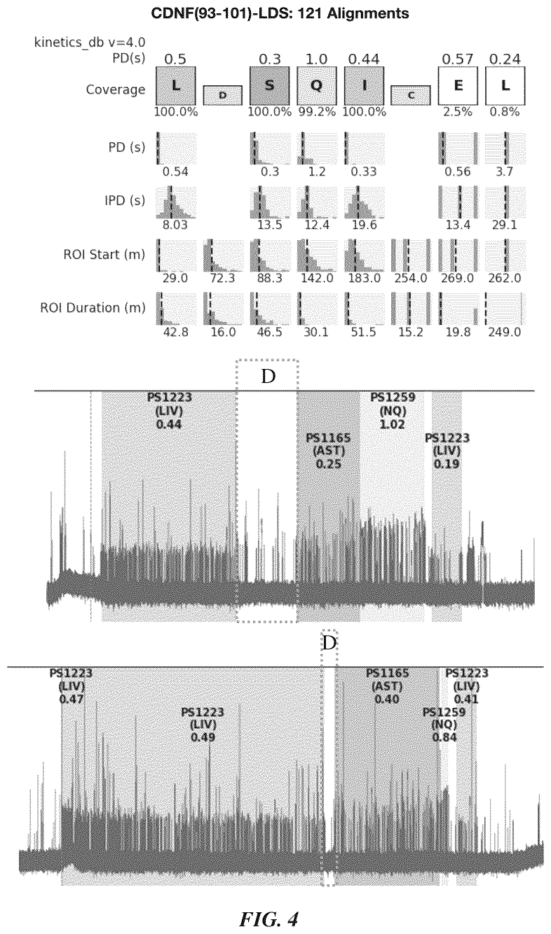

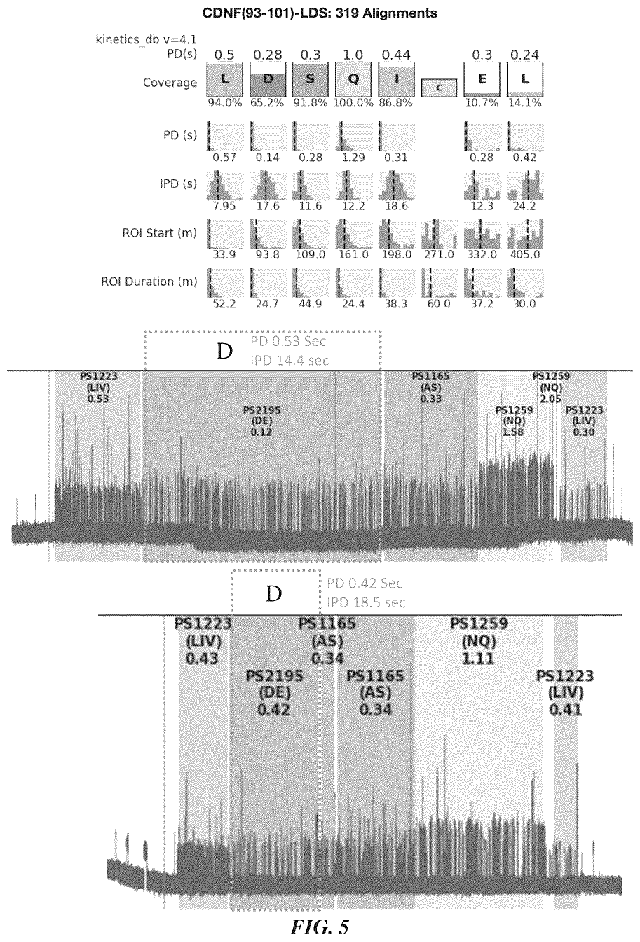

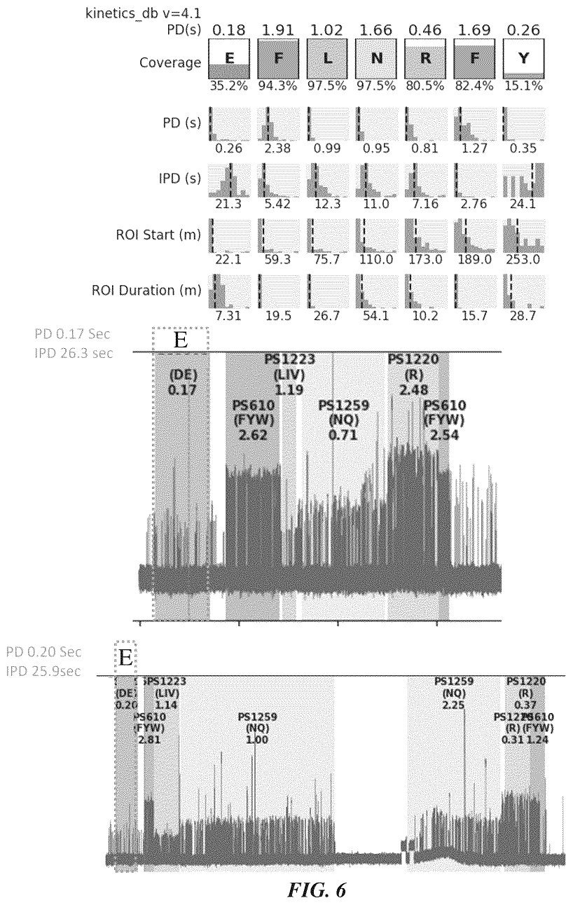

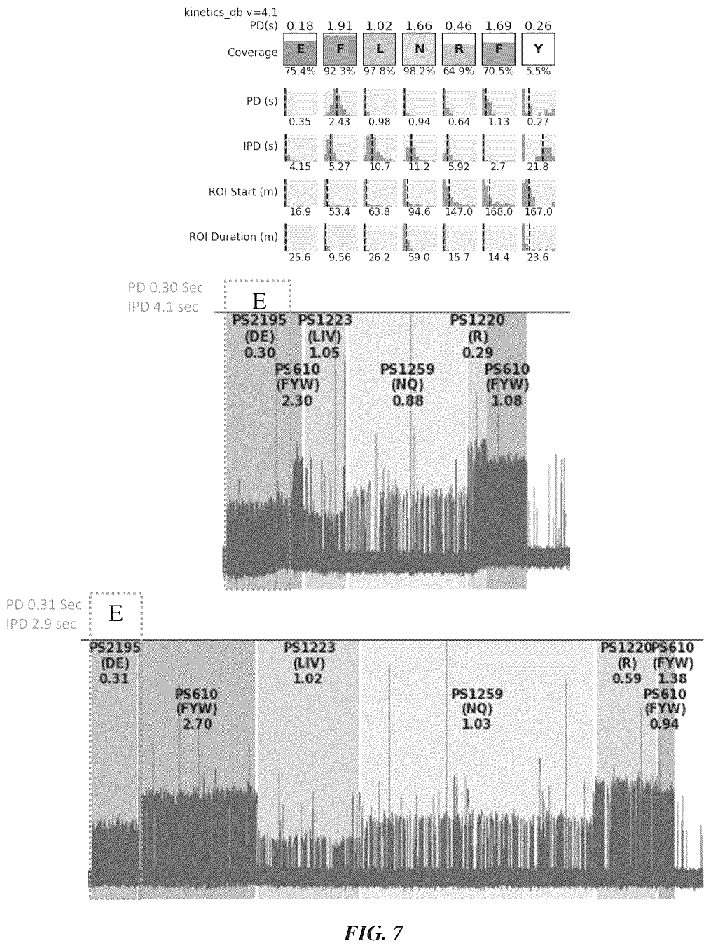

Sequencing runs were performed with CDNF libraries using a mixture of recognizers, including the DIE recognizer PS2195 and an Ntaq1-homologous variant precursor (each at 250 nM). Aspartate recognition was observed for PS2195 (FIG. 5), which was not observed for the Ntaq1-homologous variant precursor (FIG. 4). Glutamate recognition by PS2195 was found to be improved, compared to the Ntaq1-homologous variant precursor, with increased pulse duration (PD) and improved interpulse duration (IPD). FIGS. 6-7 show improved glutamate recognition by PS2195 (FIG. 7), which demonstrated a 1.35-fold improvement in PD and a 5.1-fold improvement in IPD as compared to an Ntaq1-homologous variant precursor (FIG. 6).

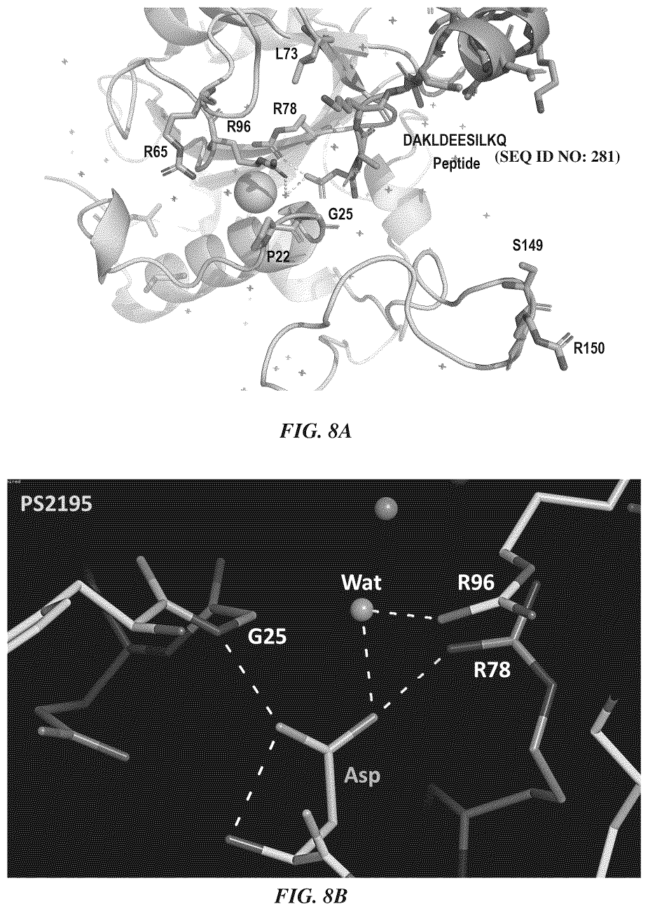

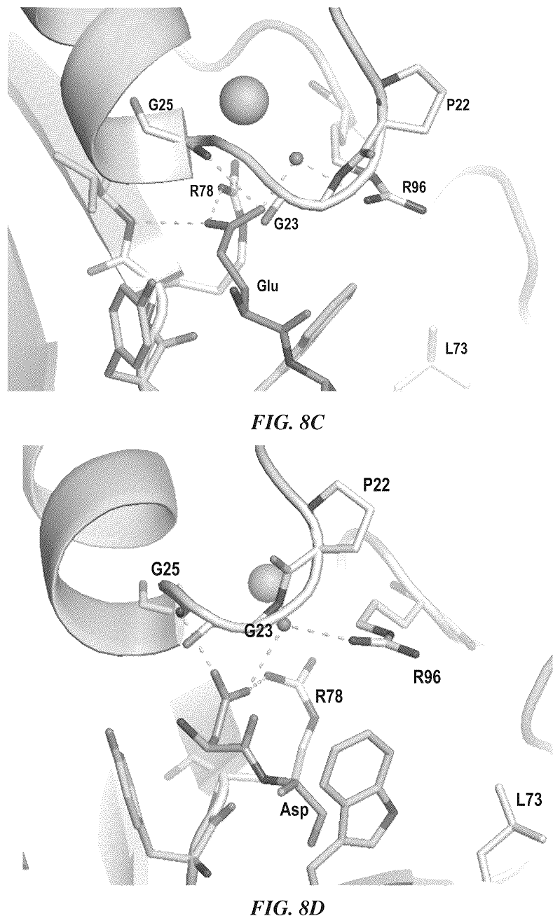

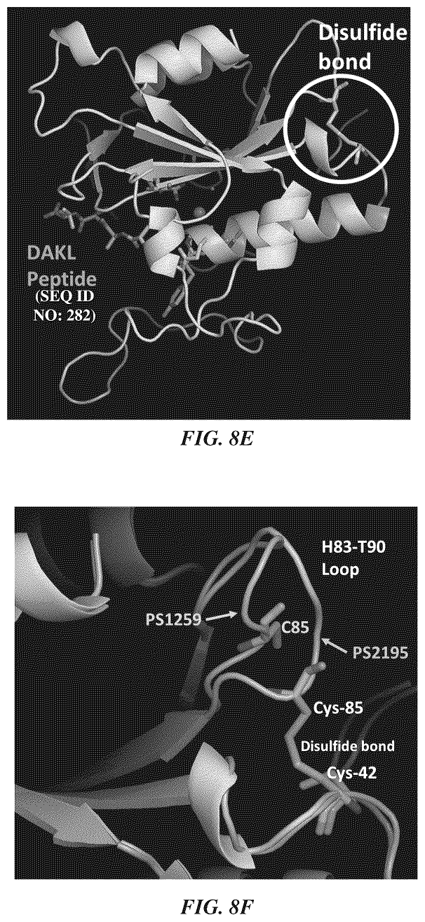

Without wishing to be bound or limited by theory, the improved glutamate recognition and new aspartate recognition of PS2195 may be rationalized in part via structure-based modeling of crystal structures of PS2915 in complex with bound peptides. FIGS. 8A-8D show the recognition pocket of PS2195 bound to aspartate- or glutamate-containing peptides. Substituted residues near the recognition pocket that were introduced into PS2195, relative to PS1259, include proline at position 22, arginine at position 96, and arginine at position 78. Without wishing to be bound or limited by theory, the Q78R and D96R mutations may allow for aspartate and glutamate recognition by multiple possible pathways, including, e.g., forming both direct and through-water interactions with the D/E side chain (indicated by dashed lines; water is shown as a “+” or sphere). In some embodiments, monovalent anion (spheres) binding sites are formed in the PS2195 recognition pocket and may, among other benefits, facilitate orientation of R78 for aspartate recognition. Without wishing to be bound or limited by theory, the S22P and C23G substitutions in PS2195 may, among other beneficial effects, increase the binding pocket size, further reducing any potential clash between an aspartate sidechain and the backbone oxygen of residue 23. Aspartate binding may be further facilitated by V73L, which in some embodiments may, among other beneficial effects, push the R65-V73 loop away from the peptide binding site, allowing PS2195 to bind both aspartate and glutamate efficiently. In some embodiments, electrostatic shielding of the negative binding pocket, among other possible beneficial effects, may be facilitated by substituted R65, R114, R122, and R150. Additionally, PS2195 contains a disulfide linkage between C42 and C85, which may in some embodiments result in an alternate conformation of the H83-T90 loop relative to PS1259 (FIGS. 8E-8F).

These data demonstrate the identification and use of Ntaq1-homologous variants for D/E recognition in protein sequencing; and suggest the importance of the mutated amino acids (at positions relative to PS1259) for improvements in aspartate and glutamate recognition by variants of PS1259 (including PS2195).

### Example 2. Development of Arginine Recognizer PS1936

This Example describes the development of PS1936 (SEQ ID NO: 158), an engineered variant of a UBR protein from Kluyveromyces marxianus (PS1122) with improved pulse duration uniformity that exhibits improved recognition of arginine on-chip. Based on analysis of binding kinetics and on-chip results, PS1936 has ˜3.5-fold higher binding affinity for N-terminal arginine than a control PS1122 variant, resulting in faster pulsing and improved pulse durations for RX dipeptides.

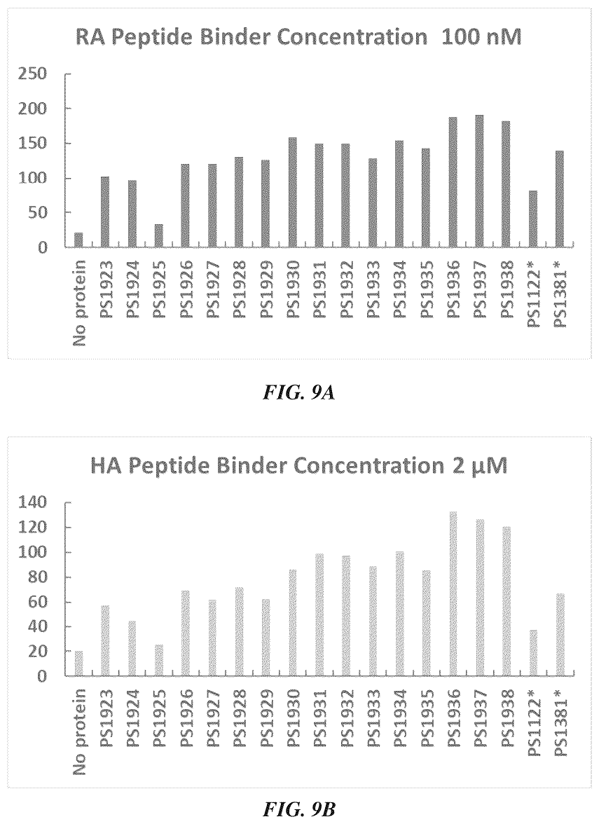

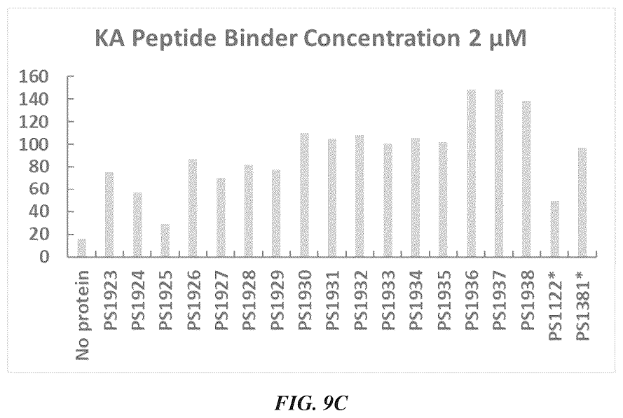

PS1122 variants were designed based on data obtained from functional assays. Fluorescence polarization assays were performed, and single point binding responses were measured at a fixed concentration of the binders (FIGS. 9A-9C). This assay measures the strength of the interaction between a binder and a fluorescein labeled peptide (XAKLDEESILKQK (SEQ ID NO: 289)). Ensemble Rapid kinetics measurements were obtained for N-terminal R, H, and K inherent binding by select variants. Binding affinities (K_{d}) were determined by polarization at 20° C. (results summarized in Table 7; dash indicates not measured). The kₒₙ rate constants and k_{off} rates were derived by stopped-flow rapid kinetic analysis at 30° C. for RA, HL, HA, and KA (results summarized in Table 8; dash indicates not measured).

TABLE 7
Binding affinities derived for PS1122 variants
by fluorescence polarization assay.

| | | RA~Kd ± | HA~Kd ± |
|---|---|---|---|
| | | std. error | std. error |
| Binders | Mutations | (nM) | (nM) |
| PS1122 | PS621 + T47L, I63E, E70T | 74 ± 13 | 3092 ± 1347 |
| PS1381 | PS1122_ + K26R, D32R | 47 ± 11 | — |
| PS1383 | PS1122 + K26R, D32R, E58Q, | 51 ± 17 | — |
| | F59K | | |
| PS1659 | PS1122 + K26R, D32K, E58Q, | 42 ± 13 | 1541 ± 229 |
| | F59K | | |
| PS1715 | PS1122 + R24G, K26R, D32R, | 37 ± 12 | 593 ± 62 |
| | K44N, L47I, F59K, N60L | | |
| PS1936 | PS1122 + L47R, T53V, F59R, | 36 ± 8 | 854 ± 63 |
| | T75E | | |

TABLE 8
Stopped-flow binding kinetics of PS1122 variants.

| | | RA | RA | HL | RA | HA | KA |
|---|---|---|---|---|---|---|---|
| Binders | Mutations | (kon/nM/s) | (koff/s) | (koff/s) | Kd nM | Kd nM | Kd nM |
| PS1122 | PS621 + T47L, I63E, E70T | 0.039 | 15.34 | 28.61 | 211 ± 34 | 8535 ± 1903 | 5582 ± 1177 |
| PS1381 | PS1122 + K26R, D32R | 0.064 | 11.9 | 23.64 | 166 ± 36 | 2752 ± 198 | 3205 ± 195 |
| PS1659 | PS1122 + K26R, D32K, | 0.086 | 7.30 | — | 61 ± 17 | 1717 ± 186 | 1434 ± 67 |
| | E58Q, F59K | | | | | | |
| PS1715 | PS1122 + R24G, K26R, D32R, | 0.056 | 9.30 | 21.60 | 97 ± 18 | 1669 ± 152 | 2139 ± 227 |
| | K44N, L47I, F59K, N60L | | | | | | |
| PS1936 | PS1122 + L47R, T53V, | 0.049 | 13.13 | 18.38 | 60 ± 12 | 1270 ± 107 | 1496 ± 104 |
| | F59R, T75E | | | | | | |
| PS1938 | PS1122 + K26R, D32P, | 0.043 | 8.54 | — | 67 ± 13 | 2122 ± 155 | 1556 ± 93 |
| | L47R, T53V, F59R, T75E | | | | | | |

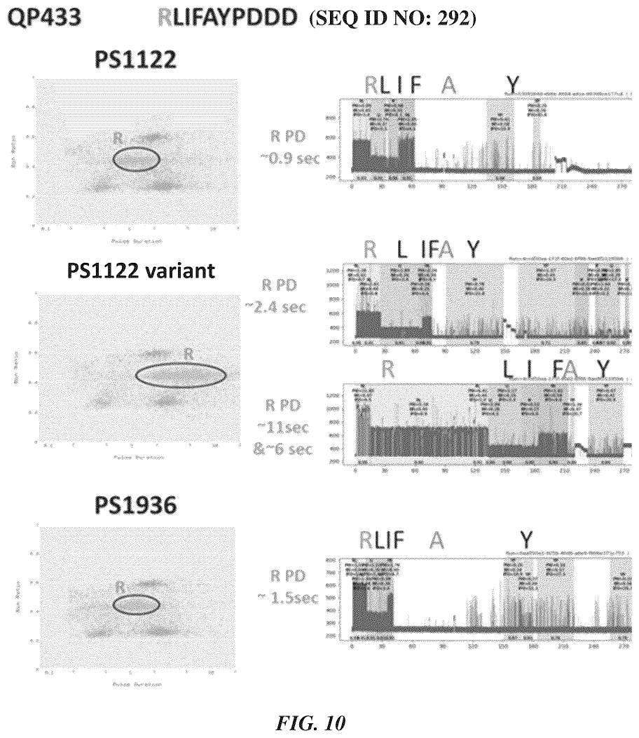

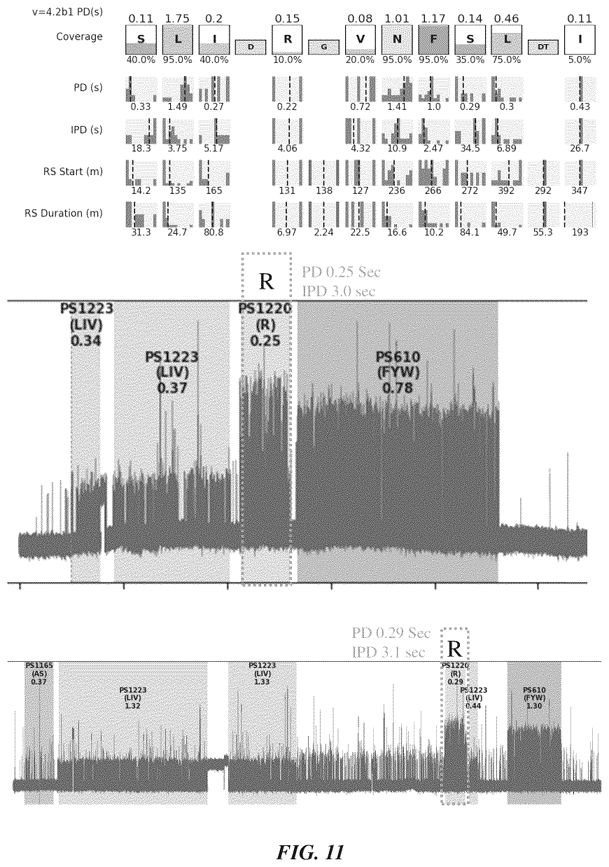

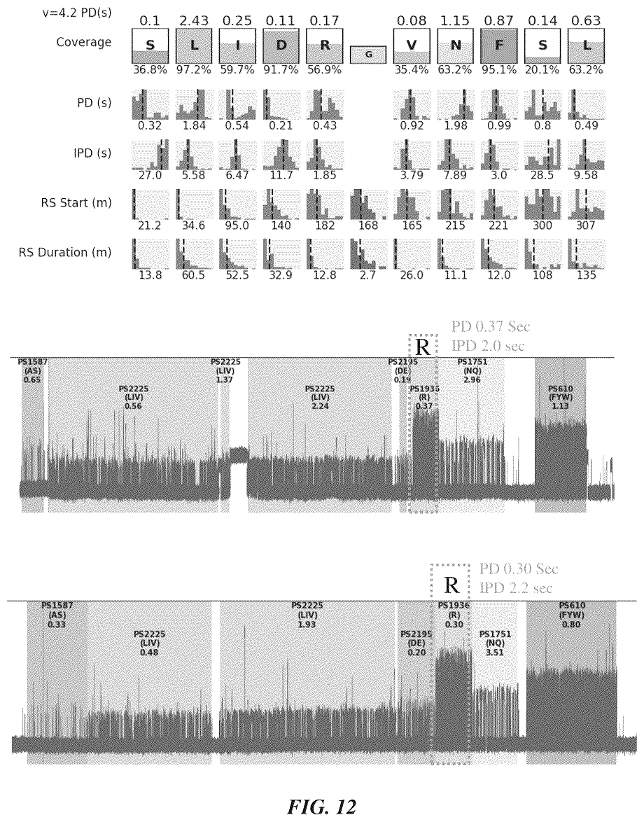

Sequencing performance of PS1122, a PS1122 variant, and PS1936 were compared using QP433 peptide (RLIFAYPDDD (SEQ ID NO: 292)). Compared to the PS1122 variant which showed multiple pulse widths that complicates deconvolution of sequence data, PS1936 showed uniform pulse width (FIG. 10). Additionally, sequencing runs were performed with CDNF peptide libraries using a mixture of recognizers, including a PS1122 variant (at 125 nM) and PS1936 (at 250 nM). Exemplary traces are shown in FIGS. 11-12. Compared to the PS1122 variant, PS1936 demonstrated a 1.9-fold improvement in pulse duration (PD) and a 2.1-fold improvement in interpulse duration (IPD) as compared to the PS1122 variant.

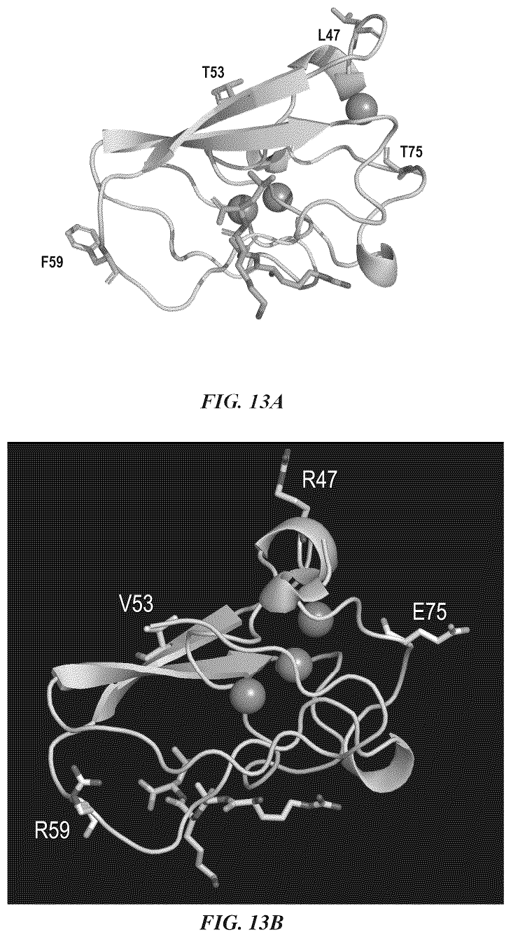

Without wishing to be bound or limited by theory, the improved performance of PS1936 may be understood in part via structure-based modeling of a crystal structure of PS1122 (precursor to PS1936) complexed with bound peptide. FIG. 13A shows the crystal structure of PS1122 bound to arginine peptide RAKL (SEQ ID NO: 288) within the recognition pocket. FIG. 13B shows a model of PS1936, which was derived from the crystal structure of PS1122, shown bound to an RAKL (SEQ ID NO: 288) peptide. Substituted residues near the recognition pocket that were introduced into PS1936, relative to PS1122, include arginine at position 59, valine at position 53, arginine at position 47, and glutamate at position 75. Notably, none of the mutations directly interact with the ligand, strongly indicating that, among other benefits, the mutations may improve the stability and solubility of the protein, which may in turn improve kinetic parameters. Substituted R47, R59, and E75 are surface residues. In the PS1122 structure, the amino acids at these positions contained non-polar or polar side chains. Without wishing to be bound or limited by theory, the mutation from non-polar or polar amino acids in PS1122 to charged amino acids in PS1936 was thought, among other beneficial effects, to reduce oligomerization sites between the protein and itself. Additionally, a T53V substitution near the center of the beta strand might, in some embodiments, improve the stability of the beta sheet due, at least in part, to its sidechain orientation favoring a beta structure.

These results demonstrate the identification and utility of UBR variants with improved kinetics and binding properties for recognition of arginine in protein sequencing. These data suggest the importance of the mutated amino acids (at positions relative to PS1122) for improvements in arginine recognition by variants of PS1122 (including PS1936).

### Example 3. Development of Glycine/Alanine/Serine Recognizer PS2459

This Example describes the development of PS2459 (SEQ ID NO: 234) by ensemble and high-throughput single molecule analyses. PS2459 is an engineered variant of an Ntaq1-homologous recognizer from Scleropages Formosus (PS1259) capable of recognizing glycine, alanine, and serine.

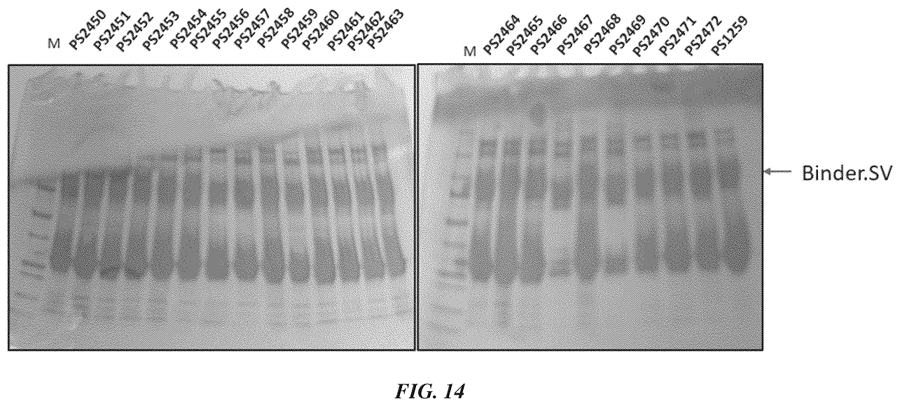

Ntaq1-homologous protein candidate recognizers were identified by development techniques including, e.g., protein engineering and directed evolution, expressed in E. coli and purified (FIG. 14). The candidates were evaluated for binding to N-terminal amino acids on the Octet binding platform. The peptides used in the assay contained (i) a penultimate alanine and an N-terminal glycine (GA); (ii) a penultimate alanine and an N-terminal asparagine (NA); (iii) a penultimate alanine and an N-terminal serine (SA); or (iv) a penultimate alanine and an N-terminal glutamine (QA). In the high-throughput assay, Octet sensors were coated with the peptide of interest and dipped in buffer containing the purified protein. The set of Octet response measurements is summarized in Table 9.

TABLE 9
Octet response for Ntaq-1 homologous variants.

| Binders | Homologs/Mutations | GA | NA | QA |
|---|---|---|---|---|
| PS2300 | PS1259 + C85T, D148M | 0.3 | 3.5 | 5.6 |
| PS2301 | PS1259 + S22T, S39N, I74Y, D148Y | 0.2 | 3.5 | 5.3 |
| PS2302 | PS1259 + D148F | 0.3 | 3.8 | 5.9 |
| PS1259 | hntaq1 homolog Scleropages formosus + C25S, H78Q | 0.5 | 4.1 | 5.8 |
| PS2132 (E) | PS1259 + S22E, Q78K, C85T, N120R | 0.7 | 0.7 | 2.3 |

| Binders | Homologs/Mutations | GA | SA | QA |
|---|---|---|---|---|
| PS2303 | PS1259 + A12L, S22E, S66V, N120R, A122R, S150V | 2.1 | 1.9 | 7.2 |
| PS2304 | PS1259 + A12L, S22E, S25G, W30Y, S66V, V73I, N120R, A149G, | 0.3 | 2 | 6.2 |
| | S150V, R154Q, P156S | | | |
| PS2305 | PS1259 + A12L, S22E, S66V, E71R, A122R, A149L, S150V, R154L | 2.8 | 2.6 | 6.7 |
| PS2306 | PS1259 + A12L, S22E, S66V, E71R, A122D, V130I, A149L, S150V, | 1 | 0.8 | 5.5 |
| | R154L | | | |
| PS2307 | PS1259 + A12L, S66I, E70K, E71R, V73I, I80V, C85R, A149V, S150G, | 0.2 | 0.2 | 4.7 |
| | R154L | | | |
| PS2308 | PS1259 + A12L, S22E, E71R, V73I, I80V, A122R, K147R, A149N, | 5.2 | 4.1 | 7.3 |
| | S150G, R154L | | | |
| PS2309 | PS1259 + A12L, E71R, V73I, I80V, N120S, A122R, A149D, S150V, | 2.7 | 2 | 6.6 |
| | R154Q | | | |
| PS2310 | PS1259 + A12T, S22E, W30Y, E71K, N120R, A122R, A149D, S150V, | 4 | 3 | 7 |
| | R154L | | | |
| PS2311 | PS1259 + A12L, S22E, C23Y, S25G, E26W, V73I, I80V, A122D, | 0.2 | 0.2 | 0.2 |
| | A149G, S150C, R154Q | | | |
| PS2312 | PS1259 + A12L, S22R, S66V, E71R, A122R, A149L, S150V, R154L | 1.5 | 1.5 | 6.7 |
| PS2313 | PS1259 + A12L, S22E, E71R, I80V, C85R, N120R, A122R, A149V | 4.9 | 3.5 | 8.3 |
| PS2314 | PS1259 + S5C, A12L, E71R, V73I, I80V, N120S, A122R, A149D, | 2.3 | 1.8 | 6.4 |
| | S150V, R154Q | | | |
| PS1259 | hntaq1 homolog Scleropages formosus + C25S, H78Q | 0.5 | 0.4 | 5.9 |
| (N/Q) | | | | |
| PS2132 (E) | PS1259 + S22E, Q78K, C85T, N120R | 0.7 | 0.7 | 2.3 |
| PS2195 | PS1259 + A12L, S22P, C23G, S25G, K65R, V73L, Q78R, D96R, K114R, | 1.1 | 1.1 | 1.1 |
| (D/E) | A122R, A149S, S150R | | | |
| PS2450 | PS1259 + A12L, S22E, K65R, S66V, E71R, A122R, S150V, R154L | 3 | 2.9 | 6.5 |
| PS2451 | PS1259 + A12L, S22E, S25Q, S66V, Q78H, N120R, A122R, S150V | 1.3 | 2.9 | 0.1 |
| PS2452 | PS1259 + A12L, S22E, S25Q, S66V, E71R, Q78H, A122R, S150V, | 2.8 | 4.3 | 0.3 |
| | R154L | | | |
| PS2453 | PS1259 + A12L, S22E, S25Q, E71R, V73I, Q78H, I80V, A122R, K147R, | 4.6 | 5.1 | 0.6 |
| | A149N, S150G, R154L | | | |
| PS2454 | PS1259 + A12L, S25Q, E71R, V73I, Q78H, I80V, N120S, A122R, | 1.9 | 2.3 | 0.4 |
| | A149D, S150V, R154Q | | | |
| PS2455 | PS1259 + A12T, S22E, S25Q, W30Y, E71K, Q78H, N120R, A122R, | 6.3 | 6.3 | 2 |
| | A149D, S150V, R154L | | | |
| PS2456 | PS1259 + A12L, S22R, S25Q, S66V, E71R, Q78H, A122R, A149L, | 2.7 | 4.6 | 1.2 |
| | S150V, R154L | | | |
| PS2457 | PS1259 + A12L, S22E, S25Q, E71R, Q78H, I80V, C85R, N120R, A122R, | 5.1 | 6 | 0.9 |
| | A149V | | | |
| PS2458 | PS1259 + S5C, A12L, S25Q, E71R, V73I, Q78H, I80V, N120S, A122R, | 2.7 | 3.3 | 1.4 |
| | A149D, S150V, R154Q | | | |
| PS2459 | PS1259 + A12L, S22E, S25Q, S66V, E71R, Q78H, N120R, A122R, | 3 | 4.6 | 0.3 |
| | S150V, R154L | | | |
| PS2460 | PS1259 + A12L, S22E, S25Q, K65R, S66V, E71R, Q78H, A122R, | 5.1 | 5.5 | 0.6 |
| | S150V, R154L | | | |
| PS2461 | PS1259 + A12L, S22E, S25Q, S66V, E71R, V73L, Q78H, A122R, | 3.6 | 4.5 | 1.4 |
| | S150V, R154L | | | |
| PS2462 | PS1259 + A12L, S22E, S25Q, S66V, E71R, Q78H, I80V, A122R, S150V, | 4.5 | 5.5 | 1.4 |
| | R154L | | | |
| PS2463 | PS1259 + A12L, S22E, S25Q, S66V, E71R, V73I, Q78H, I80V, A122R, | 4.2 | 5 | 1.1 |
| | S150V, R154L | | | |
| PS2464 | PS1259 + A12L, S22E, S25Q, S66V, E71R, A122R, S150V, R154L | 3.4 | 1.3 | 4 |
| PS2465 | PS1259 + A12L, S22E, S66V, E71R, Q78H, A122R, S150V, R154L | 3 | 3.1 | 6.2 |
| PS2466 | PS1259 + A12L, S22E, S25N, S66V, E71R, Q78H, A122R, S150V, | 2.5 | 5.1 | 5.5 |
| | R154L | | | |
| PS2467 | PS1259 + A12L, S22E, S25Q, S66V, E71R, Q78L, A122R, S150V, | 1.8 | 1.4 | 4 |
| | R154L | | | |
| PS2468 | PS1259 + A12L, S25Q, E71R, Q78H, A122R | 3.4 | 3.4 | 1.6 |
| PS2469 | PS1259 + A12L, S22E, S25Q, S66V, E71R, Q78M, A122R, S150V, | 2.9 | 1.4 | 2.7 |
| | R154L | | | |
| PS2470 | PS1259 + A12L, S22E, C23Y, S25G, E71R, V73I, A122R, A149I, | 2.3 | 5.5 | 7 |
| | S150V, R154L | | | |
| PS2471 | PS1259 + A12L, S22E, S25G, S66V, E71R, A122R, R154L | 2.8 | 4.8 | 7 |
| PS2472 | PS1259 + A12L, S22E, S25Q, S66V, E71R, Q78N, A122R, S150V, | 1.7 | 2.4 | 3.5 |
| | R154L | | | |
| PS1259 | hntaq1 homolog Scleropages formosus + C25S, H78Q | 1.4 | 1.1 | 5 |

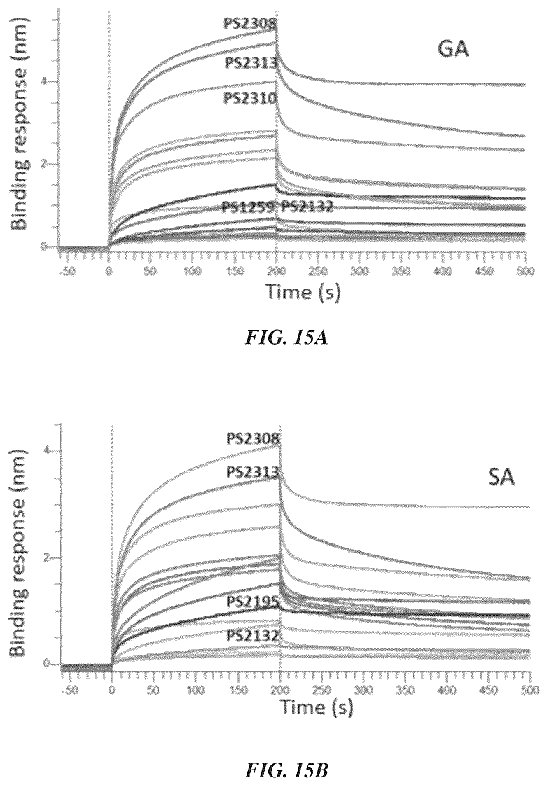

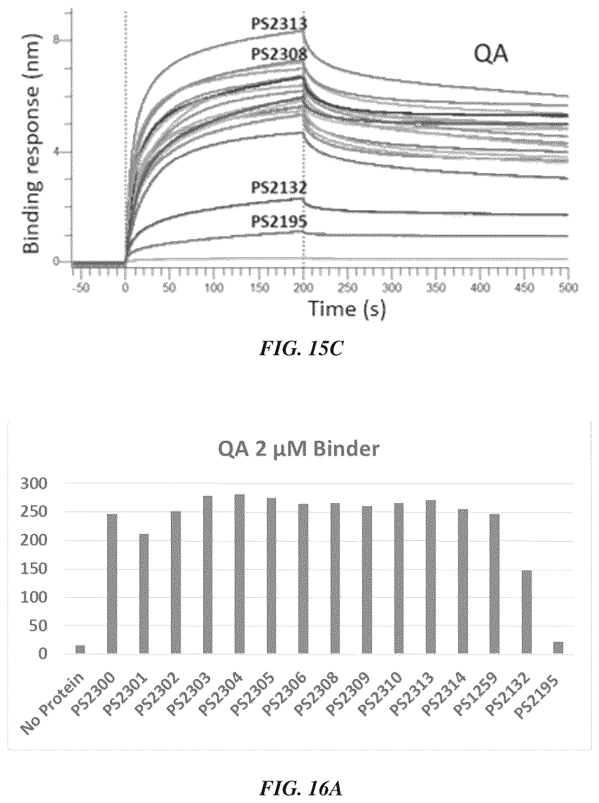

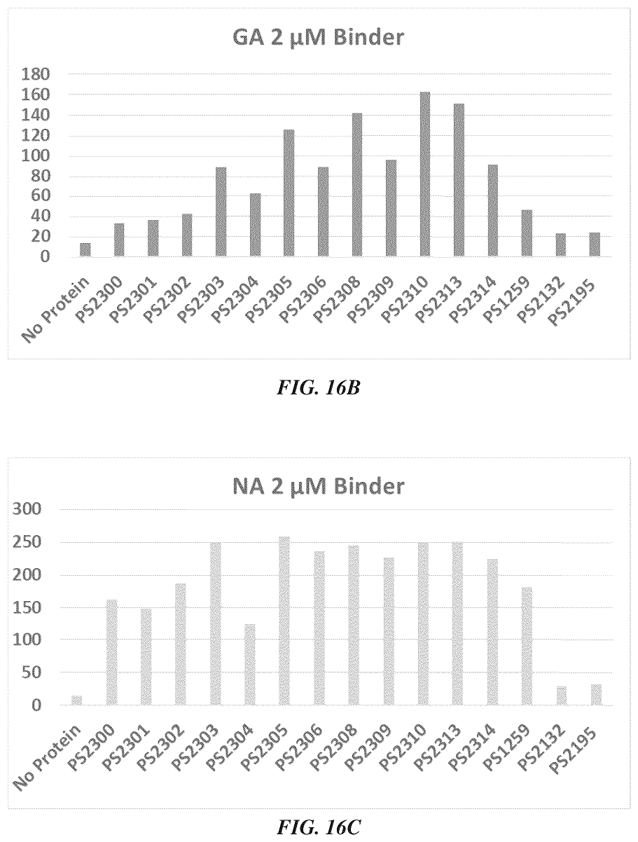

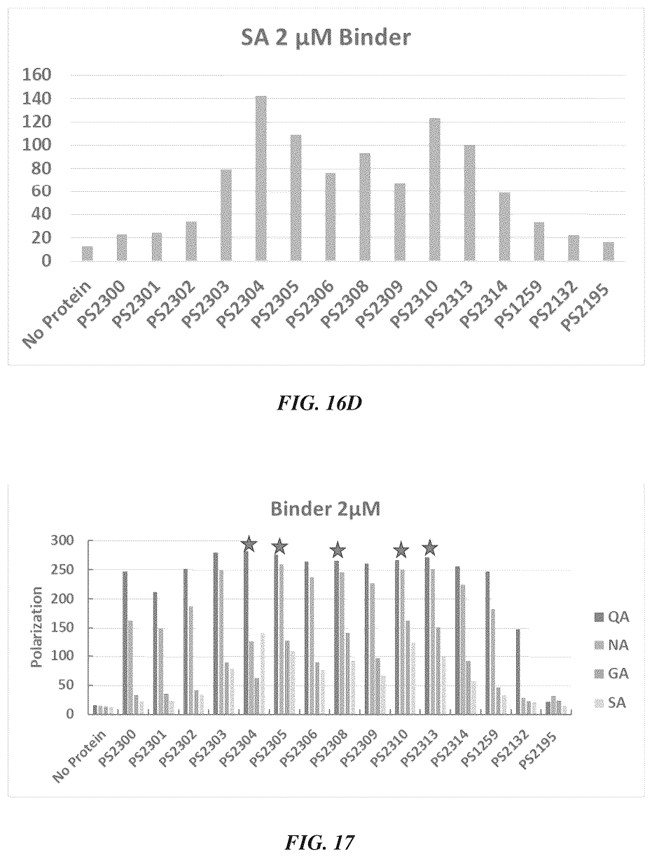

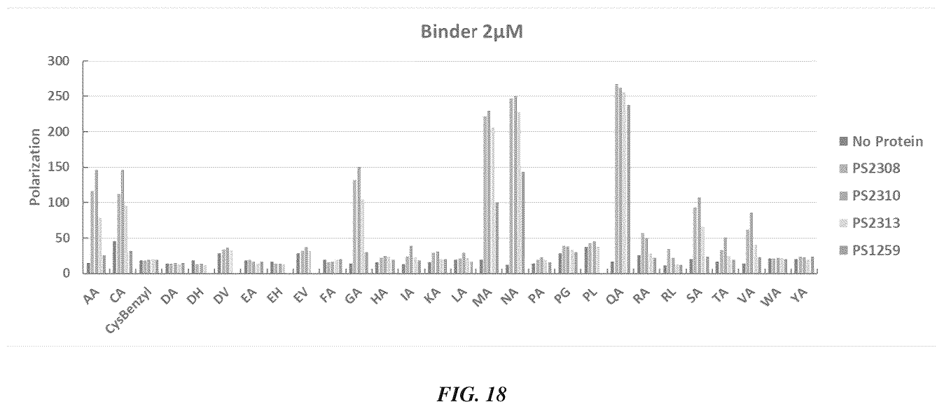

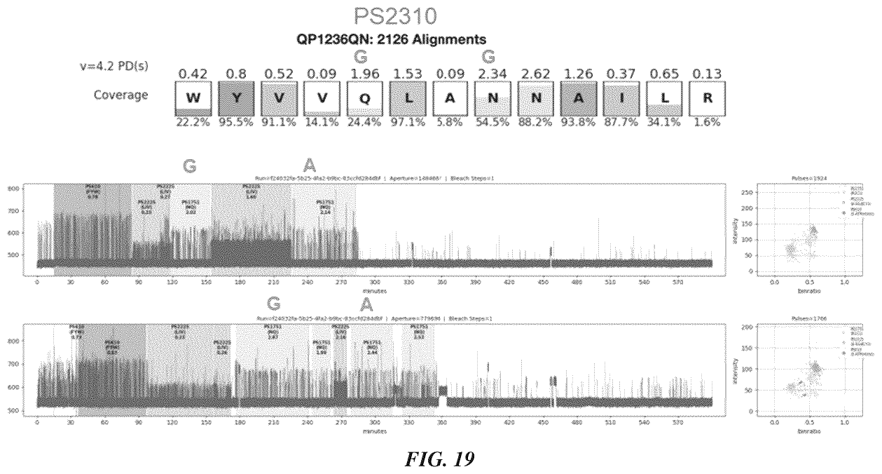

From these results, 12 variants were selected for further analysis (Table 10). The binding data representative of the binding interaction between the 12 PS1259 variants and the GA, SA, and QA peptides are shown in FIGS. 15A-15C, respectively. Fluorescence polarization assays and single point binding responses were measured at a fixed concentration (2 μM) of the binders (FIGS. 16A-17). Binding affinities (K_{d}) were determined by fluorescence polarization at 20° C. (results summarized in Table 11), and the k_{off} rates were derived by stopped-flow rapid kinetic analysis at 30° C. for GA peptides. From these results, three candidates (PS2308, PS2310, and PS2313) were selected for further analysis due to their tighter binding affinity and slower k_{off} rates. Fluorescence polarization assays for all N-terminal amino acids were measured for the three candidates, as well as PS1259 (control). In addition to strong binding interactions with glycine, the three candidates also showed strong binding interactions with alanine, cysteine, methionine, asparagine, glutamine, serine, and valine (FIG. 18). Sequencing runs performed with PS2310 showed glycine and serine recognition, as well as some alanine, valine, asparagine and glutamine recognition (FIG. 19).

TABLE 10
PS1259 variants evaluated in this Example.

| Sample ID | Mutations | Targeted Binding to |
|---|---|---|
| PS2300 | C85T, D148M | G |
| PS2301 | S22T, S39N, I74Y, D148Y | G |
| PS2302 | D148F | G |
| PS2303 | A12L, S22E, S66V, N120R, A122R, S150V | G, S |
| PS2304 | A12L, S22E, S25G, W30Y, S66V, V73I, N120R, A149G, S150V, | G, S |
| | R154Q, P156S | |
| PS2305 | A12L, S22E, S66V, E71R, A122R, A149L, S150V, R154L | G, S |
| PS2306 | A12L, S22E, S66V, E71R, A122D, V130I, A149L, S150V, R154L | G, S |
| PS2308 | A12L, S22E, E71R, V73I, I80V, A122R, K147R, A149N, S150G, | G, S |
| | R154L | |
| PS2309 | A12L, E71R, V73I, I80V, N120S, A122R, A149D, S150V, R154Q | G, S |
| PS2310 | A12T, S22E, W30Y, E71K, N120R, A122R, A149D, S150V, R154L | G, S |
| PS2313 | A12L, S22E, E71R, I80V, C85R, N120R, A122R, A149V | G, S |
| PS2314 | S5C, A12L, E71R, V73I, I80V, N120S, A122R, A149D, S150V, R154Q | G, S |

TABLE 11
Kinetics Study: Ntaq1-homologous variants (GA/SA peptides).

| | | GA Peptide Kd ± | SA Peptide Kd ± | |
|---|---|---|---|---|
| Binder | Mutations | std. error (nM) | std. error (nM) | GA (koff/s) |
| PS2304 | A12L, S22E, S25G, W30Y, | 3273 ± 275 | 587 ± 65 | 70.95 |
| | S66V, V73I, N120R, A149G, | | | |
| | S150V, R154Q, P156S | | | |
| PS2305 | A12L, S22E, S66V, E71R, | 916 ± 42 | 1175 ± 51 | 66.091 |
| | A122R, A149L, S150V, | | | |
| | R154L | | | |
| PS2308 | A12L, S22E, E71R, V73I, | 771 ± 21 | 1259 ± 53 | 45.933 |
| | I80V, A122R, K147R, A149N, | | | |
| | S150G, R154L | | | |
| PS2310 | A12T, S22E, W30Y, E71K, | 742 ± 21 | 1164 ± 25 | 39.696 |
| | N120R, A122R, A149D, | | | |
| | S150V, R154L | | | |
| PS2313 | A12L, S22E, E71R, I80V, | 783 ± 48 | 1416 ± 79 | 27.609 |
| | C85R, N120R, A122R, A149V | | | |
| PS1259 | N/A | Very weak binding | Very weak binding | 1.8962 (QA) |

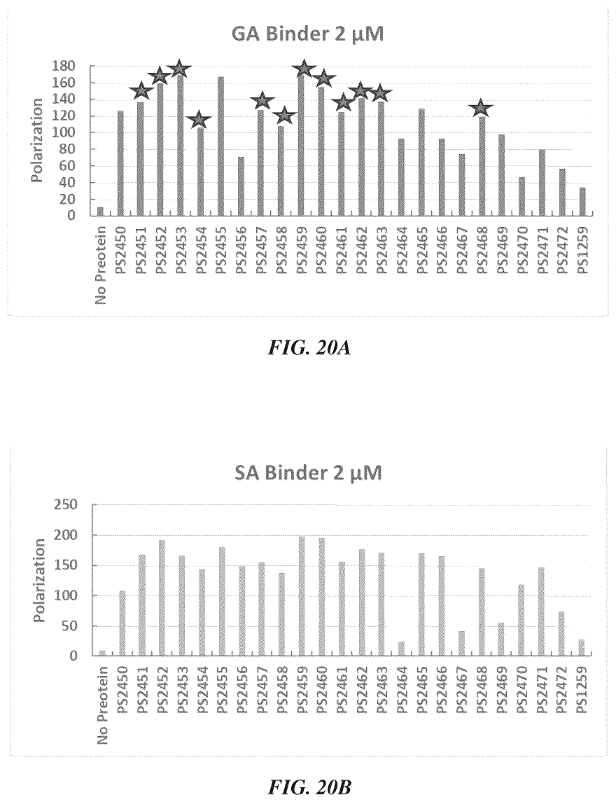

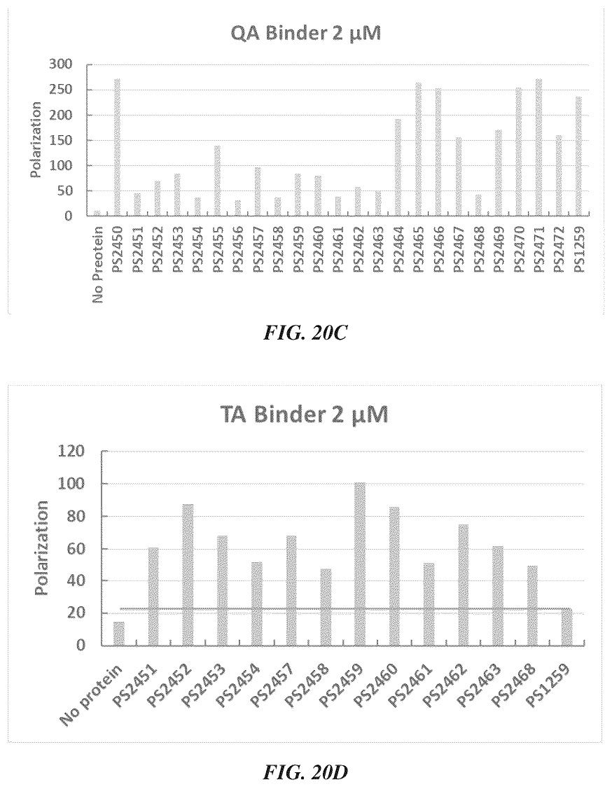

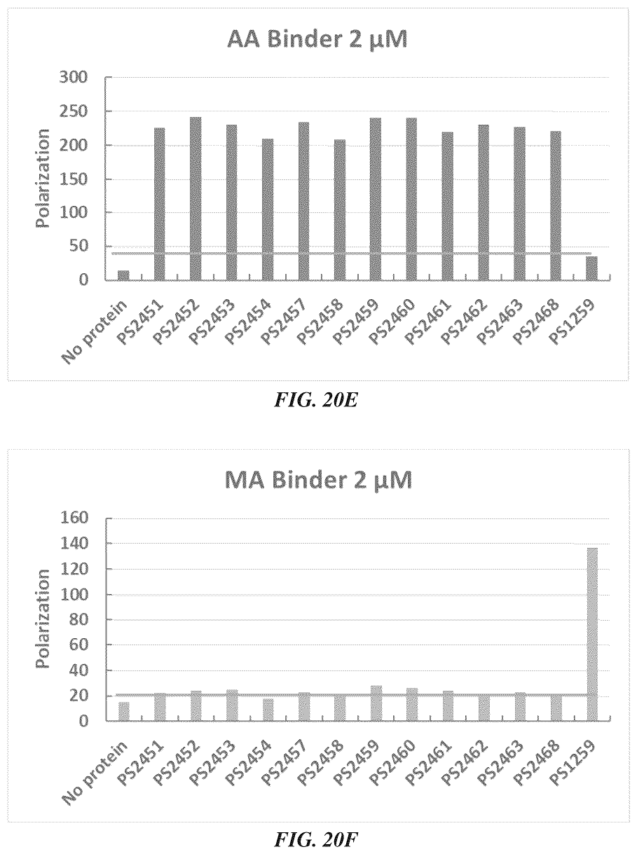

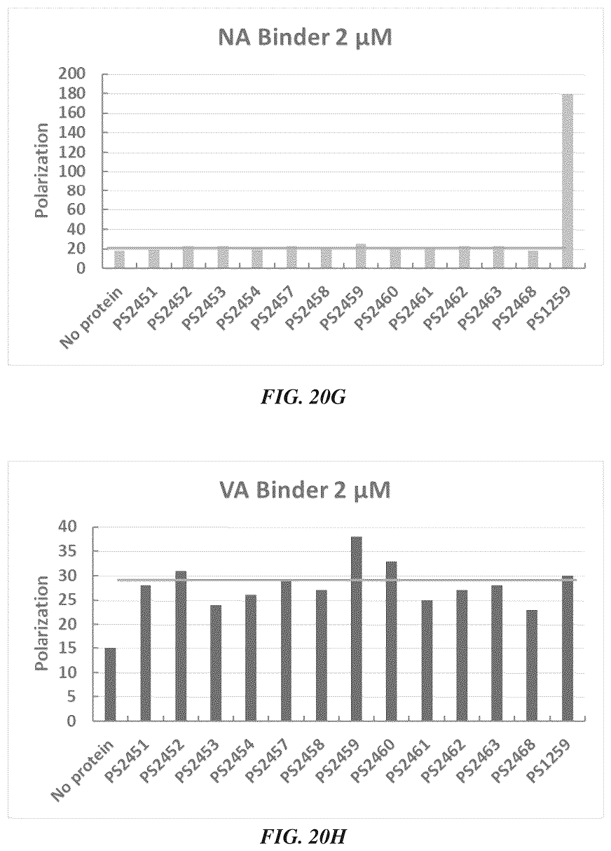

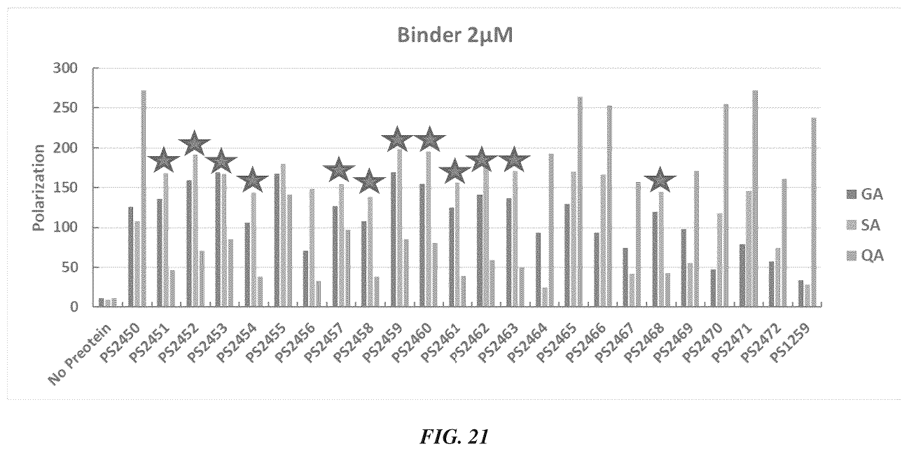

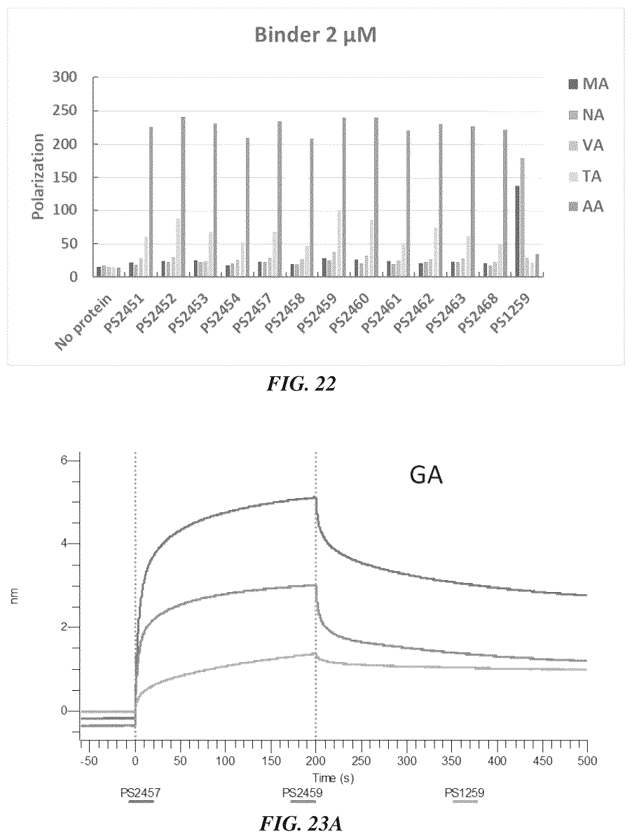

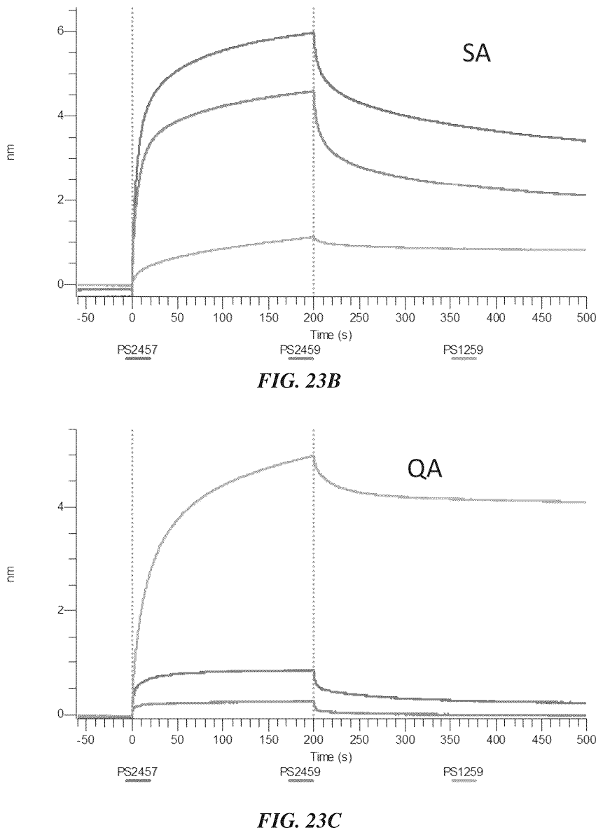

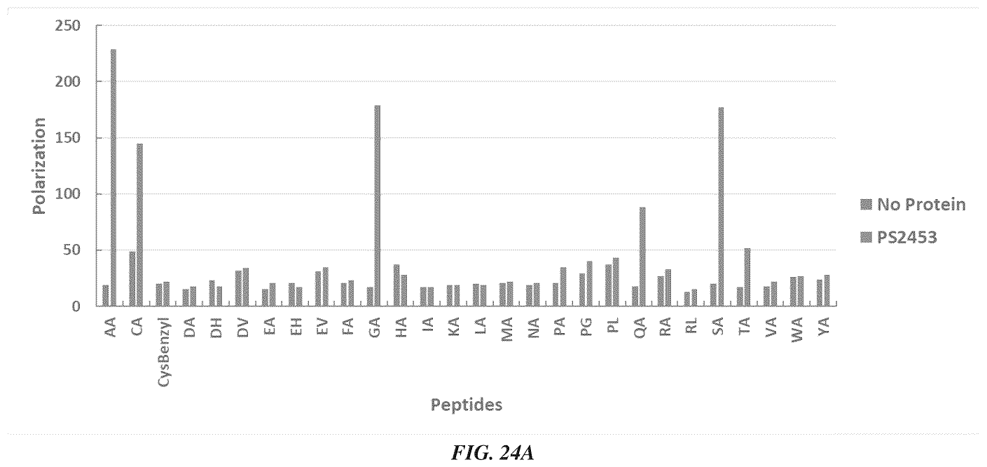

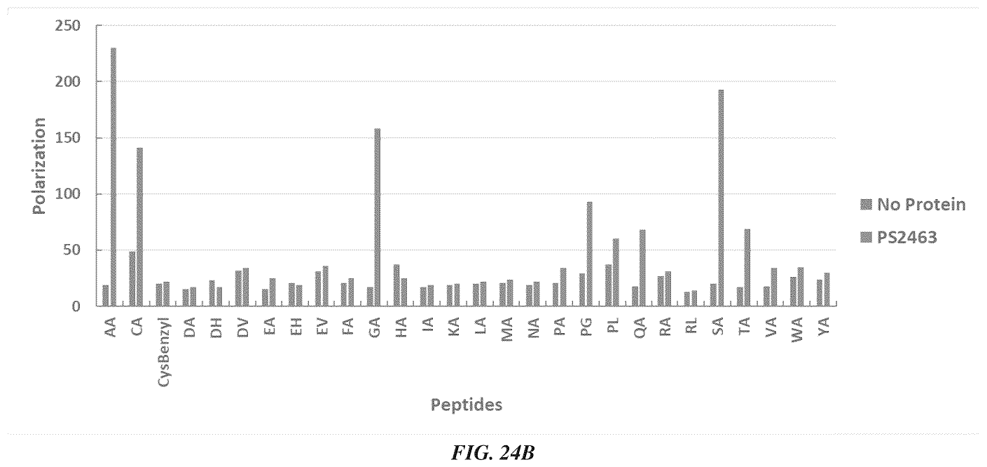

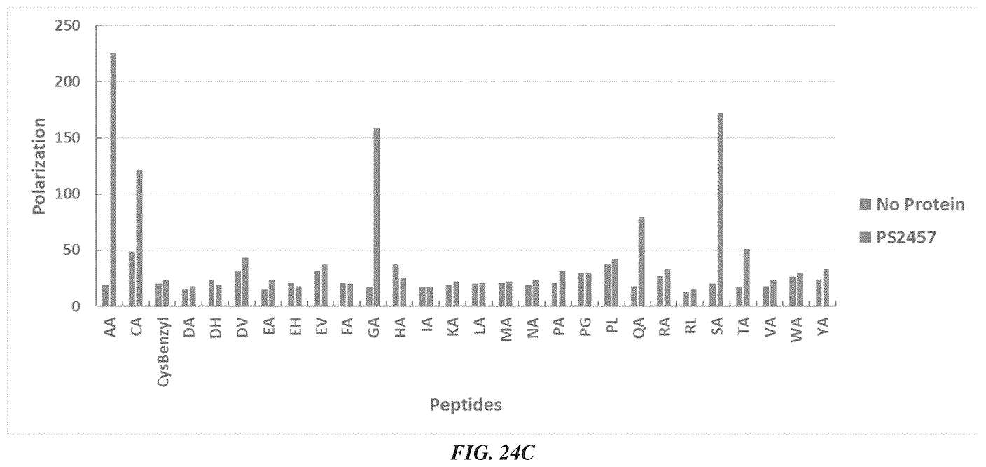

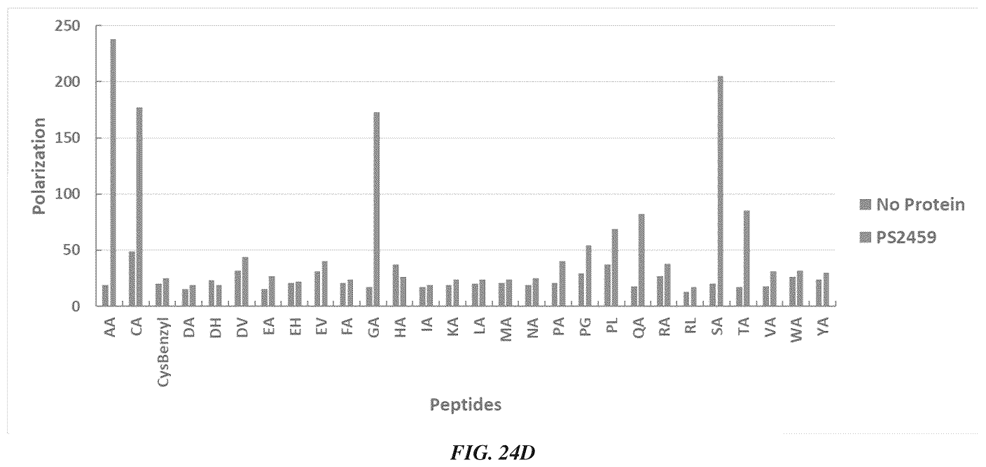

A set of 23 variants were also further analyzed for binding to glycine, serine, and glutamine (Table 12). Fluorescence polarization assays and single point binding responses to GA, SA, QA, TA, AA, MA, NA, and VA peptides were measured at a fixed concentration (2 μM) of the binders (FIGS. 20A-22). Binding affinities (K_{d}) were determined by fluorescence polarization at 20° C. (results summarized in Table 11), and the k_{off} rates were derived by stopped-flow rapid kinetic analysis at 30° C. for GA, SA, and AA peptides (results summarized in Tables 13-14). From these results, two candidates (PS2457 and PS2459) were selected for further analysis due to their tighter binding affinity and slower k_{off} rates. Binding data representative of the binding interaction between PS2457, PS2459, and PS1259 (control) and the GA, SA, and QA peptides are shown in FIGS. 23A-23C, respectively. Binding kinetics and results from fluorescence polarization assays for all N-terminal amino acids for PS2453, PS2463, PS2457, and PS2459 produced by a large-scale preparation (without streptavidin) are shown in Table 15 and FIGS. 24A-24D, respectively.

TABLE 12
PS1259 variants evaluated in this Example.

| Binders | Mutations | Targeted binding to |
|---|---|---|
| PS2450 | A12L, S22E, K65R, S66V, E71R, A122R, S150V, R154L | G, S, Q (less or nill) |
| PS2451 | A12L, S22E, S25Q, S66V, Q78H, N120R, A122R, S150V | G, S, Q (less or nill) |
| PS2452 | A12L, S22E, S25Q, S66V, E71R, Q78H, A122R, S150V, R154L | G, S, Q (less or nill) |
| PS2453 | A12L, S22E, S25Q, E71R, V73I, Q78H, I80V, A122R, K147R, A149N, | G, S, Q (less or nill) |
| | S150G, R154L | |
| PS2454 | A12L, S25Q, E71R, V73I, Q78H, I80V, N120S, A122R, A149D, S150V, | G, S, Q (less or nill) |
| | R154Q | |
| PS2455 | A12T, S22E, S25Q, W30Y, E71K, Q78H, N120R, A122R, A149D, | G, S, Q (less or nill) |
| | S150V, R154L | |
| PS2456 | A12L, S22R, S25Q, S66V, E71R, Q78H, A122R, A149L, S150V, R154L | G, S, Q (less or nill) |
| PS2457 | A12L, S22E, S25Q, E71R, Q78H, I80V, C85R, N120R, A122R, A149V | G, S, Q (less or nill) |
| PS2458 | S5C, A12L, S25Q, E71R, V73I, Q78H, I80V, N120S, A122R, A149D, | G, S, Q (less or nill) |
| | S150V, R154Q | |
| PS2459 | A12L, S22E, S25Q, S66V, E71R, Q78H, N120R, A122R, S150V, R154L | G, S, Q (less or nill) |
| PS2460 | A12L, S22E, S25Q, K65R, S66V, E71R, Q78H, A122R, S150V, R154L | G, S, Q (less or nill) |
| PS2461 | A12L, S22E, S25Q, S66V, E71R, V73L, Q78H, A122R, S150V, R154L | G, S, Q (less or nill) |
| PS2462 | A12L, S22E, S25Q, S66V, E71R, Q78H, I80V, A122R, S150V, R154L | G, S, Q (less or nill) |
| PS2463 | A12L, S22E, S25Q, S66V, E71R, V73I, Q78H, I80V, A122R, S150V, | G, S, Q (less or nill) |
| | R154L | |
| PS2464 | A12L, S22E, S25Q, S66V, E71R, A122R, S150V, R154L | G, S, Q (less or nill) |
| PS2465 | A12L, S22E, S66V, E71R, Q78H, A122R, S150V, R154L | G, S, Q (less or nill) |
| PS2466 | A12L, S22E, S25N, S66V, E71R, Q78H, A122R, S150V, R154L | G, S, Q (less or nill) |
| PS2467 | A12L, S22E, S25Q, S66V, E71R, Q78L, A122R, S150V, R154L | G, S, Q (less or nill) |
| PS2468 | A12L, S25Q, E71R, Q78H, A122R | G, S, Q (less or nill) |
| PS2469 | A12L, S22E, S25Q, S66V, E71R, Q78M, A122R, S150V, R154L | G, S, Q (less or nill) |
| PS2470 | A12L, S22E, C23Y, S25G, E71R, V73I, A122R, A149I, S150V, R154L | G, S, Q (less or nill) |
| PS2471 | A12L, S22E, S25G, S66V, E71R, A122R, R154L | G, S, Q (less or nill) |
| PS2472 | A12L, S22E, S25Q, S66V, E71R, Q78N, A122R, S150V, R154L | G, S, Q (less or nill) |

TABLE 13
Binding affinities derived for PS1259 variants by fluorescence polarization assay.

| | | GA Peptide Kd ± | SA Peptide Kd ± | AA Peptide Kd ± |
|---|---|---|---|---|
| Binders | Mutations | std. error (nM) | std. error (nM) | std. error (nM) |
| PS2451 | A12L, S22E, S25Q, S66V, Q78H, N120R, | 681 ± 20 | 545 ± 23 | 502 ± 15 |
| | A122R, S150V | | | |
| PS2452 | A12L, S22E, S25Q, S66V, E71R, Q78H, | 530 ± 18 | 520 ± 14 | 498 ± 15 |
| | A122R, S150V, R154L | | | |
| PS2453 | A12L, S22E, S25Q, E71R, V73I, Q78H, I80V, | 469 ± 11 | 526 ± 15 | 437 ± 9 |
| | A122R, K147R, A149N, S150G, R154L | | | |
| PS2454 | A12L, S25Q, E71R, V73I, Q78H, I80V, N120S, | 595 ± 37 | 460 ± 72 | 164 ± 14 |
| | A122R, A149D, S150V, R154Q | | | |
| PS2457 | A12L, S22E, S25Q, E71R, Q78H, I80V, C85R, | 262 ± 19 | 200 ± 11 | 98 ± 11 |
| | N120R, A122R, A149V | | | |
| PS2458 | S5C, A12L, S25Q, E71R, V73I, Q78H, I80V, | 658 ± 36 | 431 ± 23 | 169 ± 14 |
| | N120S, A122R, A149D, S150V, R154Q | | | |
| PS2459 | A12L, S22E, S25Q, S66V, E71R, Q78H, | 267 ± 23 | 162 ± 6 | 95 ± 13 |
| | N120R, A122R, S150V, R154L | | | |
| PS2460 | A12L, S22E, S25Q, K65R, S66V, E71R, Q78H, | 312 ± 36 | 193 ± 8 | 102 ± 13 |
| | A122R, S150V, R154L | | | |
| PS2461 | A12L, S22E, S25Q, S66V, E71R, V73L, Q78H, | 461 ± 38 | 260 ± 17 | 137 ± 13 |
| | A122R, S150V, R154L | | | |
| PS2462 | A12L, S22E, S25Q, S66V, E71R, Q78H, I80V, | 419 ± 12 | 201 ± 9 | 99 ± 10 |
| | A122R, S150V, R154L | | | |
| PS2463 | A12L, S22E, S25Q, S66V, E71R, V73I, Q78H, | 343 ± 32 | 190 ± 16 | 110 ± 14 |
| | I80V, A122R, S150V, R154L | | | |
| PS2468 | A12L, S25Q, E71R, Q78H, A122R | 588 ± 61 | 415 ± 16 | 186 ± 12 |

TABLE 14
Stopped-flow binding kinetics of PS1259 variants.

| Binders | Mutations | GA (koff/s) | SA (koff/s) | AA (koff/s) |
|---|---|---|---|---|
| PS2451 | A12L, S22E, S25Q, S66V, Q78H, N120R, | 22.87 | 4.09 | 6.78 |
| | A122R, S150V | | | |
| PS2452 | A12L, S22E, S25Q, S66V, E71R, Q78H, | 32.19 | 9.93 | 10.91 |
| | A122R, S150V, R154L | | | |
| PS2453 | A12L, S22E, S25Q, E71R, V73I, Q78H, I80V, | 16.97 | 5.39 | 6.1 |
| | A122R, K147R, A149N, S150G, R154L | | | |
| PS2454 | A12L, S25Q, E71R, V73I, Q78H, I80V, | 47.35 | 11.26 | 10.03 |
| | N120S, A122R, A149D, S150V, R154Q | | | |
| PS2457 | A12L, S22E, S25Q, E71R, Q78H, I80V, | 14.61 | 6.19 | 5.36 |
| | C85R, N120R, A122R, A149V | | | |
| PS2458 | S5C, A12L, S25Q, E71R, V73I, Q78H, I80V, | 49.05 | 14.6 | 10.69 |
| | N120S, A122R, A149D, S150V, R154Q | | | |
| PS2459 | A12L, S22E, S25Q, S66V, E71R, Q78H, | 23.62 | 5.61 | 8.17 |
| | N120R, A122R, S150V, R154L | | | |
| PS2460 | A12L, S22E, S25Q, K65R, S66V, E71R, | 34.68 | 7.07 | 8.8 |
| | Q78H, A122R, S150V, R154L | | | |
| PS2461 | A12L, S22E, S25Q, S66V, E71R, V73L, | 43.23 | 8.7 | 10.44 |
| | Q78H, A122R, S150V, R154L | | | |
| PS2462 | A12L, S22E, S25Q, S66V, E71R, Q78H, I80V, | 34.8 | 9.18 | 14.47 |
| | A122R, S150V, R154L | | | |
| PS2463 | A12L, S22E, S25Q, S66V, E71R, V73I, Q78H, | 32.88 | 7.22 | 7.39 |
| | I80V, A122R, S150V, R154L | | | |
| PS2468 | A12L, S25Q, E71R, Q78H, A122R | 42.44 | 16.905 | 13.414 |

TABLE 15
Binding kinetics for PS1259 variants obtained via large scale preparation.

| | | | | | | GA | SA | AA | TA | | |
|---|---|---|---|---|---|---|---|---|---|---|---|
| | | | | | | Peptide | Peptide | Peptide | Peptide | | |
| | | GA | | | | Kd ± std. | Kd ± std. | Kd ± std. | Kd ± std. | AA | SA |
| | | (kon/ | GA | SA | AA | error | error | error | error | (kon/ | (kon/ |
| Binder | Mutations | nM/s) | (koff/s) | (koff/s) | (koff/s) | (nM) | (nM) | (nM) | (nM) | nM/s) | nM/S) |
| PS2453 | A12L, S22E, | 0.023 | 13.2 | 4.2 | 2.7 | 334 ± 44 | 312 ± 111 | 119 ± 16 | 4330 ± 660 | | |
| LS | S25Q, E71R, | | | | | | | | | | |
| | V73I, Q78H, | | | | | | | | | | |
| | I80V, A122R, | | | | | | | | | | |
| | K147R, A149N, | | | | | | | | | | |
| | S150G, R154L | | | | | | | | | | |
| PS2457 | A12L, S22E, | 0.014 | 17.6 | 4.5 | 3.0 | 569 ± 45 | 424 ± 35 | 228 ± 36 | 5389 ± 585 | | |
| LS | S25Q, E71R, | | | | | | | | | | |
| | Q78H, I80V, | | | | | | | | | | |
| | C85R, N120R, | | | | | | | | | | |
| | A122R, A149V | | | | | | | | | | |
| PS2459 | A12L, S22E, | 0.021 | 21.8 | 4.3 | 3.5 | 443 ± 34 | 300 ± 21 | 191 ± 29 | 2931 ± 367 | 0.0125 | 0.0078 |
| LS | S25Q, S66V, | | | | | | | | | | |
| | E71R, Q78H, | | | | | | | | | | |
| | N120R, A122R, | | | | | | | | | | |
| | S150V, R154L | | | | | | | | | | |
| PS2463 | A12L, S22E, | 0.024 | 21.5 | 5.1 | 3.3 | 540 ± 25 | 313 ± 25 | 166 ± 12 | 3575 ± 355 | | |
| LS | S25Q, S66V, | | | | | | | | | | |
| | E71R, V73I, | | | | | | | | | | |
| | Q78H, I80V, | | | | | | | | | | |
| | A122R, S150V, | | | | | | | | | | |
| | R154L | | | | | | | | | | |

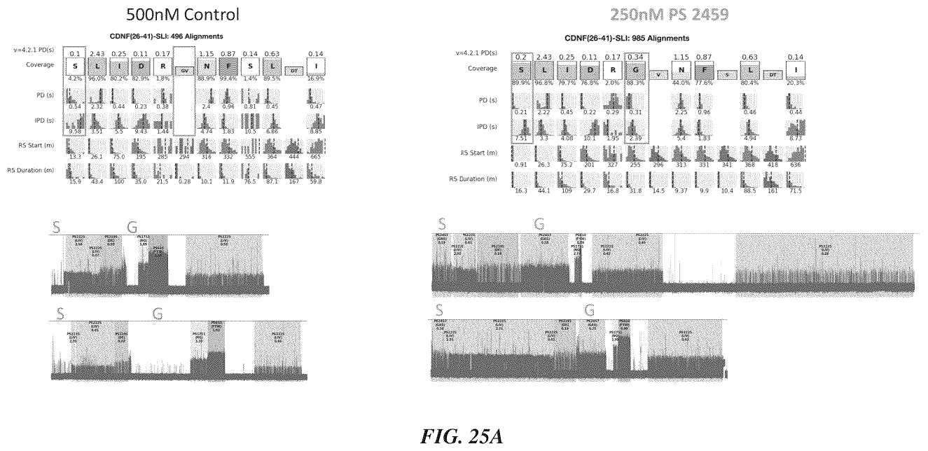

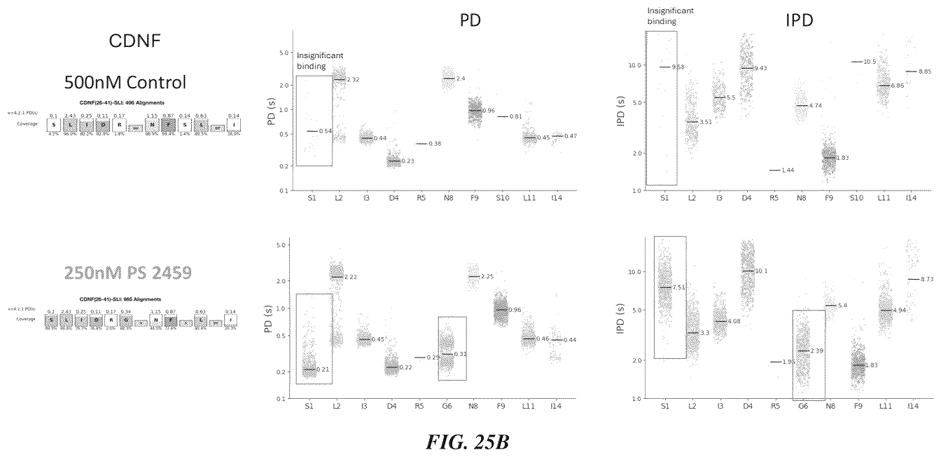

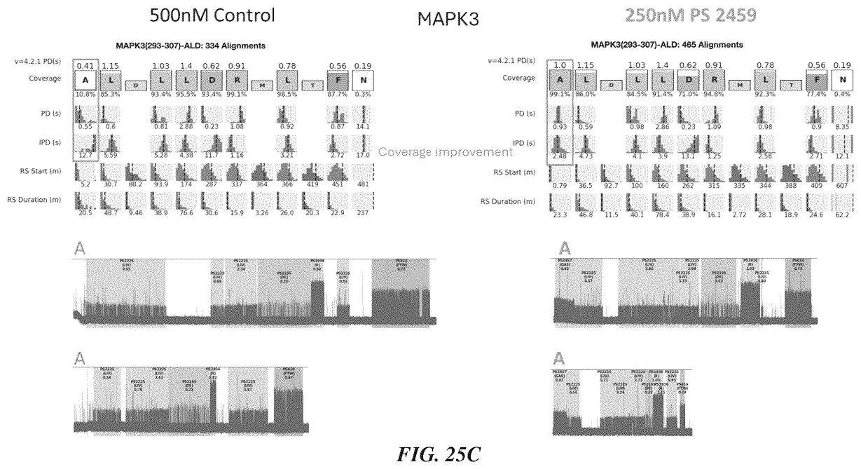

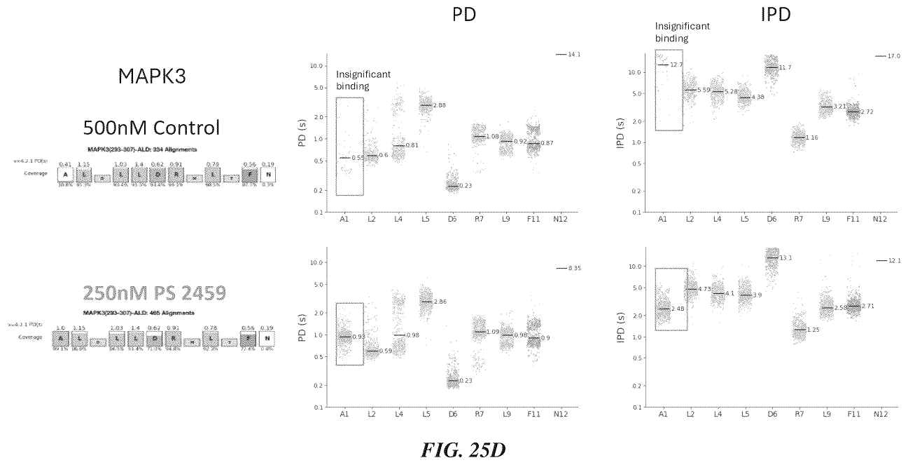

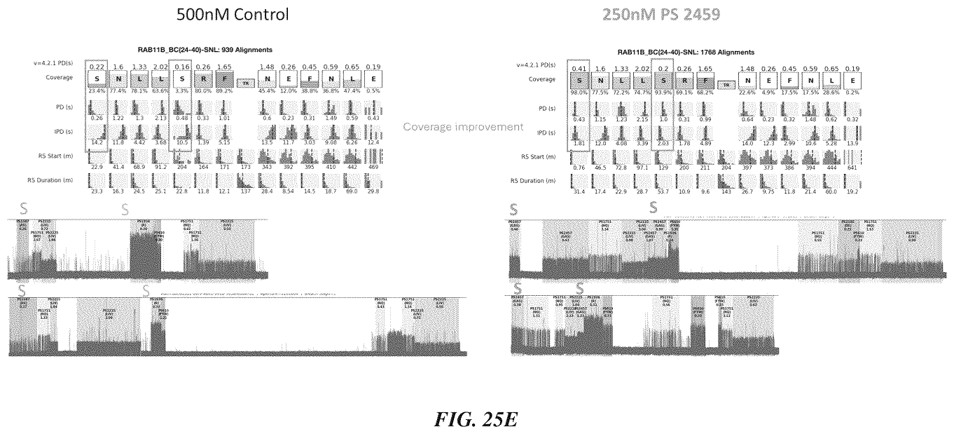

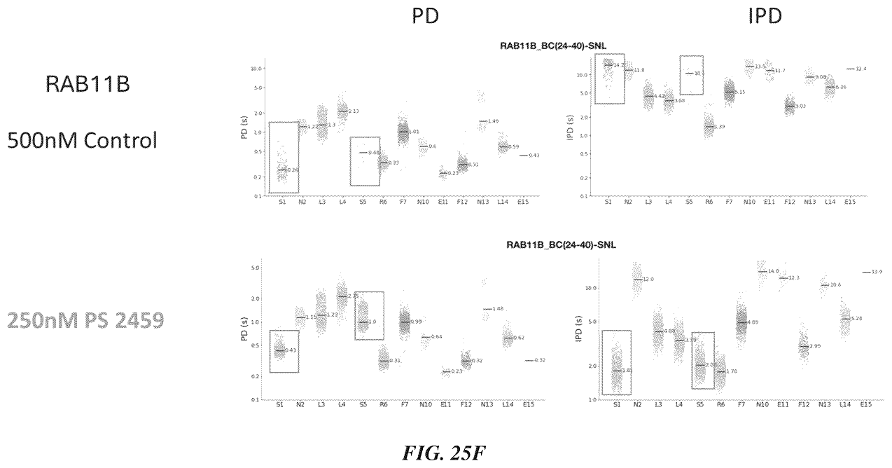

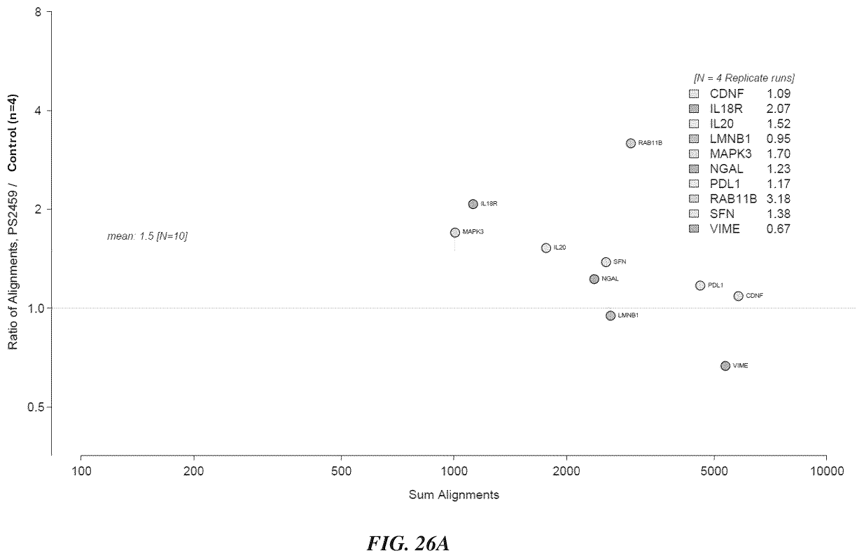

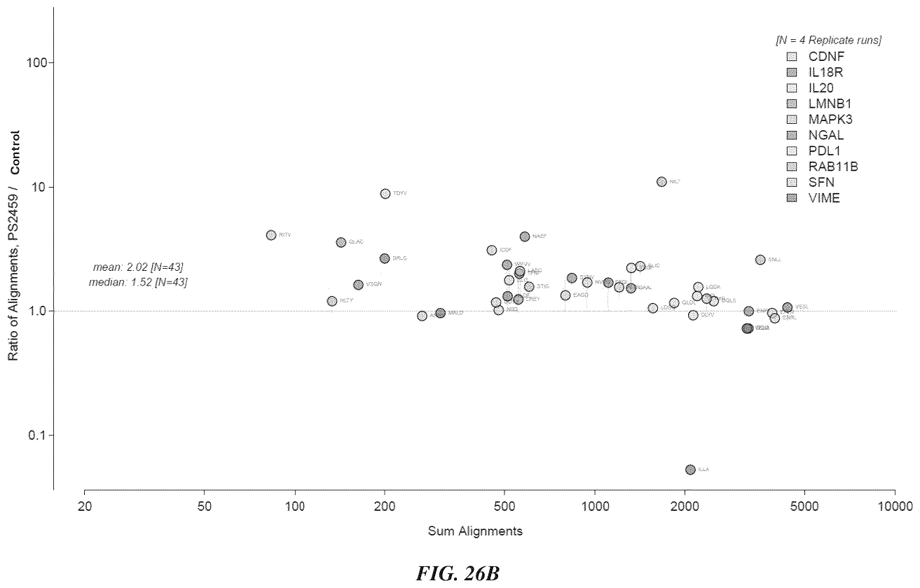

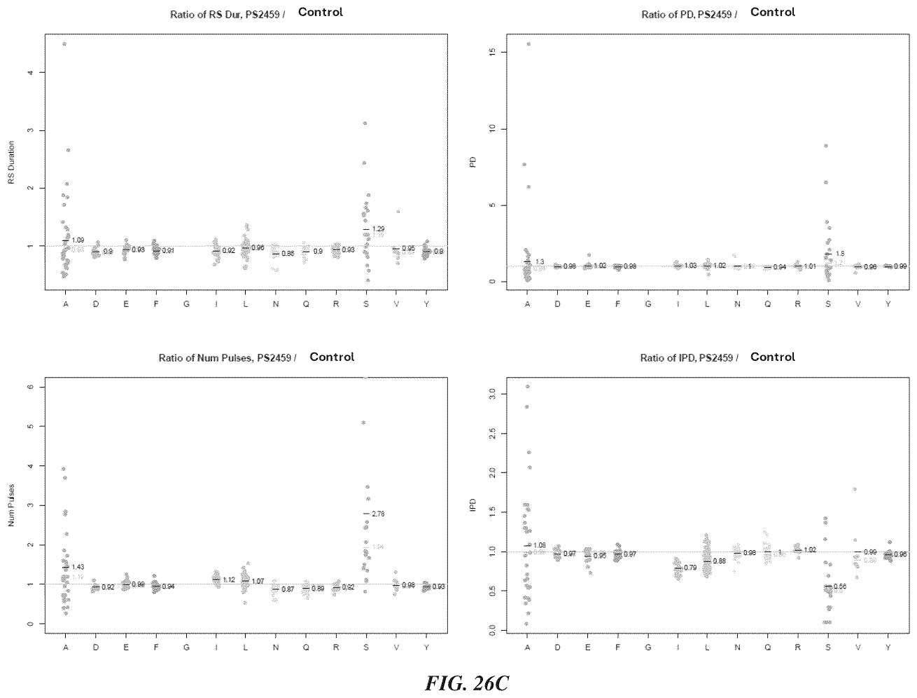

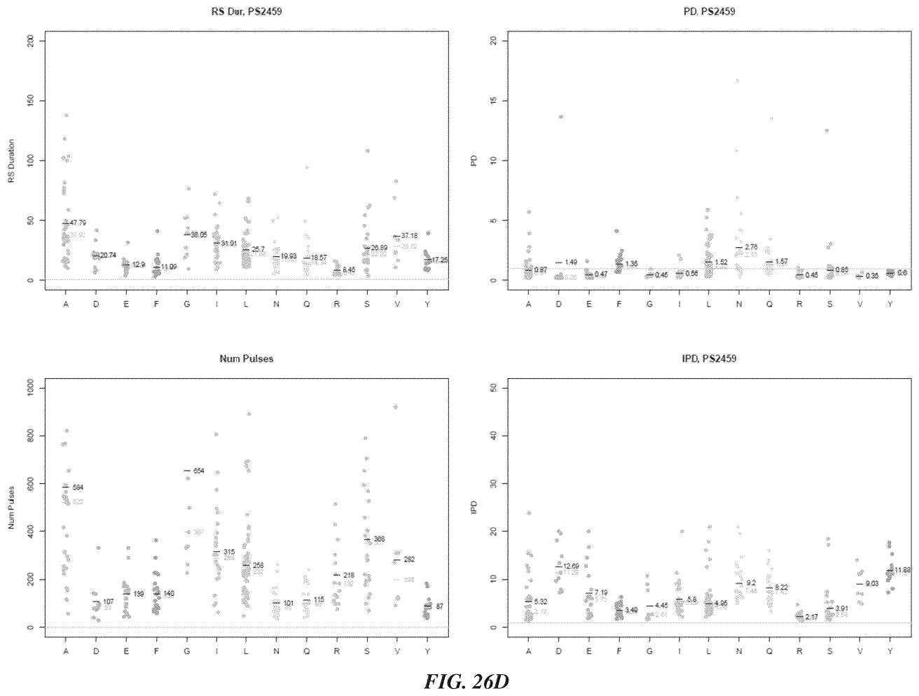

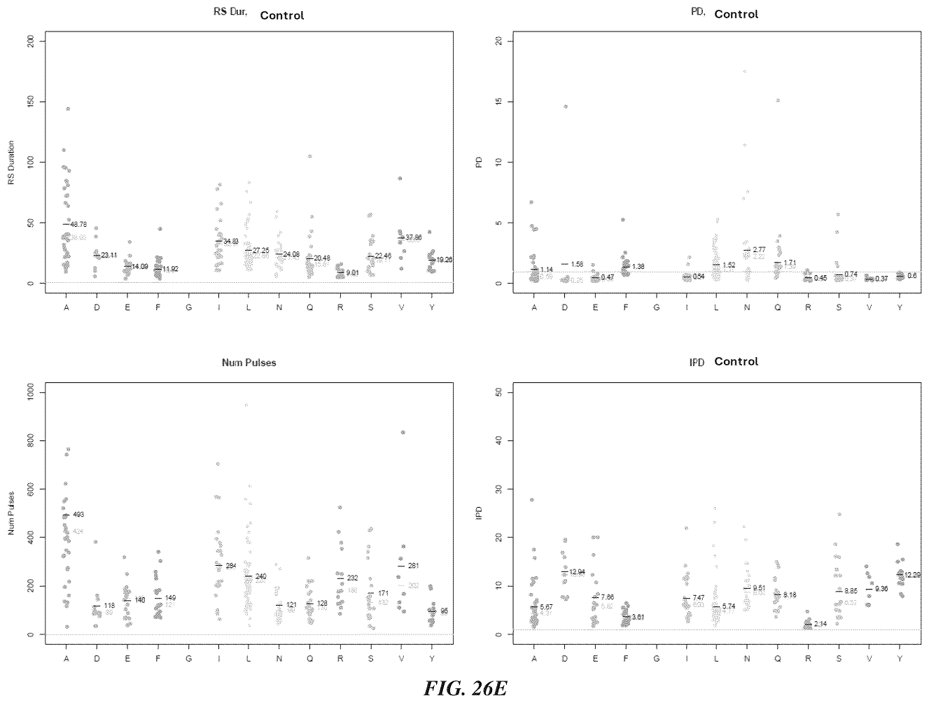

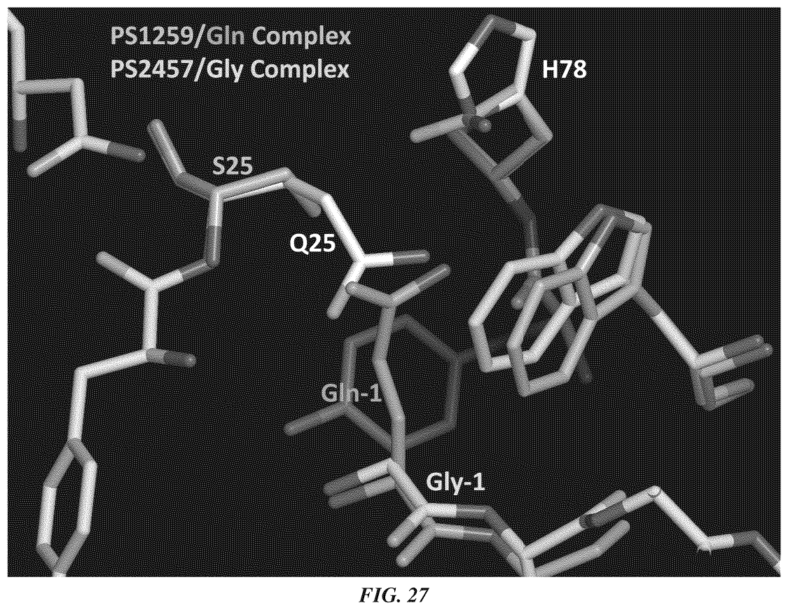

Protein sequencing runs were performed on a library mix comprising CDNF, PDL1, MAPK3, NGAL, IL18R, IL20, LMNB1, SFN, RAB11B, and VIME peptides using a mixture of recognizers: PS610 (a FWY recognizer corresponding to SEQ ID NO: 182), PS1936 (an R recognizer corresponding to SEQ ID NO: 158), PS2225 (an LIV recognizer corresponding to SEQ ID NO: 185), PS1751 (an NQ recognizer corresponding to SEQ ID NO: 184), PS2195 (a DE recognizer corresponding to SEQ ID NO: 25), and PS2459 (at 250 nM) or a nonhomologous A/S recognizer (“Control”; at 500 nM). A mixture of two aminopeptidases was also used in the sequencing runs. FIGS. 25A, 25C, and 25E shows representative traces for CDNF, MAPK3, and RAB11B, respectively. The use of a recognizer mixture comprising PS2459 showed glycine recognition as well as improved serine and alanine coverage, with increased pulse duration and decreased interpulse duration as compared to the use of a recognizer mixture comprising the nonhomologous A/S recognizer (FIGS. 25B, 25D, and 25F). Identification of amino acids by recognizers in the mixture was not affected by the inclusion of PS2459 (FIGS. 26A-26E).

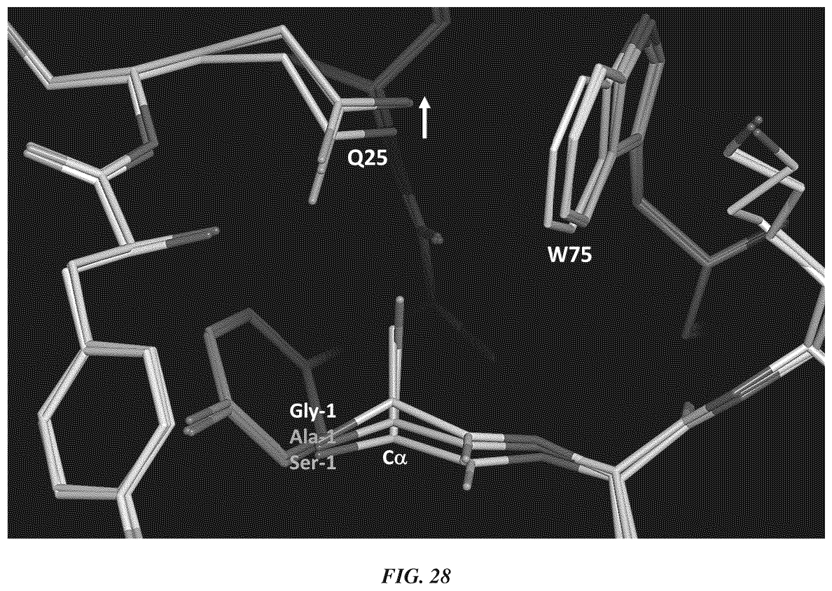

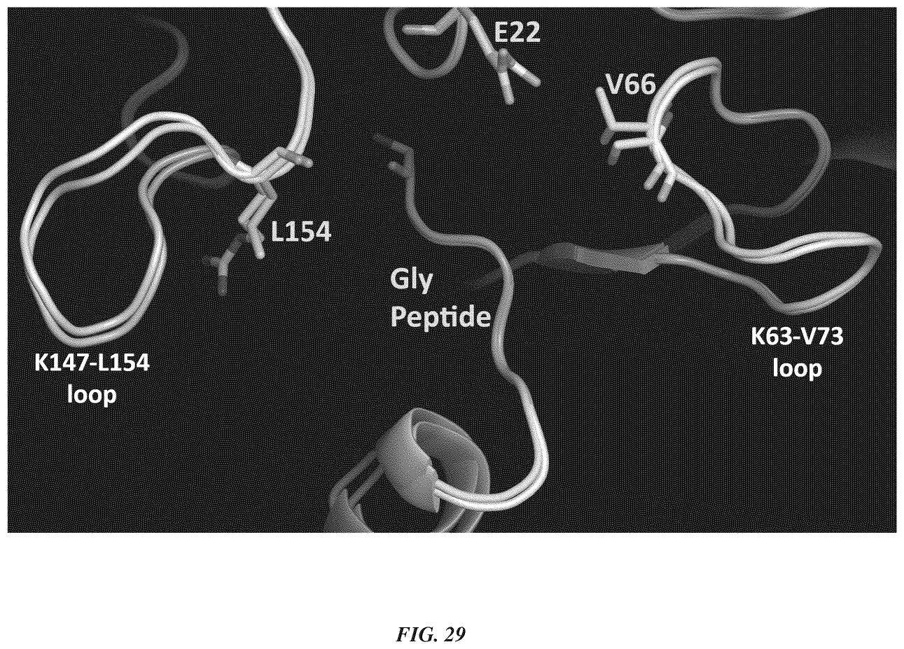

Without wishing to be bound or limited by theory, the glycine, alanine, and serine recognition of PS2459 may be rationalized in part via structure-based modeling of crystal structures of PS2457 (a PS1259 variant evaluated in this Example) and PS2459 in complex with bound peptides. FIG. 27 shows a superposition of PS2457/Glycine complex with PS1259/Glutamine complex. Substituted residues near the recognition pocket that were introduced into PS2457, relative to PS1259, include glutamine at position 25 and histidine at position 78. The substituted S25Q side chain decreases the size of the sidechain recognition pocket and blocks the binding of larger sidechains (e.g., peptides having N-terminal glutamine) through steric clash. The Q78H mutation locks the S25Q mutation into position via a direct interaction, and their combined effect results in increased specificity towards amino acids with smaller side chains (e.g., glycine, alanine, and serine). FIG. 28 shows a superposition of glycine-, alanine-, and serine-bound PS2457. The recognition of glycine, alanine, and serine results from Ca positional changes in response to the size of the bound sidechain and S25Q positional changes away from the larger alanine and serine side chains to accommodate their larger size. In addition to the S25Q and Q78H mutations (relative to PS1259) in the recognition pocket of PS2457, PS2459 has additional mutations S66V and R154L (relative to PS1259). FIG. 29 shows a superposition of PS2459 (green) with PS2457 (white) bound to a glycine peptide. The S66V and R154L mutations in PS2459 make the sidechain recognition pocket more hydrophobic and alter the surrounding loop structures, but do not alter the glycine binding pocket.

These data demonstrate the identification and use of Ntaq-1 homologous variants for G/A/S recognition in protein sequencing and suggest the importance of the mutated amino acids (at positions relative to PS1259) for improvements in glycine, alanine, and serine recognition by variants of PS1259 (including PS2459).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

1-32. (canceled)

33. A recombinant amino acid binding protein comprising an amino acid sequence that is at least 80% identical to SEQ ID NO: 1 (PS1259), wherein the amino acid sequence comprises amino acid substitutions at positions corresponding to S22, C23, and S25 of SEQ ID NO: 1.

34-36. (canceled)

37. The amino acid binding protein of claim 33, wherein the amino acid substitutions are selected from S22E, S22P, C23F, C23G, C23Q, and S25G.

38. (canceled)

39. The amino acid binding protein of claim 33, wherein the amino acid substitutions comprise S22P, C23G, and S25G.

40-42. (canceled)

43. The amino acid binding protein of claim 33, wherein the amino acid sequence comprises amino acid substitutions at positions corresponding to Q78 and D96 of SEQ ID NO: 1.

44. The amino acid binding protein of claim 43, wherein the amino acid substitutions comprise Q78R and D96R.

45. The amino acid binding protein of claim 33, wherein the amino acid sequence comprises an amino acid substitution at one or more positions corresponding to A12, K65, V73, K114, A122, A149, and S150 of SEQ ID NO: 1.

46. The amino acid binding protein of claim 45, wherein the amino acid substitution is selected from A12L, K65R, V73L, K114R, A122R, A149S, and S150R.

47. The amino acid binding protein of claim 33, wherein the amino acid sequence is at least 90%, at least 95%, at least 98%, at least 99%, 90-100%, 95-100%, 98-100%, or 100% identical to PS2195 (SEQ ID NO: 25).

48-66. (canceled)

67. A recombinant amino acid binding protein comprising an amino acid sequence that is at least 80% identical to SEQ ID NO: 1 (PS1259), wherein the amino acid sequence comprises a glutamine residue at a position corresponding to S25 of SEQ ID NO: 1.

68-69. (canceled)

70. The amino acid binding protein of claim 67, wherein the amino acid sequence comprises an amino acid substitution at one or more positions corresponding to S66, Q78, S150, and R154 of SEQ ID NO: 1.

71. The amino acid binding protein of claim 70, wherein the amino acid substitution is selected from S66V, Q78H, S150G, S150V, R154L, and R154Q.

72. The amino acid binding protein of claim 67, wherein the amino acid sequence comprises a histidine residue at a position corresponding to Q78 of SEQ ID NO: 1.

73-75. (canceled)

76. The amino acid binding protein of claim 67, wherein the amino acid sequence comprises amino acid substitutions at positions corresponding to each of A12, S22, S66, E71, Q78, N120, A122, S150, and R154 of SEQ ID NO: 1.

77. The amino acid binding protein of claim 76, wherein the amino acid substitutions comprise A12L, S22E, S66V, E71R, Q78H, N120R, A122R, S150V, and R154L.

78. (canceled)

79. The amino acid binding protein of claim 67, wherein the amino acid sequence is at least 90%, at least 95%, at least 98%, at least 99%, 90-100%, 95-100%, 98-100%, or 100% identical to PS2459 (SEQ ID NO: 234).

80-82. (canceled)

83. A recombinant amino acid binding protein comprising an amino acid sequence that is at least 80% identical to SEQ ID NO: 2 (PS1122), wherein the amino acid sequence comprises amino acid substitutions at positions corresponding to T53 and one or more selected from K26, D32, L47, F59, and T75 of SEQ ID NO: 2.

84-94. (canceled)

95. The amino acid binding protein of claim 83, wherein the amino acid sequence is at least 90%, at least 95%, at least 98%, at least 99%, 90-100%, 95-100%, 98-100%, or 100% identical to a sequence selected from any one of PS1936-PS1938 (SEQ ID NOs: 158-160).

96-141. (canceled)

142. A kit comprising one or more amino acid recognizers, wherein at least one amino acid recognizer comprises the amino acid binding protein of claim 33.

143. A method of determining at least one chemical characteristic of a polypeptide, the method comprising:
contacting a polypeptide with the amino acid binding protein of claim 33;
monitoring a signal for signal pulses corresponding to interactions between one or more amino acid recognizers and the polypeptide; and
determining at least one chemical characteristic of the polypeptide based on a characteristic pattern in the signal.

144. A kit comprising one or more amino acid recognizers, wherein at least one amino acid recognizer comprises the amino acid binding protein of claim 67.

145. A method of determining at least one chemical characteristic of a polypeptide, the method comprising:
contacting a polypeptide with the amino acid binding protein of claim 67;
monitoring a signal for signal pulses corresponding to interactions between one or more amino acid recognizers and the polypeptide; and
determining at least one chemical characteristic of the polypeptide based on a characteristic pattern in the signal.
